(19) United States
(12) Patent Application Publication
(10) Pub. No.: US 2023/0357342 A1
(43) Pub. Date: Nov. 9, 2023

(54) MODIFIED IL-18 POLYPEPTIDES

(71) Applicant: Bright Peak Therapeutics AG, Basel (CH)

(72) Inventors: Vijaya Raghavan PATTABIRAMAN, Volketswil (CH); Bertolt KREFT, Kleinmachow (DE); Arnaud GOEPFERT, Sierentz (FR); Tiziano ONGARO, Basel (CH); Jean-Philippe CARRALOT, Saint-Louis (FR); Philipp MOOSMANN, Basel (CH); Régis BOEHRINGER, Basel (CH); Benoit HORNSPERGER, Altkirch (FR); Roy MEODED, Basel (CH); Kea MARTIN, Istein (DE); Camille DELON, Saint-Louis (FR); Andrew CHI, San Diego, CA (US)

(21) Appl. No.: 18/113,409

(22) Filed: Feb. 23, 2023

Related U.S. Application Data
(60) Provisional application No. 63/313,210, filed on Feb. 23, 2022.
(60) Provisional application No. 63/313,127, filed on Feb. 23, 2022.
(60) Provisional application No. 63/313,222, filed on Feb. 23, 2022.
(60) Provisional application No. 63/479,529, filed on Jan. 11, 2023.
(60) Provisional application No. 63/313,248, filed on Feb. 23, 2022.

(51) Int. Cl. C07K 14/54

(52) U.S. Cl. CPC C07K 14/54

(57) ABSTRACT

The present disclosure relates a modified interleukin 18 (IL-18) polypeptide, methods of making the same, and methods of using the modified IL-18 polypeptides for treatment of diseases including cancer. The modified IL-18 polypeptide can exhibit an ability to induce interferon gamma (IFNγ) production when in contact with a cell, exhibit a diminished ability to be inhibited by IL-18 binding protein (IL-18BP) compared to wild type IL-18 (WT IL-18), and exhibit a ratio of half-maximal inhibitory concentration (IC50) by IL-18BP to half-maximal effective concentration (EC50) to induce IFNγ production which is greater than that of WT IL-18.

### CROSS-REFERENCE

This application claims priority to U.S. Provisional Patent Application Nos. 63/313,210, filed Feb. 23, 2022, 63/313,127, filed Feb. 23, 2022, 63/313,222, filed Feb. 23, 2022, 63/313,248, filed Feb. 23, 2022, and 63/479,529, Filed Jan. 11, 2023, all of which are incorporated in full herein by reference.

### BACKGROUND

Immunotherapies utilize the immune system of a subject to aid in the treatment of ailments. Immunotherapies can be designed to activate or suppress the immune system depending on the nature of the disease being treated. The goal of immunotherapies for the treatment of cancer is to stimulate the immune system so that it recognizes and destroys tumors or other cancerous tissue. One method of activating the immune system to attack cancer cells in the body of a subject is cytokine therapy. Cytokines are proteins produced in the body that are important in cell signaling and in modulating the immune system. Some cytokine therapy utilizes these properties of cytokines to enhance the immune system of a subject to kill cancer cells.

### BRIEF SUMMARY

In one aspect, provided herein, is a modified interleukin 18 (IL-18) polypeptide. The modified IL-18 polypeptide exhibits an ability to induce interferon gamma (IFNγ) production when in contact with a cell. In some embodiments, the modified IL-18 polypeptide exhibits an enhanced ability to induce IFNγ production in a cell compared to wild type IL-18 (WT IL-18). In some embodiments, the modified IL-18 polypeptide exhibits an enhanced ability for signaling through an IL-18 receptor compared to WT IL-18. In some embodiments, the modified IL-18 polypeptide exhibits a diminished ability to be inhibited by IL-18 binding protein (IL-18BP) compared to WT IL-18. In certain embodiments, the modified IL-18 polypeptide exhibits i) an enhanced ability to induce IFNγ production in a cell compared to WT IL-18, ii) an enhanced ability for signaling through an IL-18 receptor compared to WT IL-18, and iii) a diminished ability to be inhibited by IL-18BP compared to WT IL-18.

In some embodiments, the modified IL-18 polypeptide exhibits a ratio of half-maximal inhibitory concentration (IC50) by IL-18BP to half-maximal effective concentration (EC50) to induce IFNγ production which is at least 50,000. In some embodiments, the modified IL-18 polypeptide can exhibit a ratio of half-maximal inhibitory concentration (IC50) by IL-18BP to half-maximal effective concentration (EC50) to induce IFNγ production which is at least 298,000. In certain embodiments, the ratio of IC50 by IL-18BP to EC50 to induce IFNγ production for the modified IL-18BP is at least 50,000, at least 100,000, at least 200,000, at least 299,000, at least 300,000, at least 400,000, at least 500,000, at least 750,000, at least 1,000,000, at least 1,250,000, at least 1,500,000, or at least 1,750,000. In certain embodiments, the ratio of IC50 by IL-18BP to EC50 to induce IFNγ production for the modified IL-18BP is at least 50,000. In certain embodiments, the ratio of IC50 by IL-18BP to EC50 to induce IFNγ production for the modified IL-18BP is at least 100,000. In certain embodiments, the ratio of IC50 by IL-18BP to EC50 to induce IFNγ production for the modified IL-18BP is at least 200,000. In certain embodiments, the ratio of IC50 by IL-18BP to EC50 to induce IFNγ production for the modified IL-18BP is at least 299,000. In certain embodiments, the ratio of IC50 by IL-18BP to EC50 to induce IFNγ production for the modified IL-18BP is at least 300,000. In certain embodiments, the ratio of IC50 by IL-18BP to EC50 to induce IFNγ production for the modified IL-18BP is at least 400,000. In certain embodiments, the ratio of IC50 by IL-18BP to EC50 to induce IFNγ production for the modified IL-18BP is at least 500,000. In certain embodiments, the ratio of IC50 by IL-18BP to EC50 to induce IFNγ production for the modified IL-18BP is at least 750,000. In certain embodiments, the ratio of IC50 by IL-18BP to EC50 to induce IFNγ production for the modified IL-18BP is at least 1,000,000. In certain embodiments, the ratio of IC50 by IL-18BP to EC50 to induce IFNγ production for the modified IL-18BP is at least 1,250,000. In certain embodiments, the ratio of IC50 by IL-18BP to EC50 to induce IFNγ production for the modified IL-18BP is at least 1,500,000. In certain embodiments, the ratio of IC50 by IL-18BP to EC50 to induce IFNγ production for the modified IL-18BP is at least 1,750,000. EC50 to induce IFNγ production can be measured using an IFNγ Induction NK-92 Cellular Assay (e.g., as provided herein in the Examples) and IC50 by IL-18BP can be measured by IL-18 Binding Protein-mediated Inhibition of IFNγ secretion in NK-92 Cellular Assay (e.g., as provided herein in the Examples).

In some embodiments, the modified IL-18 polypeptide exhibits a half-maximal effective concentration (EC50) to induce IFNγ production in a cell which is at least 25-fold lower than the EC50 for wild type IL-18 (WT IL-18). In certain embodiments, the EC50 to induce IFNγ production in a cell for the modified IL-18 polypeptide is at least 30-fold lower, 40-fold, 50-fold, 60-fold, 70-fold, or 80-fold lower than the EC50 for WT IL-18. In certain embodiments, the EC50 to induce IFNγ production in a cell for the modified IL-18 polypeptide is at least 30-fold lower than the EC50 for WT IL-18. In certain embodiments, the EC50 to induce IFNγ production in a cell for the modified IL-18 polypeptide is at least 40-fold lower than the EC50 for WT IL-18. In certain embodiments, the EC50 to induce IFNγ production in a cell for the modified IL-18 polypeptide is at least 50-fold lower than the EC50 for WT IL-18. In certain embodiments, the EC50 to induce IFNγ production in a cell for the modified IL-18 polypeptide is at least 60-fold lower than the EC50 for WT IL-18. In certain embodiments, the EC50 to induce IFNγ production in a cell for the modified IL-18 polypeptide is at least 70-fold lower than the EC50 for WT IL-18. In certain embodiments, the EC50 to induce IFNγ production in a cell for the modified IL-18 polypeptide is at least 80-fold lower than the EC50 for WT IL-18. EC50 to induce IFNγ production in a cell can be measured using IFNγ Induction NK-92 Cellular Assay (e.g., as provided in the Examples herein)

In some embodiments, the modified IL-18 polypeptide exhibits a half maximal effective concentration (EC50) for signaling through an IL-18 receptor which is at least 4.6-fold lower than the EC50 for WT IL-18. In certain embodiments, the EC50 for signaling through the IL-18 receptor for the modified IL-18 polypeptide is at least 4.7-fold, 5-fold, 10-fold, 20-fold, 30-fold, 40-fold, or 50-fold lower than the EC50 for WT IL-18. In certain embodiments, the EC50 for signaling through the IL-18 receptor for the modified IL-18 polypeptide is at least 4.7-fold lower than the EC50 for WT IL-18. In certain embodiments, the EC50 for signaling through the IL-18 receptor for the modified IL-18 polypeptide is at least 5-fold lower than the EC50 for WT IL-18. In certain embodiments, the EC50 for signaling through the IL-18 receptor for the modified IL-18 polypeptide is at least 10-fold lower than the EC50 for WT IL-18. In certain embodiments, the EC50 for signaling through the IL-18 receptor, for the modified IL-18 polypeptide is at least 20-fold lower than the EC50 for WT IL-18. In certain embodiments, the EC50 for signaling through the IL-18 receptor, for the modified IL-18 polypeptide is at least 30-fold lower than the EC50 for WT IL-18. In certain embodiments, the EC50 for signaling through the IL-18 receptor for the modified IL-18 polypeptide is at least 40-fold lower than the EC50 for WT IL-18. In certain embodiments, the EC50 for signaling through the IL-18 receptor for the modified IL-18 polypeptide is at least 50-fold lower than the EC50 for WT IL-18. EC50 for signaling through the IL-18 receptor can be measured using HEK-Blue IL18R reporter assay (e.g., as provided in the Examples herein).

In some embodiments, the modified IL-18 polypeptide exhibits a dissociation constant (K_{D}) with the IL-18 receptor alpha subunit at least 50-fold lower than the K_{D} for wild type IL-18. In certain embodiments, the modified IL-18 polypeptide exhibits a K_{D} with the IL-18 receptor alpha subunit at least 52-fold, 60-fold, 70-fold, 80-fold, 90-fold, or 100-fold lower than the K_{D} for wild type IL-18. In certain embodiments, the modified IL-18 polypeptide exhibits a K_{D} with the IL-18 receptor alpha subunit at least 52-fold lower than the K_{D} for wild type IL-18. In certain embodiments, the modified IL-18 polypeptide exhibits a K_{D} with the IL-18 receptor alpha subunit at least 60-fold lower than the K_{D} for wild type IL-18. In certain embodiments, the modified IL-18 polypeptide exhibits a K_{D} with the IL-18 receptor alpha subunit at least 70-fold lower than the K_{D} for wild type IL-18. In certain embodiments, the modified IL-18 polypeptide exhibits a K_{D} with the IL-18 receptor alpha subunit at least 80-fold lower than the K_{D} for wild type IL-18. In certain embodiments, the modified IL-18 polypeptide exhibits a K_{D} with the IL-18 receptor alpha subunit at least 90-fold lower than the K_{D} for wild type IL-18. In certain embodiments, the modified IL-18 polypeptide exhibits a K_{D} with the IL-18 receptor alpha subunit at least 100-fold lower than the K_{D} for wild type IL-18.

In some embodiments, the modified IL-18 polypeptide exhibits a dissociation constant (K_{D}) with IL-18 binding protein (IL-18BP) which is greater than that of wild type IL-18. In some embodiments, the modified IL-18 polypeptide exhibits a dissociation constant (K_{D}) with IL-18BP of at least 250 nM. In certain embodiments, the K_{D} with IL-18BP, for the modified IL-18 polypeptide is at least 2 nM, at least 5 nM, at least 10 nM, at least 20 nM, at least 50 nM, at least 100 nM, at least 200 nM, at least 300 nM, at least 400 nM, at least 500 nM, at least 750 nM, at least 1000 nM, at least 2500 nM, at least 5000 nM, or at least 10000 nM. In certain embodiments, the K_{D} with IL-18BP, for the modified IL-18 polypeptide is at least 2 nM. In certain embodiments, the K_{D} with IL-18BP, for the modified IL-18 polypeptide is at least 5 nM. In certain embodiments, the K_{D} with IL-18BP, for the modified IL-18 polypeptide is at least 10 nM. In certain embodiments, the K_{D} with IL-18BP, for the modified IL-18 polypeptide is at least 50 nM. In certain embodiments, the K_{D} with IL-18BP, for the modified IL-18 polypeptide is at least 100 nM. In certain embodiments, the K_{D} with IL-18BP, for the modified IL-18 polypeptide is at least 200 nM. In certain embodiments, the K_{D} with IL-18BP, for the modified IL-18 polypeptide is at least 300 nM. In certain embodiments, the K_{D} with IL-18BP, for the modified IL-18 polypeptide is at least 400 nM. In certain embodiments, the K_{D} with IL-18BP, for the modified IL-18 polypeptide is at least 500 nM. In certain embodiments, the K_{D} with IL-18BP, for the modified IL-18 polypeptide is at least 750 nM. In certain embodiments, the K_{D} with IL-18BP, for the modified IL-18 polypeptide is at least 1000 nM. In certain embodiments, the K_{D} with IL-18BP, for the modified IL-18 polypeptide is at least 2500 nM. In certain embodiments, the K_{D} with IL-18BP, for the modified IL-18 polypeptide is at least 5000 nM. In certain embodiments, the K_{D} with IL-18BP, for the modified IL-18 polypeptide is at least 10000 nM. The K_{D} with IL-18BP can be measured using IL-18BP Binding alphaLISA Assay (e.g., as provided in the Examples herein).

In certain embodiments, the modified IL-18 polypeptide exhibits any one of, any combination of, or all of the following properties:
i) a ratio of IC50 by IL-18BP, to EC50 to induce IFNγ production, which is at least 50,000, at least 100,000, at least 200,000, at least 298,000, at least 299,000, at least 300,000, at least 400,000, at least 500,000, at least 750,000, at least 1,000,000, at least 1,250,000, at least 1,500,000, or at least 1,750,000;
ii) a EC50 to, induce IFNγ production in a cell, which is at least 25-fold, at least 30-fold, 40-fold, 50-fold, 60-fold, 70-fold, or 80-fold lower than the EC50 for WT IL-18;
iii) a EC50 for signaling through an IL-18 receptor which is at least 4.6-fold lower, at least 4.7-fold, 5-fold, 10-fold, 20-fold, 30-fold, 40-fold, or 50-fold lower than the EC50 for WT IL-18;
iv) a K_{D} with the IL-18 receptor alpha subunit at least 50-fold, at least 52-fold, 60-fold, 70-fold, 80-fold, 90-fold, or 100-fold lower than wild type IL-18; and
v) a K_{D} with IL-18BP of at least 2 nM, at least 5 nM, at least 10 nM, at least 20 nM, at least 50 nM, at least 100 nM, at least 200 nM, at least 250 nM, at least 500 nM, at least 750 nM, at least 1000 nM, at least 2500 nM, at least 5000 nM, or at least 10000 nM.

In certain embodiments, the modified IL-18 polypeptide comprises a substitution at residue V11. In certain embodiments, the modified IL-18 polypeptide comprises V11I substitution. Unless specifically mentioned otherwise, the residue position numbering is provided in this paragraph, and elsewhere in this disclosure is based on SEQ ID NO: 1, as a reference sequence. Unless specifically mentioned otherwise, the amino acid substitutions provided in this paragraph, and elsewhere in this disclosure is with respect to SEQ ID NO: 1, as a reference sequence. In certain embodiments, the modified IL-18 polypeptide comprises a substitution at a residue selected from Y1, F2, E6, K8, S10, D17, T34, D35, S36, D37, D40, N41, I49, M51, K53, D54, S55, Q103, S105, G108, H109, D110, and D132. In certain embodiments, the modified IL-18 polypeptide comprises a substitution at residue E6. In certain embodiments, the modified IL-18 polypeptide comprises E6K substitution. In certain embodiments, the modified IL-18 polypeptide comprises a substitution at residue M51. In certain embodiments, the modified IL-18 polypeptide comprises M51G substitution. In certain embodiments, the modified IL-18 polypeptide comprises a substitution at residue K53. In certain embodiments, the modified IL-18 polypeptide comprises K53A substitution. In certain embodiments, the modified IL-18 polypeptide comprises a substitution at residue T63. In certain embodiments, the modified IL-18 polypeptide comprises a T63A substitution. In certain embodiments, the modified IL-18 polypeptide comprises i) substitution at residue V11, and ii) substitution at residue E6, M51 and/or K53. In certain embodiments, the modified IL-18 polypeptide comprises i) V11I substitution, and ii) E6K, M51G, and/or K53A substitution. In certain embodiments, the modified IL-18 polypeptide comprises substitutions at residues E6, V11, K53 and T63. In certain embodiments, the modified IL-18 polypeptide comprises E6K, V11I, K53A and T63A substitutions. In certain embodiments, the modified IL-18 polypeptide comprises substitutions at residues V11, M51, K53. In certain embodiments, the modified IL-18 polypeptide comprises V11I, M51G, and K53A substitutions. In certain embodiments, the modified IL-18 polypeptide comprises substitutions at residues E6, V11, M51, K53 and T63. In certain embodiments, the modified IL-18 polypeptide comprises E6K, V11I, M51G, K53A and T63A substitutions. In certain embodiments, the modified IL-18 polypeptide comprises a substitution at residue C38, C68, C76, and/or C127. In certain embodiments, the modified IL-18 polypeptide comprises a substitution at residue C38. In certain embodiments, the modified IL-18 polypeptide comprises a substitution at residue C68. In certain embodiments, the modified IL-18 polypeptide comprises a substitution at residue C76. In certain embodiments, the modified IL-18 polypeptide comprises a substitution at residue C127. In certain embodiments, the modified IL-18 polypeptide comprises a C38A, C38S, C68A, C68S, C76A, C76S, C127A, and/or C127S substitution. In certain embodiments, the modified IL-18 polypeptide comprises a C38A substitution. In certain embodiments, the modified IL-18 polypeptide comprises C38S. In certain embodiments, the modified IL-18 polypeptide comprises a C68A substitution. In certain embodiments, the modified IL-18 polypeptide comprises C68S. In certain embodiments, the modified IL-18 polypeptide comprises a C76A substitution. In certain embodiments, the modified IL-18 polypeptide comprises C76S. In certain embodiments, the modified IL-18 polypeptide comprises a C127A substitution. In certain embodiments, the modified IL-18 polypeptide comprises C127S. In certain embodiments, the modified IL-18 polypeptide comprises substitutions at each of residues C38, C76, and C127, wherein each of the substitutions at residues C38, C76, and C127 is for a serine or alanine. In certain embodiments, the modified IL-18 polypeptide comprises C38A, C76A, and C127A substitutions. In certain embodiments, the modified IL-18 polypeptide comprises C38S, C76S and C127S substitutions. In certain embodiments, the modified IL-18 polypeptide comprises substitutions at residues E6, V11, C38, K53, T63, C76, and C127. In certain embodiments, the modified IL-18 polypeptide comprises E6K, V11I, C38A, K53A, T63A, C76A, and C127A substitutions. In certain embodiments, the modified Il-18 polypeptide comprises substitutions at residues V11, C38, M51, K53, T63, C76, and C127. In certain embodiments, the modified IL-18 polypeptide comprises V11, C38A, M51G, K53A, T63A, C76A, and C127A substitutions. In certain embodiments, the modified Il-18 polypeptide comprises substitutions at residues V11, C38, M51, K53, C76, and C127. In certain embodiments, the modified IL-18 polypeptide comprises V11I, C38A, M51G, K53A, C76A, and C127A substitutions. In certain embodiments, the modified IL-18 polypeptide comprises substitutions at residues E6, V11, C38, M51, K53, T63, C76, and C127. In certain embodiments, the modified Il-18 polypeptide comprises E6K, V11I, C38A, M51G, K53A, T63A, C76A, and C127A substitutions. In certain embodiments, the modified IL-18 polypeptide comprises at least one glycine residue attached to the N-terminus of the IL-18 polypeptide. In certain embodiments, the modified IL-18 polypeptide comprises a chain of 1 to 10 glycine residues attached to the N-terminus of the IL-18 polypeptide. In certain embodiments, the modified IL-18 polypeptide comprises a chain of 1, 2, 3, 4, 5, 6, 7, 8, 9, or 10, or any range therebetween glycine residues attached to the N-terminus of the IL-18 polypeptide. In certain embodiments, the modified IL-18 polypeptide comprises a glycine residue attached to the N-terminus of the IL-18 polypeptide. In certain embodiments, the modified IL-18 polypeptide comprises a chain of 2 glycine residues attached to the N-terminus of the IL-18 polypeptide. In certain embodiments, the modified IL-18 polypeptide comprises a chain of 3 glycine residues attached to the N-terminus of the IL-18 polypeptide. In certain embodiments, the modified IL-18 polypeptide comprises a chain of 4 glycine residues attached to the N-terminus of the IL-18 polypeptide. In certain embodiments, the modified IL-18 polypeptide comprises a chain of 5 glycine residues attached to the N-terminus of the IL-18 polypeptide. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) substitutions at residues E6, V11, C38, K53, T63, C76, and C127. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) E6K, V11, C38A, K53A, T63A, C76A, and C127A substitutions. In certain embodiments, the modified IL-18 polypeptide comprises i) a glycine residue attached to the N-terminus of the IL-18 polypeptide, and ii) substitutions at residues E6, V11, C38, K53, T63, C76, and C127. In certain embodiments, the modified IL-18 polypeptide comprises i) a glycine residue attached to the N-terminus of the IL-18 polypeptide, and ii) E6K, V11I, C38A, K53A, T63A, C76A, and C127A substitutions. In certain embodiments, the modified IL-18 polypeptide comprises i) a chain of 4 glycine residues attached to the N-terminus of the IL-18 polypeptide, and ii) substitutions at residues E6, V11, C38, K53, T63, C76, and C127. In certain embodiments, the modified IL-18 polypeptide comprises i) a chain of 4 glycine residues attached to the N-terminus of the IL-18 polypeptide, and ii) E6K, V11I, C38A, K53A, T63A, C76A, and C127A substitutions. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) substitutions at residues E6, V11, C38, M51, K53, T63, C76, and C127. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) E6K, V11I, C38A, M51G, K53A, T63A, C76A, and C127A substitutions. In certain embodiments, the modified IL-18 polypeptide comprises i) a glycine residue attached to the N-terminus of the IL-18 polypeptide, and ii) substitutions at residues E6, V1, C38, M51, K53, T63, C76, and C127. In certain embodiments, the modified IL-18 polypeptide comprises i) a glycine residue attached to the N-terminus of the IL-18 polypeptide, and ii) E6K, V11I, C38A, M51G, K53A, T63A, C76A, and C127A substitutions.

In certain embodiments, the modified IL-18 polypeptide comprises an amino acid sequence having at least 80%, 85%, 90%, 91%, 92%, 93%, 94%, or 95% sequence identity with the sequence set forth in SEQ ID NO: 1. In certain embodiments, the modified IL-18 polypeptide comprises an amino acid sequence having at least 80% sequence identity with the sequence set forth in SEQ ID NO: 1. In certain embodiments, the modified IL-18 polypeptide comprises an amino acid sequence having at least 90% sequence identity with the sequence set forth in SEQ ID NO: 1. In certain embodiments, the modified IL-18 polypeptide comprises an amino acid sequence having at least 95% sequence identity with the sequence set forth in SEQ ID NO: 1. In certain embodiments, the modified IL-18 polypeptide comprises an amino acid sequence having at least 80%, 85%, 90%, 95%, 98%, 99%, or 100% sequence identity to the sequence set forth in any one of SEQ ID NO: 2-33. In certain embodiments, the modified IL-18 polypeptide comprises an amino acid sequence having at least 80% sequence identity with the sequence set forth in any one of SEQ ID NO: 2-33. In certain embodiments, the modified IL-18 polypeptide comprises an amino acid sequence having at least 90% sequence identity with the sequence set forth in any one of SEQ ID NO: 2-33. In certain embodiments, the modified IL-18 polypeptide comprises an amino acid sequence having at least 95% sequence identity with the sequence set forth in any one of SEQ ID NO: 2-33. In certain embodiments, the modified IL-18 polypeptide comprises an amino acid sequence having at least 98% sequence identity with the sequence set forth in any one of SEQ ID NO: 2-33. In certain embodiments, the modified IL-18 polypeptide comprises the amino acid sequence set forth in any one of SEQ ID NO: 2-33. In certain embodiments, the modified IL-18 polypeptide comprises an amino acid sequence having at least 80%, 85%, 90%, 95%, 98%, 99%, or 100% sequence identity to the sequence set forth in any one of SEQ ID NO: 24-33. In certain embodiments, the modified IL-18 polypeptide comprises an amino acid sequence having at least 80% sequence identity with the sequence set forth in any one of SEQ ID NO: 24-33. In certain embodiments, the modified IL-18 polypeptide comprises an amino acid sequence having at least 90% sequence identity with the sequence set forth in any one of SEQ ID NO: 24-33. In certain embodiments, the modified IL-18 polypeptide comprises an amino acid sequence having at least 95% sequence identity with the sequence set forth in any one of SEQ ID NO: 24-33. In certain embodiments, the modified IL-18 polypeptide comprises an amino acid sequence having at least 98% sequence identity with the sequence set forth in any one of SEQ ID NO: 24-33. In certain embodiments, the modified IL-18 polypeptide comprises the amino acid sequence set forth in any one of SEQ ID NO: 24-33. In certain embodiments, the modified IL-18 polypeptide comprises an amino acid sequence having at least 80%, 85%, 90%, 95%, 98%, 99%, or 100% sequence identity with the sequence set forth in any one of SEQ ID NO: 204-244. In certain embodiments, the modified IL-18 polypeptide comprises an amino acid sequence having at least 80% sequence identity with the sequence set forth in any one of SEQ ID NO: 204-244. In certain embodiments, the modified IL-18 polypeptide comprises an amino acid sequence having at least 90% sequence identity with the sequence set forth in any one of SEQ ID NO: 204-244. In certain embodiments, the modified IL-18 polypeptide comprises an amino acid sequence having at least 95% sequence identity with the sequence set forth in any one of SEQ ID NO: 204-244. In certain embodiments, the modified IL-18 polypeptide comprises an amino acid sequence having at least 98% sequence identity with the sequence set forth in any one of SEQ ID NO: 204-244. In certain embodiments, the modified IL-18 polypeptide comprises the amino acid sequence set forth in any one of SEQ ID NO: 204-244. In certain embodiments, the modified IL-18 polypeptide comprises an amino acid sequence having at least 80%, 85%, 90%, 95%, 98%, 99%, or 100% sequence identity with the sequence set forth in SEQ ID NO: 30. In certain embodiments, the modified IL-18 polypeptide comprises an amino acid sequence having at least 80% sequence identity with the sequence set forth in SEQ ID NO: 30. In certain embodiments, the modified IL-18 polypeptide comprises an amino acid sequence having at least 90% sequence identity with the sequence set forth in SEQ ID NO: 30. In certain embodiments, the modified IL-18 polypeptide comprises an amino acid sequence having at least 95% sequence identity with the sequence set forth in SEQ ID NO: 30. In certain embodiments, the modified IL-18 polypeptide comprises an amino acid sequence having at least 98% sequence identity with the sequence set forth in SEQ ID NO: 30. In certain embodiments, the modified IL-18 polypeptide comprises the amino acid sequence set forth in SEQ ID NO: 30. In certain embodiments, the modified IL-18 polypeptide comprises an amino acid sequence having at least 80%, 85%, 90%, 95%, 98%, 99%, or 100% sequence identity with the sequence set forth in SEQ ID NO: 207. In certain embodiments, the modified IL-18 polypeptide comprises an amino acid sequence having at least 80% sequence identity with the sequence set forth in SEQ ID NO: 207. In certain embodiments, the modified IL-18 polypeptide comprises an amino acid sequence having at least 90% sequence identity with the sequence set forth in SEQ ID NO: 207. In certain embodiments, the modified IL-18 polypeptide comprises an amino acid sequence having at least 95% sequence identity with the sequence set forth in SEQ ID NO: 30. In certain embodiments, the modified IL-18 polypeptide comprises an amino acid sequence having at least 98% sequence identity with the sequence set forth in SEQ ID NO: 207. In certain embodiments, the modified IL-18 polypeptide comprises the amino acid sequence set forth in SEQ ID NO: 207. In certain embodiments, the modified IL-18 polypeptide comprises an amino acid sequence having at least 80%, 85%, 90%, 95%, 98%, 99%, or 100% sequence identity with the sequence set forth in SEQ ID NO: 239. In certain embodiments, the modified IL-18 polypeptide comprises an amino acid sequence having at least 80% sequence identity with the sequence set forth in SEQ ID NO: 239. In certain embodiments, the modified IL-18 polypeptide comprises an amino acid sequence having at least 90% sequence identity with the sequence set forth in SEQ ID NO: 239. In certain embodiments, the modified IL-18 polypeptide comprises an amino acid sequence having at least 95% sequence identity with the sequence set forth in SEQ ID NO: 239. In certain embodiments, the modified IL-18 polypeptide comprises an amino acid sequence having at least 98% sequence identity with the sequence set forth in SEQ ID NO: 239. In certain embodiments, the modified IL-18 polypeptide comprises the amino acid sequence set forth in SEQ ID NO: 239. In certain embodiments, the modified IL-18 polypeptide comprises an amino acid sequence having at least 80%, 85%, 90%, 95%, 98%, 99%, or 100% sequence identity with the sequence set forth in SEQ ID NO: 241. In certain embodiments, the modified IL-18 polypeptide comprises an amino acid sequence having at least 80% sequence identity with the sequence set forth in SEQ ID NO: 241. In certain embodiments, the modified IL-18 polypeptide comprises an amino acid sequence having at least 90% sequence identity with the sequence set forth in SEQ ID NO: 241. In certain embodiments, the modified IL-18 polypeptide comprises an amino acid sequence having at least 95% sequence identity with the sequence set forth in SEQ ID NO: 241. In certain embodiments, the modified IL-18 polypeptide comprises an amino acid sequence having at least 98% sequence identity with the sequence set forth in SEQ ID NO: 241. In certain embodiments, the modified IL-18 polypeptide comprises the amino acid sequence set forth in SEQ ID NO: 241. In certain embodiments, the modified IL-18 polypeptide comprises an amino acid sequence having at least 80%, 85%, 90%, 95%, 98%, 99%, or 100% sequence identity with the sequence set forth in SEQ ID NO: 242. In certain embodiments, the modified IL-18 polypeptide comprises an amino acid sequence having at least 80% sequence identity with the sequence set forth in SEQ ID NO: 242. In certain embodiments, the modified IL-18 polypeptide comprises an amino acid sequence having at least 90% sequence identity with the sequence set forth in SEQ ID NO: 242. In certain embodiments, the modified IL-18 polypeptide comprises an amino acid sequence having at least 95% sequence identity with the sequence set forth in SEQ ID NO: 242. In certain embodiments, the modified IL-18 polypeptide comprises an amino acid sequence having at least 98% sequence identity with the sequence set forth in SEQ ID NO: 242. In certain embodiments, the modified IL-18 polypeptide comprises the amino acid sequence set forth in SEQ ID NO: 242. In certain embodiments, the modified IL-18 polypeptide comprises an amino acid sequence having at least 80%, 85%, 90%, 95%, 98%, 99%, or 100% sequence identity with the sequence set forth in SEQ ID NO: 244. In certain embodiments, the modified IL-18 polypeptide comprises an amino acid sequence having at least 80% sequence identity with the sequence set forth in SEQ ID NO: 244. In certain embodiments, the modified IL-18 polypeptide comprises an amino acid sequence having at least 90% sequence identity with the sequence set forth in SEQ ID NO: 244. In certain embodiments, the modified IL-18 polypeptide comprises an amino acid sequence having at least 95% sequence identity with the sequence set forth in SEQ ID NO: 244. In certain embodiments, the modified IL-18 polypeptide comprises an amino acid sequence having at least 98% sequence identity with the sequence set forth in SEQ ID NO: 244. In certain embodiments, the modified IL-18 polypeptide comprises the amino acid sequence set forth in SEQ ID NO: 244.

In one aspect, provided herein, is a polymer modified IL-18 polypeptide. The polymer modified IL-18 polypeptide comprise a polymer covalently attached to a residue of the IL-18 polypeptide. In certain embodiments, the polymer is attached to a cysteine of the modified IL-18 polypeptide. In certain embodiments, the polymer is attached at residue 68 of the modified IL-18 polypeptide, wherein residue position numbering is based on SEQ ID NO: 1 as a reference sequence. In certain embodiments, the polymer is attached to any one of residues 79-120, wherein residue position numbering of the modified IL-18 polypeptide is based on SEQ ID NO: 1 as a reference sequence. In certain embodiments, the polymer is attached at residues 85. In certain embodiments, the polymer is attached at residues 86. In certain embodiments, the polymer is attached at residues 95. The polymer modified IL-18 polypeptide may display a lower binding affinity (resulting in a higher K_{D}) for an IL-18 receptor alpha/beta heterodimer (IL-18Rα/β) which is at most eight fold less than the binding affinity displayed by an identical modified IL-18 polypeptide with no polymer attached. In some embodiments, the polymer is covalently attached to a residue selected from residue 79, residue 80, residue 81, residue 82, residue 83, residue 84, residue 85, residue 86, residue 87, residue 88, residue 89, residue 90, residue 91, residue 92, residue 93, residue 94, residue 95, residue 96, residue 97, residue 98, residue 99, residue 100, residue 101, residue 102, residue 103, residue 104, residue 105, residue 106, residue 107, residue 108, residue 109, residue 110, residue 111, residue 112, residue 113, residue 114, residue 115, residue 116, residue 117, residue 118, residue 119, and residue 120. In some embodiments, the polymer is covalently attached at residue 85. In some embodiments, the polymer is covalently attached at residue 86. In some embodiments, the polymer is covalently attached at residue 95. In some embodiments, the polymer is covalently attached at residue 98. In some embodiments, the polymer is covalently attached at residue E85, E85C, E85D, E85Q, E85K, E85N, or E85Y. In some embodiments, the polymer is covalently attached at residue M86C, M86D, M86Q, M86K, M86N, M86E, or M86Y. In some embodiments, the polymer is covalently attached at residue T95C, T95D, T95Q, T95K, T95N, T95E, T95Y, D98C, D98Q, D98K, D98N, D98E, or D98Y.

In some embodiments, the polymer is attached to a natural amino acid residue. In some embodiments, the natural amino acid residue is selected from asparagine, aspartic acid, cysteine, glutamic acid, glutamine, lysine, and tyrosine. In some embodiments, the polymer is attached to an unnatural amino acid residue. In some embodiments, one of residues 79-120 is substituted for a cysteine. In some embodiments, the polymer comprises a conjugation handle or a reaction product of a conjugation handle with a complementary conjugation handle. In some embodiments, the conjugation handle or the reaction product of the conjugation handle with the complementary conjugation handle comprises an azide moiety, an alkyne moiety, or reaction product of an azide-alkyne cycloaddition reaction.

In some embodiments, the polymer is a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer comprises poly(alkylene oxide). In some embodiments, the poly(alkylene oxide) is polyethylene glycol (PEG).

In some embodiments, the modified IL-18 polypeptide comprises one or more amino acid substitutions at residue Y1, F2, E6, V11, C38, K53, D54, S55, T63, C76, C127, or any combination thereof. In some embodiments, the modified IL-18 polypeptide comprises a Y01G, F02A, E06K, V11I, C38S, C38A, C38Q, D54A, S55A, T63A, C76S, C76A, C127S, C127A amino acid substitution, or any combination thereof. In some embodiments, the modified IL-18 polypeptide comprises a Y01G, F02A, E06K, V11I, C38S, C38A, C38Q, K53A, D54A, S55A, T63A, C76S, C76A, C127S, C127A amino acid substitution, or any combination thereof. In some embodiments, the modified IL-18 polypeptide comprises E06K and K53A amino acid substitutions. In some embodiments, the modified IL-18 polypeptide comprises a V11I amino acid substitution. In some embodiments, the modified IL-18 polypeptide comprises a T63A amino acid substitution. In some embodiments, the modified IL-18 polypeptide comprises the modified IL-18 polypeptide comprises an N-terminal extension. In some embodiments, the modified IL-18 polypeptide comprises a polypeptide sequence having at least about 80% sequence identity to any one of SEQ ID NOs: 2-244. In some embodiments, the modified IL-18 polypeptide comprises a polypeptide sequence having at least about 80% sequence identity to any one of SEQ ID NOs: 2-67. In some embodiments, the polypeptide sequence is at least about 80% identical to SEQ ID NO: 4 or SEQ ID NO: 30. In some embodiments, the polypeptide sequence is at least about 80% identical to SEQ ID NO: 4. In some embodiments, the polypeptide sequence is at least about 90% identical to SEQ ID NO: 4. In some embodiments, the polypeptide sequence is at least about 95% identical to SEQ ID NO: 4. In some embodiments, the polypeptide sequence is at least about 80% identical to SEQ ID NO: 30. In some embodiments, the polypeptide sequence is at least about 90% identical to SEQ ID NO: 30. In some embodiments, the polypeptide sequence is at least about 95% identical to SEQ ID NO: 30. In some embodiments, the modified IL-18 polypeptide is recombinant.

In some embodiments, the modified IL-18 polypeptide comprises one or more amino acid substitutions selected from: (a) a homoserine residue located at any one of residues 26-36; (b) a homoserine residue located at any one of residues 45-67; (c) a homoserine residue located at any one of residues 70-80; (d) a homoserine residue located at any one of residues 100-130; (e) a norleucine or O-methyl-homoserine residue located at any one of residues 28-38; (f) a norleucine or O-methyl-homoserine residue located at any one of residues 46-56; (g) a norleucine or O-methyl-homoserine residue located at any one of residues 54-64; (h) a norleucine or O-methyl-homoserine residue located at any one of residues 80-90; (i) a norleucine or O-methyl-homoserine residue located at any one of residues 108-118; and (j) a norleucine or O-methyl-homoserine residue located at any one of residues 145-155; wherein residue position numbering of the modified IL-18 polypeptide is based on SEQ ID NO: 1 as a reference sequence. In some embodiments, the modified IL-18 polypeptide comprises one or more amino acid substitutions selected from homoserine (Hse) 31, norleucine (Nle) 33, O-methyl-homoserine (Omh) 33, Hse50, Nle51, Omh51, Hse57, Nle60, Hse63, Omh60, Hse75, Nle86, Omh86, Hse116, Nle113, Omh113, Hse 121 Nle150, and Omh150.

In some embodiments, the modified IL-18 polypeptide modulates IFNγ production, and wherein an EC₅₀ (nM) of the modified IL-18 polypeptide's ability to induce IFNγ is less than 10-fold higher than, less than 5-fold higher than, or less than an EC₅₀ (nM) of an IL-18 polypeptide of SEQ ID NO: 1. In some embodiments, the EC₅₀ (nM) of the modified IL-18 polypeptide's ability to induce IFNγ is less than 5-fold higher than the EC₅₀ (nM) of SEQ ID NO: 1. In some embodiments, the EC₅₀ (nM) of the modified IL-18 polypeptide's ability to induce IFNγ is less than the EC₅₀ (nM) an IL-18 polypeptide of SEQ ID NO: 1. In some embodiments, the EC₅₀ (nM) of the modified IL-18 polypeptide's ability to induce IFNγ is at least about 10-fold less than the EC₅₀ (nM) of SEQ ID NO: 1. In some embodiments, the modified IL-18 polypeptide exhibits less than a 10-fold lower affinity, less than a 5-fold lower affinity, or a greater affinity to an IL-18 receptor alpha subunit (IL-18Rα) than to IL-18 binding protein (IL-18BP) as measured by K_{D}, and wherein [K_{D} IL-18Rα]/[K_{D} IL-18BP] is greater than 0.1. In some embodiments, the modified IL-18 polypeptide binds to IL-18 receptor alpha (IL-18Rα). In some embodiments, the modified IL-18 polypeptide binds to IL-18Rα with a K_{D} of less than about 200 nM, less than about 100 nM, less than about 80 nM, less than about 70 nM, less than about 60 nM, or less than about 50 nM. In some embodiments, the modified IL-18 polypeptide binds to IL-18Rα with a K_{D} of less than about 50 nM. In some embodiments, the modified IL-18 polypeptide binds to an IL-18 receptor alpha/beta (IL-18Rα/β) heterodimer. In some embodiments, the modified IL-18 polypeptide binds to the IL-18Rα/β heterodimer with a K_{D} of less than about 25 nM. In some embodiments, the modified IL-18 polypeptide binds to the IL-18Rα/β heterodimer with a K_{D} of less than about 10 nM. In some embodiments, the modified IL-18 polypeptide is conjugated to an additional peptide.

In one aspect, provided herein, is a host cell comprising a modified IL-18 polypeptide provided herein. In some embodiments, a method of producing a modified IL-18 polypeptide is provided herein, comprising expressing the modified IL-18 polypeptide in a host cell. In some embodiments, the host cell is a prokaryotic cell or a eukaryotic cell. In some embodiments, the host cell is a mammalian cell, an avian cell, a fungal cell, or an insect cell. In some embodiments, the host cell is a CHO cell, a COS cell, or a yeast cell.

In another aspect, provided herein, is a pharmaceutical composition comprising a modified IL-18 polypeptide provided herein and a pharmaceutically acceptable carrier or excipient. In an aspect, provided herein, is a pharmaceutical composition comprising a polymer modified IL-18 polypeptide provided herein and a pharmaceutically acceptable carrier or excipient. In some embodiments, the pharmaceutical composition comprising the modified IL-18 polypeptide, and/or the polymer modified IL-18 polypeptide, is in a lyophilized form.

In another aspect is a method of treating cancer in a subject in need thereof comprising administering to the subject a pharmaceutically effective amount of, a modified IL-18 polypeptide, or a pharmaceutical composition comprising a modified IL-18 polypeptide, provided herein. In an aspect is a method of treating cancer in a subject in need thereof comprising administering to the subject a pharmaceutically effective amount of, a polymer modified IL-18 polypeptide, or a pharmaceutical composition comprising a polymer modified IL-18 polypeptide, provided herein. In some embodiments, the cancer is a solid cancer. In some embodiments, the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer. In some embodiments, the solid cancer is a carcinoma or a sarcoma. In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia. In some embodiments, the method comprises reconstituting a lyophilized form of the modified IL-18 polypeptide or the pharmaceutical composition.

Another aspect provides a method of making a modified IL-18 polypeptide provided herein comprising synthesizing two or more fragments of the modified IL-18 polypeptide, ligating the fragments, and folding the ligated fragments. In some embodiments, at least one of the fragments of the IL-18 polypeptide comprises a conjugation handle. In some embodiments, the method comprises attaching the polymer to the folded, ligated fragments by a reaction with the conjugation handle. In an aspect provided herein a method of making a polymer modified IL-18 polypeptide, comprising synthesizing two or more fragments of the modified IL-18 polypeptide, ligating the fragments, folding the ligated fragments, and attaching a polymer to the folded, ligated fragments by a reaction with a conjugation handle, wherein at least one of the fragments of the IL-18 polypeptide comprises the conjugation handle. In some embodiments, one of the fragments comprises the polymer.

Yet another aspect herein provides modified IL-18 polypeptide having at least about 80%, at least about 85%, at least about 90%, at least about 95%, at least about 96%, at least about 97%, at least about 98%, or at least about 99% sequence identity to the sequence set forth in any one of SEQ ID NOs: 2-33. An aspect herein provides modified IL-18 polypeptide having at least about 80%, at least about 85%, at least about 90%, at least about 95%, at least about 96%, at least about 97%, at least about 98%, or at least about 99% sequence identity to the sequence set forth in any one of SEQ ID NOs: 204-244.

Another aspect provides a modified IL-18 polypeptide having at least about 80%, at least about 85%, at least about 90%, at least about 95%, at least about 96%, at least about 97%, at least about 98%, or at least about 99% sequence identity to the sequence set forth in any one of SEQ ID NOs: 24-33.

Another aspect provides a modified IL-18 polypeptide having at least about 80%, at least about 85%, at least about 90%, at least about 95%, at least about 96%, at least about 97%, at least about 98%, or at least about 99% sequence identity to the sequence set forth in SEQ ID NO: 30. In some embodiments, the IL-18 polypeptide comprises a polymer covalently attached. In some embodiments, the polymer is covalently attached at residue 68, 69, or 70. In some embodiments, the polymer is covalently attached at residue 68.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing, of which:

FIG. 1 illustrates the mechanism of action of IL-18 on IFNγ and IL-18BP production, and IL-18 inhibitory activity by IL-18BP.

FIG. 2 illustrates the coupling of a dibenzocyclooctyne (DBCO) polyethylene glycol (PEG) with a modified IL-18 polypeptide comprising an azide.

FIG. 3 illustrates the binding of a modified IL-18 polypeptide comprising a polymer with IL-18Rα.

FIG. 4A shows the IFNγ induction ability of a modified IL-18 polypeptide compared to a wild type IL-18 polypeptide.

FIG. 4B shows IL-18BP inhibition of a modified IL-18 polypeptide compared to a wild type IL-18 polypeptide.

FIG. 5 shows a schematic representation of coupling of a bifunctional probe to an IL-18 polypeptide provided herein.

FIG. 6 shows a schematic representation of coupling of a poly(ethylene glycol) moiety to an IL-18 polypeptide activated with a bifunctional probe.

FIG. 7 shows a schematic of a synthetic route which can be used to synthesize an IL-18 polypeptide modified with a polymer.

FIG. 8 shows an additional schematic of a synthetic route which can be used to synthesize an IL-18 polypeptide.

FIG. 9 shows an additional schematic of a synthetic route which can be used to synthesize an IL-18 polypeptide.

FIG. 10 shows an additional schematic of a synthetic route which can be used to synthesize an IL-18 polypeptide.

FIG. 11 shows an exemplary purification result of an IL-18 polypeptide as provided herein.

FIG. 12 shows an exemplary Analytical SEC-HPLC chromatogram of purified SEQ ID NO: 207 (detection: 220 nm).

FIG. 13 shows an exemplary ESI-Q-TOF-HRMS spectra of purified SEQ ID NO: 207.

FIG. 14 shows an exemplary Analytical SEC-HPLC chromatogram of purified SEQ ID NO: 239 (detection: 220 nm).

FIG. 15 shows an exemplary ESI-Q-TOF-HRMS spectra of purified SEQ ID NO: 239.

FIG. 16 shows an exemplary Analytical SEC-HPLC chromatogram of purified SEQ ID NO: 244 (detection: 220 nm).

FIG. 17 shows an exemplary ESI-Q-TOF-HRMS spectra of purified SEQ ID NO: 244.

FIG. 18 shows an exemplary Analytical SEC-HPLC chromatogram of purified SEQ ID NO: 242 (detection: 220 nm).

FIG. 19 shows an exemplary ESI-Q-TOF-HRMS spectra of purified SEQ ID NO: 242.

FIG. 20 shows an exemplary Analytical SEC-HPLC chromatogram of purified SEQ ID NO: 241 (detection: 220 nm).

FIG. 21 shows an exemplary ESI-Q-TOF-HRMS spectra of purified SEQ ID NO: 242.

FIG. 22 shows an exemplary Analytical SEC-HPLC chromatogram of purified SEQ ID NO: 30 (detection: 220 nm).

FIG. 23 shows an exemplary ESI-Q-TOF-HRMS spectra of purified SEQ ID NO: 30.

FIG. 24 shows plots measuring the binding activity of wild type IL-18 and of modified IL-18 polypeptides to human IL-18 receptor alpha (CD218a) in surface plasmon resonance experiments, where the x-axis is time, and the y-axis is relative response. The modified IL-18 polypeptides tested in these experiments are native IL-18 wild-type (SEQ ID NO: 01), SEQ ID NO: 57, SEQ ID NO: 59, and SEQ ID NO: 30.

FIG. 25 shows plots measuring the binding activity of wild type IL-18 and of modified IL-18 polypeptides to heterodimeric human IL-18 receptor alpha (CD218a) and IL-18R accessory protein (IL-18RAP/CDw218b) in surface plasmon resonance experiments, where the x-axis is time, and the y-axis is relative response. The modified IL-18 polypeptides tested in these experiments are native IL-18 wild-type (SEQ ID NO: 01), SEQ ID NO: 57, SEQ ID NO: 59, and SEQ ID NO: 30.

FIG. 26 shows plots measuring the binding activity of wild type IL-18 and of modified IL-18 polypeptides to human IL-18 binding protein (IL-18BP) in surface plasmon resonance experiments, where the x-axis is time, and the y-axis is relative response. The modified IL-18 polypeptides tested in these experiments are native IL-18 wild-type (SEQ ID NO: 01), SEQ ID NO: 57, SEQ ID NO: 59, and SEQ ID NO: 30.

FIG. 27 shows plots measuring the ability of wild type IL-18 and of modified IL-18 polypeptides to bind to the human IL-18 Binding Protein (IL-18BP). The figure shows mean free IL-18BP AlphaLISA signal on the y-axis, and dosage of of wild type IL-18 and of modified IL-18 polypeptides on the x-axis. The unconjugated IL-18 variants are native IL-18 wild-type (SEQ ID NO: 01), SEQ ID NO: 57, SEQ ID NO: 59, SEQ ID NO: 30, SEQ ID NO: 207, SEQ ID NO: 239, SEQ ID NO: 241, SEQ ID NO: 242, and SEQ ID NO: 244.

FIG. 28 shows plots measuring ability of wild type IL-18 and of modified IL-18 polypeptides to stimulate the secretion of IFNgamma by NK-92 cells. The figure shows mean IFNg alphaLISA signal on the y-axis and dosage of the IL-18 polypeptides on the x-axis. The IL-18 polypeptides are native IL-18 wild-type (SEQ ID NO: 01), SEQ ID NO: 57, SEQ ID NO: 59, and SEQ ID NO: 30.

FIG. 29 shows plots measuring the ability of the human IL-18 Binding Protein to inhibit the secretion of IFNgamma by NK92 cells stimulated with 2 nM of wild type IL-18 and of modified IL-18 polypeptides. The figure shows mean IFNg alphaLISA signal on the y-axis, and dosage of the human IL-18 Binding Protein on the x-axis. The IL-18 polypeptides are native IL-18 wild-type (SEQ ID NO: 01), SEQ ID NO: 57, SEQ ID NO: 59, and SEQ ID NO: 30.

FIG. 30 shows plots measuring ability of wild type IL-18 and of modified IL-18 polypeptides to induce the NF-κB/AP-1-inducible secreted embryonic alkaline phosphatase (SEAP) reporter gene in Hek Blue cells expressing the IL-18 receptor. The figure shows mean SEAP reporter signal (OD 620 nm) on the y-axis, and dosage of the IL-18 polypeptides on the x-axis. The IL-18 polypeptides are native IL-18 wild-type (SEQ ID NO: 01), SEQ ID NO: 57, SEQ ID NO: 59, SEQ ID NO: 207, SEQ ID NO: 239, SEQ ID NO: 241, SEQ ID NO: 242, and SEQ ID NO: 244.

FIG. 31 shows plots measuring ability of wild type IL-18 and of modified IL-18 polypeptides to stimulate the secretion of IFNgamma by human Peripheral Blood Mononuclear Cells (PBMCs). The figure shows mean IFNg signal on the y-axis and dosage of the IL-18 polypeptides on the x-axis. The IL-18 polypeptides are native IL-18 wild-type (SEQ ID NO: 01), SEQ ID NO: 57, SEQ ID NO: 59, and SEQ ID NO: 30.

FIGS. 32A-B show effect of attaching 30 kDa PEG to IL-18 variants on IL-18 mediated IFNγ secretion in human peripheral blood mononuclear cells. EC50 for IL-18 mediated IFNγ secretion for IL-18 of SEQ ID NO: 59, 4 and 9 is shown in FIG. 32A, and of SEQ ID NO: 5 and 6 is shown in FIG. 32B.

### DETAILED DESCRIPTION

Immune responses to tumors are primarily the function of T helper type 1 (Th1) lymphocytes. Th1 responses include the secretion of cytokines IL-2, IL-12, IL-18, IFNγ, and the generation of specific cytotoxic T lymphocytes that recognize specific tumor antigens. The Th1 response is a vital arm of host defense against many microorganisms. However, the Th1 response is also associated with autoimmune diseases and organ transplant rejection.

Interleukin 18 (IL-18) is a pro-inflammatory cytokine that elicits biological activities that initiate or promote host defense and inflammation following infection or injury. IL-18 has been implicated in autoimmune diseases, myocardial function, emphysema, metabolic syndromes, psoriasis, inflammatory bowel disease, hemophagocytic syndromes, macrophage activation syndrome, sepsis, and acute kidney injury. In some models of disease, IL-18 plays a protective role.

IL-18 also plays a major role in the production of IFNγ from T-cells and natural killer cells. IFNγ is a Th1 cytokine mainly produced by T cells, NK cells, and macrophages and is critical for innate and adaptive immunity against viral, some bacterial, and protozoal infections. IFNγ is also an important activator of macrophages and inducer of Class II major histocompatibility complex (MHC) molecule expression.

IL-18 forms a signaling complex by binding to the IL-18 alpha chain (IL-18Rα), which is the ligand binding chain for mature IL-18. However, the binding affinity of IL-18 to IL-18Rα is low. In cells that express the co-receptor, IL-18 receptor beta chain (IL-18Rβ), a high affinity heterodimer complex is formed, which then activates cell signaling.

In order to improve the therapeutic potential of IL-18, addition of a half-life-extending polymer (e.g., polyethylene glycol (PEG)) can be desirous in order to improve pharmacokinetic (PK) or pharmacodynamics (PD) properties of IL-18. However, addition of a polymer to certain residues of IL-18 has been observed to result in reduced affinity to one or more subunits of the IL-18 receptor (e.g., the alpha subunit, the beta subunit, or the alpha/beta heterodimer) and result in a concomitant reduced bioactivity of the IL-18 (e.g., reduced potency in inducing production of IFNγ compared to WT IL-18). For example, PCT Publication Number WO2004091517, which is hereby incorporated by reference as if set forth in its entirety, shows that PEGylation of cysteine residues (natural or substituted) at positions 38, 68, 78, 121, 144, and 157 of IL-18 in some instances may result in substantial loss of affinity to IL-18Rα and ability to induce IFNγ production in immune cells compared to both WT IL-18 and to the non-PEGylated version of the modified IL-18. Thus, identification of alternative sites for polymer addition that reduce the deactivation of IL-18 relative to other sites would provide substantial advantages.

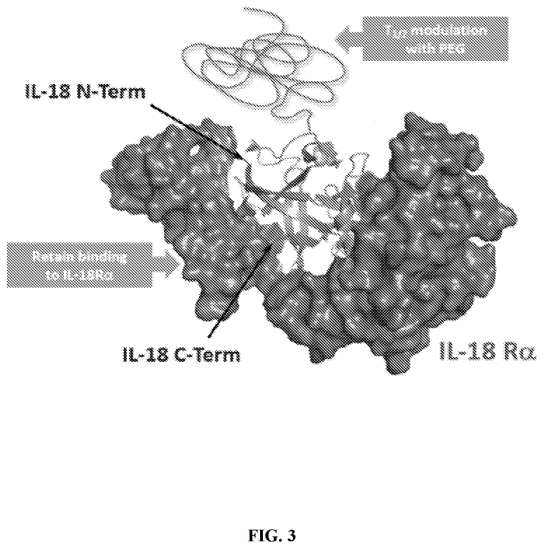

In order to overcome these deficiencies, provided herein are IL-18 polypeptides modified with polymers that substantially retain binding to IL-18R and bioactivity. In some embodiments, the addition of polymers to residues of the IL-18 polypeptide as provided herein allows for a half-life extended IL-18 with binding to IL-18R and/or bioactivity similar to or only slightly reduced compared to WT IL-18. In some embodiments, the addition of polymers to residues of the IL-18 polypeptide as provided herein allows for a half-life extended IL-18 with binding to IL-18R and/or bioactivity similar to or only slightly reduced compared to the IL-18 with no polymer attached. An exemplary picture of a modified IL-18 polypeptide with a polymer provided herein binding to an IL-18 receptor alpha subunit is shown in FIG. 3.

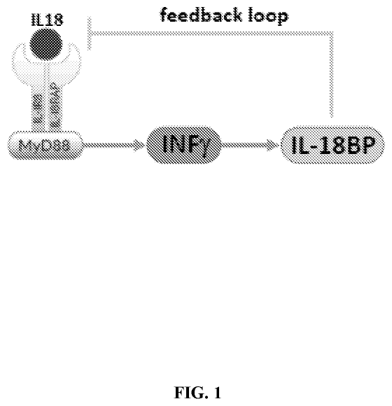

While extending the half-life of an IL-18 polypeptide with a polymer in a way that does not substantially diminish binding to IL-18R can offer advantages over WT and other variant IL-18s, other considerations can also affect the therapeutic potential of modified IL-18 polypeptides. For example, the activity of IL-18 is balanced by the presence of a high affinity, naturally occurring IL-18 binding protein (IL-18BP). IL-18BP binds IL-18 and neutralizes the biological activity of IL-18. Cell surface IL-18Rα competes with IL-18BP for IL-18 binding. Increased disease severity can be associated with an imbalance of IL-18 to IL-18BP such that levels of free IL-18 are elevated in the circulation. FIG. 1 illustrates the mechanism of action of IL-18, IFNγ production, IL-18BP production, and inhibition of IL-18 activity by IL-18BP. IL-18 induces IFNγ production, which in turn induces IL-18BP production. IL-18BP then competes with IL-18Rα to inhibit IL-18 activity. This feedback loop of IL-18BP production after stimulation of IFNγ production has limited the effectiveness of IL-18 as a treatment modality in previous efforts.

Thus, in some embodiments, IL-18 polypeptides modified with polymers provided herein also exhibit reduced binding affinity to IL-18BP compared to WT IL-18. In some embodiments, the presence of the polymer results in reduced binding of the modified IL-18 polypeptide to IL-18 BP. In some embodiments, the modified IL-18 polypeptide contains one or more amino acid substitutions that reduce the interaction of the modified IL-18 with IL-18BP. In some embodiments, the modified IL-18 polypeptide contains one or more amino acid substitutions that reduce the ability of the IL-18 to be neutralized by IL-18BP and which enhance the IL-18's ability to bind to IL-18R. In some embodiments, IL-18 polypeptides modified with polymers provided herein contain one or more amino acid substitutions that allow the IL-18 polypeptide to have an optimal mix of half-life, reduced IL-18BP neutralization, and activity against IL-18R.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

### I. Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Referred to herein are polymers which are “attached” or “covalently attached” to residues of IL-18 polypeptides. As used herein, “attached” or “covalently attached” means that the polymer is tethered to the indicated reside, and such tethering can include a linking group (i.e., a linker). Thus, for a polymer “attached” or “covalently attached” to a residue, it is expressly contemplated that such linking groups are also encompassed.

As used herein, an “alpha-keto amino acid” or the phrase “alpha-keto” before the name of an amino acid refers to an amino acid or amino acid derivative having a ketone functional group positioned between the carbon bearing the amino group and the carboxylic acid of an amino acid. Alpha-keto amino acids of the instant disclosure have a structure as set forth in the following formula:

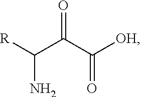

wherein R is the side chain of any natural or unnatural amino acid. The R functionality can be in either the L or D orientation in accordance with standard amino acid nomenclature. In preferred embodiments, alpha-keto amino acids are in the L orientation. When the phrase “alpha-keto” is used before the name of a traditional natural amino acid (e.g., alpha-keto leucine, alpha-keto phenylalanine, etc.) or a common unnatural amino acid (e.g., alpha-keto norleucine, alpha-keto O-methyl-homoserine, etc.), it is intended that the alpha-keto amino acid referred to matches the above formula with the side chain of the referred to amino acid. When an alpha-keto amino acid residue is set forth in a peptide or polypeptide sequence herein, it is intended that a protected version of the relevant alpha-keto amino acid is also encompassed (e.g., for a sequence terminating in a C-terminal alpha-keto amino acid, the terminal carboxylic acid group may be appropriately capped with a protecting group such as a tert-butyl group, or the ketone group with an acetal protecting group). Other protecting groups encompassed are well known in the art.

Binding affinity refers to the strength of a binding interaction between a single molecule and its ligand/binding partner. A higher binding affinity refers to a higher strength bond than a lower binding affinity. In some instances, binding affinity is measured by the dissociation constant (KD) between the two relevant molecules. When comparing KD values, a binding interaction with a lower value will have a higher binding affinity than a binding interaction with a higher value. For a protein-ligand interaction, KD is calculated according to the following formula:

$${K}_{D} = \frac{[ L ] [ P ]}{[ L P ]}$$

where [L] is the concentration of the ligand, [P] is the concentration of the protein, and [LP] is the concentration of the ligand/protein complex.

Referred to herein are certain amino acid sequences (e.g., polypeptide sequences) which have a certain percent sequence identity to a reference sequence or refer to a residue at a position corresponding to a position of a reference sequence. Sequence identity is measured by protein-protein BLAST algorithm using parameters of Matrix BLOSUM62, Gap Costs Existence:11, Extension:1, and Compositional Adjustments Conditional Compositional Score Matrix Adjustment. This alignment algorithm is also used to assess if a residue is at a “corresponding” position through an analysis of the alignment of the two sequences being compared.

Referred to herein are polymers which are “attached” or “covalently attached” to residues of IL-18 polypeptides. As used herein, “attached” or “covalently attached” means that the polymer is tethered to the indicated reside, and such tethering can include a linking group (i.e., a linker). Thus, for a polymer “attached” or “covalently attached” to a residue, it is expressly contemplated that such linking groups are also encompassed.

Unless otherwise specified, is contemplated that “protected” versions of amino acids (e.g., those containing a chemical protecting group affixed to a functionality of the amino acid, particularly a side chain of the amino acid but also at another point of the amino acid) qualify as the same amino acid as the “unprotected” version for sequence identity purposes, particularly for chemically synthesized polypeptides. It is also contemplated that such protected versions are also encompassed by the SEQ ID NOs provided herein. Non-limiting examples of protecting groups which may be encompassed include fluorenylmethyloxycarbonyl (Fmoc), triphenylmethyl (trityl or trt), tert-Butyloxycarbonyl (Boc), 2,2,4,6,7-pentamethyldihydrobenzofuran-5-sulfonyl (Pbf), acetamidomethyl (Acm), tert-butyl (tBu or OtBu), 2,2-dimethyl-1-(4-methoxyphenyl)propane-1,3-diol ketal or acetal, and 2,2-dimethyl-1-(2-nitrophenyl)propane-1,3-diol ketal or acetal. Other protecting groups well known in the art are also encompassed. Similarly, modified versions of natural amino acids are also intended to qualify as natural version of the amino acid for sequence identity purposes. For example, an amino acid comprising a side chain heteroatom which can be covalently modified (e.g., to add a conjugation handle, optionally through a linker), such as a lysine, glutamine, glutamic acid, asparagine, aspartic acid, cysteine, or tyrosine, which has been covalently modified would be counted as the base amino acid (see, e.g., Structure 2 below, which would be counted as a lysine for sequence identity and SEQ ID purposes). Similarly, an amino acid comprising another group added to the C or N-terminus would be counted as the base amino acid.

In some instances, peptides provided herein may be depsipeptides. For example, a depsipeptide linkage result from certain ligation reactions described herein (e.g., KAHA ligations) during the synthesis of synthetic IL-18s and relevant precursor peptides. In particular, hydroxyl containing amino acids (e.g., serine, threonine, and homoserine) form depsipeptide linkages with the adjacent amino acid on the N-terminal side. Thus, when a sequence ID lists an amino acid sequence, it is also contemplated that a depsipeptide version of the sequence is also encompassed, particularly at homoserine residues.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” suitable for the disclosure may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0, 2, 3, 4, or 4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

Certain formulas and other illustrations provided herein depict triazole reaction products resulting from azide-alkyne cycloaddition reactions. While such formulas generally depict only a single regioisomer of the resulting triazole formed in the reaction, it is intended that the formulas encompass both resulting regioisomers. Thus, while the formulas depict only a single regioisomer

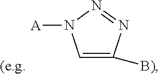

it is intended that the other regioisomer

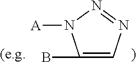

is is also encompassed.

The term “optional” or “optionally” denotes that a subsequently described event or circumstance can but need not occur, and that the description includes instances where the event or circumstance occurs and instances in which it does not.

The term “moiety” refers to a specific segment or functional group of a molecule. Chemical moieties are often recognized chemical entities embedded in or appended to a molecule.

As used herein, “conjugation handle” refers to a reactive group capable of forming a bond upon contacting a complementary reactive group. In some instances, a conjugation handle preferably does not have a substantial reactivity with other molecules which do not comprise the intended complementary reactive group. Non-limiting examples of conjugation handles, their respective complementary conjugation handles, and corresponding reaction products can be found in the table below. While table headings place certain reactive groups under the title “conjugation handle” or “complementary conjugation handle,” it is intended that any reference to a conjugation handle can instead encompass the complementary conjugation handles listed in the table (e.g., a trans-cyclooctene can be a conjugation handle, in which case tetrazine would be the complementary conjugation handle). In some instances, amine conjugation handles and conjugation handles complementary to amines are less preferable for use in biological systems owing to the ubiquitous presence of amines in biological systems and the increased likelihood for off-target conjugation.

Table of Conjugation Handles

| Conjugation | | Reaction |
|---|---|---|
| Handle | Complementary Conjugation Handle | Product |
| Sulfhydryl | alpha-halo-carbonyl (e.g., bromoacet- | thioether |
| | amide), alpha-beta unsaturated carbonyl | |
| | (e.g., maleimide, acrylamide) | |
| Azide | alkyne (e.g., terminal alkyne, substituted | triazole |
| | cyclooctyne (e.g., dibenzocycloocytne | |
| | (DBCO), difluorocyclooctyne, | |
| | bicyclo[6.1.0]nonyne, etc.)) | |
| Phosphine | Azide/ester pair | amide |
| Tetrazine | trans-cyoclooctene | dihydropy- |
| | | ridazine |
| Amine | Activated ester (e.g., N-hydroxysuccini- | amide |
| | mide ester, pentaflurophenyl ester) | |
| isocyanate | amine | urea |
| epoxide | amine | alkylamine |
| hydroxyl amine | aldehyde, ketone | oxime |
| hydrazide | aldehyde, ketone | hydrazone |
| potassium acyl | O-substituted hydroxylamine (e.g., O- | amide |
| trifluoroborate | carbamoylhydroxylamine) | |

Throughout the instant application, prefixes are used before the term “conjugation handle” to denote the functionality to which the conjugation handle is linked. For example, a “protein conjugation handle” is a conjugation handle attached to a protein (either directly or through a linker), an “antibody conjugation handle” is a conjugation handle attached to an antibody (either directly or through a linker), and a “linker conjugation handle” is a conjugation handle attached to a linker group (e.g., a bifunctional linker used to link a synthetic protein and an antibody)

As used herein, the term “number average molecular weight” (Mn) means the statistical average molecular weight of all the individual units in a sample, and is defined by Formula (1):

$$M n = \frac{\sum {N}_{i} {M}_{i}}{\sum {N}_{i}} \qquad Formula ( 1 )$$

where Mᵢ is the molecular weight of a unit and Nᵢ is the number of units of that molecular weight.

As used herein, the term “weight average molecular weight” (Mw) means the number defined by Formula (2):

$$M w = \frac{\sum {N}_{i} {M}_{i}^{2}}{\sum {N}_{i} {M}_{i}} \qquad Formula ( 2 )$$

where Mᵢ is the molecular weight of a unit and Nᵢ is the number of units of that molecular weight.

As used herein, “peak molecular weight” (Mp) means the molecular weight of the highest peak in a given analytical method (e.g. mass spectrometry, size exclusion chromatography, dynamic light scattering, analytical centrifugation, etc.).

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C1-C10 alkyl, likewise, for example, an alkyl comprising up to 6 carbon atoms is a C1-C6 alkyl. Alkyls (and other moieties defined herein) comprising other numbers of carbon atoms are represented similarly. Alkyl groups include, but are not limited to, C1-C10 alkyl, C₁-C₉ alkyl, Ci-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, «-propyl, 1-methyl ethyl (/-propyl), «-butyl, /-butyl, 5-butyl, «-pentyl, 1,1-dimethyl ethyl (/-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted. “Alkylene” or “alkylene chain” refers to a straight or branched divalent hydrocarbon chain linking the rest of the molecule to a radical group. In some embodiments, the alkylene is —CFF—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—. Unless stated otherwise specifically in the specification, an alkylene group may be optionally substituted.

The term “alkenylene” or “alkenylene chain” refers to a straight or branched divalent hydrocarbon chain in which at least one carbon-carbon double bond is present linking the rest of the molecule to a radical group. In some embodiments, the alkenylene is —CH═CH—, —CEhCEUCH—, or —CH═CHCH2-. In some embodiments, the alkenylene is —CH═CH—. In some embodiments, the alkenylene is —CH2CH═CH—. In some embodiments, the alkenylene is —CH═CHCH2-.

The term “alkynyl” refers to a type of alkyl group in which at least one carbon-carbon triple bond is present. In one embodiment, an alkenyl group has the formula —C≡C—R^{X}, wherein R^{x} refers to the remaining portions of the alkynyl group. In some embodiments, R^{x} is H or an alkyl. In some embodiments, an alkynyl is selected from ethynyl, propynyl, butynyl, pentynyl, hexynyl, and the like. Non-limiting examples of an alkynyl group include —C^{∘}CH, —C≡CCH3, —C≡CCH₂CH, and —CH₂C≡CH.

The term “alkynyl” refers to a type of alkyl group in which at least one carbon-carbon triple bond is present. In one embodiment, an alkenyl group has the formula —C≡C—R^{X}, wherein R^{x} refers to the remaining portions of the alkynyl group. In some embodiments, R^{x} is H or an alkyl. In some embodiments, an alkynyl is selected from ethynyl, propynyl, butynyl, pentynyl, hexynyl, and the like. Non-limiting examples of an alkynyl group include —C^{∘}CH, —C≡CCH3, —C≡CCH₂CH, and —CH₂C≡CH.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —OCH₂CH₂OCH₂CH₂OCH₂CH₂O—.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” refers to an aryl group that includes one or more ring heteroatoms selected from nitrogen, oxygen, and sulfur. In some embodiments, heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7, 8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO2H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(Ci-C₄alkyl)₂, —S(═O)₂NH₂, —S(═O)₂NH(Ci-C₄alkyl), —S(═O)₂N(Ci-C₄alkyl)₂, C₁-C₄alkyl, C₃-C₆cycloalkyl, C₁-C₄fluoroalkyl, C₁-C₄heteroalkyl, C₁-C₄alkoxy, Ci-C₄fluoroalkoxy, —SCi-C₄alkyl, —S(═O)C₁-C₄alkyl, and —S(═O)₂Ci-C₄alkyl. In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

### II. Modified IL-18 Polypeptides

Certain aspects of the present disclosure relate to modified IL-18 polypeptides useful as therapeutic agents. The modified IL-18 polypeptides provided herein can be used as immunotherapies or as parts of other immunotherapy regimens. The modified IL-18 polypeptides provided herein exhibit a reduced binding to IL-18 binding protein (IL-18BP) compared to WT IL-18, and/or an improved activity of IL-18 receptor (IL-18R) signaling in the presence of IL-18BP compared to WT IL-18. In certain embodiments, the modified IL-18 polypeptides provided herein exhibit a reduced binding to IL-18 binding protein (IL-18BP) compared to WT IL-18, and an improved activity of IL-18 receptor (IL-18R) signaling in the presence of IL-18BP compared to WT IL-18.

IIa. Site Specific Modification of the Modified IL-18 Polypeptide

In some embodiments, a modified IL-18 polypeptide described herein comprises one or more modifications at one or more amino acid residues. As used herein, “modifications” of the modified IL-18 polypeptide are relative to SEQ ID NO: 1. In some embodiments, the residue position numbering of the modified IL-18 polypeptide is based on SEQ ID NO: 1 as a reference sequence.

Modifications to the polypeptides described herein encompass mutations, addition of various functionalities, deletion of amino acids, addition of amino acids, or any other alteration of the wild-type version of the protein or protein fragment. Functionalities which may be added to polypeptides include polymers, linkers, alkyl groups, detectable molecules such as chromophores or fluorophores, reactive functional groups, or any combination thereof. In some embodiments, functionalities are added to individual amino acids of the polypeptides. In some embodiments, functionalities are added site-specifically to the polypeptides.

In some embodiments, the modified IL-18 polypeptides described herein contain 1, 2, 3, 4, 5, 6, 7, 8, 9, or 10 modified amino acid residues.

In some embodiments, a modified IL-18 polypeptide described herein comprises at least 1, at least 2, at least 3, at least 4, at least 5, at least 6, at least 7, or at least 9 amino acid substitutions, wherein the amino acid substitutions are relative to SEQ ID NO: 1. In some embodiments, the modified IL-18 polypeptide comprises 1 to 9 amino acid substitutions. In some embodiments, the modified IL-18 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the modified IL-18 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the modified IL-18 polypeptide comprises at most 4 amino acid substitutions, 5 amino acid substitutions, 6 amino acid substitutions, 7 amino acid substitutions, or 9 amino acid substitutions.

In some embodiments, a modified IL-18 polypeptide described herein comprises at least 1, at least 2, at least 3, at least 4, at least 5, at least 6, at least 7, or at least 9 additional natural amino acid substitutions, wherein the natural amino acid substitutions are relative to SEQ ID NO: 1. In some embodiments, the modified IL-18 polypeptide comprises 1 to 9 natural amino acid substitutions. In some embodiments, the modified IL-18 polypeptide comprises 1 or 2 natural amino acid substitutions, 1 to 3 natural amino acid substitutions, 1 to 4 natural amino acid substitutions, 1 to 5 natural amino acid substitutions, 1 to 6 natural amino acid substitutions, 1 to 7 natural amino acid substitutions, 1 to 8 natural amino acid substitutions, 2 to 3 natural amino acid substitutions, 2 to 4 natural amino acid substitutions, 2 to 5 natural amino acid substitutions, 2 to 6 natural amino acid substitutions, 2 to 7 natural amino acid substitutions, 2 to 8 natural amino acid substitutions, 2 to 9 natural amino acid substitutions, 3 or 4 natural amino acid substitutions, 3 to 5 natural amino acid substitutions, 3 to 6 natural amino acid substitutions, 3 to 7 natural amino acid substitutions, 3 to 9 natural amino acid substitutions, 4 or 5 natural amino acid substitutions, 4 to 6 natural amino acid substitutions, 4 to 7 amino acid substitutions, 4 to 9 natural amino acid substitutions, 5 or 6 natural amino acid substitutions, 5 to 7 amino acid substitutions, 5 to 9 natural amino acid substitutions, 6 or 7 natural amino acid substitutions, 6 to 9 natural amino acid substitutions, or 7 to 9 natural amino acid substitutions. In some embodiments, the modified IL-18 polypeptide comprises 3 natural amino acid substitutions, 4 natural amino acid substitutions, 5 amino acid substitutions, 6 natural amino acid substitutions, 7 natural amino acid substitutions, or 9 natural amino acid substitutions. In some embodiments, the modified IL-18 polypeptide comprises at most 4 natural amino acid substitutions, 5 natural amino acid substitutions, 6 natural amino acid substitutions, 7 natural amino acid substitutions, or 9 natural amino acid substitutions.

In some embodiments, the modified IL-18 polypeptide further comprises up to 10 non-canonical amino acid substitutions. In some embodiments, the modified IL-18 polypeptide comprises 1, 2, 3, 4, 5, 6, 7, 8, 9 or 10 additional unnatural amino acid substitutions.

In some embodiments, the modified IL-18 polypeptide further comprises unnatural amino acid substitutions at residues M33, M51, N60, M86, M113, and/or M150. In some embodiments, the unnatural amino acid residues substituted for the methionines are each independently norleucine or O-methyl-homoserine. In some embodiments, the modified IL-18 polypeptide further unnatural amino acid substitutions at residues 31, 75, and 116. In some embodiments, the modified IL-18 polypeptide further comprises homoserine (Hse) 31, Hse 75, and Hse 116. In some embodiments, the modified IL-18 polypeptide further unnatural amino acid substitutions at residues 31, 63, and 116. In some embodiments, the modified IL-18 polypeptide further comprises homoserine (Hse) 31, Hse 63, and Hse 116. In some embodiments, the modified IL-18 polypeptide further unnatural amino acid substitutions at residues 31, 63, 75, and 116. In some embodiments, the modified IL-18 polypeptide further comprises homoserine (Hse) 31, Hse 63, Hse 75, and Hse 116. In some embodiments, the modified IL-18 polypeptide further unnatural amino acid substitutions at residues 31, 67, 75, and 116. In some embodiments, the modified IL-18 polypeptide further comprises homoserine (Hse) 31, Hse 67, Hse 75, and Hse 116. In some embodiments, the modified IL-18 polypeptide further unnatural amino acid substitutions at residues 31, 57, 75, and 116. In some embodiments, the modified IL-18 polypeptide further comprises homoserine (Hse) 31, Hse 57, Hse 75, and Hse 116. In some embodiments, the modified IL-18 polypeptide further unnatural amino acid substitutions at residues 31, 50, 75, and 116. In some embodiments, the modified IL-18 polypeptide further comprises homoserine (Hse) 31, Hse 50, Hse 75, and Hse 116. In some embodiments, the modified IL-18 polypeptide further unnatural amino acid substitutions at residues 31, 50, 75, and 121. In some embodiments, the modified IL-18 polypeptide further comprises homoserine (Hse) 31, Hse 50, Hse 75, and Hse 121.

In certain embodiments, the modified IL-18 polypeptide comprises at least one glycine residue attached to the N-terminus of the IL-18 polypeptide. In certain embodiments, the modified IL-18 polypeptide comprises a chain of glycine residues attached to the N-terminus of the polypeptide, wherein the chain of glycine residues comprises 1 to 15 glycine residues. In certain embodiments, the modified IL-18 polypeptide comprises a chain of 1 to 10 glycine residues attached to the N-terminus of the IL-18 polypeptide. In certain embodiments, the modified IL-18 polypeptide comprises a chain of 1, 2, 3, 4, 5, 6, 7, 8, 9, or 10, or any range therebetween glycine residues attached to the N-terminus of the IL-18 polypeptide. In certain embodiments, the modified IL-18 polypeptide comprises a glycine residue attached to the N-terminus of the IL-18 polypeptide. In certain embodiments, the modified IL-18 polypeptide comprises a chain of 2 glycine residues attached to the N-terminus of the IL-18 polypeptide. In certain embodiments, the modified IL-18 polypeptide comprises a chain of 3 glycine residues attached to the N-terminus of the IL-18 polypeptide. In certain embodiments, the modified IL-18 polypeptide comprises a chain of 4 glycine residues attached to the N-terminus of the IL-18 polypeptide. In certain embodiments, the modified IL-18 polypeptide comprises a chain of 5 glycine residues attached to the N-terminus of the IL-18 polypeptide. In certain embodiments, the modified IL-18 polypeptide comprises a chain of glycine residues attached to the N-terminus of the polypeptide, wherein the chain of glycine residues comprises 1 to 2, 1 to 3, 1 to 4, 1 to 5, 1 to 6, 1 to 7, 1 to 8, 1 to 9, 1 to 10, 1 to 12, 1 to 15, 2 to 3, 2 to 4, 2 to 5, 2 to 6, 2 to 7, 2 to 8, 2 to 9, 2 to 10, 2 to 12, 2 to 15, 3 to 4, 3 to 5, 3 to 6, 3 to 7, 3 to 8, 3 to 9, 3 to 10, 3 to 12, 3 to 15, 4 to 5, 4 to 6, 4 to 7, 4 to 8, 4 to 9, 4 to 10, 4 to 12, 4 to 15, 5 to 6, 5 to 7, 5 to 8, 5 to 9, 5 to 10, 5 to 12, 5 to 15, 6 to 7, 6 to 8, 6 to 9, 6 to 10, 6 to 12, 6 to 15, 7 to 8, 7 to 9, 7 to 10, 7 to 12, 7 to 15, 8 to 9, 8 to 10, 8 to 12, 8 to 15, 9 to 10, 9 to 12, 9 to 15, 10 to 12, 10 to 15, or 12 to 15 glycine residues. In certain embodiments, the modified IL-18 polypeptide comprises a chain of glycine residues attached to the N-terminus of the polypeptide, wherein the chain of glycine residues comprises 1, 2, 3, 4, 5, 6, 7, 8, 9, 10, 12, or 15 glycine residues. In certain embodiments, the modified IL-18 polypeptide comprises a chain of glycine residues attached to the N-terminus of the polypeptide, wherein the chain of glycine residues comprises at least 1, 2, 3, 4, 5, 6, 7, 8, 9, 10, or 12 glycine residues. In certain embodiments, the modified IL-18 polypeptide comprises a chain of glycine residues attached to the N-terminus of the polypeptide, wherein the chain of glycine residues comprises at most 2, 3, 4, 5, 6, 7, 8, 9, 10, 12, or 15 glycine residues.

In certain embodiments, the modified IL-18 polypeptide comprises a substitution at residue Y1. In certain embodiments, the modified IL-18 polypeptide can comprises Y1M substitution. Unless specifically mentioned otherwise, the residue position numbering is provided in this paragraph, and elsewhere in this disclosure is based on SEQ ID NO: 1, as a reference sequence. Unless specifically mentioned otherwise, the amino acid substitutions provided in this paragraph, and elsewhere in this disclosure is with respect to SEQ ID NO: 1, as a reference sequence. In certain embodiments, the modified IL-18 polypeptide comprises a substitution at residue F2. In certain embodiments, the modified IL-18 polypeptide can comprises F2A substitution. In certain embodiments, the modified IL-18 polypeptide comprises a substitution at residue E6. In certain embodiments, the modified IL-18 polypeptide comprises E6K substitution. In certain embodiments, the modified IL-18 polypeptide comprises E6R substitution. In certain embodiments, the modified IL-18 polypeptide comprises a substitution at residue K8. In certain embodiments, the modified IL-18 polypeptide comprises K8L substitution. In certain embodiments, the modified IL-18 polypeptide comprises K8E substitution. In certain embodiments, the modified IL-18 polypeptide comprises K8R substitution. In certain embodiments, the modified IL-18 polypeptide comprises a substitution at residue V11. In certain embodiments, the modified IL-18 polypeptide can comprises V11I substitution. In certain embodiments, the modified IL-18 polypeptide comprises a substitution at residue E31. In certain embodiments, the modified IL-18 polypeptide comprises E31A substitution. In certain embodiments, the modified IL-18 polypeptide comprises a substitution at residue T34. In certain embodiments, the modified IL-18 polypeptide comprises T34A substitution. In certain embodiments, the modified IL-18 polypeptide comprises a substitution at residue D35. In certain embodiments, the modified IL-18 polypeptide comprises D35A substitution. In certain embodiments, the modified IL-18 polypeptide comprises a substitution at residue S36. In certain embodiments, the modified IL-18 polypeptide comprises S36A substitution. In certain embodiments, the modified IL-18 polypeptide comprises a substitution at residue D37. In certain embodiments, the modified IL-18 polypeptide comprises D37A substitution. In certain embodiments, the modified IL-18 polypeptide comprises a substitution at residue D40. In certain embodiments, the modified IL-18 polypeptide comprises D40A substitution. In certain embodiments, the modified IL-18 polypeptide comprises a substitution at residue N41. In certain embodiments, the modified IL-18 polypeptide comprises N41A substitution. In certain embodiments, the modified IL-18 polypeptide comprises a substitution at residue I49. In certain embodiments, the modified IL-18 polypeptide comprises I49E substitution. In certain embodiments, the modified IL-18 polypeptide comprises I49M substitution. In certain embodiments, the modified IL-18 polypeptide comprises I49R substitution. In certain embodiments, the modified IL-18 polypeptide comprises a substitution at residue K53. In certain embodiments, the modified IL-18 polypeptide comprises K53A substitution. In certain embodiments, the modified IL-18 polypeptide comprises a substitution at residue D54. In certain embodiments, the modified IL-18 polypeptide comprises D54A substitution. In certain embodiments, the modified IL-18 polypeptide comprises a substitution at residue S55. In certain embodiments, the modified IL-18 polypeptide comprises S55A substitution. In certain embodiments, the modified IL-18 polypeptide comprises S55T substitution. In certain embodiments, the modified IL-18 polypeptide comprises S55H substitution. In certain embodiments, the modified IL-18 polypeptide comprises S55R substitution. In certain embodiments, the modified IL-18 polypeptide comprises a substitution at residue T63. In certain embodiments, the modified IL-18 polypeptide comprises T63A substitution. In certain embodiments, the modified IL-18 polypeptide comprises a substitution at residue Q103. In certain embodiments, the modified IL-18 polypeptide comprises Q103R substitution. In certain embodiments, the modified IL-18 polypeptide comprises Q103E substitution. In certain embodiments, the modified IL-18 polypeptide comprises Q103K substitution. In certain embodiments, the modified IL-18 polypeptide comprises a substitution at residue G108. In certain embodiments, the modified IL-18 polypeptide comprises G108A substitution. In certain embodiments, the modified IL-18 polypeptide comprises a substitution at residue H109. In certain embodiments, the modified IL-18 polypeptide comprises H109A substitution. In certain embodiments, the modified IL-18 polypeptide comprises a substitution at residue D110. In certain embodiments, the modified IL-18 polypeptide comprises D110A substitution. In certain embodiments, the modified IL-18 polypeptide comprises a substitution at residue D132. In certain embodiments, the modified IL-18 polypeptide comprises D132A substitution. In certain embodiments, the modified IL-18 polypeptide comprises a substitution at residue V153. In certain embodiments, the modified IL-18 polypeptide comprises V153R substitution. In certain embodiments, the modified IL-18 polypeptide comprises V153E substitution. In certain embodiments, the modified IL-18 polypeptide comprises V153Y substitution. In certain embodiments, the modified IL-18 polypeptide comprises a substitution at residue C38. In certain embodiments, the modified IL-18 polypeptide comprises C38A substitution. In certain embodiments, the modified IL-18 polypeptide comprises C38S substitution. In certain embodiments, the modified IL-18 polypeptide comprises a substitution at residue C68. In certain embodiments, the modified IL-18 polypeptide comprises C68A substitution. In certain embodiments, the modified IL-18 polypeptide comprises C68S substitution. In certain embodiments, the modified IL-18 polypeptide comprises a substitution at residue C76. In certain embodiments, the modified IL-18 polypeptide comprises C76A substitution. In certain embodiments, the modified IL-18 polypeptide comprises C76S substitution. In certain embodiments, the modified IL-18 polypeptide comprises a substitution at residue C127. In certain embodiments, the modified IL-18 polypeptide comprises C127A substitution. In certain embodiments, the modified IL-18 polypeptide comprises C127S substitution. In certain embodiments, the modified IL-18 polypeptide comprises a substitution at residue C38, C68, C76, and/or C127. In certain embodiments, the modified IL-18 polypeptide comprises a C38A, C38S, C68A, C68S, C76A, C76S, C127A, and/or C127S substitution. In certain embodiments, the modified IL-18 polypeptide comprises C38A, C76A, and C127A substitutions. In certain embodiments, the modified IL-18 polypeptide comprises C38S, C76S and C127S substitutions.

In certain embodiments, the modified IL-18 polypeptide comprises i) a substitution at residue V11 and ii) at least one additional substitution at a residue selected from Y1, F2, E6, K8, S10, D17, T34, D35, S36, D37, D40, N41, 149, M51, K53, D54, S55, Q103, S105, G108, H109, D110, and D132. In certain embodiments, the modified IL-18 polypeptide comprises i) a substitution at residue V11 and ii) at least one additional substitution at a residue selected from Y1, F2, E6, K8, S10, D17, T34, D35, S36, D37, D40, N41, 149, M51, K53, D54, S55, Q103, S105, G108, H109, D110, D132 and V153. In certain embodiments, the modified IL-18 polypeptide comprises i) V11I substitution and ii) at least one additional substitution selected from Y1M, F2A, E6K, E6R, K8L, K8E, K8R, T34A, D35A, S36A, D37A, D40A, N41A, I49E, I49M, I49R, M51G, K53A, D54A, S55A, S55T, S55H, S55R, S55H, Q103R, Q103E, Q103K, S105K, S105I, G108A, H109A, D110A, and D132A. In certain embodiments, the modified IL-18 polypeptide comprises i) V11I substitution and ii) at least one additional substitution selected from Y1M, F2A, E6K, E6R, K8L, K8E, K8R, T34A, D35A, S36A, D37A, D40A, N41A, I49E, I49M, I49R, M51G, K53A, D54A, S55A, S55T, S55H, S55R, S55H, Q103R, Q103E, Q103K, S105K, S105I, G108A, H109A, D110A, D132A, V153R, V153E and V153Y. In certain embodiments, the modified IL-18 polypeptide comprises i) substitution at residue V11, and ii) substitution at residue E6, M51 and/or K53. In certain embodiments, the modified IL-18 polypeptide comprises i) V11I substitution, and ii) E6K, M51G, and/or K53A substitution. In certain embodiments, the modified IL-18 polypeptide comprises substitutions at residues E6, V11, K53 and T63. In certain embodiments, the modified IL-18 polypeptide comprises E6K, V11I, K53A and T63A substitutions. In certain embodiments, the modified IL-18 polypeptide comprises substitutions at residues V11, M51, K53. In certain embodiments, the modified IL-18 polypeptide comprises V11I, M51G, and K53A substitutions. In certain embodiments, the modified IL-18 polypeptide comprises substitutions at residues E6, V11, M51, K53 and T63. In certain embodiments, the modified IL-18 polypeptide comprises E6K, V11I, M51G, K53A and T63A substitutions. In certain embodiments, the modified IL-18 polypeptide comprises substitutions at residues E6, V11, C38, K53, T63, C76, and C127. In certain embodiments, the modified Il-18 polypeptide comprises E6K, V11I, C38A, K53A, T63A, C76A, and C127A substitutions. In certain embodiments, the modified IL-18 polypeptide comprises substitutions at residues V11, C38, M51, K53, T63, C76, and C127. In certain embodiments, the modified Il-18 polypeptide comprises V11I, C38A, M51G, K53A, T63A, C76A, and C127A substitutions. In certain embodiments, the modified IL-18 polypeptide comprises substitutions at residues V11, C38, M51, K53, C76, and C127. In certain embodiments, the modified Il-18 polypeptide comprises V11I, C38A, M51G, K53A, C76A, and C127A substitutions. In certain embodiments, the modified IL-18 polypeptide comprises substitutions at residues E6, V11, C38, M51, K53, T63, C76, and C127. In certain embodiments, the modified IL-18 polypeptide comprises E6K, V11I, C38A, M51G, K53A, T63A, C76A, and C127A substitutions. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) substitutions at residues E6, V11, C38, K53, T63, C76, and C127. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) E6K, V11I, C38A, K53A, T63A, C76A, and C127A substitutions. In certain embodiments, the modified IL-18 polypeptide comprises i) a glycine residue attached to the N-terminus of the IL-18 polypeptide, and ii) substitutions at residues E6, V11, C38, K53, T63, C76, and C127. In certain embodiments, the modified 11-18 polypeptide comprises i) a glycine residue attached to the N-terminus of the IL-18 polypeptide, and ii) E6K, V11I, C38A, K53A, T63A, C76A, and C127A substitutions. In certain embodiments, the modified IL-18 polypeptide comprises i) a chain of 4 glycine residues attached to the N-terminus of the IL-18 polypeptide, and ii) substitutions at residues E6, V11, C38, K53, T63, C76, and C127. In certain embodiments, the modified IL-18 polypeptide comprises i) a chain of 4 glycine residues attached to the N-terminus of the IL-18 polypeptide, and ii) E6K, V11I, C38A, K53A, T63A, C76A, and C127A substitutions. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) substitutions at residues E6, V11, C38, M51, K53, T63, C76, and C127. In certain embodiments, the modified IL-18 polypeptide comprises i) a chain of 1, 2, 3, 4, 5, 6, 7, 8, 9, or 10, or any range therebetween glycine residues attached to the N-terminus of the IL-18 polypeptide, and ii) E6K, V11I, C38A, M51G, K53A, T63A, C76A, and C127A substitutions. In certain embodiments, the modified IL-18 polypeptide comprises i) a glycine residue attached to the N-terminus of the IL-18 polypeptide, and ii) substitutions at residues E6, V11, C38, M51, K53, T63, C76, and C127. In certain embodiments, the modified Il-18 polypeptide comprises i) a glycine residue attached to the N-terminus of the IL-18 polypeptide, and ii) E6K, V11I, C38A, M51G, K53A, T63A, C76A, and C127A substitutions. In certain embodiments, the modified IL-18 polypeptide comprises substitutions at residues V11, C38, K53, C76, and C127. In certain embodiments, the modified IL-18 polypeptide comprises V11I, C38A, K53A, C76A, and C127A substitutions. In certain embodiments, the modified IL-18 polypeptide comprises substitutions at residues V11, C38, K53, T63, C76, and C127. In certain embodiments, the modified IL-18 polypeptide comprises V11I, C38A, K53A, T63A, C76A, and C127A substitutions. In certain embodiments, the modified IL-18 polypeptide comprises substitutions at residues V11, C38, K53, S55, C76, and C127. In certain embodiments, the modified IL-18 polypeptide comprises V11I, C38A, K53A, S55A, C76A, and C127A substitutions. In certain embodiments, the modified IL-18 polypeptide comprises substitutions at residues V11, C38, K53, M51, C76, and C127. In certain embodiments, the modified IL-18 polypeptide comprises V11I, C38A, K53A, M51G, C76A, and C127A substitutions. In certain embodiments, the modified IL-18 polypeptide comprises substitutions at residues V11, C38, K53, D54, C76, and C127. In certain embodiments, the modified IL-18 polypeptide comprises V11I, C38A, K53A, D54A, C76A, and C127A substitutions. In certain embodiments, the modified IL-18 polypeptide comprises substitutions at residues F2, V11, C38, K53, C76, and C127. In certain embodiments, the modified IL-18 polypeptide comprises F2A, V11I, C38A, K53A, C76A, and C127A substitutions. In certain embodiments, the modified IL-18 polypeptide comprises substitutions at residues V11, E31, C38, K53, C76, and C127. In certain embodiments, the modified IL-18 polypeptide comprises V11I, E31A, C38A, K53A, C76A, and C127A substitutions. In certain embodiments, the modified IL-18 polypeptide comprises substitutions at residues V11, T34, C38, K53, C76, and C127. In certain embodiments, the modified IL-18 polypeptide comprises V11I, T34A, C38A, K53A, C76A, and C127A substitutions. In certain embodiments, the modified IL-18 polypeptide comprises substitutions at residues V11, D35, C38, K53, C76, and C127. In certain embodiments, the modified IL-18 polypeptide comprises V11I, D35A, C38A, K53A, C76A, and C127A substitutions. In certain embodiments, the modified IL-18 polypeptide comprises substitutions at residues V11, S36, C38, K53, C76, and C127. In certain embodiments, the modified IL-18 polypeptide comprises V11I, S36A, C38A, K53A, C76A, and C127A substitutions. In certain embodiments, the modified IL-18 polypeptide comprises substitutions at residues V11, D37, C38, K53, C76, and C127. In certain embodiments, the modified IL-18 polypeptide comprises V11I, D37A, C38A, K53A, C76A, and C127A substitutions. In certain embodiments, the modified IL-18 polypeptide comprises substitutions at residues V11, E31, D37, C38, K53, C76, and C127. In certain embodiments, the modified IL-18 polypeptide comprises V11I, E31A, D37A, C38A, K53A, C76A, and C127A substitutions. In certain embodiments, the modified IL-18 polypeptide comprises substitutions at residues V11, C38, D40, K53, C76, and C127. In certain embodiments, the modified IL-18 polypeptide comprises V11I, C38A, D40A, K53A, C76A, and C127A substitutions. In certain embodiments, the modified IL-18 polypeptide comprises substitutions at residues V11, C38, N41, K53, C76, and C127. In certain embodiments, the modified IL-18 polypeptide comprises V11I, C38A, N41A, K53A, C76A, and C127A substitutions. In certain embodiments, the modified IL-18 polypeptide comprises substitutions at residues V11, C38, K53, C76, D132, and C127. In certain embodiments, the modified IL-18 polypeptide comprises V11I, C38A, K53A, C76A, D132A, and C127A substitutions. In certain embodiments, the modified IL-18 polypeptide comprises substitutions at residues V11, C38, K53, C76, G108, and C127. In certain embodiments, the modified IL-18 polypeptide comprises V11I, C38A, K53A, C76A, G108A, and C127A substitutions. In certain embodiments, the modified IL-18 polypeptide comprises substitutions at residues V11, C38, K53, C76, H109 and C127. In certain embodiments, the modified IL-18 polypeptide comprises V11I, C38A, K53A, C76A, H109A and C127A substitutions. In certain embodiments, the modified IL-18 polypeptide comprises substitutions at residues V11, C38, K53, C76, D110 and C127. In certain embodiments, the modified IL-18 polypeptide comprises V11I, C38A, K53A, C76A, D110A and C127A substitutions. In certain embodiments, the modified IL-18 polypeptide comprises substitutions at residues K8, V11, C38, C76, Q103, and C127. In certain embodiments, the modified IL-18 polypeptide comprises K8R, V11I, C38A, C76A, Q103E and C127A substitutions. In certain embodiments, the modified IL-18 polypeptide comprises K8E, V11I, C38A, C76A, Q103R and C127A substitutions. In certain embodiments, the modified IL-18 polypeptide comprises substitutions at residues V11, C38, C76, Q103, and C127. In certain embodiments, the modified IL-18 polypeptide comprises V11I, C38A, C76A, Q103K and C127A substitutions. In certain embodiments, the modified IL-18 polypeptide comprises substitutions at residues V11, C38, S55, C76, and C127. In certain embodiments, the modified IL-18 polypeptide comprises V11I, C38A, S55H, C76A, and C127A substitutions. In certain embodiments, the modified IL-18 polypeptide comprises V11I, C38A, S55R, C76A, and C127A substitutions. In certain embodiments, the modified IL-18 polypeptide comprises V11I, C38A, S55T, C76A, and C127A substitutions. In certain embodiments, the modified IL-18 polypeptide comprises substitutions at residues V11, C38, C76, S105, and C127. In certain embodiments, the modified IL-18 polypeptide comprises V11I, C38A, C76A, S105I and C127A substitutions. In certain embodiments, the modified IL-18 polypeptide comprises V11I, C38A, C76A, S105K, and C127A substitutions. In certain embodiments, the modified IL-18 polypeptide comprises substitutions at residues E6, V11, C38, K53, T63, C76, and C127. In certain embodiments, the modified IL-18 polypeptide comprises E6K, V11I, C38A, K53A, T63A, C76A, and C127A substitutions. In certain embodiments, the modified IL-18 polypeptide comprises substitutions at residues E6, K8, V11, C38, K53, T63, C76, and C127. In certain embodiments, the modified IL-18 polypeptide comprises E6K, K8L, V11I, C38A, K53A, T63A, C76A, and C127A substitutions. In certain embodiments, the modified IL-18 polypeptide comprises substitutions at residues E6, V11, C38, I49, K53, T63, C76, and C127. In certain embodiments, the modified IL-18 polypeptide comprises E6K, V11I, C38A, I49E, K53A, T63A, C76A, and C127A substitutions. In certain embodiments, the modified IL-18 polypeptide comprises E6K, V11I, C38A, I49M, K53A, T63A, C76A, and C127A substitutions. In certain embodiments, the modified IL-18 polypeptide comprises E6K, V11I, C38A, I49R, K53A, T63A, C76A, and C127A substitutions. In certain embodiments, the modified IL-18 polypeptide comprises substitutions at residues E6, V11, C38, K53, T63, C76, Q103, and C127. In certain embodiments, the modified IL-18 polypeptide comprises E6K, V11I, C38A, K53A, T63A, C76A, Q103R, and C127A substitutions. In certain embodiments, the modified IL-18 polypeptide comprises substitutions at residues E6, K8, V11, C38, K53, T63, C76, Q103, and C127. In certain embodiments, the modified IL-18 polypeptide comprises E6K, K8E, V11I, C38A, K53A, T63A, C76A, Q103R, and C127A substitutions. In certain embodiments, the modified IL-18 polypeptide comprises substitutions at residues E6, V11, C38, K53, T63, C76, V153, and C127. In some embodiments, the modified IL-18 polypeptide comprises at least seven modifications to the sequence of SEQ ID NO: 1, wherein the seven modifications comprise E6K, V11, C38A, K53A, T63A, C76A, and C127A.

In certain embodiments, the modified IL-18 polypeptide comprises E6K, V11I, C38A, K53A, T63A, C76A, V153R and C127A substitutions. In certain embodiments, the modified IL-18 polypeptide comprises E6K, V11I, C38A, K53A, T63A, C76A, V153E and C127A substitutions. In certain embodiments, the modified IL-18 polypeptide comprises E6K, V11I, C38A, K53A, T63A, C76A, V153Y, and C127A substitutions. In certain embodiments, the modified IL-18 polypeptide comprises substitutions at residues E6, V11, C38, M51, K53, T63, C76, and C127. In certain embodiments, the modified IL-18 polypeptide comprises E6K, V11I, C38A, M51G, K53A, T63A, C76A, and C127A substitutions. In certain embodiments, the modified IL-18 polypeptide comprises E6R, V11I, C38A, K53A, T63A, C76A, and C127A substitutions. In certain embodiments, the modified IL-18 polypeptide comprises substitutions at residues Y1, E6, V11, C38, K53, T63, C76, and C127. In certain embodiments, the modified IL-18 polypeptide comprises Y1M, E6K, V11, C38A, K53A, T63A, C76A, and C127A substitutions.

In one aspect, provided herein, is a modified IL-18 polypeptide, comprising a polymer covalently attached to residue 68 of the IL-18 polypeptide, and further comprising E6K, V11I, C38A, K53A, T63A, C76A, and C127A amino acid substitutions, wherein residue position numbering is based on SEQ ID NO: 1 as a reference sequence. In some embodiments, the modified IL-18 polypeptide has an amino acid sequence at least 80%, at least 85%, at least 90%, at last 91%, at least 92%, at least 93%, at least 94%, or at least 95% identical to the sequence set forth in SEQ ID NO: 1. In some embodiments, the modified IL-18 polypeptide comprises the sequence set forth in SEQ ID NO: 30.

In certain embodiments, the modified IL-18 polypeptide comprises an amino acid sequence having at least 80%, 85%, 90%, 91%, 92%, 93%, 94% or 95% sequence identity to the sequence set forth in SEQ ID NO: 1. In certain embodiments, the modified IL-18 polypeptide comprises an amino acid sequence having at least 80% sequence identity to the sequence set forth in SEQ ID NO: 1. In certain embodiments, the modified IL-18 polypeptide comprises an amino acid sequence having at least 85% sequence identity to the sequence set forth in SEQ ID NO: 1. In certain embodiments, the modified IL-18 polypeptide comprises an amino acid sequence having at least 90% sequence identity to the sequence set forth in SEQ ID NO: 1. In certain embodiments, the modified IL-18 polypeptide comprises an amino acid sequence having at least 91% sequence identity to the sequence set forth in SEQ ID NO: 1. In certain embodiments, the modified IL-18 polypeptide comprises an amino acid sequence having at least 92% sequence identity to the sequence set forth in SEQ ID NO: 1. In certain embodiments, the modified IL-18 polypeptide comprises an amino acid sequence having at least 93% sequence identity to the sequence set forth in SEQ ID NO: 1. In certain embodiments, the modified IL-18 polypeptide comprises an amino acid sequence having at least 94% sequence identity to the sequence set forth in SEQ ID NO: 1. In certain embodiments, the modified IL-18 polypeptide comprises an amino acid sequence having at least 95% sequence identity to the sequence set forth in SEQ ID NO: 1. In certain embodiments, the modified IL-18 polypeptide comprises an amino acid sequence having at least 80%, 85%, 90%, 95%, 98%, 99%, or 100% sequence identity to the sequence set forth in SEQ ID NO: 2. In certain embodiments, the modified IL-18 polypeptide comprises the amino acid sequence set forth in SEQ ID NO: 2. In certain embodiments, the modified IL-18 polypeptide comprises an amino acid sequence having at least 80%, 85%, 90%, 95%, 98%, 99%, or 100% sequence identity to the sequence set forth in SEQ ID NO: 3. In certain embodiments, the modified IL-18 polypeptide comprises the amino acid sequence set forth in SEQ ID NO: 3. In certain embodiments, the modified IL-18 polypeptide comprises an amino acid sequence having at least 80%, 85%, 90%, 95%, 98%, 99%, or 100% sequence identity to the sequence set forth in SEQ ID NO: 4. In certain embodiments, the modified IL-18 polypeptide comprises the amino acid sequence set forth in SEQ ID NO: 4. In certain embodiments, the modified IL-18 polypeptide comprises an amino acid sequence having at least 80%, 85%, 90%, 95%, 98%, 99%, or 100% sequence identity to the sequence set forth in SEQ ID NO: 5. In certain embodiments, the modified IL-18 polypeptide comprises the amino acid sequence set forth in SEQ ID NO: 5. In certain embodiments, the modified IL-18 polypeptide comprises an amino acid sequence having at least 80%, 85%, 90%, 95%, 98%, 99%, or 100% sequence identity to the sequence set forth in SEQ ID NO: 6. In certain embodiments, the modified IL-18 polypeptide comprises the amino acid sequence set forth in SEQ ID NO: 6. In certain embodiments, the modified IL-18 polypeptide comprises an amino acid sequence having at least 80%, 85%, 90%, 95%, 98%, 99%, or 100% sequence identity to the sequence set forth in SEQ ID NO: 7. In certain embodiments, the modified IL-18 polypeptide comprises the amino acid sequence set forth in SEQ ID NO: 7. In certain embodiments, the modified IL-18 polypeptide comprises an amino acid sequence having at least 80%, 85%, 90%, 95%, 98%, 99%, or 100% sequence identity to the sequence set forth in SEQ ID NO: 8. In certain embodiments, the modified IL-18 polypeptide comprises the amino acid sequence set forth in SEQ ID NO: 8. In certain embodiments, the modified IL-18 polypeptide comprises an amino acid sequence having at least 80%, 85%, 90%, 95%, 98%, 99%, or 100% sequence identity to the sequence set forth in SEQ ID NO: 9. In certain embodiments, the modified IL-18 polypeptide comprises the amino acid sequence set forth in SEQ ID NO: 9. In certain embodiments, the modified IL-18 polypeptide comprises an amino acid sequence having at least 80%, 85%, 90%, 95%, 98%, 99%, or 100% sequence identity to the sequence set forth in SEQ ID NO: 10. In certain embodiments, the modified IL-18 polypeptide comprises the amino acid sequence set forth in SEQ ID NO: 10. In certain embodiments, the modified IL-18 polypeptide comprises an amino acid sequence having at least 80%, 85%, 90%, 95%, 98%, 99%, or 100% sequence identity to the sequence set forth in SEQ ID NO: 11. In certain embodiments, the modified IL-18 polypeptide comprises the amino acid sequence set forth in SEQ ID NO: 11. In certain embodiments, the modified IL-18 polypeptide comprises an amino acid sequence having at least 80%, 85%, 90%, 95%, 98%, 99%, or 100% sequence identity to the sequence set forth in SEQ ID NO: 12. In certain embodiments, the modified IL-18 polypeptide comprises the amino acid sequence set forth in SEQ ID NO: 12. In certain embodiments, the modified IL-18 polypeptide comprises an amino acid sequence having at least 80%, 85%, 90%, 95%, 98%, 99%, or 100% sequence identity to the sequence set forth in SEQ ID NO: 13. In certain embodiments, the modified IL-18 polypeptide comprises the amino acid sequence set forth in SEQ ID NO: 13. In certain embodiments, the modified IL-18 polypeptide comprises an amino acid sequence having at least 80%, 85%, 90%, 95%, 98%, 99%, or 100% sequence identity to the sequence set forth in SEQ ID NO: 14. In certain embodiments, the modified IL-18 polypeptide comprises the amino acid sequence set forth in SEQ ID NO: 14. In certain embodiments, the modified IL-18 polypeptide comprises an amino acid sequence having at least 80%, 85%, 90%, 95%, 98%, 99%, or 100% sequence identity to the sequence set forth in SEQ ID NO: 15. In certain embodiments, the modified IL-18 polypeptide comprises the amino acid sequence set forth in SEQ ID NO: 15. In certain embodiments, the modified IL-18 polypeptide comprises an amino acid sequence having at least 80%, 85%, 90%, 95%, 98%, 99%, or 100% sequence identity to the sequence set forth in SEQ ID NO: 16. In certain embodiments, the modified IL-18 polypeptide comprises the amino acid sequence set forth in SEQ ID NO: 16. In certain embodiments, the modified IL-18 polypeptide comprises an amino acid sequence having at least 80%, 85%, 90%, 95%, 98%, 99%, or 100% sequence identity to the sequence set forth in SEQ ID NO: 17. In certain embodiments, the modified IL-18 polypeptide comprises the amino acid sequence set forth in SEQ ID NO: 17. In certain embodiments, the modified IL-18 polypeptide comprises an amino acid sequence having at least 80%, 85%, 90%, 95%, 98%, 99%, or 100% sequence identity to the sequence set forth in SEQ ID NO: 18. In certain embodiments, the modified IL-18 polypeptide comprises the amino acid sequence set forth in SEQ ID NO: 18. In certain embodiments, the modified IL-18 polypeptide comprises an amino acid sequence having at least 80%, 85%, 90%, 95%, 98%, 99%, or 100% sequence identity to the sequence set forth in SEQ ID NO: 19. In certain embodiments, the modified IL-18 polypeptide comprises the amino acid sequence set forth in SEQ ID NO: 19. In certain embodiments, the modified IL-18 polypeptide comprises an amino acid sequence having at least 80%, 85%, 90%, 95%, 98%, 99%, or 100% sequence identity to the sequence set forth in SEQ ID NO: 20. In certain embodiments, the modified IL-18 polypeptide comprises the amino acid sequence set forth in SEQ ID NO: 20. In certain embodiments, the modified IL-18 polypeptide comprises an amino acid sequence having at least 80%, 85%, 90%, 95%, 98%, 99%, or 100% sequence identity to the sequence set forth in SEQ ID NO: 21. In certain embodiments, the modified IL-18 polypeptide comprises the amino acid sequence set forth in SEQ ID NO: 21. In certain embodiments, the modified IL-18 polypeptide comprises an amino acid sequence having at least 80%, 85%, 90%, 95%, 98%, 99%, or 100% sequence identity to the sequence set forth in SEQ ID NO: 22. In certain embodiments, the modified IL-18 polypeptide comprises the amino acid sequence set forth in SEQ ID NO: 22. In certain embodiments, the modified IL-18 polypeptide comprises an amino acid sequence having at least 80%, 85%, 90%, 95%, 98%, 99%, or 100% sequence identity to the sequence set forth in SEQ ID NO: 23. In certain embodiments, the modified IL-18 polypeptide comprises the amino acid sequence set forth in SEQ ID NO: 23. In certain embodiments, the modified IL-18 polypeptide comprises an amino acid sequence having at least 80%, 85%, 90%, 95%, 98%, 99%, or 100% sequence identity to the sequence set forth in SEQ ID NO: 24. In certain embodiments, the modified IL-18 polypeptide comprises the amino acid sequence set forth in SEQ ID NO: 24. In certain embodiments, the modified IL-18 polypeptide comprises an amino acid sequence having at least 80%, 85%, 90%, 95%, 98%, 99%, or 100% sequence identity to the sequence set forth in SEQ ID NO: 25. In certain embodiments, the modified IL-18 polypeptide comprises the amino acid sequence set forth in SEQ ID NO: 25. In certain embodiments, the modified IL-18 polypeptide comprises an amino acid sequence having at least 80%, 85%, 90%, 95%, 98%, 99%, or 100% sequence identity to the sequence set forth in SEQ ID NO: 26. In certain embodiments, the modified IL-18 polypeptide comprises the amino acid sequence set forth in SEQ ID NO: 26. In certain embodiments, the modified IL-18 polypeptide comprises an amino acid sequence having at least 80%, 85%, 90%, 95%, 98%, 99%, or 100% sequence identity to the sequence set forth in SEQ ID NO: 27. In certain embodiments, the modified IL-18 polypeptide comprises the amino acid sequence set forth in SEQ ID NO: 27. In certain embodiments, the modified IL-18 polypeptide comprises an amino acid sequence having at least 80%, 85%, 90%, 95%, 98%, 99%, or 100% sequence identity to the sequence set forth in SEQ ID NO: 28. In certain embodiments, the modified IL-18 polypeptide comprises the amino acid sequence set forth in SEQ ID NO: 28. In certain embodiments, the modified IL-18 polypeptide comprises an amino acid sequence having at least 80%, 85%, 90%, 95%, 98%, 99%, or 100% sequence identity to the sequence set forth in SEQ ID NO: 29. In certain embodiments, the modified IL-18 polypeptide comprises the amino acid sequence set forth in SEQ ID NO: 29. In certain embodiments, the modified IL-18 polypeptide comprises an amino acid sequence having at least 80%, 85%, 90%, 95%, 98%, 99%, or 100% sequence identity to the sequence set forth in SEQ ID NO: 30. In certain embodiments, the modified IL-18 polypeptide comprises an amino acid sequence having at least 80% sequence identity to the sequence set forth in SEQ ID NO: 30. In certain embodiments, the modified IL-18 polypeptide comprises an amino acid sequence having at least 85% sequence identity to the sequence set forth in SEQ ID NO: 30. In certain embodiments, the modified IL-18 polypeptide comprises an amino acid sequence having at least 90% sequence identity to the sequence set forth in SEQ ID NO: 30. In certain embodiments, the modified IL-18 polypeptide comprises an amino acid sequence having at least 95% sequence identity to the sequence set forth in SEQ ID NO: 30. In certain embodiments, the modified IL-18 polypeptide comprises an amino acid sequence having at least 98% sequence identity to the sequence set forth in SEQ ID NO: 30. In certain embodiments, the modified IL-18 polypeptide comprises an amino acid sequence having at least 99% sequence identity to the sequence set forth in SEQ ID NO: 30. In certain embodiments, the modified IL-18 polypeptide comprises the amino acid sequence set forth in SEQ ID NO: 30. In certain embodiments, the modified IL-18 polypeptide comprises an amino acid sequence having at least 80%, 85%, 90%, 95%, 98%, 99%, or 100% sequence identity to the sequence set forth in SEQ ID NO: 31. In certain embodiments, the modified IL-18 polypeptide comprises the amino acid sequence set forth in SEQ ID NO: 31. In certain embodiments, the modified IL-18 polypeptide comprises an amino acid sequence having at least 80%, 85%, 90%, 95%, 98%, 99%, or 100% sequence identity to the sequence set forth in SEQ ID NO: 32. In certain embodiments, the modified IL-18 polypeptide comprises the amino acid sequence set forth in SEQ ID NO: 32. In certain embodiments, the modified IL-18 polypeptide comprises an amino acid sequence having at least 80%, 85%, 90%, 95%, 98%, 99%, or 100% sequence identity to the sequence set forth in SEQ ID NO: 33. In certain embodiments, the modified IL-18 polypeptide comprises the amino acid sequence set forth in SEQ ID NO: 33.

In certain embodiments, the modified IL-18 polypeptide comprises an amino acid sequence having at least 80%, 85%, 90%, 95%, 98%, 99%, or 100% sequence identity to the sequence set forth in SEQ ID NO: 204. In certain embodiments, the modified IL-18 polypeptide comprises the amino acid sequence set forth in SEQ ID NO: 204. In certain embodiments, the modified IL-18 polypeptide comprises an amino acid sequence having at least 80%, 85%, 90%, 95%, 98%, 99%, or 100% sequence identity to the sequence set forth in SEQ ID NO: 205. In certain embodiments, the modified IL-18 polypeptide comprises the amino acid sequence set forth in SEQ ID NO: 205. In certain embodiments, the modified IL-18 polypeptide comprises an amino acid sequence having at least 80%, 85%, 90%, 95%, 98%, 99%, or 100% sequence identity to the sequence set forth in SEQ ID NO: 206. In certain embodiments, the modified IL-18 polypeptide comprises the amino acid sequence set forth in SEQ ID NO: 206. In certain embodiments, the modified IL-18 polypeptide comprises an amino acid sequence having at least 80%, 85%, 90%, 95%, 98%, 99%, or 100% sequence identity to the sequence set forth in SEQ ID NO: 207. In certain embodiments, the modified IL-18 polypeptide comprises an amino acid sequence having at least 80% sequence identity to the sequence set forth in SEQ ID NO: 207. In certain embodiments, the modified IL-18 polypeptide comprises an amino acid sequence having at least 85% sequence identity to the sequence set forth in SEQ ID NO: 207. In certain embodiments, the modified IL-18 polypeptide comprises an amino acid sequence having at least 90% sequence identity to the sequence set forth in SEQ ID NO: 207. In certain embodiments, the modified IL-18 polypeptide comprises an amino acid sequence having at least 95% sequence identity to the sequence set forth in SEQ ID NO: 207. In certain embodiments, the modified IL-18 polypeptide comprises an amino acid sequence having at least 98% sequence identity to the sequence set forth in SEQ ID NO: 207. In certain embodiments, the modified IL-18 polypeptide comprises an amino acid sequence having at least 99% sequence identity to the sequence set forth in SEQ ID NO: 207. In certain embodiments, the modified IL-18 polypeptide comprises the amino acid sequence set forth in SEQ ID NO: 207. In certain embodiments, the modified IL-18 polypeptide comprises an amino acid sequence having at least 80%, 85%, 90%, 95%, 98%, 99%, or 100% sequence identity to the sequence set forth in SEQ ID NO: 208. In certain embodiments, the modified IL-18 polypeptide comprises the amino acid sequence set forth in SEQ ID NO: 208. In certain embodiments, the modified IL-18 polypeptide comprises an amino acid sequence having at least 80%, 85%, 90%, 95%, 98%, 99%, or 100% sequence identity to the sequence set forth in SEQ ID NO: 209. In certain embodiments, the modified IL-18 polypeptide comprises the amino acid sequence set forth in SEQ ID NO: 209. In certain embodiments, the modified IL-18 polypeptide comprises an amino acid sequence having at least 80%, 85%, 90%, 95%, 98%, 99%, or 100% sequence identity to the sequence set forth in SEQ ID NO: 210. In certain embodiments, the modified IL-18 polypeptide comprises the amino acid sequence set forth in SEQ ID NO: 210. In certain embodiments, the modified IL-18 polypeptide comprises an amino acid sequence having at least 80%, 85%, 90%, 95%, 98%, 99%, or 100% sequence identity to the sequence set forth in SEQ ID NO: 211. In certain embodiments, the modified IL-18 polypeptide comprises the amino acid sequence set forth in SEQ ID NO: 211. In certain embodiments, the modified IL-18 polypeptide comprises an amino acid sequence having at least 80%, 85%, 90%, 95%, 98%, 99%, or 100% sequence identity to the sequence set forth in SEQ ID NO: 212. In certain embodiments, the modified IL-18 polypeptide comprises the amino acid sequence set forth in SEQ ID NO: 212. In certain embodiments, the modified IL-18 polypeptide comprises an amino acid sequence having at least 80%, 85%, 90%, 95%, 98%, 99%, or 100% sequence identity to the sequence set forth in SEQ ID NO: 213. In certain embodiments, the modified IL-18 polypeptide comprises the amino acid sequence set forth in SEQ ID NO: 213. In certain embodiments, the modified IL-18 polypeptide comprises an amino acid sequence having at least 80%, 85%, 90%, 95%, 98%, 99%, or 100% sequence identity to the sequence set forth in SEQ ID NO: 214. In certain embodiments, the modified IL-18 polypeptide comprises the amino acid sequence set forth in SEQ ID NO: 214. In certain embodiments, the modified IL-18 polypeptide comprises an amino acid sequence having at least 80%, 85%, 90%, 95%, 98%, 99%, or 100% sequence identity to the sequence set forth in SEQ ID NO: 215. In certain embodiments, the modified IL-18 polypeptide comprises the amino acid sequence set forth in SEQ ID NO: 215. In certain embodiments, the modified IL-18 polypeptide comprises an amino acid sequence having at least 80%, 85%, 90%, 95%, 98%, 99%, or 100% sequence identity to the sequence set forth in SEQ ID NO: 216. In certain embodiments, the modified IL-18 polypeptide comprises the amino acid sequence set forth in SEQ ID NO: 216. In certain embodiments, the modified IL-18 polypeptide comprises an amino acid sequence having at least 80%, 85%, 90%, 95%, 98%, 99%, or 100% sequence identity to the sequence set forth in SEQ ID NO: 217. In certain embodiments, the modified IL-18 polypeptide comprises the amino acid sequence set forth in SEQ ID NO: 217. In certain embodiments, the modified IL-18 polypeptide comprises an amino acid sequence having at least 80%, 85%, 90%, 95%, 98%, 99%, or 100% sequence identity to the sequence set forth in SEQ ID NO: 218. In certain embodiments, the modified IL-18 polypeptide comprises the amino acid sequence set forth in SEQ ID NO: 218. In certain embodiments, the modified IL-18 polypeptide comprises an amino acid sequence having at least 80%, 85%, 90%, 95%, 98%, 99%, or 100% sequence identity to the sequence set forth in SEQ ID NO: 219. In certain embodiments, the modified IL-18 polypeptide comprises the amino acid sequence set forth in SEQ ID NO: 219. In certain embodiments, the modified IL-18 polypeptide comprises an amino acid sequence having at least 80%, 85%, 90%, 95%, 98%, 99%, or 100% sequence identity to the sequence set forth in SEQ ID NO: 220. In certain embodiments, the modified IL-18 polypeptide comprises the amino acid sequence set forth in SEQ ID NO: 220. In certain embodiments, the modified IL-18 polypeptide comprises an amino acid sequence having at least 80%, 85%, 90%, 95%, 98%, 99%, or 100% sequence identity to the sequence set forth in SEQ ID NO: 221. In certain embodiments, the modified IL-18 polypeptide comprises the amino acid sequence set forth in SEQ ID NO: 221. In certain embodiments, the modified IL-18 polypeptide comprises an amino acid sequence having at least 80%, 85%, 90%, 95%, 98%, 99%, or 100% sequence identity to the sequence set forth in SEQ ID NO: 222. In certain embodiments, the modified IL-18 polypeptide comprises the amino acid sequence set forth in SEQ ID NO: 222. In certain embodiments, the modified IL-18 polypeptide comprises an amino acid sequence having at least 80%, 85%, 90%, 95%, 98%, 99%, or 100% sequence identity to the sequence set forth in SEQ ID NO: 223. In certain embodiments, the modified IL-18 polypeptide comprises the amino acid sequence set forth in SEQ ID NO: 223. In certain embodiments, the modified IL-18 polypeptide comprises an amino acid sequence having at least 80%, 85%, 90%, 95%, 98%, 99%, or 100% sequence identity to the sequence set forth in SEQ ID NO: 224. In certain embodiments, the modified IL-18 polypeptide comprises the amino acid sequence set forth in SEQ ID NO: 224. In certain embodiments, the modified IL-18 polypeptide comprises an amino acid sequence having at least 80%, 85%, 90%, 95%, 98%, 99%, or 100% sequence identity to the sequence set forth in SEQ ID NO: 225. In certain embodiments, the modified IL-18 polypeptide comprises the amino acid sequence set forth in SEQ ID NO: 225. In certain embodiments, the modified IL-18 polypeptide comprises an amino acid sequence having at least 80%, 85%, 90%, 95%, 98%, 99%, or 100% sequence identity to the sequence set forth in SEQ ID NO: 226. In certain embodiments, the modified IL-18 polypeptide comprises the amino acid sequence set forth in SEQ ID NO: 226. In certain embodiments, the modified IL-18 polypeptide comprises an amino acid sequence having at least 80%, 85%, 90%, 95%, 98%, 99%, or 100% sequence identity to the sequence set forth in SEQ ID NO: 227. In certain embodiments, the modified IL-18 polypeptide comprises the amino acid sequence set forth in SEQ ID NO: 227. In certain embodiments, the modified IL-18 polypeptide comprises an amino acid sequence having at least 80%, 85%, 90%, 95%, 98%, 99%, or 100% sequence identity to the sequence set forth in SEQ ID NO: 228. In certain embodiments, the modified IL-18 polypeptide comprises the amino acid sequence set forth in SEQ ID NO: 228. In certain embodiments, the modified IL-18 polypeptide comprises an amino acid sequence having at least 80%, 85%, 90%, 95%, 98%, 99%, or 100% sequence identity to the sequence set forth in SEQ ID NO: 229. In certain embodiments, the modified IL-18 polypeptide comprises the amino acid sequence set forth in SEQ ID NO: 229. In certain embodiments, the modified IL-18 polypeptide comprises an amino acid sequence having at least 80%, 85%, 90%, 95%, 98%, 99%, or 100% sequence identity to the sequence set forth in SEQ ID NO: 230. In certain embodiments, the modified IL-18 polypeptide comprises the amino acid sequence set forth in SEQ ID NO: 230. In certain embodiments, the modified IL-18 polypeptide comprises an amino acid sequence having at least 80%, 85%, 90%, 95%, 98%, 99%, or 100% sequence identity to the sequence set forth in SEQ ID NO: 231. In certain embodiments, the modified IL-18 polypeptide comprises the amino acid sequence set forth in SEQ ID NO: 231. In certain embodiments, the modified IL-18 polypeptide comprises an amino acid sequence having at least 80%, 85%, 90%, 95%, 98%, 99%, or 100% sequence identity to the sequence set forth in SEQ ID NO: 232. In certain embodiments, the modified IL-18 polypeptide comprises the amino acid sequence set forth in SEQ ID NO: 232. In certain embodiments, the modified IL-18 polypeptide comprises an amino acid sequence having at least 80%, 85%, 90%, 95%, 98%, 99%, or 100% sequence identity to the sequence set forth in SEQ ID NO: 233. In certain embodiments, the modified IL-18 polypeptide comprises the amino acid sequence set forth in SEQ ID NO: 233. In certain embodiments, the modified IL-18 polypeptide comprises an amino acid sequence having at least 80%, 85%, 90%, 95%, 98%, 99%, or 100% sequence identity to the sequence set forth in SEQ ID NO: 234. In certain embodiments, the modified IL-18 polypeptide comprises the amino acid sequence set forth in SEQ ID NO: 234. In certain embodiments, the modified IL-18 polypeptide comprises an amino acid sequence having at least 80%, 85%, 90%, 95%, 98%, 99%, or 100% sequence identity to the sequence set forth in SEQ ID NO: 235. In certain embodiments, the modified IL-18 polypeptide comprises the amino acid sequence set forth in SEQ ID NO: 235. In certain embodiments, the modified IL-18 polypeptide comprises an amino acid sequence having at least 80%, 85%, 90%, 95%, 98%, 99%, or 100% sequence identity to the sequence set forth in SEQ ID NO: 236. In certain embodiments, the modified IL-18 polypeptide comprises the amino acid sequence set forth in SEQ ID NO: 236. In certain embodiments, the modified IL-18 polypeptide comprises an amino acid sequence having at least 80%, 85%, 90%, 95%, 98%, 99%, or 100% sequence identity to the sequence set forth in SEQ ID NO: 237. In certain embodiments, the modified IL-18 polypeptide comprises the amino acid sequence set forth in SEQ ID NO: 237. In certain embodiments, the modified IL-18 polypeptide comprises an amino acid sequence having at least 80%, 85%, 90%, 95%, 98%, 99%, or 100% sequence identity to the sequence set forth in SEQ ID NO: 238. In certain embodiments, the modified IL-18 polypeptide comprises the amino acid sequence set forth in SEQ ID NO: 238. In certain embodiments, the modified IL-18 polypeptide comprises an amino acid sequence having at least 80%, 85%, 90%, 95%, 98%, 99%, or 100% sequence identity to the sequence set forth in SEQ ID NO: 239. In certain embodiments, the modified IL-18 polypeptide comprises an amino acid sequence having at least 80% sequence identity to the sequence set forth in SEQ ID NO: 239. In certain embodiments, the modified IL-18 polypeptide comprises an amino acid sequence having at least 85% sequence identity to the sequence set forth in SEQ ID NO: 239. In certain embodiments, the modified IL-18 polypeptide comprises an amino acid sequence having at least 90% sequence identity to the sequence set forth in SEQ ID NO: 239. In certain embodiments, the modified IL-18 polypeptide comprises an amino acid sequence having at least 95% sequence identity to the sequence set forth in SEQ ID NO: 239. In certain embodiments, the modified IL-18 polypeptide comprises an amino acid sequence having at least 98% sequence identity to the sequence set forth in SEQ ID NO: 239. In certain embodiments, the modified IL-18 polypeptide comprises an amino acid sequence having at least 99% sequence identity to the sequence set forth in SEQ ID NO: 239. In certain embodiments, the modified IL-18 polypeptide comprises the amino acid sequence set forth in SEQ ID NO: 239. In certain embodiments, the modified IL-18 polypeptide comprises an amino acid sequence having at least 80%, 85%, 90%, 95%, 98%, 99%, or 100% sequence identity to the sequence set forth in SEQ ID NO: 240. In certain embodiments, the modified IL-18 polypeptide comprises the amino acid sequence set forth in SEQ ID NO: 240. In certain embodiments, the modified IL-18 polypeptide comprises an amino acid sequence having at least 80%, 85%, 90%, 95%, 98%, 99%, or 100% sequence identity to the sequence set forth in SEQ ID NO: 241. In certain embodiments, the modified IL-18 polypeptide comprises an amino acid sequence having at least 80% sequence identity to the sequence set forth in SEQ ID NO: 241. In certain embodiments, the modified IL-18 polypeptide comprises an amino acid sequence having at least 85% sequence identity to the sequence set forth in SEQ ID NO: 241. In certain embodiments, the modified IL-18 polypeptide comprises an amino acid sequence having at least 90% sequence identity to the sequence set forth in SEQ ID NO: 241. In certain embodiments, the modified IL-18 polypeptide comprises an amino acid sequence having at least 95% sequence identity to the sequence set forth in SEQ ID NO: 241. In certain embodiments, the modified IL-18 polypeptide comprises an amino acid sequence having at least 98% sequence identity to the sequence set forth in SEQ ID NO: 241. In certain embodiments, the modified IL-18 polypeptide comprises an amino acid sequence having at least 99% sequence identity to the sequence set forth in SEQ ID NO: 241. In certain embodiments, the modified IL-18 polypeptide comprises the amino acid sequence set forth in SEQ ID NO: 241. In certain embodiments, the modified IL-18 polypeptide comprises an amino acid sequence having at least 80%, 85%, 90%, 95%, 98%, 99%, or 100% sequence identity to the sequence set forth in SEQ ID NO: 242. In certain embodiments, the modified IL-18 polypeptide comprises an amino acid sequence having at least 80% sequence identity to the sequence set forth in SEQ ID NO: 242. In certain embodiments, the modified IL-18 polypeptide comprises an amino acid sequence having at least 85% sequence identity to the sequence set forth in SEQ ID NO: 242. In certain embodiments, the modified IL-18 polypeptide comprises an amino acid sequence having at least 90% sequence identity to the sequence set forth in SEQ ID NO: 242. In certain embodiments, the modified IL-18 polypeptide comprises an amino acid sequence having at least 95% sequence identity to the sequence set forth in SEQ ID NO: 242. In certain embodiments, the modified IL-18 polypeptide comprises an amino acid sequence having at least 98% sequence identity to the sequence set forth in SEQ ID NO: 242. In certain embodiments, the modified IL-18 polypeptide comprises an amino acid sequence having at least 99% sequence identity to the sequence set forth in SEQ ID NO: 242. In certain embodiments, the modified IL-18 polypeptide comprises the amino acid sequence set forth in SEQ ID NO: 242. In certain embodiments, the modified IL-18 polypeptide comprises an amino acid sequence having at least 80%, 85%, 90%, 95%, 98%, 99%, or 100% sequence identity to the sequence set forth in SEQ ID NO: 243. In certain embodiments, the modified IL-18 polypeptide comprises the amino acid sequence set forth in SEQ ID NO: 243. In certain embodiments, the modified IL-18 polypeptide comprises an amino acid sequence having at least 80%, 85%, 90%, 95%, 98%, 99%, or 100% sequence identity to the sequence set forth in SEQ ID NO: 244. In certain embodiments, the modified IL-18 polypeptide comprises an amino acid sequence having at least 80% sequence identity to the sequence set forth in SEQ ID NO: 244. In certain embodiments, the modified IL-18 polypeptide comprises an amino acid sequence having at least 85% sequence identity to the sequence set forth in SEQ ID NO: 244. In certain embodiments, the modified IL-18 polypeptide comprises an amino acid sequence having at least 90% sequence identity to the sequence set forth in SEQ ID NO: 244. In certain embodiments, the modified IL-18 polypeptide comprises an amino acid sequence having at least 95% sequence identity to the sequence set forth in SEQ ID NO: 244. In certain embodiments, the modified IL-18 polypeptide comprises an amino acid sequence having at least 98% sequence identity to the sequence set forth in SEQ ID NO: 244. In certain embodiments, the modified IL-18 polypeptide comprises an amino acid sequence having at least 99% sequence identity to the sequence set forth in SEQ ID NO: 244. In certain embodiments, the modified IL-18 polypeptide comprises the amino acid sequence set forth in SEQ ID NO: 244.

In one aspect, provided herein, is a modified interleukin-18 (IL-18) polypeptide, comprising: a polymer attached to any one of residues 68-70, wherein residue position numbering of the modified IL-18 polypeptide is based on SEQ ID NO: 1 as a reference sequence. In one aspect, provided herein, is a modified interleukin-18 (IL-18) polypeptide, comprising: a polymer attached to residue 68, wherein residue position numbering of the modified IL-18 polypeptide is based on SEQ ID NO: 1 as a reference sequence. In some embodiments, the polymer provided herein can be attached to a residue selected from residue 68, 69, or 70. In some embodiments, the polymer is attached to residue 68. In some embodiments, the polymer is attached to residue 68, 69, or 70 of an IL-18 polypeptide having at least about 80%, at least about 85%, at least about 90%, at least about 95%, at least about 96%, at least about 97%, at least about 98%, at least about 99%, or an identical sequence to any one of SEQ ID NOs: 13-33. In some embodiments, the polymer is attached to residue 68, 69, or 70 of an IL-18 polypeptide having at least about 80%, at least about 85%, at least about 90%, at least about 95%, at least about 96%, at least about 97%, at least about 98%, at least about 99%, or an identical sequence to any one of SEQ ID NOs: 24-33. In some embodiments, the residue is attached to residue.

A modified IL-18 polypeptide as described herein can comprise one or more unnatural amino acids. “Unnatural” amino acids can refer to amino acid residues in D- or L-form that are not among the 20 canonical amino acids generally incorporated into naturally occurring proteins. In some embodiments, one or more amino acids of the modified IL-18 polypeptides are substituted with one or more unnatural amino acids. Unnatural amino acids include, but are not limited to L-azidolysine, p-azido-L-phenylalanine, and L-biphenylalanine.

Exemplary unnatural amino acids also include p-acetyl-L-phenylalanine, p-iodo-L-phenylalanine, p-propargyloxyphenylalanine, p-propargyl-phenylalanine, L-3-(2-naphthyl) alanine, 3-methyl-phenylalanine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, p-Boronophenylalanine, p-bromophenylalanine, p-amino-L-phenylalanine, isopropyl-L-phenylalanine, an analogue of a tyrosine amino acid; an analogue of a glutamine amino acid; an analogue of a phenylalanine amino acid; an analogue of a serine amino acid; an analogue of a threonine amino acid; a 3-amino acid; a cyclic amino acid other than proline or histidine; an aromatic amino acid other than phenylalanine, tyrosine or tryptophan; or a combination thereof. In some embodiments, the unnatural amino acids are selected from β-amino acids, homoamino acids, and cyclic amino acids. In some embodiments, the unnatural amino acids comprise β-alanine, β-aminopropionic acid, piperidinic acid, aminocaprioic acid, aminoheptanoic acid, aminopimelic acid, desmosine, diaminopimelic acid, N^{α}-ethylglycine, N^{α}-ethylaspargine, isodesmosine, allo-isoleucine, N^{α}-methylglycine, N^{α}-methylisoleucine, N^{α}-methylvaline, γ-carboxyglutamate, N^{α}-acetylserine, N^{α}-formylmethionine, 3-methylhistidine, and/or other similar amino acids.

In some embodiments, amino acid residues of the modified IL-18 polypeptides are substituted with a conjugation handle. In some embodiments, the amino acid residues comprise an amino, azide, allyl, ester, and/or amide functional groups. In some embodiments, the conjugation handles can serve as useful anchor points to attach additional moieties to the modified IL-18 polypeptides. In some embodiments, the amino acid residues have a structure built from precursors Structure 1, Structure 2, Structure 3, or Structure 4:

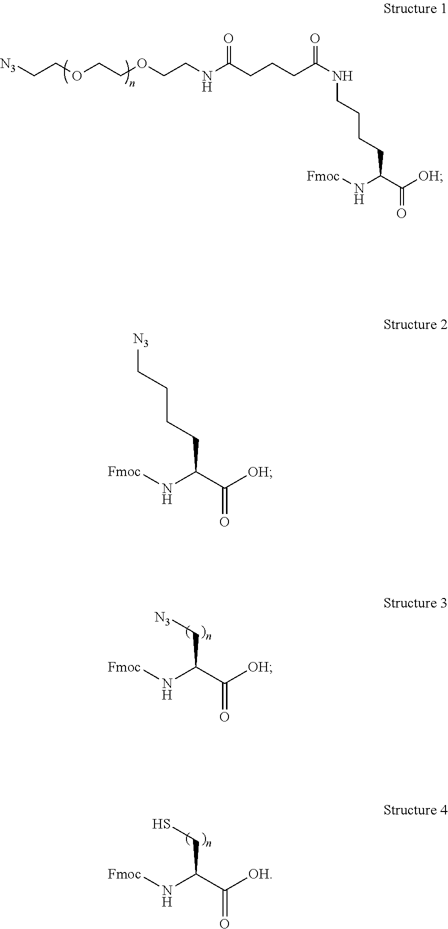

In some embodiments, the modified IL-18 polypeptide contains a substitution for modified natural amino acid residues which can be used for attachment of additional functional groups which can be used to facilitate conjugation reaction or attachment of various payloads to the modified IL-18 polypeptide (e.g., polymers). The substitution can be for a naturally occurring amino acid which is more amenable to attachment of additional functional groups (e.g., aspartic acid/asparagine cysteine, glutamic acid/glutamine, lysine, serine, threonine, or tyrosine), a derivative of a modified version of any naturally occurring amino acid, or any unnatural amino acid (e.g., an amino acid containing a desired reactive group, such as a CLICK chemistry reagent such as an azide, alkyne, etc.). Non-limiting examples of modified natural amino acid residues include the modified lysine, glutamic acid, aspartic acid, and cysteine provided below:

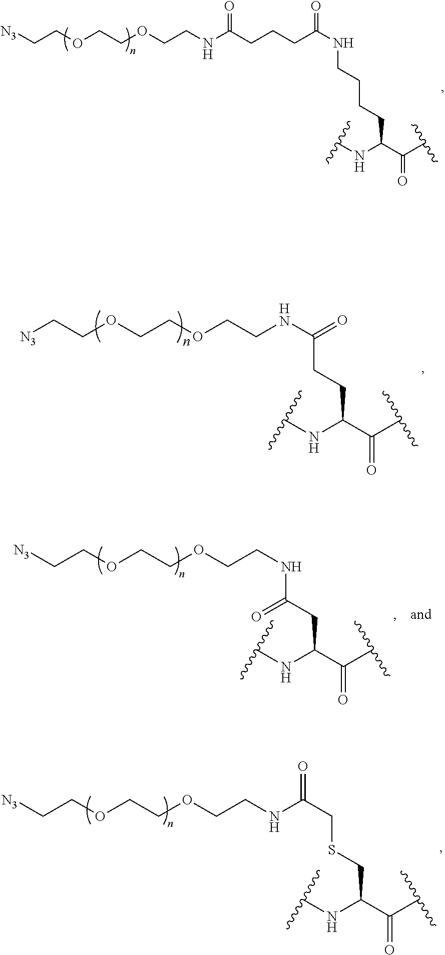

wherein each n is an integer from 1-30. Other examples of natural amino acids which can be similarly modified include those with heteroatoms capable of easily forming a bond with a suitable group to link the polymeric group to the amino acid (e.g., tyrosine, serine, threonine). These non-limiting examples of modified amino acid residues can be used at any location at which it is desirable to add an additional functionality (e.g., a polymer) to the modified IL-18 polypeptide.

In some embodiments, any of structures 1-4, the modified lysine, the modified glutamic acid, the modified aspartic acid, or the modified cysteine provided above can be substituted for a different residue of the modified IL-18 polypeptide to allow for conjugation at a different site of the IL-18 polypeptide. The azide functionality may also be replaced with another suitable conjugation handle. The conjugation handles provided herein can be any suitable reactive group capable of reacting with a complementary reactive group. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cyclooctenes, or a metal-mediated process such as olefin metathesis and Suzuki-Miyaura, 0-substituted hydroxylamine, potassium acyltrifluoroborate or Sonogashira cross-coupling.

In some embodiments, the conjugation handle comprises a reagent for a “copper-free” alkyne azide triazole-forming reaction. Non-limiting examples of alkynes for said alkyne azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof).

In some embodiments, the conjugation handle comprises a reactive group selected from azide, alkyne, tetrazine, halide, sulfhydryl, disulfide, maleimide, activated ester, alkene, aldehyde, ketone, imine, hydrazine, acyltrifluoroborate, hydroxylamine (e.g., 0-substituted hydroxylamine), phosphine, trans-cyclooctene, and hydrazide. In some embodiments, the conjugation handle and complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research, volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; U.S. Ser. No. 10/266,502B2; and US20190204330A1, each of which is incorporated by reference in its entirety.

In some embodiments, a group attached to the modified IL-18 polypeptide (e.g., a polymer moiety or an additional polypeptide) comprises a conjugation handle or a reaction product of a conjugation handle with a complementary conjugation handle. In some embodiments, the reaction product of the conjugation handle with the complementary conjugation handle results from a KAT ligation (reaction of potassium acyltrifluoroborate with hydroxylamine), a Staudinger ligation (reaction of an azide with a phosphine), a tetrazine cycloaddition (reaction of a tetrazine with a trans-cyclooctene), or a Huisgen cycloaddition (reaction of an alkyne with an azide). In some embodiments, the group attached to the IL-18 polypeptide (e.g., the polymer or the additional polypeptide) will comprise a reaction product of a conjugation handle with a complementary conjugation handle which was used to attach the group to the modified IL-18 polypeptide.

### III. Polymer Modified IL-18 Polypeptides

Certain aspects of the present disclosure relate to polymer modified IL-18 polypeptides useful as therapeutic agents. Polymer modified IL-18 polypeptides provided herein can be used as immunotherapies or as parts of other immunotherapy regimens. In some embodiments, the polymer modified IL-18 polypeptides provided herein exhibit an improved serum half-life compared to wild type IL-18 (WT IL-18), reduced binding to IL-18 binding protein (IL-18BP) compared to WT IL-18, and/or an improved activity of IL-18 receptor (IL-18R) signaling in the presence of IL-18BP compared to WT IL-18.

In one aspect, provided herein are polymer modified IL-18 polypeptides comprising a polymer covalently attached at a residue which has a minimal impact on the binding of the modified IL-18 polypeptide for the IL-18 receptor (IL-18R). In some embodiments, addition of the polymer to the modified IL-18 polypeptide results in a polymer modified IL-18 polypeptide with no more than 10-fold reduced binding affinity to IL-18R as compared to the modified IL-18 polypeptide without the polymer (e.g., at most 10-fold, 9-fold, 8-fold, 7-fold, 6-fold, 5-fold, 4-fold, 3-fold, 2-fold reduced affinity or substantially the same affinity). In some embodiments, the polymer modified IL-18 polypeptide with the polymer attached displays an enhanced binding affinity to IL-18R as compared do WT IL-18, or an only slightly reduced affinity to IL-18R as compared to WT IL-18. In some embodiments, the polymer modified IL-18 polypeptides have an increased affinity for the IL-18Rα/β heterodimer compared to WT IL-18. In one aspect, the polymer modified IL-18 polypeptides described herein have no significant decrease in affinity for the IL-18Rα/β heterodimer compared to WT IL-18.

Such polymer modified IL-18 polypeptides display may display binding characteristics for IL-18 receptor subunits that differ from wild-type IL-18 (e.g., a higher affinity or modestly lower affinity for the IL-18 receptor alpha subunit (IL-18Rα) or the IL-18 receptor beta subunit (IL-18RP)). In some embodiments, the binding affinity between the polymer modified IL-18 polypeptides and IL-18Rα is the same as or higher than the binding affinity between a wild-type IL-18 and IL-18Rα. In some embodiments, the binding affinity between the polymer modified IL-18 polypeptides and IL-18Rα is the same as or only moderately lower than the binding affinity between a wild type IL-18 and IL-18Rα)). In some embodiments, the binding affinity between the polymer modified IL-18 polypeptides and IL-18RP is the same as or higher than the binding affinity between a wild-type IL-18 and IL-18RP. In some embodiments, the binding affinity between the polymer modified IL-18 polypeptides and IL-18RP is the same as or only moderately lower than the binding affinity between a wild type IL-18 and IL-18RP.

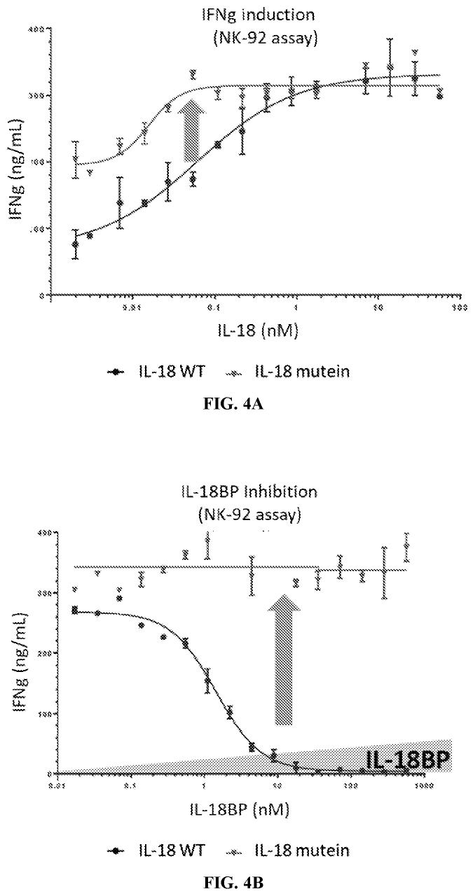

In some embodiments, a polymer modified IL-18 polypeptide provided herein displays an ability to induce interferon gamma (IFNγ) production after administration to a subject. In some embodiments, the ability to induce IFNγ of the polymer modified IL-18 polypeptide is comparable to that of a wild type IL-18 (e.g., displays an EC50 for IFNγ induction that is within about 10-fold of that of a wild type IL-18). An exemplary IL-18 polypeptide provided herein displaying this characteristic is shown in FIG. 4A, which shows a comparison of IFNγ production (ng/mL, y-axis) as a function of concentration of a wild type versus modified IL-18 polypeptide (mutein) (nM, x-axis).

In one aspect, provided herein, is a polymer modified interleukin-18 (IL-18) polypeptide, comprising: a polymer attached to a residue of the modified IL-18 polypeptide, wherein the polymer modified IL-18 polypeptide displays a dissociation constant (K_{D}) for an IL-18 receptor alpha/beta heterodimer (IL-18Rα/β) which is at most eight fold greater than the binding affinity displayed by a corresponding modified IL-18 polypeptide with no polymer attached. In some embodiments, the dissociation constant for the IL-18Rα/R interaction for the polymer modified IL-18 polypeptide is at most two fold, four fold, or six fold greater than the dissociation constant displayed by a corresponding modified IL-18 polypeptide with no polymer attached.

In one aspect, provided herein, is a polymer modified interleukin-18 (IL-18) polypeptide, comprising: a polymer attached to a residue of the modified IL-18 polypeptide, wherein the polymer modified IL-18 polypeptide displays a dissociation constant (K_{D}) for an IL-18 receptor alpha (IL-18Rα) which is at most 9-fold greater than the dissociation constant displayed by a corresponding modified IL-18 polypeptide with no polymer attached. In some embodiments, the dissociation constant for the IL-18Rα interaction of the polymer modified IL-18 polypeptide is at most two fold, four fold, or six fold greater than the dissociation constant displayed by a corresponding modified IL-18 polypeptide with no polymer attached.

In one aspect, provided herein, is a polymer modified interleukin-18 (IL-18) polypeptide, comprising: a polymer attached to any one of residues 79-120, wherein residue position numbering of the modified IL-18 polypeptide is based on SEQ ID NO: 1 as a reference sequence. In some embodiments, the polymer is attached to residue 85, 86, 95, or 98.

In some embodiments, a polymer modified IL-18 polypeptide provided herein also display a reduced binding IL-18 binding protein (IL-18BP). In some embodiments, the reduced binding to IL-18BP is the result of the attachment polymer and/or additional modifications to the modified IL-18 polypeptide (e.g., additional amino acid substitutions). In some embodiments, a polymer modified IL-18 polypeptide provided herein can induce IFNγ even in the presence of IL-18BP (e.g., the ability of the modified IL-18 polypeptide to induce IFNγ is not substantially inhibited by the presence of IL-18BP) (nM, x-axis). An example of an IL-18 polypeptide with this property compared to wild type IL-18 is shown in FIG. 4B, which shows IFNγ production (ng/mL, y-axis) as a function of IL-18BP concentration (nM, x-axis) in a sample treated with the same level of wild type IL-18 (circles) or a modified IL-18 polypeptide provided herein (inverted triangles). Notably, the modified IL-18 polypeptide provided herein showed no inhibition in its ability to induce IFNγ in the presence of IL-18BP, whereas the wild type IL-18 displayed substantial reduction in this ability as the concentration of IL-18BP increased.

A modified IL-18 polypeptide as described herein can comprise one or more unnatural amino acids. “Unnatural” amino acids can refer to amino acid residues in D- or L-form that are not among the 20 canonical amino acids generally incorporated into naturally occurring proteins. In some embodiments, one or more amino acids of the modified IL-18 polypeptides are substituted with one or more unnatural amino acids. Unnatural amino acids include, but are not limited to L-azidolysine, p-azido-L-phenylalanine and L-biphenylalanine.

Exemplary unnatural amino acids also include p-acetyl-L-phenylalanine, p-iodo-L-phenylalanine, p-propargyloxyphenylalanine, p-propargyl-phenylalanine, L-3-(2-naphthyl) alanine, 3-methyl-phenylalanine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, p-Boronophenylalanine, p-bromophenylalanine, p-amino-L-phenylalanine, isopropyl-L-phenylalanine, an analogue of a tyrosine amino acid; an analogue of a glutamine amino acid; an analogue of a phenylalanine amino acid; an analogue of a serine amino acid; an analogue of a threonine amino acid; a 3-amino acid; a cyclic amino acid other than proline or histidine; an aromatic amino acid other than phenylalanine, tyrosine or tryptophan; or a combination thereof. In some embodiments, the unnatural amino acids are selected from 3-amino acids, homoamino acids, and cyclic amino acids. In some embodiments, the unnatural amino acids comprise β-alanine, β-aminopropionic acid, piperidinic acid, aminocaprioic acid, aminoheptanoic acid, aminopimelic acid, desmosine, diaminopimelic acid, N^{α}-ethylglycine, N^{α}-ethylaspargine, isodesmosine, allo-isoleucine, N^{α}-methylglycine, N^{α}-methylisoleucine, N^{α}-methylvaline, γ-carboxyglutamate, N^{α}-acetylserine, N^{α}-formylmethionine, 3-methylhistidine, and/or other similar amino acids.

In some embodiments, amino acid residues of the modified IL-18 polypeptides are substituted with a conjugation handle. In some embodiments, the amino acid residues comprise an amino, azide, allyl, ester, and/or amide functional groups. In some embodiments, the conjugation handles can serve as useful anchor points to attach additional moieties to the modified IL-18 polypeptides. In some embodiments, the amino acid residues have a structure built from precursors Structure 1, Structure 2, Structure 3, or Structure 4:

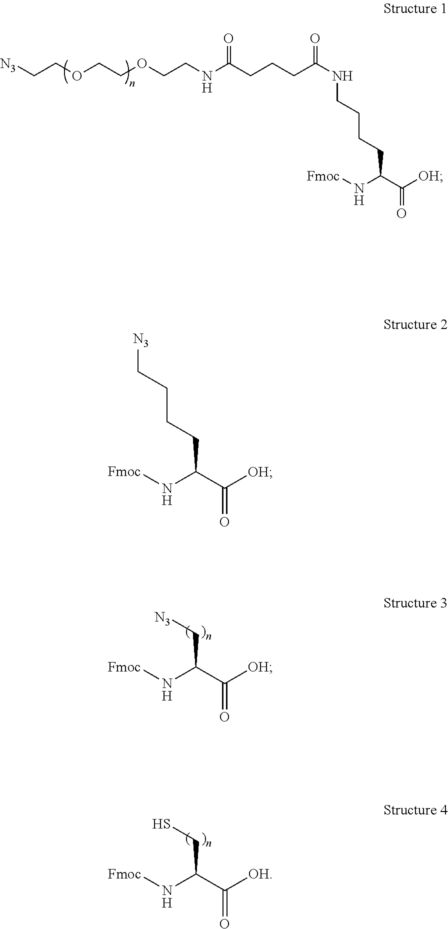

In some embodiments, the polymer modified IL-18 polypeptide contains a substitution for modified natural amino acid residues which can be used for attachment of additional functional groups which can be used to facilitate conjugation reaction or attachment of various payloads to the modified IL-18 polypeptide (e.g., polymers). The substitution can be for a naturally occurring amino acid which is more amenable to attachment of additional functional groups (e.g., aspartic acid/asparagine cysteine, glutamic acid/glutamine, lysine, serine, threonine, or tyrosine), a derivative of a modified version of any naturally occurring amino acid, or any unnatural amino acid (e.g., an amino acid containing a desired reactive group, such as a CLICK chemistry reagent such as an azide, alkyne, etc.). Non-limiting examples of modified natural amino acid residues include the modified lysine, glutamic acid, aspartic acid, and cysteine provided below:

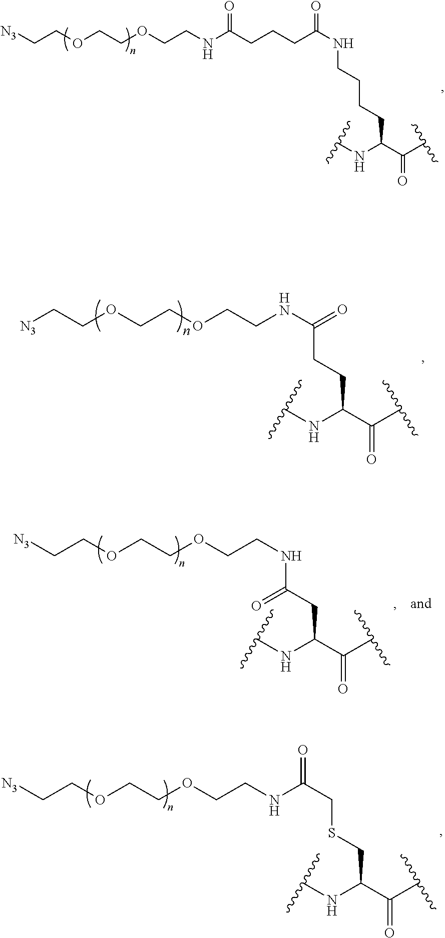

wherein each n is an integer from 1-30. Other examples of natural amino acids which can be similarly modified include those with heteroatoms capable of easily forming a bond with a suitable group to link the polymeric group to the amino acid (e.g., tyrosine, serine, threonine). These non-limiting examples of modified amino acid residues can be used at any location at which it is desirable to add an additional functionality (e.g., a polymer) to the modified IL-18 polypeptide.

In some embodiments, any of structures 1-4, the modified lysine, the modified glutamic acid, the modified aspartic acid, or the modified cysteine provided above can be substituted for a different residue of the modified IL-18 polypeptide (e.g., any residues 79-120 using SEQ ID NO: 1 as a reference sequence) to allow for conjugation at a different site of the IL-18 polypeptide. The azide functionality may also be replaced with another suitable conjugation handle.

The conjugation handles provided herein can be any suitable reactive group capable of reacting with a complementary reactive group. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cyclooctenes, or a metal-mediated process such as olefin metathesis and Suzuki-Miyaura, 0-substituted hydroxylamine, potassium acyltrifluoroborate or Sonogashira cross-coupling.

In some embodiments, the conjugation handle comprises a reagent for a “copper-free” alkyne azide triazole-forming reaction. Non-limiting examples of alkynes for said alkyne azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof).

In some embodiments, the conjugation handle comprises a reactive group selected from azide, alkyne, tetrazine, halide, sulfhydryl, disulfide, maleimide, activated ester, alkene, aldehyde, ketone, imine, hydrazine, acyltrifluoroborate, hydroxylamine (e.g., 0-substituted hydroxylamine), phosphine, trans-cyclooctene, and hydrazide. In some embodiments, the conjugation handle and complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research, volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; U.S. Ser. No. 10/266,502B2; and US20190204330A1, each of which is incorporated by reference in its entirety.

In some embodiments, a group attached to the modified IL-18 polypeptide (e.g., a polymer moiety or an additional polypeptide) comprises a conjugation handle or a reaction product of a conjugation handle with a complementary conjugation handle. In some embodiments, the reaction product of the conjugation handle with the complementary conjugation handle results from a KAT ligation (reaction of potassium acyltrifluoroborate with hydroxylamine), a Staudinger ligation (reaction of an azide with a phosphine), a tetrazine cycloaddition (reaction of a tetrazine with a trans-cyclooctene), or a Huisgen cycloaddition (reaction of an alkyne with an azide). In some embodiments, the group attached to the IL-18 polypeptide (e.g., the polymer or the additional polypeptide) will comprise a reaction product of a conjugation handle with a complementary conjugation handle which was used to attach the group to the modified IL-18 polypeptide.

IIIa. Points of Attachment of Polymer Moieties to Modified IL-18 Polypeptides

The polymer modified IL-18 polypeptides described herein contain one or more polymers. In some embodiments, a polymer modified IL-18 polypeptide is conjugated to one polymer moiety.

In some embodiments, the polymer modified IL-18 polypeptide comprises a polymer covalently attached to a residue of a modified IL-18 polypeptide described herein. In some embodiments, the polymer is covalently attached to a residue in the region of residues 79-120. In some embodiments, the polymer is covalently attached to a residue in the region of residues 79-100. In some embodiments, the polymer is covalently attached to a residue in the region of residues 81-110, residues 83-100, or residues 85-98. In some embodiments, the polymer is covalently attached at a residue in the region of residues 83-88, residues 84-87, or residues 85-86. In some embodiments, the polymer is covalently attached at a residue in the region of residues 94-102, residues 95-101, residues 96-100, or residues 97-99. In some embodiments, the polymer is covalently attached at residue 85, residue 86, residue 95, or residue 98.

In some embodiments, the residue to which the polymer attached is a natural amino acid residue. In some embodiments, the residue to which the polymer is covalently attached is selected from cysteine, aspartate, asparagine, glutamate, glutamine, serine, threonine, lysine, and tyrosine. In some embodiments, the residue to which the polymer is covalently attached is selected from asparagine, aspartic acid, cysteine, glutamic acid, glutamine, lysine, and tyrosine. In some embodiments, the polymer is covalently attached to a cysteine. In some embodiments, the polymer is covalently attached to a lysine. In some embodiments, the polymer is covalently attached to a glutamine. In some embodiments, the polymer is covalently attached to an asparagine. In some embodiments, the residue to which the polymer is attached is the natural amino acid in that position in SEQ ID NO: 1. In some embodiments, the polymer is attached to a different natural amino acid which is substituted at the relevant position. In some embodiments, the polymer is covalently attached to site-specifically to a natural amino acid.

In some embodiments, the polymer is attached at an unnatural amino acid residue. In some embodiments, the unnatural amino acid residue comprises a conjugation handle. In some embodiments, the conjugation handle facilitates the addition of the polymer to the modified IL-18 polypeptide. The conjugation handle can be any of the conjugation handles provided herein. In some embodiments, the polymer is covalently attached site-specifically to the unnatural amino acid.

In some embodiments, the polymer is covalently attached at residue in the region of residues 79-120. In some embodiments, the polymer is covalently attached at residue 79, residue 80, residue 81, residue 82, residue 83, residue 84, residue 85, residue 86, residue 87, residue 88, residue 89, residue 90, residue 91, residue 92, residue 93, residue 94, residue 95, residue 96, residue 97, residue 98, residue 99, residue 100, residue 101, residue 102, residue 103, residue 104, residue 105, residue 106, residue 107, residue 108, residue 109, residue 110, residue 111, residue 112, residue 113, residue 114, residue 115, residue 116, residue 117, residue 118, residue 119, or residue 120. In some embodiments, the polymer is covalently attached at residue 85, residue 86, residue 95, or residue 98.

In some embodiments, the polymer is covalently attached at residue 85. In some embodiments, the polymer is covalently attached at residue E85, E85C, E85D, E85Q, E85K, E85N, or E85Y. In some embodiments, the polymer is covalently attached at residue E85. In some embodiments, the polymer is covalently attached residue E85C. In some embodiments, the polymer is covalently attached to an unnatural amino acid at residue 85.

In some embodiments, the polymer is covalently attached at residue 86. In some embodiments, the polymer is covalently attached at residue M86C, M86D, M86Q, M86K, M86N, M86E, or M86Y. In some embodiments, the polymer is covalently attached M86C. In some embodiments, the polymer is covalently attached to an unnatural amino acid at residue 86.

In some embodiments, the polymer is covalently attached at residue 95. In some embodiments, the polymer is covalently attached at residue T95, T95C, T95D, T95Q, T95K, T95N, T95E, or T95Y. In some embodiments, the polymer is covalently attached at residue T95C, T95D, T95Q, T95K, T95N, T95E, or T95Y. In some embodiments, the polymer is covalently attached at residue T95C. In some embodiments, the polymer is covalently attached to an unnatural amino acid at residue 95.

In some embodiments, the polymer is covalently attached at residue 98. In some embodiments, the polymer is covalently attached at residue D98, D98C, D98Q, D98K, D98N, D98E, or D98Y. In some embodiments, the polymer is covalently attached at residue D98C. In some embodiments, the polymer is covalently attached to an unnatural amino acid at residue 98.

In some embodiments, the polymer is covalently attached through a modified amino acid α. In some embodiments, the modified amino acid α is an amino-acid-PEG-azide group. In some embodiments, the modified amino acid α is a glutamate, aspartate, lysine, cysteine, or tyrosine modified to incorporate an azide group linked to the amino acid through a PEG spacer. In some embodiments, the modified amino acid α has a structure selected from:

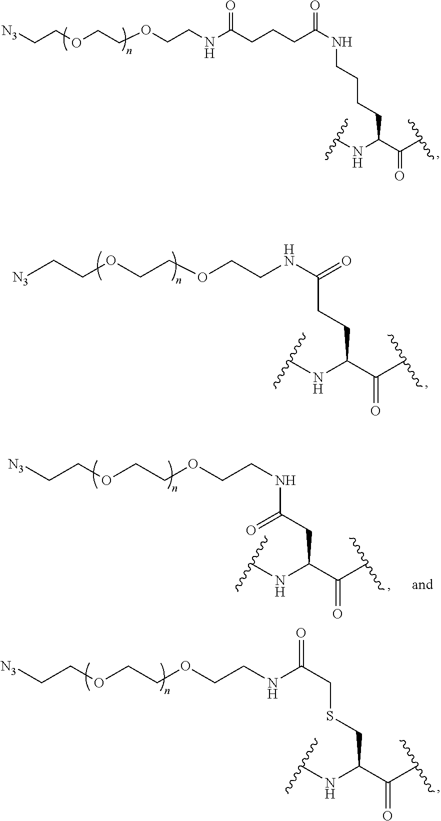

wherein each n is independently an integer from 1-30. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 10. In some embodiments, n is 8. In some embodiments, n is 6. In some embodiments, n is 12.

In some embodiments, the modified amino acid α is located at a position on the modified IL-18 polypeptide in the region of residues 79-120. In some embodiments, the modified amino acid α is located at a position on the modified IL-18 polypeptide selected from residue 85, residue 86, or residue 98. In some embodiments, the modified amino acid α is located at residue 85 of the modified IL-18 polypeptide. In some embodiments, the modified amino acid α is located at residue 86 of the modified IL-18 polypeptide. In some embodiments, the modified amino acid α is located at residue 95 of the modified IL-18 polypeptide. In some embodiments, the modified amino acid α is located at residue 98 of the modified IL-18 polypeptide.

In some embodiments, the modified IL-18 polypeptide comprises a polymer covalently attached to a modified lysine residue. In some embodiments, the modified lysine residue comprises a conjugation handle. In some embodiments, the modified lysine residue comprises an azide. In some embodiments, the modified lysine residue has a structure of Structure B, wherein Structure B is

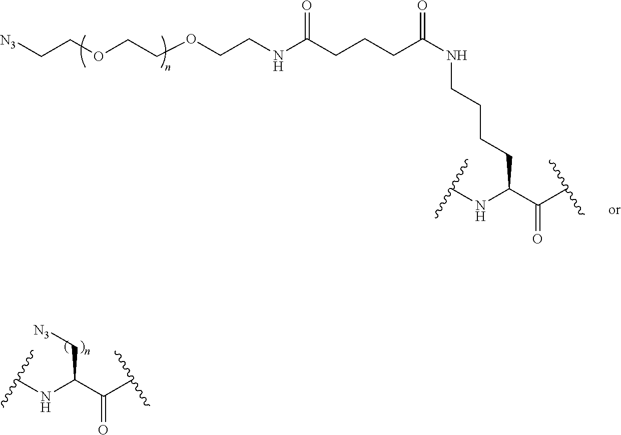

wherein each n is independently an integer from 1-30. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 1, 2, 3, 4, 5, 6, 7, or 8. In some embodiments, n is 3. In some embodiments, n is 4. In some embodiments, n is 5. In some embodiments, n is 6. In some embodiments, n is 8. In some embodiments, n is 10. In some embodiments, n is 12.

In some embodiments, the modified lysine of Structure B is located at a position on the modified IL-18 polypeptide in the region of residues 79-120. In some embodiments, the modified lysine of Structure B is located at a position on the modified IL-18 polypeptide selected from residue 85, residue 86, and residue 98. In some embodiments, the modified lysine of Structure B is located at residue 85 of the modified IL-18 polypeptide. In some embodiments, the modified lysine of Structure B is located at residue 86 of the modified IL-18 polypeptide. In some embodiments, the modified lysine of Structure B is located at residue 95 of the modified IL-18 polypeptide. In some embodiments, the modified lysine of Structure B is located at residue 98 of the modified IL-18 polypeptide.

In some embodiments, the modified IL-18 polypeptide comprises more than one polymer covalently attached thereto. In some embodiments, the modified IL-18 polypeptide comprises a second polymer covalently attached. In some embodiments, the modified IL-18 polypeptide comprises a second polymer covalently attached at a residue provided herein.

IIIb. Polymers Attached to Modified IL-18 Polypeptides

In one aspect, described herein is a polymer modified polypeptide that comprises a modified IL-18 polypeptide, wherein the polymer modified IL-18 polypeptide comprises a covalently attached polymer. In some embodiments, described herein is a polymer modified IL-18 polypeptide comprising one or more polymers covalently attached to a modified IL-18 polypeptide. In some embodiments, the polymer modified IL-18 polypeptide comprises 1, 2, 3, 4, 5, 6, 7, 8, 9, 10, or more polymers covalently attached to the modified IL-18 polypeptide.

In some embodiments, the polymer comprises a conjugation handle which can be used to further attach an additional moiety to the polymer modified IL-18 polypeptide (e.g., the addition of an additional polypeptide, such as an antibody). Any suitable reactive group capable of reacting with a complementary reactive group attached to another moiety can be used as the conjugation handle. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cyclooctenes, potassium acyltrifluoroborate ligations (e.g., with O-substituted hydroxylamines) or a metal-mediated process such as olefin metathesis and Suzuki-Miyaura or Sonogashira cross-coupling.

In some embodiments, the conjugation handle comprises a reagent for a “copper-free” alkyne azide triazole-forming reaction. Non-limiting examples of alkynes for said alkyne azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof).

In some embodiments, the conjugation handle comprises a reactive group selected from azide, alkyne, tetrazine, halide, sulfhydryl, disulfide, maleimide, activated ester, alkene, aldehyde, ketone, imine, hydrazine, acyltrifluoroborate, hydroxylamine, phosphine, trans-cyclooctene, and hydrazide. In some embodiments, the conjugation handle and the complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research, volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; U.S. Ser. No. 10/266,502B2; and US20190204330A1, each of which is incorporated by reference in its entirety.

In some embodiments, the polymer comprises a conjugation handle or a reaction product of a conjugation handle with a complementary conjugation handle. In some embodiments, the reaction product of the conjugation handle with the complementary conjugation handle results from a KAT ligation (reaction of potassium acyltrifluoroborate with hydroxylamine), a Staudinger ligation (reaction of an azide with a phosphine), a tetrazine cycloaddition (reaction of a tetrazine with a trans-cyclooctene), or a Huisgen cycloaddition (reaction of an alkyne with an azide). In some embodiments, the polymer comprises a reaction product of a conjugation handle with a complementary conjugation handle which was used to attach the polymer to the modified IL-18 polypeptide.

In some embodiments, the polymer comprises an azide moiety. In some embodiments, the polymer comprises an alkyne moiety. In some embodiments, the polymer comprises an azide moiety, an alkyne moiety, or reaction product of an azide-alkyne cycloaddition reaction. In some embodiments, the reaction product of the azide-alkyne cycloaddition reaction is a 1,2,3-triazole.

In some embodiments, the polymer is attached to the modified IL-18 polypeptide through use of a bifunctional linker. In some embodiments, the bifunctional linker reacts with a reactive group of an amino acid residue on the modified IL-18 polypeptide (e.g., a cysteine sulfhydryl) to form a covalent bond. In some embodiments, in a second step, the second reactive group of the bifunctional a linker (e.g., a conjugation handle such as an azide or alkyne) is then used to attach a second moiety, such as the polymer.

In some embodiments, the polymer is a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is a polysaccharide. In some embodiments, the water-soluble polymer comprises poly(alkylene oxide). In some embodiments, the poly(alkylene oxide) is polyethylene glycol (PEG).

In some embodiments, the polyethylene glycol has a weight average molecular weight of about 0.1 kDa to about 50 kDa. In some embodiments, the polyethylene glycol has a weight average molecular weight of about 0.5 kDa to about 50 kDa. In some embodiments, the polyethylene glycol has a weight average molecular weight of about 10 kDa, about 20 kDa, or about 30 kDa. In some embodiments, the polyethylene glycol has a weight average molecular weight of about 30 kDa. about 20 kDa to about 40 kDa, about 20 kDa to about 50 kDa, or about 40 kDa to about 50 kDa. In some embodiments, the polyethylene glycol has a weight average molecular weight of about 0.5 kDa, about 1 kDa, about 5 kDa, about 10 kDa, about 20 kDa, about 40 kDa, or about 50 kDa. In some embodiments, the polyethylene glycol has a weight average molecular weight of at least about 0.1 kDa, about 0.5 kDa, about 1 kDa, about 5 kDa, about 10 kDa, about 20 kDa, or about 40 kDa. In some embodiments, the polyethylene glycol has a weight average molecular weight of at most about 5 kDa, about 10 kDa, about 20 kDa, about 40 kDa, or about 50 kDa.

In some embodiments, the attached polymer has a weight average molecular weight of about 6,000 Daltons to about 50,000 Daltons. In some embodiments, the polymer has a weight average molecular weight of about 6,000 Daltons to about 10,000 Daltons, about 6,000 Daltons to about 25,000 Daltons, about 6,000 Daltons to about 50,000 Daltons, about 10,000 Daltons to about 25,000 Daltons, about 10,000 Daltons to about 50,000 Daltons, or about 25,000 Daltons to about 50,000 Daltons. In some embodiments, the polymer has a weight average molecular weight of about 6,000 Daltons, about 10,000 Daltons, about 25,000 Daltons, or about 50,000 Daltons. In some embodiments, the polymer has a weight average molecular weight of at least about 6,000 Daltons, about 10,000 Daltons, or about 25,000 Daltons. In some embodiments, the polymer has a weight average molecular weight of at most about 10,000 Daltons, about 25,000 Daltons, or about 50,000 Daltons.

In some embodiments, the attached polymer has a weight average molecular weight of about 120 Daltons to about 1,000 Daltons. In some embodiments, the polymer has a weight average molecular weight of about 100 Daltons to about 250 Daltons, about 100 Daltons to about 300 Daltons, about 100 Daltons to about 400 Daltons, about 100 Daltons to about 500 Daltons, about 100 Daltons to about 1,000 Daltons, about 250 Daltons to about 300 Daltons, about 250 Daltons to about 400 Daltons, about 250 Daltons to about 500 Daltons, about 250 Daltons to about 1,000 Daltons, about 300 Daltons to about 400 Daltons, about 300 Daltons to about 500 Daltons, about 300 Daltons to about 1,000 Daltons, about 400 Daltons to about 500 Daltons, about 400 Daltons to about 1,000 Daltons, or about 500 Daltons to about 1,000 Daltons. In some embodiments, the polymer has a weight average molecular weight of about 100 Daltons, about 250 Daltons, about 300 Daltons, about 400 Daltons, about 500 Daltons, or about 1,000 Daltons. In some embodiments, the polymer has a weight average molecular weight of at least about 100 Daltons, about 250 Daltons, about 300 Daltons, about 400 Daltons, or about 500 Daltons. In some embodiments, the polymer has a weight average molecular weight of at most about 250 Daltons, about 300 Daltons, about 400 Daltons, about 500 Daltons, or about 1,000 Daltons.

In some embodiments, the polymer comprises one or more linker groups. In some embodiments, the linker groups used to attach the polymeric portion of the polymer to the modified IL-18 polypeptide. In some embodiments, the one or more linker groups comprise bifunctional linkers such as an amide group, an ester group, an ether group, a thioether group, a carbonyl group and alike. In some embodiments, the one or more linker groups comprise an amide linker group. In some embodiments, the modified poly(alkylene oxide) comprises one or more spacer groups. In some embodiments, the spacer groups comprise a substituted or unsubstituted C1-C6 alkylene group. In some embodiments, the spacer groups comprise —CH₂—, —CH₂CH₂—, or —CH₂CH₂CH₂—. In some embodiments, the linker group is the product of a biorthogonal reaction (e.g., biocompatible and selective reactions). In some embodiments, the bioorthogonal reaction is a Cu(I)-catalyzed or “copper-free” alkyne-azide triazole-forming reaction, the Staudinger ligation, inverse-electron-demand Diels-Alder (IEDDA) reaction, alkyne-nitrone cycloaddition chemistry, or a metal-mediated process such as olefin metathesis and Suzuki-Miyaura or Sonogashira cross-coupling. In some embodiments, the first polymer is attached to the IL-18 polypeptide via click chemistry.

In some embodiments, the polymer comprises a linker comprising a structure of Formula (X)

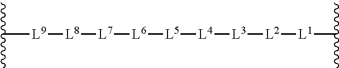

wherein each of L¹, L², L³, L⁴, L⁵, L⁶, L⁷, L⁸, and L⁹ is independently —O—, —NR^{L}—, —(C₁-C₆ alkylene)NR^{L}—, —NR^{L}(C₁-C₆ alkylene)-, —N(R^{L})₂_{⁺}—, —C₁-C₆ alkylene)N(R^{L})₂_{⁺}—, —N(R^{L})₂_{⁺}—(C₁-C₆ alkylene)-, —OP(═O)(OR^{L})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O) (C₁-C₆ alkylene)-, —C(═O)O—, —OC(═O)—, —OC(═O)O—, —C(═O)NR^{L}—, —C(═O)NR^{L}(C₁-C₆ alkylene)-, —(C₁-C₆ alkylene)C(═O)NR^{L}—, —NR^{L}C(═O—(C₁-C₆ alkylene)NR^{L}C(═O)—, —NR^{L}C(═O)(C₁-C₆ alkylene)-, —OC(═O)NR^{L}—, —NR^{L}C(═O)O—NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)_{qa}—, a reaction product of a conjugation handle and a complementary conjugation handle, or absent; (C₁-C₆ alkylene)
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
wherein each

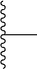

is a point of attachment to the modified IL-18 polypeptide or the polymeric portion of the polymer.

In some embodiments, the polymer comprises a linker comprising a structure of Formula (X′)

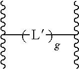

wherein each L′ is independently —O—, —NR^{L}—, —(C₁-C₆ alkylene)NR^{L}—, —NR^{L}(C₁-C₆ alkylene)-, —N(R^{L})₂_{⁺}—, C₁-C₆ alkylene)N(R^{L})₂_{⁺}—, —N(R^{L})₂_{⁺}—(C₁-C₆ alkylene)-, —OP(═O)(OR^{L})O—, —S—, (C₁-C₆ alkylene)S—, —S(C₁-C₆ alkylene)-, —S(═O)—, —S(═O)₂—, —C(═O)—, —(C₁-C₆ alkylene)C(═O)—, —C(═O) (C₁-C₆ alkylene)-, —C(═O)O—, —OC(═O)—, —OC(═O)O—, —C(═O)NR^{L}—, —C(═O)NR^{L}(C₁-C₆ alkylene)-, —(C₁-C₆ alkylene)C(═O)NR^{L}—, —NR^{L}C(═O—(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)_{qa}—, a reaction product of a conjugation handle and a complementary conjugation handle, or absent; (C₁-C₆ alkylene) each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
g is an integer from 1-100,
wherein each

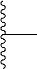

is a point of attachment to the modified IL-18 polypeptide or the polymeric portion of the polymer.

In some embodiments, a polymer modified IL-18 polypeptide provided herein comprises a polymer which includes a linker selected from Table 1. In Table 1, each

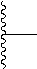

is a point of attachment to either the modified IL-18 polypeptide (e.g., an amino group of the modified IL-18 polypeptide) or to the polymeric portion of the polymer.

TABLE 1
Exemplary linker structures for modified IL-18 polypeptides

| Linker | |
|---|---|
| Identifier | Linker Structure |
| Formula A | 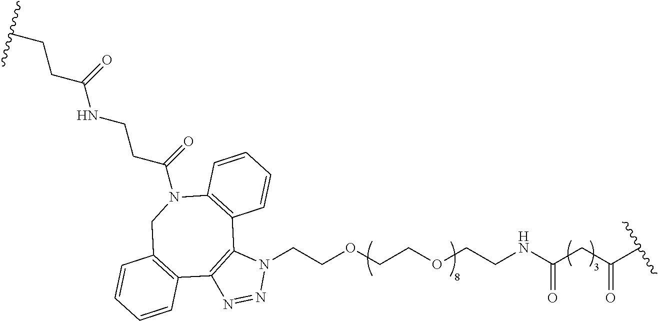 |
| Formula B | 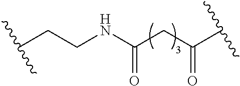 |
| Formula C | 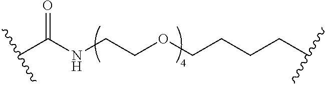 |
| Formula D | 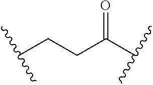 [SMILES: *CCC(*)=O] |
| Formula E | 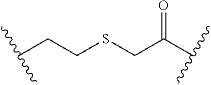 [SMILES: *CCSCC(*)=O] |
| Formula F | 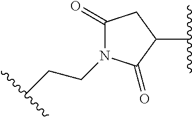 [SMILES: *CCN1C(=O)CC(*)C1=O] |
| Formula G | 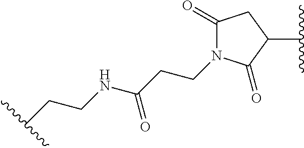 [SMILES: *CCNC(=O)CCN1C(=O)CC(*)C1=O] |
| Formula H | 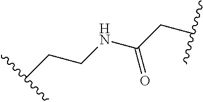 [SMILES: *CCNC(=O)C*] |

In some embodiments, the water-soluble polymer is linear or branched. In some embodiments, the water-soluble polymer is branched and includes a plurality of polyethylene glycol chains. In some embodiments, the water-soluble polymer comprises from 1 to 10 polyethylene glycol chains. In some embodiments, the water-soluble polymer comprises 1 polyethylene glycol chains to 2 polyethylene glycol chains, 1 polyethylene glycol chains to 4 polyethylene glycol chains, 1 polyethylene glycol chains to 6 polyethylene glycol chains, 1 polyethylene glycol chains to 10 polyethylene glycol chains, 2 polyethylene glycol chains to 4 polyethylene glycol chains, 2 polyethylene glycol chains to 6 polyethylene glycol chains, 2 polyethylene glycol chains to 10 polyethylene glycol chains, 4 polyethylene glycol chains to 6 polyethylene glycol chains, 4 polyethylene glycol chains to 10 polyethylene glycol chains, or 6 polyethylene glycol chains to 10 polyethylene glycol chains.

In some embodiments, the polymer attached to the modified IL-18 polypeptide comprises a conjugation handle. In some embodiments, the conjugation handle attached to the polymer can be used to link an additional moiety (e.g., an additional polypeptide, such as an antibody or an additional cytokine, or a larger polymer) to the polymer modified IL-18 polypeptide to form a conjugate composition. In some embodiments, the polymer comprises an alkyne or azide conjugation handle. In some embodiments, the conjugation handle is attached to a terminal atom of the polymer. Non-limiting examples of polymers with conjugation handles attached are shown in modified amino acids a above. In some embodiments, the polymer comprises a structure of Formula (I):

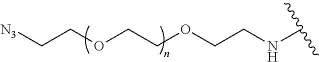

wherein n is an integer from 2-30. In some embodiments, n is an integer from 2-10. In some embodiments, n is 2, 4, 6, 8, or 10. In some embodiments, n is 8.

In some embodiments, addition of the polymer to the modified IL-18 polypeptide increases the stability of the modified IL-18 polypeptide in vivo or in vitro. In some embodiments, addition of the polymer increases one or more pharmacokinetic (PK) properties of the modified IL-18 polypeptide. In some embodiments, the polymer modified IL-18 polypeptide of the disclosure comprises a covalently attached polymer for plasma or serum half-life extension. In some embodiments, a plasma or serum half-life of the polymer modified IL-18 polypeptide of the disclosure is at least 1.5-fold, 2-fold, 3-fold, 4-fold, 5-fold, 6-fold, 7-fold, 8-fold, 9-fold, or 10-fold longer compared to a plasma or serum half-life of a wild-type IL-18 polypeptide. In some embodiments, a plasma or serum half-life of the polymer modified IL-18 polypeptide of the disclosure is 1.5-fold to 10-fold longer compared to a plasma or serum half-life of a wild-type IL-18 polypeptide. In some embodiments, a half-life of the polymer modified IL-18 polypeptide is at least 10% longer than a half-life of a wild-type IL-18 polypeptide. In some embodiments, the half-life of the polymer modified IL-18 polypeptide is at least 30% longer than the half-life of wild-type IL-18 polypeptide.

In some embodiments, a plasma or serum half-life of a polymer modified IL-18 polypeptide described herein is at least 1.5-fold, 2-fold, 3-fold, 4-fold, 5-fold, 6-fold, 7-fold, 8-fold, 9-fold, or 10-fold longer compared to a plasma or serum half-life of the modified IL-18 polypeptide without the polymer attached. In some embodiments, a plasma or serum half-life of the polymer modified IL-18 polypeptide of the disclosure is 1.5-fold to 10-fold longer compared to a plasma or serum half-life of the modified IL-18 polypeptide without the polymer attached. In some embodiments, a half-life of the polymer modified IL-18 polypeptide is at least 10% longer than a half-life of the modified IL-18 polypeptide without the polymer. In some embodiments, the half-life of the polymer modified IL-18 polypeptide is at least 30% longer than the half-life of the modified IL-18 polypeptide without the polymer.

IIIc. Additional Site-Specific Modifications of Modified IL-18 Polypeptides

In some embodiments, an IL-18 polypeptide modified with a polymer described herein comprises one or more additional modifications at one or more amino acid residues. As used herein, “additional modifications” are modifications to the IL-18 polypeptide in addition to polymer attachment to a residue and/or substitution of the residue to which the polymer is attached.

Similarly, “additional substitutions” are substitutions at a residue other than the residue to which the polymer is attached, which may or may not be substituted relative to SEQ ID NO: 1. In some embodiments, the additional modifications are at residues to which the polymer is not attached. In some embodiments, the residue position numbering of the modified IL-18 polypeptide is based on SEQ ID NO: 1 as a reference sequence.

Additional modifications to the polypeptides described herein encompass mutations, addition of various functionalities, deletion of amino acids, addition of amino acids, or any other alteration of the wild-type version of the protein or protein fragment. Functionalities which may be added to polypeptides include polymers, linkers, alkyl groups, detectable molecules such as chromophores or fluorophores, reactive functional groups, or any combination thereof. In some embodiments, functionalities are added to individual amino acids of the polypeptides. In some embodiments, functionalities are added site-specifically to the polypeptides.

In some embodiments, the modified IL-18 polypeptides described herein contain 1, 2, 3, 4, 5, 6, 7, 8, 9, or 10 additional modified amino acid residues.

In some embodiments, a modified IL-18 polypeptide provided herein comprises an amino acid sequence of any one of SEQ ID NOs: 2-203 provided herein. In some embodiments, the modified IL-18 polypeptide comprises an amino acid sequence at least 85% identical to the sequence of any one of SEQ ID NOs: 2-203. In some embodiments, a modified IL-18 polypeptide provided herein comprises an amino acid sequence of any one of SEQ ID NOs: 2-33 provided herein. In some embodiments, the modified IL-18 polypeptide comprises an amino acid sequence at least 85% identical to the sequence of any one of SEQ ID NOs: 2-33. In some embodiments, a modified IL-18 polypeptide provided herein comprises an amino acid sequence of any one of SEQ ID NOs: 68-163 provided herein. In some embodiments, the modified IL-18 polypeptide comprises an amino acid sequence at least 85% identical to the sequence of any one of SEQ ID NOs: 68-163. In some embodiments, the modified IL-18 polypeptide comprises an amino acid sequence of SEQ ID NO: 30. In some embodiments, the modified IL-18 polypeptide comprises an amino acid sequence at least 85% identical to the sequence of SEQ ID NO: 30. In some embodiments, the modified IL-18 polypeptide comprises an amino acid sequence of SEQ ID NO: 59. In some embodiments, the modified IL-18 polypeptide comprises an amino acid sequence at least 85% identical to the sequence of SEQ ID NO: 59. In some embodiments, the modified IL-18 polypeptide comprises an amino acid sequence of SEQ ID NO: 2. In some embodiments, the modified IL-18 polypeptide comprises an amino acid sequence at least 85% identical to the sequence of SEQ ID NO: 2.

In some embodiments, a polymer modified IL-18 polypeptide comprising a polymer described herein comprises at least 1, at least 2, at least 3, at least 4, at least 5, at least 6, at least 7, or at least 9 additional amino acid substitutions, wherein the amino acid substitutions are relative to SEQ ID NO: 1. In some embodiments, the modified IL-18 polypeptide comprises 1 to 9 amino acid substitutions. In some embodiments, the modified IL-18 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the modified IL-18 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the modified IL-18 polypeptide comprises at most 4 amino acid substitutions, 5 amino acid substitutions, 6 amino acid substitutions, 7 amino acid substitutions, or 9 amino acid substitutions.

In some embodiments, a polymer modified IL-18 polypeptide comprising a polymer described herein comprises at least 1, at least 2, at least 3, at least 4, at least 5, at least 6, at least 7, or at least 9 additional natural amino acid substitutions, wherein the natural amino acid substitutions are relative to SEQ ID NO: 1. In some embodiments, the modified IL-18 polypeptide comprises 1 to 9 natural amino acid substitutions. In some embodiments, the modified IL-18 polypeptide comprises 1 or 2 natural amino acid substitutions, 1 to 3 natural amino acid substitutions, 1 to 4 natural amino acid substitutions, 1 to 5 natural amino acid substitutions, 1 to 6 natural amino acid substitutions, 1 to 7 natural amino acid substitutions, 1 to 8 natural amino acid substitutions, 2 to 3 natural amino acid substitutions, 2 to 4 natural amino acid substitutions, 2 to 5 natural amino acid substitutions, 2 to 6 natural amino acid substitutions, 2 to 7 natural amino acid substitutions, 2 to 8 natural amino acid substitutions, 2 to 9 natural amino acid substitutions, 3 or 4 natural amino acid substitutions, 3 to 5 natural amino acid substitutions, 3 to 6 natural amino acid substitutions, 3 to 7 natural amino acid substitutions, 3 to 9 natural amino acid substitutions, 4 or 5 natural amino acid substitutions, 4 to 6 natural amino acid substitutions, 4 to 7 amino acid substitutions, 4 to 9 natural amino acid substitutions, 5 or 6 natural amino acid substitutions, 5 to 7 amino acid substitutions, 5 to 9 natural amino acid substitutions, 6 or 7 natural amino acid substitutions, 6 to 9 natural amino acid substitutions, or 7 to 9 natural amino acid substitutions. In some embodiments, the modified IL-18 polypeptide comprises 3 natural amino acid substitutions, 4 natural amino acid substitutions, 5 amino acid substitutions, 6 natural amino acid substitutions, 7 natural amino acid substitutions, or 9 natural amino acid substitutions. In some embodiments, the modified IL-18 polypeptide comprises at most 4 natural amino acid substitutions, 5 natural amino acid substitutions, 6 natural amino acid substitutions, 7 natural amino acid substitutions, or 9 natural amino acid substitutions. In some embodiments, the modified IL-18 polypeptide further comprises up to 10 non-canonical amino acid substitutions. In some embodiments, the modified IL-18 polypeptide comprises 1, 2, 3, 4, 5, 6, 7, 8, 9 or 10 additional unnatural amino acid substitutions. In some embodiments, the modified IL-18 polypeptide further comprises unnatural amino acid substitutions at residues M33, M51, N60, M86, M113, and/or M150. In some embodiments, the unnatural amino acid residues substituted for the methionines are each independently norleucine or O-methyl-homoserine. In some embodiments, the modified IL-18 polypeptide further unnatural amino acid substitutions at residues 31, 75, and 116. In some embodiments, the modified IL-18 polypeptide further comprises homoserine (Hse) 31, Hse 75, and Hse 116. In some embodiments, the modified IL-18 polypeptide further unnatural amino acid substitutions at residues 31, 63, and 116. In some embodiments, the modified IL-18 polypeptide further comprises homoserine (Hse) 31, Hse 63, and Hse 116. In some embodiments, the modified IL-18 polypeptide further unnatural amino acid substitutions at residues 31, 63, 75, and 116. In some embodiments, the modified IL-18 polypeptide further comprises homoserine (Hse) 31, Hse 63, Hse 75, and Hse 116. In some embodiments, the modified IL-18 polypeptide further unnatural amino acid substitutions at residues 31, 67, 75, and 116. In some embodiments, the modified IL-18 polypeptide further comprises homoserine (Hse) 31, Hse 67, Hse 75, and Hse 116. In some embodiments, the modified IL-18 polypeptide further unnatural amino acid substitutions at residues 31, 57, 75, and 116. In some embodiments, the modified IL-18 polypeptide further comprises homoserine (Hse) 31, Hse 57, Hse 75, and Hse 116. In some embodiments, the modified IL-18 polypeptide further unnatural amino acid substitutions at residues 31, 50, 75, and 116. In some embodiments, the modified IL-18 polypeptide further comprises homoserine (Hse) 31, Hse 50, Hse 75, and Hse 116. In some embodiments, the modified IL-18 polypeptide further unnatural amino acid substitutions at residues 31, 50, 75, and 121. In some embodiments, the modified IL-18 polypeptide further comprises homoserine (Hse) 31, Hse 50, Hse 75, and Hse 121.

In some embodiments, a polymer modified IL-18 polypeptide comprising a polymer described herein comprises at least 1, at least 2, at least 3, at least 4, at least 5, at least 6, at least 7, or at least 9 additional amino acid substitutions, wherein the amino acid substitutions are relative to any one of SEQ ID NOs: 68, 92, 116, 140, or 170. In some embodiments, a polymer modified IL-18 polypeptide comprising a polymer described herein comprises at least 1, at least 2, at least 3, at least 4, at least 5, at least 6, at least 7, or at least 9 additional amino acid substitutions, wherein the amino acid substitutions are relative to any one of SEQ ID NOs: 68, 92, 116, or 140. In some embodiments, the modified IL-18 polypeptide comprises 1 to 9 amino acid substitutions. In some embodiments, the modified IL-18 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the modified IL-18 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the modified IL-18 polypeptide comprises at most 4 amino acid substitutions, 5 amino acid substitutions, 6 amino acid substitutions, 7 amino acid substitutions, or 9 amino acid substitutions.

In some embodiments, the polymer modified IL-18 polypeptides comprise one or more modifications in addition to a modification needed to attach a polymer (e.g., an amino acid substitution at a residue to which the polymer is not attached). In some embodiments, the additional modification is in the range of amino acid residues 1-127, based on the sequence of human IL-18³⁷⁻¹⁹³ (SEQ ID NO: 1). SEQ ID NO: 1 reflects the bioactive form of IL-18. Endogenously, IL-18 is initially expressed with an additional 36 amino acid segment at the N-terminus which is cleaved by caspases to mediate biologic activity. In some embodiments, one modification is in the range of amino acid residues 6-63 based on SEQ ID NO: 1. In some embodiments, one modification is at amino acid residue 6. In some embodiments, one modification is in the range of amino acid residues 53-63. In some embodiments, one modification is at amino acid residue 53. In some embodiments, one modification is at amino acid residue 63.

In some embodiments, the modified IL-18 polypeptide comprises at least one additional modification to the amino acid sequence of SEQ ID NO: 1 selected from: Y01X, F02X, E06X, S10X, V11X, D17X, C38X, M51X, K53X, D54X, S55X, T63X, C68X, C76X, AND C127X, wherein each X is independently a natural or non-natural amino acid. In some embodiments, the modified IL-18 polypeptide comprises at least one additional modification to the amino acid sequence of SEQ ID NO: 1 selected from: Y01G, F02A, E06K, S10T, V11, D17N, C38S, C38A, C38Q, M51G, K53A, D54A, S55A, T63A, C68S, C68A, E69C, K70C, C76S, C76A, C127A, and C127S.

In one aspect, described herein is a modified interleukin-18 (IL-18) polypeptide with a polymer as provided herein, comprising a modified IL-18 polypeptide comprising E06K and K53A, wherein residue position numbering of the modified IL-18 polypeptide is based on SEQ ID NO: 1 as a reference sequence. In some embodiments, the modified IL-18 polypeptide further comprises T63A. In some embodiments, the modified IL-18 polypeptide further comprises at least one of Y01X, S55X, F02X, D54X, C38X, C68X, E69X, K70X, C76X, or C127X, wherein each X is independently an amino acid or an amino acid derivative. In some embodiments, the modified IL-18 polypeptide further comprises at least one of Y01G, S55A, F02A, D54A, C38S, C38A, C68S, C68A, K70C, C76S, C76A, C127S, or C127A.

In some embodiments, the modified IL-18 peptide comprises at least one additional modification to the amino acid sequence of SEQ ID NO: 1, wherein the modification is E06X, K53X, S55X, or T63X, wherein X is a natural or non-natural amino acid. In some embodiments, the modified IL-18 peptide comprises at least two additional modifications to the amino acid sequence of SEQ ID NO: 1, wherein the modifications comprise E06X and K53X; E06X and S55X; K53X and S55X; E06X and T63X; or K53X and T63X, wherein X is a natural or non-natural amino acid. In some embodiments, the modified IL-18 peptide comprises at least three additional modifications to the amino acid sequence of SEQ ID NO: 1, wherein the modifications comprise E06X, K53X, and S55X; or E06X, K53X, and T63X, wherein X is a natural or non-natural amino acid. In some embodiments, the modified IL-18 peptide comprises at least four additional modifications to the amino acid sequence of SEQ ID NO: 1, wherein the modifications comprise E06X, K53X, S55X, and T63X; E06X, K53X, S55X, and Y01X; E06X, K53X, S55X, and F02X; E06X, K53X, S55X, and D54X; E06X, K53X, S55X, and M51X; or C38X, C68X, C76X, and C127X, wherein X is a natural or non-natural amino acid. In each embodiment wherein a plurality of amino acids residues are replaced with a natural or non-natural amino acid X, each X is independently the same or a different amino acid.

In some embodiments, the modified IL-18 peptide comprises at least one additional modification to the amino acid sequence of SEQ ID NO: 1, wherein the modification is E06K, V11I, K53A, S55A, or T63A. In some embodiments, the modified IL-18 peptide comprises at least two additional modifications to the amino acid sequence of SEQ ID NO: 1, wherein the modifications comprise E06K and K53A; E06K and S55A; K53A and S55A; E06K and T63A; or K53A and T63A. In some embodiments, the modified IL-18 peptide comprises at least three additional modifications to the amino acid sequence of SEQ ID NO: 1, wherein the modifications comprise E06K, K53A, and S55A; E06K, V11I, and K53A; E06K, C38A, and K53A; or E06K, K53A, and T63A. In some embodiments, the modified IL-18 peptide comprises at least four additional modifications to the amino acid sequence of SEQ ID NO: 1, wherein the modifications comprise E06K, K53A, S55A, and T63A; E06K, K53A, S55A, and Y01G; E06K, K53A, S55A, and F02A; E06K, K53A, S55A, and D54A; E06K, K53A, S55A, and M51G; or C38S, C68S, C76S, and C127S. In some embodiments, the modified IL-18 peptide comprises at least six modifications to the amino acid sequence of SEQ ID NO: 1, wherein the modifications comprise E06K, K53A, C38S, C68S, C76S, and C127S; or K53A, T63A, C38S, C68S, C76S, and C127S. In some embodiments, the modified IL-18 peptide comprises at least eight modifications to the amino acid sequence of SEQ ID NO: 1, wherein the modifications comprise Y01G, F02A, E06K, M51G, K53A, D54A, S55A, and T63A. In some embodiments, the modified IL-18 peptide comprises at least eight additional modifications to the amino acid sequence of SEQ ID NO: 1, wherein the modifications comprise Y01G, F02A, E06K, M51G, K53A, D54A, S55A, and T63A.

In one aspect, provided herein, is a modified IL-18 polypeptide with a polymer as provided herein (e.g., a polymer attached to a residue as provided herein), further comprising E06K and K53A, wherein residue position numbering of the modified IL-18 polypeptide is based on SEQ ID NO: 1 as a reference sequence. In some embodiments, the modified IL-18 polypeptide comprises an amino acid sequence at least 80%, at least 85%, at least 90%, at least 95%, or at least 98% identical to the amino acid sequence of SEQ ID NO: 30. In some embodiments, the modified IL-18 polypeptide comprises an amino acid sequence at least 80%, at least 85%, at least 90%, at least 95%, or at least 98% identical to the amino acid sequence of SEQ ID NO: 59. In some embodiments, the modified IL-18 polypeptide further comprises an amino acid substitution at one or more cysteine residues. In some embodiments, the modified IL-18 polypeptide comprises one or more cysteines substituted with either serine or alanine. In some embodiments, the modified IL-18 polypeptide comprises amino acid substitutions at each cysteine residue of SEQ ID NO: 1. In some embodiments, each cysteine residue is substituted with serine or alanine. In some embodiments, the modified IL-18 polypeptide comprises a polymer covalently attached to an amino acid residue. In some embodiments, the modified IL-18 polypeptide comprises amino acid substitutions at 1, 2, 3, 4, 5, or 6 methionine residues. In some embodiments, each substitution at a methionine residue is for an O-methyl-L-homoserine residue or a norleucine residue. In some embodiments, each methionine residue is substituted with an O-methyl-L-homoserine residue. In some embodiments, the modified IL-18 polypeptide comprises homoserine residues at positions 31, 116, and one of 63 and 75. In some embodiments, the modified IL-18 polypeptide comprises homoserine residues at positions 31, 116, 75, and one of 50, 57, 63, and 67. In some embodiments, the modified IL-18 polypeptide comprises homoserine residues at positions 31, 121, 75, and one of 50, 57, 63, and 67.

In some embodiments, a modified IL-18 polypeptide provided herein comprises an amino acid sequence having an amino acid substitution at residue C68 of SEQ ID NO: 30. In some embodiments, the amino acid substitution is a C68S or C68A substitution. In some embodiments, the modified IL-18 polypeptide further comprises a polymer attached at a residue in the region of residues 79-120, such as residue 85, 86, 95, or 98 as provided herein. In some embodiments, the IL-18 polypeptide comprises a cysteine at residue 85, 86, 95, or 98.

In some embodiments, a modified IL-18 polypeptide provided herein comprises an amino acid sequence having an amino acid substitution at residue C68 of SEQ ID NO: 59. In some embodiments, the amino acid substitution is a C68S or C68A substitution. In some embodiments, the modified IL-18 polypeptide further comprises a polymer attached at a residue in the region of residues 79-120, such as residue 85, 86, 95, or 98 as provided herein. In some embodiments, the IL-18 polypeptide comprises a cysteine at residue 85, 86, 95, or 98.

In some embodiments, the modified IL-18 polypeptide comprises a polypeptide sequence having at least about 80%, at least about 85%, at least about 90%, at least about 95%, at least about 98%, at least about 99%, or about 100% sequence identity to SEQ ID NO: 2-12. In some embodiments, the modified IL-18 polypeptide comprises a polypeptide sequence having at least about 80%, at least about 85%, at least about 90%, at least about 95%, at least about 98%, or at least about 99% sequence identity to SEQ ID NO: 13-23. In some embodiments, the modified IL-18 polypeptide comprises a polypeptide sequence having at least about 80%, at least about 85%, at least about 90%, at least about 95%, at least about 98%, or at least about 99% sequence identity to SEQ ID NO: 24-33, optionally containing at least 2 additional amino acid substitutions to the given sequence, wherein one of the amino acid substitutions is at residue C68 (e.g., C68A or C68S), and the other amino acid substitution is at a residue to which the polymer is attached (e.g., residue 85, 86, 95, or 98). In some embodiments, the polypeptide sequence is at least about 80% identical to SEQ ID NO: 30 or SEQ ID NO: 59. In some embodiments, the polypeptide sequence is at least about 80% identical to SEQ ID NO: 30. In some embodiments, the polypeptide sequence is at least about 90% identical to SEQ ID NO: 30. In some embodiments, the polypeptide sequence is at least about 95% identical to SEQ ID NO: 30. In some embodiments, the polypeptide sequence is at least about 80% identical to SEQ ID NO: 59. In some embodiments, the polypeptide sequence is at least about 90% identical to SEQ ID NO: 59. In some embodiments, the polypeptide sequence is at least about 95% identical to SEQ ID NO: 59. In some embodiments, the modified IL-18 polypeptide is recombinant.

In some embodiments, the modified IL-18 polypeptide is synthetic. In some embodiments, the modified IL-18 polypeptide comprises any of the amino acid substitutions present in a synthetic IL-18 polypeptide as provided herein (e.g., one or more homoserine, O-methyl-homoserine, or norleucine residues as provided herein). Any recombinant modified IL-18 polypeptide provided herein may also be prepared as a corresponding synthetic IL-18 polypeptide.

In some embodiments, the modified IL-18 polypeptide comprises one or more amino acid substitutions selected from: (a) a homoserine residue located at any one of residues 26-36; (b) a homoserine residue located at any one of residues 50-70; (c) a homoserine residue located at any one of residues 70-80; (d) a homoserine residue located at any one of residues 110-125; (e) a norleucine or O-methyl-homoserine residue located at any one of residues 28-38; (f) a norleucine or O-methyl-homoserine residue located at any one of residues 46-56; (g) a norleucine or O-methyl-homoserine residue located at any one of residues 54-64; (h) a norleucine or O-methyl-homoserine residue located at any one of residues 80-90; (i) a norleucine or O-methyl-homoserine residue located at any one of residues 108-118; and (j) a norleucine or O-methyl-homoserine residue located at any one of residues 145-155, wherein residue position numbering of the modified IL-18 polypeptide is based on SEQ ID NO: 1 as a reference sequence.

In some embodiments, the modified IL-18 polypeptide comprises one or more amino acid substitutions selected from: (a) a homoserine residue located at any one of residues 26-36; (b) a homoserine residue located at any one of residues 50-70; (c) a homoserine residue located at any one of residues 60-80; (d) a homoserine residue located at any one of residues 110-125; (e) a O-methyl-homoserine residue located at any one of residues 28-38; (f) a O-methyl-homoserine residue located at any one of residues 46-56; (g) a O-methyl-homoserine residue located at any one of residues 54-64; (h) a O-methyl-homoserine residue located at any one of residues 80-90; (i) a O-methyl-homoserine residue located at any one of residues 108-118; and (j) a O-methyl-homoserine residue located at any one of residues 145-155, wherein residue position numbering of the modified IL-18 polypeptide is based on SEQ ID NO: 1 as a reference sequence.

In some embodiments, the modified IL-18 peptide comprises an amino acid substitution with O-methyl-L-homoserine. In some embodiments, the modified IL-18 peptide comprises an amino acid substitution with O-methyl-L-homoserine at positions Met 33, Met 51, Met 60, Met 86, Met 113, or Met 150. In some embodiments, the modified IL-18 polypeptide comprises one or more amino acid substitutions selected from homoserine (Hse) 31, norleucine (Nle) 33, O-methyl-homoserine (Omh) 33, Hse50, Nle51, Omh51, Hse57, Nle60, Omh60, Hse63, Hse 67, Hse75, Nle86, Omh86, Hse116, Nle113, Omh113, Hse 121, Nle150, and Omh150. In some embodiments, each methionine except M86 is substituted with Nle or Omh and residue 86 is attached to the polymer.

In some embodiments, the modified IL-18 polypeptides described herein contain a linker moiety. In some embodiments, the linker moiety includes, but is not limited to, a polymer, linker, spacer, or combinations thereof. When added to certain amino acid residues, the linker moiety can modulate the activity or other properties of the modified IL-18 polypeptide compared to wild-type IL-18.

In some embodiments, a modified IL-18 polypeptide is linked with an additional polypeptide. In some embodiments, the modified IL-18 polypeptide and the additional polypeptide form a fusion polypeptide. In some embodiments, the synthetic IL-18 polypeptide is attached to the additional polypeptide through a non-covalent interaction. In some embodiments, the non-covalent interaction is an interaction biotin with streptavidin or avidin. In some embodiments, the modified IL-18 polypeptide and the additional polypeptide are conjugated together (e.g., using a chemical linker, such as a polymer as provided herein). In some embodiments, the additional polypeptide is an antibody, antibody fragment, single chain variable fragments (ScFv), peptide aptamer, cyclic peptide, branched peptide, growth factor, peptide hormone, chemokine, or cytokine. In some embodiments, the additional polypeptide is attached to the polymer provided herein (e.g., the polymer attached in the region of residues 79-120). In some embodiments, the additional polypeptide comprises an antibody or binding fragment thereof. In some embodiments, the antibody comprises a humanized antibody, a murine antibody, a chimeric antibody, a bispecific antibody, any fragment thereof, or any combination thereof. In some embodiments, the antibody is a monoclonal antibody or any fragment thereof. In some embodiments, the additional polypeptide is a cytokine. In some embodiments, the additional polypeptide is a half-life extension polypeptide (e.g., albumin).

### IV. Biological Activity of Modified IL-18 Polypeptides

IVa. Binding Affinity

### Affinity for IL-18 Receptor Alpha/Beta Heterodimer (Il-18Rαβ)

In some embodiments, the polymer modified IL-18 polypeptide with polymer attached displays at most an only slightly diminished affinity for IL-18Rαβ compared to WT IL-18 (SEQ ID NO: 1). In some embodiments, the polymer modified IL-18 polypeptide exhibits at most a 2-fold lower, at most a 3-fold lower, at most a 4-fold lower, at most 5-fold lower, at most a 10-fold lower, at most a 15-fold lower, at most a 20-fold lower, at most a 30-fold lower, at most a 40-fold lower, at most a 50-fold lower, at most a 75-fold lower, or at most a 100-fold lower affinity for IL-18 Rαβ as compared to the affinity of WT IL-18 for IL-18Rαβ. In some embodiments, the modified IL-18 polypeptide exhibits at most at most a 10-fold lower affinity for IL-18Rαβ as compared to the affinity of WT IL-18 for IL-18Rαβ. In some embodiments, the polymer modified IL-18 polypeptide exhibits at most at most a 20-fold lower affinity for IL-18Rαβ as compared to the affinity of WT IL-18 for IL-18Rαβ. In some embodiments, the polymer modified IL-18 polypeptide exhibits at most at most a 50-fold lower affinity for IL-18Rαβ as compared to the affinity of WT IL-18 for IL-18Rαβ. In some embodiments, the polymer modified IL-18 polypeptide exhibits at most at most a 100-fold lower affinity for IL-18Rαβ as compared to the affinity of WT IL-18 for IL-18Rαβ.

In some embodiments, the modified IL-18 polypeptide without a polymer attached provided herein exhibits an enhanced or only slightly reduced affinity for IL-18Rαβ compared to WT IL-18. In some embodiments, the modified IL-18 polypeptide without a polymer exhibits an enhanced affinity for IL-18Rαβ compared to WT IL-18 (e.g., at least a 2-fold higher, at least a 5-fold higher, at least a 10-fold higher, or at least a 20-fold higher affinity). In some embodiments, the modified IL-18 polypeptide without polymer attached exhibits at least a 30-fold higher, at least a 40-fold higher, or at least a 50-fold higher affinity. In some embodiments, the modified IL-18 polypeptide attached provided herein exhibits an enhanced or only slightly reduced affinity for IL-18Rαβ compared to WT IL-18. In some embodiments, the modified IL-18 polypeptide exhibits an enhanced affinity for IL-18Rαβ compared to WT IL-18 (e.g., at least a 2-fold higher, at least a 5-fold higher, at least a 10-fold higher, at least a 20-fold higher affinity at least a 30-fold higher, at least a 40-fold higher, or at least a 50-fold higher affinity).

In some embodiments, the polymer modified IL-18 polypeptide provided herein with polymer attached exhibits at most only a slight reduction in binding to IL-18Rαβ as measured by K_{D}. In some embodiments, the polymer modified IL-18 polypeptide exhibits a K_{D} with IL-18Rαβ of at most about 20 nM, at most about 30 nM, at most about 50 nM, at most about 75 nM, at most about 100 nM, or at most about 200 nM. In some embodiments, the polymer modified IL-18 polypeptide exhibits a K_{D} with IL-18Rαβ of at most about 20 nM. In some embodiments, the polymer modified IL-18 polypeptide exhibits a K_{D} with IL-18Rαβ of at most about 30 nM. In some embodiments, the polymer modified IL-18 polypeptide exhibits a K_{D} with IL-18Rαβ of at most about 40 nM. In some embodiments, the polymer modified IL-18 polypeptide exhibits a K_{D} with IL-18Rαβ of at most about 50 nM.

In some embodiments, the polymer modified IL-18 polypeptide with the polymer attached as provided herein (e.g., covalently attached to a residue in the region of residues 79-120, such as residue 85, 86, or 98) displays at most an only slightly diminished affinity for IL-18Rαβ compared to the corresponding modified IL-18 polypeptide without the polymer attached. In some embodiments, the polymer modified IL-18 polypeptide exhibits at most a 2-fold lower, at most a 3-fold lower, at most a 4-fold lower, at most 5-fold lower, at most a 10-fold lower, at most a 15-fold lower, at most a 20-fold lower, at most a 30-fold lower, at most a 40-fold lower, at most a 50-fold lower, at most a 75-fold lower, or at most a 100-fold lower affinity for IL-18Rαβ as compared to the affinity of the corresponding modified IL-18 polypeptide without the polymer attached for IL-18Rαβ. In some embodiments, the polymer modified IL-18 polypeptide exhibits at most a 2-fold lower affinity for IL-18Rαβ as compared to the affinity of the corresponding modified IL-18 polypeptide without the polymer attached. In some embodiments, the polymer modified IL-18 polypeptide exhibits at most a 3-fold lower affinity for IL-18Rαβ as compared to the affinity of the corresponding modified IL-18 polypeptide without the polymer attached. In some embodiments, the polymer modified IL-18 polypeptide exhibits at most a 4-fold lower affinity for IL-18Rαβ as compared to the affinity of the corresponding modified IL-18 polypeptide without the polymer attached. In some embodiments, the polymer modified IL-18 polypeptide exhibits at most a 5-fold lower affinity for IL-18Rαβ as compared to the affinity of the corresponding modified IL-18 polypeptide without the polymer attached. In some embodiments, the polymer modified IL-18 polypeptide exhibits at most a 10-fold lower affinity for IL-18Rαβ as compared to the affinity of the corresponding modified IL-18 polypeptide without the polymer attached.

### Affinity for IL-18 Receptor Alpha (IL-18Rα)

The modified IL-18 polypeptide provided herein can exhibit higher binding affinity for IL-18 receptor alpha subunit, compared to WT IL-18 (SEQ ID NO: 1). In certain embodiments, the modified IL-18 polypeptide provided herein exhibits higher binding affinity for IL-18 receptor alpha subunit, compared to WT IL-18 by at least 50-fold, at least 52-fold, at least 60-fold, at least 70-fold, at least 80-fold, at least 90-fold, at least 100-fold, at least 120-fold, at least 150-fold, at least 200-fold, at least 300-fold, or at least 500-fold. The modified IL-18 polypeptide can exhibit a dissociation constant (K_{D}) with the IL-18 receptor alpha subunit at least 50-fold lower than the K_{D} for WT IL-18. In certain embodiments, the modified IL-18 polypeptide exhibits a K_{D} with the IL-18 receptor alpha subunit at least 52-fold, 60-fold, 70-fold, 80-fold, 90-fold, or 100-fold lower than the K_{D} for WT IL-18. In certain embodiments, the modified IL-18 polypeptide exhibits a K_{D} with the IL-18 receptor alpha subunit at least 52-fold lower than the K_{D} for WT IL-18. In certain embodiments, the modified IL-18 polypeptide exhibits a K_{D} with the IL-18 receptor alpha subunit at least 60-fold lower than the K_{D} for WT IL-18. In certain embodiments, the modified IL-18 polypeptide exhibits a K_{D} with the IL-18 receptor alpha subunit at least 70-fold lower than the K_{D} for WT IL-18. In certain embodiments, the modified IL-18 polypeptide exhibits a K_{D} with the IL-18 receptor alpha subunit at least 80-fold lower than the K_{D} for WT IL-18. In certain embodiments, the modified IL-18 polypeptide exhibits a K_{D} with the IL-18 receptor alpha subunit at least 90-fold lower than the K_{D} for WT IL-18. In certain embodiments, the modified IL-18 polypeptide exhibits a K_{D} with the IL-18 receptor alpha subunit at least 100-fold lower than the K_{D} for WT IL-18. In certain embodiments, the modified IL-18 polypeptide exhibits K_{D} with the IL-18 receptor alpha subunit, lower than the K_{D} for WT IL-18 by 50-fold to 500-fold. In certain embodiments, the modified IL-18 polypeptide exhibits a K_{D} with the IL-18 receptor alpha subunit, lower than the K_{D} for WT IL-18 by 50-fold to 52-fold, 50-fold to 60-fold, 50-fold to 70-fold, 50-fold to 80-fold, 50-fold to 90-fold, 50-fold to 100-fold, 50-fold to 120-fold, 50-fold to 150-fold, 50-fold to 200-fold, 50-fold to 300-fold, 50-fold to 500-fold, 52-fold to 60-fold, 52-fold to 70-fold, 52-fold to 80-fold, 52-fold to 90-fold, 52-fold to 100-fold, 52-fold to 120-fold, 52-fold to 150-fold, 52-fold to 200-fold, 52-fold to 300-fold, 52-fold to 500-fold, 60-fold to 70-fold, 60-fold to 80-fold, 60-fold to 90-fold, 60-fold to 100-fold, 60-fold to 120-fold, 60-fold to 150-fold, 60-fold to 200-fold, 60-fold to 300-fold, 60-fold to 500-fold, 70-fold to 80-fold, 70-fold to 90-fold, 70-fold to 100-fold, 70-fold to 120-fold, 70-fold to 150-fold, 70-fold to 200-fold, 70-fold to 300-fold, 70-fold to 500-fold, 80-fold to 90-fold, 80-fold to 100-fold, 80-fold to 120-fold, 80-fold to 150-fold, 80-fold to 200-fold, 80-fold to 300-fold, 80-fold to 500-fold, 90-fold to 100-fold, 90-fold to 120-fold, 90-fold to 150-fold, 90-fold to 200-fold, 90-fold to 300-fold, 90-fold to 500-fold, 100-fold to 120-fold, 100-fold to 150-fold, 100-fold to 200-fold, 100-fold to 300-fold, 100-fold to 500-fold, 120-fold to 150-fold, 120-fold to 200-fold, 120-fold to 300-fold, 120-fold to 500-fold, 150-fold to 200-fold, 150-fold to 300-fold, 150-fold to 500-fold, 200-fold to 300-fold, 200-fold to 500-fold, or 300-fold to 500-fold. In certain embodiments, the modified IL-18 polypeptide exhibits a K_{D} with the IL-18 receptor alpha subunit, lower than the K_{D} for WT IL-18 by 50-fold, 52-fold, 60-fold, 70-fold, 80-fold, 90-fold, 100-fold, 120-fold, 150-fold, 200-fold, 300-fold, or 500-fold. In certain embodiments, the modified IL-18 polypeptide exhibits a K_{D} with the IL-18 receptor alpha subunit, lower than the K_{D} for WT IL-18 by at least 50-fold, 52-fold, 60-fold, 70-fold, 80-fold, 90-fold, 100-fold, 120-fold, 150-fold, 200-fold, or 300-fold.

In some embodiments, the polymer modified IL-18 polypeptide with polymer attached displays at most an only slightly diminished affinity for IL-18Rα compared to WT IL-18 (SEQ ID NO: 1). In some embodiments, the polymer modified IL-18 polypeptide exhibits at most a 2-fold lower, at most a 3-fold lower, at most a 4-fold lower, at most 5-fold lower, at most a 10-fold lower, at most a 15-fold lower, at most a 20-fold lower, at most a 30-fold lower, at most a 40-fold lower, at most a 50-fold lower, at most a 75-fold lower, at most a 100-fold lower affinity, at most a 200-fold lower affinity, or at most a 500-fold lower affinity for IL-18Rα as compared to the affinity of WT IL-18 for IL-18Rα. In some embodiments, the polymer modified IL-18 polypeptide exhibits at most at most a 10-fold lower affinity for IL-18Rα as compared to the affinity of WT IL-18 for IL-18Rα. In some embodiments, the polymer modified IL-18 polypeptide exhibits at most at most a 20-fold lower affinity for IL-18Rα as compared to the affinity of WT IL-18 for IL-18Rα. In some embodiments, the polymer modified IL-18 polypeptide exhibits at most at most a 50-fold lower affinity for IL-18Rα as compared to the affinity of WT IL-18 for IL-18Rα. In some embodiments, the polymer modified IL-18 polypeptide exhibits at most at most a 100-fold lower affinity for IL-18Rα as compared to the affinity of WT IL-18 for IL-18Rα.

In some embodiments, the polymer modified IL-18 polypeptide provided herein exhibits at most only a slight reduction in binding to IL-18Rα compared to as measured by K_{D}. In some embodiments, the polymer modified IL-18 polypeptide exhibits a K_{D} with IL-18Rα of at most about 20 nM, at most about 30 nM, at most about 50 nM, at most about 75 nM, at most about 100 nM, at most about 200 nM, at most about 500 nM, at most about 1000 nM, at most about 2000 nM, at most about 3000 nM, at most about 5000 nM. In some embodiments, the polymer modified IL-18 polypeptide exhibits a K_{D} with IL-18Rα of at most about 100 nM. In some embodiments, the polymer modified IL-18 polypeptide exhibits a K_{D} with IL-18Rα of at most about 200 nM. In some embodiments, the polymer modified IL-18 polypeptide exhibits a K_{D} with IL-18Rα of at most about 500 nM. In some embodiments, the polymer modified IL-18 polypeptide exhibits a K_{D} with IL-18Rα of at most about 1000 nM. In some embodiments, the polymer modified IL-18 polypeptide exhibits a K_{D} with IL-18Rα of at most about 5000 nM.

In some embodiments, the polymer modified IL-18 polypeptide with the polymer attached as provided herein (e.g., covalently attached to a residue in the region of residues 79-100, such as residue 85, 86, or 98) displays at most an only slightly diminished affinity for IL-18Rα compared to the corresponding modified IL-18 polypeptide without the polymer attached. In some embodiments, the polymer modified IL-18 polypeptide exhibits at most a 2-fold lower, at most a 3-fold lower, at most a 4-fold lower, at most 5-fold lower, at most a 10-fold lower, at most a 15-fold lower, at most a 20-fold lower, at most a 30-fold lower, at most a 40-fold lower, at most a 50-fold lower, at most a 75-fold lower, at most a 100-fold lower, at most a 200-fold lower, or at most a 500-fold lower affinity for IL-18Rαβ as compared to the affinity of the corresponding modified IL-18 polypeptide without the polymer attached for IL-18Rα. In some embodiments, the polymer modified IL-18 polypeptide exhibits at most a 2-fold lower affinity for IL-18Rα as compared to the affinity of the corresponding modified IL-18 polypeptide without the polymer attached. In some embodiments, the polymer modified IL-18 polypeptide exhibits at most a 3-fold lower affinity for IL-18Rα as compared to the affinity of the corresponding modified IL-18 polypeptide without the polymer attached. In some embodiments, the polymer modified IL-18 polypeptide exhibits at most a 4-fold lower affinity for IL-18Rα as compared to the affinity of the corresponding modified IL-18 polypeptide without the polymer attached. In some embodiments, the polymer modified IL-18 polypeptide exhibits at most a 5-fold lower affinity for IL-18Rα as compared to the affinity of the corresponding modified IL-18 polypeptide without the polymer attached. In some embodiments, the polymer modified IL-18 polypeptide exhibits at most a 10-fold lower affinity for IL-18Rα as compared to the affinity of the corresponding modified IL-18 polypeptide without the polymer attached.

### Affinity for IL-18BP

In some embodiments, the modified IL-18 polypeptide provided herein exhibits reduced affinity for IL-18 binding protein (IL-18BP) compared to WT IL-18 (SEQ ID NO: 1). In some embodiments, the modified IL-18 polypeptide exhibits at least 2-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, or at least 200-fold, lower affinity for IL-18BP compared to WT IL-18. In some embodiments, the modified IL-18 polypeptide exhibits at least 2-fold lower affinity for IL-18BP compared to WT IL-18. In some embodiments, the modified IL-18 polypeptide exhibits at least 2-fold lower affinity for IL-18BP compared to WT IL-18. In some embodiments, the modified IL-18 polypeptide exhibits at least 5-fold lower affinity for IL-18BP compared to WT IL-18. In some embodiments, the modified IL-18 polypeptide exhibits at least 10-fold lower affinity for IL-18BP compared to WT IL-18. In some embodiments, the modified IL-18 polypeptide exhibits at least 20-fold lower affinity for IL-18BP compared to WT IL-18. In some embodiments, the modified IL-18 polypeptide exhibits at least 30-fold lower affinity for IL-18BP compared to WT IL-18. In some embodiments, the modified IL-18 polypeptide exhibits at least 40-fold lower affinity for IL-18BP compared to WT IL-18. In some embodiments, the modified IL-18 polypeptide exhibits at least 50-fold lower affinity for IL-18BP compared to WT IL-18. In some embodiments, the modified IL-18 polypeptide exhibits at least 60-fold lower affinity for IL-18BP compared to WT IL-18. In some embodiments, the modified IL-18 polypeptide exhibits at least 70-fold lower affinity for IL-18BP compared to WT IL-18. In some embodiments, the modified IL-18 polypeptide exhibits at least 80-fold lower affinity for IL-18BP compared to WT IL-18. In some embodiments, the modified IL-18 polypeptide exhibits at least 90-fold lower affinity for IL-18BP compared to WT IL-18. In some embodiments, the modified IL-18 polypeptide exhibits at least 100-fold lower affinity for IL-18BP compared to WT IL-18. In some embodiments, the modified IL-18 polypeptide exhibits at least 200-fold lower affinity for IL-18BP compared to WT IL-18.

In some embodiments, the polymer modified IL-18 polypeptide provided herein exhibit reduced affinity for IL-18 binding protein (IL-18BP) compared to WT IL-18 (SEQ ID NO: 1). In some embodiments, polymer the modified IL-18 polypeptide exhibits at least 2-fold, at least 5-fold, at least 10-fold, at least 20-fold, at least 30-fold, at least 40-fold, at least 50-fold, at least 60-fold, at least 70-fold, at least 80-fold, at least 90-fold, or at least 100-fold lower affinity for IL-18BP compared to WT IL-18. In some embodiments, the polymer modified IL-18 polypeptide exhibits at least a 10-fold lower affinity for IL-18BP compared to WT IL-18. In some embodiments, the polymer modified IL-18 polypeptide exhibits at least a 20-fold lower affinity for IL-18BP compared to WT IL-18. In some embodiments, the polymer modified IL-18 polypeptide exhibits at least a 50-fold lower affinity for IL-18BP compared to WT IL-18. In some embodiments, the polymer modified IL-18 polypeptide exhibits at least an 80-fold lower affinity for IL-18BP compared to WT IL-18. In some embodiments, the polymer modified IL-18 polypeptide exhibits at least a 100-fold lower affinity for IL-18BP compared to WT IL-18.

In some embodiments, the polymer modified IL-18 polypeptide provided herein exhibits a reduced binding to IL-18BP to WT IL-18 as measured by K_{D}. In some embodiments, the polymer modified IL-18 polypeptide exhibits a K_{D} with IL-18BP of at least about 1 nM, at least about 5 nM, at least about 10 nM, at least about 15 nM, at least about 20 nM, at least about 25 nM, at least about 50 nM, at least about 100 nM, at least about 200 nM, at least about 300 nM, at least about 400 nM, or at least about 500 nM. In some embodiments, the polymer modified IL-18 polypeptide exhibits a K_{D} with IL-18BP of at least about 1 nM. In some embodiments, the polymer modified IL-18 polypeptide exhibits a K_{D} with IL-18BP of at least about 5 nM. In some embodiments, the polymer modified IL-18 polypeptide exhibits a K_{D} with IL-18BP of at least about 50 nM. In some embodiments, the polymer modified IL-18 polypeptide exhibits a K_{D} with IL-18BP of at least about 100 nM. In some embodiments, the polymer modified IL-18 polypeptide exhibits a K_{D} with IL-18BP of at least about 500 nM.

### Ratios of Affinities for IL-18BP and Various IL-18 Receptors

In some embodiments, the modified IL-18 polypeptide exhibits a wide window in which the modified IL-18 polypeptide will bind to IL-18Rαβ even in the presence of IL-18 BP. In some embodiments, this window can be measured by a ratio of K_{D} of the IL-18/IL-18BP interaction over K_{D} of the IL-18/IL-18Rαβ interaction, where a larger number indicates a larger window in which the modified IL-18 polypeptide is expected to be active in vivo. In some embodiments, the modified IL-18 polypeptide exhibits a ratio of K_{D} of the IL-18/IL-18BP interaction over K_{D} of the IL-18/IL-18Rαβ interaction of at least about 1, at least about 2, at least about 3, at least about 4, at least about 5, at least about 10, at least about 15, at least about 20, at least about 25, at least about 30, at least about 35, at least about 40, at least about 45, or at least about 50. In some embodiments, the modified IL-18 polypeptide exhibits a ratio of K_{D} of the IL-18/IL-18BP interaction over K_{D} of the IL-18/IL-18Rαβ interaction of at least about 2. In some embodiments, the modified IL-18 polypeptide exhibits a ratio of K_{D} of the IL-18/IL-18BP interaction over K_{D} of the IL-18/IL-18Rαβ interaction of at least about 5. In some embodiments, the modified IL-18 polypeptide exhibits a ratio of K_{D} of the IL-18/IL-18BP interaction over K_{D} of the IL-18/IL-18Rαβ interaction of at least about 10. In some embodiments, the modified IL-18 polypeptide exhibits a ratio of K_{D} of the IL-18/IL-18BP interaction over K_{D} of the IL-18/IL-18Rαβ interaction of at least about 25. In some embodiments, the modified IL-18 polypeptide exhibits a ratio of K_{D} of the IL-18/IL-18BP interaction over K_{D} of the IL-18/IL-18Rαβ interaction of at least about 30. In some embodiments, the modified IL-18 polypeptide exhibits a ratio of K_{D} of the IL-18/IL-18BP interaction over K_{D} of the IL-18/IL-18Rαβ interaction of at least about 40. In some embodiments, the modified IL-18 polypeptide exhibits a ratio of K_{D} of the IL-18/IL-18BP interaction over K_{D} of the IL-18/IL-18Rαβ interaction of at least about 45. In some embodiments, the modified IL-18 polypeptide exhibits a ratio of K_{D} of the IL-18/IL-18BP interaction over K_{D} of the IL-18/IL-18Rαβ interaction of at least about 50.

In some embodiments, the polymer modified IL-18 polypeptide exhibits a wide window in which the polymer modified IL-18 polypeptide will bind to IL-18R(XP even in the presence of IL-18 BP. In some embodiments, this window can be measured by a ratio of K_{D} of the IL-18/IL-18BP interaction over K_{D} of the IL-18/IL-18Rαβ interaction, where a larger number indicates a larger window in which the polymer modified IL-18 polypeptide is expected to be active in vivo. In some embodiments, the polymer modified IL-18 polypeptide exhibits a ratio of K_{D} Of the IL-18/IL-18BP interaction over K_{D} of the IL-18/IL-18Rαβ interaction of at least about 1, at least about 2, at least about 3, at least about 4, at least about 5, at least about 10, at least about 15, at least about 20, at least about 25, at least about 30, at least about 35, at least about 40, at least about 45, or at least about 50. In some embodiments, the polymer modified IL-18 polypeptide exhibits a ratio of K_{D} of the IL-18/IL-18BP interaction over K_{D} of the IL-18/IL-18Rαβ interaction of at least about 2. In some embodiments, the polymer modified IL-18 polypeptide exhibits a ratio of K_{D} of the IL-18/IL-18BP interaction over K_{D} of the IL-18/IL-18Rαβ interaction of at least about 5. In some embodiments, the polymer modified IL-18 polypeptide exhibits a ratio of K_{D} of the IL-18/IL-18BP interaction over K_{D} of the IL-18/IL-18Rαβ interaction of at least about 10. In some embodiments, the polymer modified IL-18 polypeptide exhibits a ratio of K_{D} of the IL-18/IL-18BP interaction over K_{D} of the IL-18/IL-18Rαβ interaction of at least about 25. In some embodiments, the polymer modified IL-18 polypeptide exhibits a ratio of K_{D} of the IL-18/IL-18BP interaction over K_{D} of the IL-18/IL-18Rαβ interaction of at least about 30. In some embodiments, the polymer modified IL-18 polypeptide exhibits a ratio of K_{D} of the IL-18/IL-18BP interaction over K_{D} of the IL-18/IL-18Rαβ interaction of at least about 40. In some embodiments, the polymer modified IL-18 polypeptide exhibits a ratio of K_{D} of the IL-18/IL-18BP interaction over K_{D} of the IL-18/IL-18Rαβ interaction of at least about 45. In some embodiments, the polymer modified IL-18 polypeptide exhibits a ratio of K_{D} of the IL-18/IL-18BP interaction over K_{D} of the IL-18/IL-18Rαβ interaction of at least about 50.

In some embodiments, the ratio of K_{D} of the IL-18/IL-18BP over K_{D} of IL-18/IL-18Rαβ for the polymer modified IL-18 polypeptide with the polymer attached at a residue provided herein (e.g., covalently attached to a residue in the region of residues 79-120, such as residue 85, 86, or 98) is increased compared to WT IL-18. In some embodiments, the ratio is increased by at least 2-fold, at least 3-fold, at least 4-fold, at least 5-fold, at least 10-fold, at least 15-fold, or at least 20-fold compared to the ratio for WT IL-18.

IVb. Functional Activity

The modified IL-18 polypeptide provided herein exhibits a higher ability to signal through the IL-18 receptor (IL-18R) as compared to WT IL-18. The modified IL-18 polypeptide also displays a reduced ability to be inhibited by IL-18BP compared to WT IL-18.

In some embodiments, a polymer modified IL-18 polypeptide with polymer attached (e.g., at one of residues 79-120) provided herein display one or more activities associated with WT IL-18. In some embodiments, the polymer modified IL-18 polypeptide exhibits a similar ability to signal through the IL-18 receptor (IL-18R) as compared to a corresponding modified IL-18 polypeptide without the polymer attached (e.g., substantially the same or only slightly reduced ability). In some embodiments, the polymer modified IL-18 polypeptide exhibits a similar ability to signal through IL-18R as compared to WT IL-18. In some embodiments, the polymer modified IL-18 polypeptide's ability to signal through IL-18R is reduced compared to WT IL-18 by only a small amount.

In some embodiments, the polymer modified IL-18 polypeptide also displays a reduced ability to be inhibited by IL-18BP compared to WT IL-18. In some embodiments, the polymer modified IL-18 polypeptide with polymer attached displays a reduced ability to be inhibited by IL-18BP compared to a corresponding modified IL-18 polypeptide without the polymer attached.

### IFNγ Production Stimulation Activity (EC₅₀)

In some embodiments, the modified IL-18 polypeptide modulates IFNγ production when in contact with a cell (e.g., an immune cell, such as an NK cell). In some embodiments, the modified IL-18 polypeptide's ability to modulate IFNγ production is measured as a half-maximal effective concentration (EC₅₀). The modified IL-18 polypeptide exhibits higher ability to induce IFNγ production in a cell compared to WT IL-18. In certain embodiments, the EC50 to induce IFNγ production in a cell for the modified IL-18 polypeptide is lower than the EC50 for WT IL-18 by 25-fold to 200-fold. In certain embodiments, the EC50 to induce IFNγ production in a cell for the modified IL-18 polypeptide is lower than the EC50 for WT IL-18 by 25-fold to 30-fold, 25-fold to 40-fold, 25-fold to 50-fold, 25-fold to 60-fold, 25-fold to 70-fold, 25-fold to 80-fold, 25-fold to 90-fold, 25-fold to 100-fold, 25-fold to 150-fold, 25-fold to 200-fold, 30-fold to 40-fold, 30-fold to 50-fold, 30-fold to 60-fold, 30-fold to 70-fold, 30-fold to 80-fold, 30-fold to 90-fold, 30-fold to 100-fold, 30-fold to 150-fold, 30-fold to 200-fold, 40-fold to 50-fold, 40-fold to 60-fold, 40-fold to 70-fold, 40-fold to 80-fold, 40-fold to 90-fold, 40-fold to 100-fold, 40-fold to 150-fold, 40-fold to 200-fold, 50-fold to 60-fold, 50-fold to 70-fold, 50-fold to 80-fold, 50-fold to 90-fold, 50-fold to 100-fold, 50-fold to 150-fold, 50-fold to 200-fold, 60-fold to 70-fold, 60-fold to 80-fold, 60-fold to 90-fold, 60-fold to 100-fold, 60-fold to 150-fold, 60-fold to 200-fold, 70-fold to 80-fold, 70-fold to 90-fold, 70-fold to 100-fold, 70-fold to 150-fold, 70-fold to 200-fold, 80-fold to 90-fold, 80-fold to 100-fold, 80-fold to 150-fold, 80-fold to 200-fold, 80-fold to 500-fold, 90-fold to 100-fold, 90-fold to 150-fold, 90-fold to 200-fold, 100-fold to 150-fold, 100-fold to 200-fold, or 150-fold to 200-fold. In certain embodiments, the EC50 to induce IFNγ production in a cell for the modified IL-18 polypeptide is lower than the EC50 for WT IL-18 by 25-fold, 30-fold, 40-fold, 50-fold, 60-fold, 70-fold, 80-fold, 90-fold, 100-fold, 150-fold, or 200-fold. In certain embodiments, the EC50 to induce IFNγ production in a cell for the modified IL-18 polypeptide is lower than the EC50 for WT IL-18 by at least 25-fold, 30-fold, 40-fold, 50-fold, 60-fold, 70-fold, 80-fold, 90-fold, 100-fold, 150-fold, or 200-fold. In certain embodiments, the EC50 to induce IFNγ production in a cell for the modified IL-18 polypeptide is at least 30-fold lower than the EC50 for WT IL-18. In certain embodiments, the EC50 to induce IFNγ production in a cell for the modified IL-18 polypeptide is at least 40-fold lower than the EC50 for WT IL-18. In certain embodiments, the EC50 to induce IFNγ production in a cell for the modified IL-18 polypeptide is at least 50-fold lower than the EC50 for WT IL-18. In certain embodiments, the EC50 to induce IFNγ production in a cell for the modified IL-18 polypeptide is at least 60-fold lower than the EC50 for WT IL-18. In certain embodiments, the EC50 to induce IFNγ production in a cell for the modified IL-18 polypeptide is at least 70-fold lower than the EC50 for WT IL-18. In certain embodiments, the EC50 to induce IFNγ production in a cell for the modified IL-18 polypeptide is at least 80-fold lower than the EC50 for WT IL-18.

In certain embodiments, the EC50 for signaling through the IL-18 receptor, for the modified IL-18 polypeptide is lower than the EC50 for WT IL-18 by 4.6-fold to 200-fold. In certain embodiments, the EC50 for signaling through the IL-18 receptor, for the modified IL-18 polypeptide is lower than the EC50 for WT IL-18 by 4.6-fold to 4.7-fold, 4.6-fold to 5-fold, 4.6-fold to 10-fold, 4.6-fold to 20-fold, 4.6-fold to 30-fold, 4.6-fold to 40-fold, 4.6-fold to 50-fold, 4.6-fold to 75-fold, 4.6-fold to 100-fold, 4.6-fold to 200-fold, 4.7-fold to 5-fold, 4.7-fold to 10-fold, 4.7-fold to 20-fold, 4.7-fold to 30-fold, 4.7-fold to 40-fold, 4.7-fold to 50-fold, 4.7-fold to 75-fold, 4.7-fold to 100-fold, 4.7-fold to 200-fold, 5-fold to 10-fold, 5-fold to 20-fold, 5-fold to 30-fold, 5-fold to 40-fold, 5-fold to 50-fold, 5-fold to 75-fold, 5-fold to 100-fold, 5-fold to 200-fold, 10-fold to 20-fold, 10-fold to 30-fold, 10-fold to 40-fold, 10-fold to 50-fold, 10-fold to 75-fold, 10-fold to 100-fold, 10-fold to 200-fold, 20-fold to 30-fold, 20-fold to 40-fold, 20-fold to 50-fold, 20-fold to 75-fold, 20-fold to 100-fold, 20-fold to 200-fold, 30-fold to 40-fold, 30-fold to 50-fold, 30-fold to 75-fold, 30-fold to 100-fold, 30-fold to 200-fold, 40-fold to 50-fold, 40-fold to 75-fold, 40-fold to 100-fold, 40-fold to 200-fold, 50-fold to 75-fold, 50-fold to 100-fold, 50-fold to 200-fold, 75-fold to 100-fold, 75-fold to 200-fold, or 100-fold to 200-fold. In certain embodiments, the EC50 for signaling through the IL-18 receptor, for the modified IL-18 polypeptide is lower than the EC50 for WT IL-18 by 4.6-fold, 4.7-fold, 5-fold, 10-fold, 20-fold, 30-fold, 40-fold, 50-fold, 75-fold, 100-fold, or 200-fold. In certain embodiments, the EC50 for signaling through the IL-18 receptor, for the modified IL-18 polypeptide is lower than the EC50 for WT IL-18 by at least 4.6-fold, 4.7-fold, 5-fold, 10-fold, 20-fold, 30-fold, 40-fold, 50-fold, 75-fold, 100-fold, or 200-fold. In certain embodiments, the EC50 for signaling through the IL-18 receptor, for the modified IL-18 polypeptide is at least 4.7-fold lower than the EC50 for WT IL-18. In certain embodiments, the EC50 for signaling through the IL-18 receptor, for the modified IL-18 polypeptide is at least 5-fold lower than the EC50 for WT IL-18. In certain embodiments, the EC50 for signaling through the IL-18 receptor, for the modified IL-18 polypeptide is at least 10-fold lower than the EC50 for WT IL-18. In certain embodiments, the EC50 for signaling through the IL-18 receptor, for the modified IL-18 polypeptide is at least 20-fold lower than the EC50 for WT IL-18. In certain embodiments, the EC50 for signaling through the IL-18 receptor, for the modified IL-18 polypeptide is at least 30-fold lower than the EC50 for WT IL-18. In certain embodiments, the EC50 for signaling through the IL-18 receptor, for the modified IL-18 polypeptide is at least 40-fold lower than the EC50 for WT IL-18. In certain embodiments, the EC50 for signaling through the IL-18 receptor, for the modified IL-18 polypeptide is at least 50-fold lower than the EC50 for WT IL-18. EC50 for signaling through the IL-18 receptor can be measured using HEK-Blue IL18R Reporter Assay (e.g., as provided herein in the Examples).

In some embodiments, an EC₅₀ (nM) of the polymer modified IL-18 polypeptide's ability to induce IFNγ is less than 10-fold higher than, less than 5-fold higher than, or less than an EC₅₀ (nM) of an IL-18 polypeptide of SEQ ID NO: 1. In some embodiments, the EC₅₀ of the polymer modified IL-18 polypeptide's ability to induce IFNγ is less than 10-fold higher than an EC₅₀ (nM) of an IL-18 polypeptide of SEQ ID NO: 1. In some embodiments, the EC₅₀ of the polymer modified IL-18 polypeptide's ability to induce IFNγ is less than 5-fold higher than an EC₅₀ (nM) of an IL-18 polypeptide of SEQ ID NO: 1. In some embodiments, the EC₅₀ of the polymer modified IL-18 polypeptide's ability to induce IFNγ is less than an EC₅₀ (nM) of an IL-18 polypeptide of SEQ ID NO: 1. In some embodiments, the EC₅₀ of the modified IL-18 polypeptide's ability to induce IFNγ is less than 10-fold higher than, less than 8-fold higher than, less than 6-fold higher than, less than 5-fold higher than, less than 4-fold higher than, less than 3-fold higher than, or less than 2-fold higher than an EC₅₀ (nM) of an IL-18 polypeptide of SEQ ID NO: 1. In some embodiments, the EC₅₀ of the polymer modified IL-18 polypeptide's ability to induce IFNγ is measured by an IFNγ induction cellular assay.

In some embodiments, an EC₅₀ of the polymer modified IL-18 polypeptide's ability to induce IFNγ production is less than about 100 nM, less than about 75 nM, less than about 50 nM, less than about 40 nM, less than about 30 nM, less than about 20 nM, less than about 15 nM, or less than about 10 nM. In some embodiments, an EC₅₀ of the polymer modified IL-18 polypeptide's ability to induce IFNγ production is less than about 100 nM. In some embodiments, an EC₅₀ of the polymer modified IL-18 polypeptide's ability to induce IFNγ production is less than about 80 nM. In some embodiments, an EC₅₀ of the polymer modified IL-18 polypeptide's ability to induce IFNγ production is less than about 50 nM. In some embodiments, an EC₅₀ of the polymer modified IL-18 polypeptide's ability to induce IFNγ production is less than about 10 nM.

In some embodiments, the polymer modified IL-18 polypeptide with a polymer attached provided herein (e.g., a polymer attached in the region of restudies 79-120, such as residue 85, 86, or 98) displays a similar or only slightly reduced ability to induce IFNγ production compared to a corresponding modified IL-18 polypeptide without the polymer attached. In some embodiments, an EC₅₀ (nM) of IFNγ production of the polymer modified IL-18 polypeptide with the polymer attached is at most about 100-fold higher than, at most about 50-fold higher than, at most about 10-fold higher than, at most about 9-fold higher than, at most about 8-fold, higher than at most about 7-fold higher than, at most about 6-fold higher than, at most about 5-fold higher than, at most about 4-fold higher than, at most about 3-fold higher than, or at most about 2-fold higher than an EC₅₀ of the modified IL-18 polypeptide without the polymer attached. In some embodiments, an EC₅₀ (nM) of IFNγ production of the polymer modified IL-18 polypeptide with the polymer attached is at most about 10-fold higher than an EC₅₀ of the modified IL-18 polypeptide without the polymer attached. In some embodiments, an EC₅₀ (nM) of IFNγ production of the polymer modified IL-18 polypeptide with the polymer attached is at most about 5-fold higher than an EC₅₀ of the modified IL-18 polypeptide without the polymer attached. In some embodiments, an EC₅₀ (nM) of IFNγ production of the polymer modified IL-18 polypeptide with the polymer attached is at most about 4-fold higher than an EC₅₀ of the modified IL-18 polypeptide without the polymer attached. In some embodiments, an EC₅₀ (nM) of IFNγ production of the polymer modified IL-18 polypeptide with the polymer attached is at most about 3-fold higher than an EC₅₀ of the modified IL-18 polypeptide without the polymer attached. In some embodiments, an EC₅₀ (nM) of IFNγ production of the polymer modified IL-18 polypeptide with the polymer attached is at most about 2-fold higher than an EC₅₀ of the modified IL-18 polypeptide without the polymer attached.

### Inhibition by IL-18BP (IC₅₀)

In some embodiments, the modified IL-18 exhibits a reduced ability to have its IFNγ induction activity inhibited by IL-18BP compared to WT IL-18. In some embodiments, the modified IL-18 displays a half-maximal inhibitory concentration (IC₅₀) by IL-18BP which is at least about 10-fold higher than, at least about 20-fold higher than, at least about 50-fold higher than, at least about 75-fold higher than, at least about 100-fold higher than, at least about 200-fold higher than, at least about 300-fold higher than, at least about 400-fold higher than, at least about 500-fold higher than, at least about 600-fold higher than, at least about 700-fold higher than, at least about 800-fold higher than, at least about 900-fold higher than, or at least about 1000-fold higher than an IC₅₀ of WT IL-18's inhibition by IL-18BP. In some embodiments, the modified IL-18 displays a half-maximal inhibitory concentration (IC₅₀) by IL-18BP which is at least about 100-fold higher than an IC₅₀ of WT IL-18's inhibition by IL-18BP. In some embodiments, the modified IL-18 displays a half-maximal inhibitory concentration (IC₅₀) by IL-18BP which is at least about 500-fold higher than an IC₅₀ of WT IL-18's inhibition by IL-18BP. In some embodiments, the modified IL-18 displays a half-maximal inhibitory concentration (IC₅₀) by IL-18BP which is at least about 1000-fold higher than an IC₅₀ of WT IL-18's inhibition by IL-18BP.

In some embodiments, the modified IL-18 displays a half-maximal inhibitory concentration (IC₅₀) by IL-18BP which is at about 10 nM, at least about 50 nM, at least about 100 nM, at least about 200 nM, at least about 300 nM, at least about 400 nM, at least about 500 nM, at least about 600 nM, at least about 700 nM, at least about 800 nM, at least about 900 nM, or at least about 1000 nM. In some embodiments, the modified IL-18 displays a half-maximal inhibitory concentration (IC₅₀) by IL-18BP which is at least about 10 nM. In some embodiments, the modified IL-18 displays a half-maximal inhibitory concentration (IC₅₀) by IL-18BP which is at least about 100 nM. In some embodiments, the modified IL-18 displays a half-maximal inhibitory concentration (IC₅₀) by IL-18BP which is at least about 1000 nM.

In certain embodiments, the dissociation constant (K_{D}) with IL-18BP, for the modified IL-18 polypeptide is 250 nM to 10,000 nM. In certain embodiments, the K_{D} with IL-18BP, for the modified IL-18 polypeptide is 2 nM to 10 nM, 2 nM to 20 nM, 2 nM to 50 nM, 2 nM to 100 nM, 2 nM to 200 nM, 2 nM to 500 nM, 10 nM to 50 nM, 10 nM to 100 nM, 10 nM to 500 nM, 20 nM to 50 nM, 20 nM to 100 nM, 20 nM to 500 nM, 250 nM to 500 nM, 250 nM to 750 nM, 250 nM to 1,000 nM, 250 nM to 1,250 nM, 250 nM to 1,500 nM, 250 nM to 2,000 nM, 250 nM to 3,000 nM, 250 nM to 4,000 nM, 250 nM to 5,000 nM, 250 nM to 7,500 nM, 250 nM to 10,000 nM, 500 nM to 750 nM, 500 nM to 1,000 nM, 500 nM to 1,250 nM, 500 nM to 1,500 nM, 500 nM to 2,000 nM, 500 nM to 3,000 nM, 500 nM to 4,000 nM, 500 nM to 5,000 nM, 500 nM to 7,500 nM, 500 nM to 10,000 nM, 750 nM to 1,000 nM, 750 nM to 1,250 nM, 750 nM to 1,500 nM, 750 nM to 2,000 nM, 750 nM to 3,000 nM, 750 nM to 4,000 nM, 750 nM to 5,000 nM, 750 nM to 7,500 nM, 750 nM to 10,000 nM, 1,000 nM to 1,250 nM, 1,000 nM to 1,500 nM, 1,000 nM to 2,000 nM, 1,000 nM to 3,000 nM, 1,000 nM to 4,000 nM, 1,000 nM to 5,000 nM, 1,000 nM to 7,500 nM, 1,000 nM to 10,000 nM, 1,250 nM to 1,500 nM, 1,250 nM to 2,000 nM, 1,250 nM to 3,000 nM, 1,250 nM to 4,000 nM, 1,250 nM to 5,000 nM, 1,250 nM to 7,500 nM, 1,250 nM to 10,000 nM, 1,500 nM to 2,000 nM, 1,500 nM to 3,000 nM, 1,500 nM to 4,000 nM, 1,500 nM to 5,000 nM, 1,500 nM to 7,500 nM, 1,500 nM to 10,000 nM, 2,000 nM to 3,000 nM, 2,000 nM to 4,000 nM, 2,000 nM to 5,000 nM, 2,000 nM to 7,500 nM, 2,000 nM to 10,000 nM, 3,000 nM to 4,000 nM, 3,000 nM to 5,000 nM, 3,000 nM to 7,500 nM, 3,000 nM to 10,000 nM, 4,000 nM to 5,000 nM, 4,000 nM to 7,500 nM, 4,000 nM to 10,000 nM, 5,000 nM to 7,500 nM, 5,000 nM to 10,000 nM, or 7,500 nM to 10,000 nM. In certain embodiments, the K_{D} with IL-18BP, for the modified IL-18 polypeptide is 250 nM, 500 nM, 750 nM, 1,000 nM, 1,250 nM, 1,500 nM, 2,000 nM, 3,000 nM, 4,000 nM, 5,000 nM, 7,500 nM, or 10,000 nM. In certain embodiments, the K_{D} with IL-18BP, for the modified IL-18 polypeptide is at least 250 nM, 500 nM, 750 nM, 1,000 nM, 1,250 nM, 1,500 nM, 2,000 nM, 3,000 nM, 4,000 nM, 5,000 nM, or 7,500 nM. In certain embodiments, the K_{D} with IL-18BP, for the modified IL-18 polypeptide is at least 2 nM. In certain embodiments, the K_{D} with IL-18BP, for the modified IL-18 polypeptide is at least 5 nM. In certain embodiments, the K_{D} with IL-18BP, for the modified IL-18 polypeptide is at least 10 nM. In certain embodiments, the K_{D} with IL-18BP, for the modified IL-18 polypeptide is at least 20 nM. In certain embodiments, the K_{D} with IL-18BP, for the modified IL-18 polypeptide is at least 50 nM. In certain embodiments, the K_{D} with IL-18BP, for the modified IL-18 polypeptide is at least 100 nM. In certain embodiments, the K_{D} with IL-18BP, for the modified IL-18 polypeptide is at least 200 nM. In certain embodiments, the K_{D} with IL-18BP, for the modified IL-18 polypeptide is at least 300 nM. In certain embodiments, the K_{D} with IL-18BP, for the modified IL-18 polypeptide is at least 400 nM. In certain embodiments, the K_{D} with IL-18BP, for the modified IL-18 polypeptide is at least 500 nM. In certain embodiments, the K_{D} with IL-18BP, for the modified IL-18 polypeptide is at least 750 nM. In certain embodiments, the K_{D} with IL-18BP, for the modified IL-18 polypeptide is at least 1000 nM. In certain embodiments, the K_{D} with IL-18BP, for the modified IL-18 polypeptide is at least 2500 nM. In certain embodiments, the K_{D} with IL-18BP, for the modified IL-18 polypeptide is at least 5000 nM. In certain embodiments, the K_{D} with IL-18BP, for the modified IL-18 polypeptide is at least 10000 nM. The K_{D} with IL-18BP can be measured using IL-18BP Binding alphaLISA Assay (e.g., as provided herein in the Examples).

In some embodiments, the polymer modified IL-18 exhibits a reduced ability to have its IFNγ induction activity inhibited by IL-18BP compared to WT IL-18. In some embodiments, the polymer modified IL-18 displays a half-maximal inhibitory concentration (IC₅₀) by IL-18BP which is at least about 10-fold higher than, at least about 20-fold higher than, at least about 50-fold higher than, at least about 75-fold higher than, at least about 100-fold higher than, at least about 200-fold higher than, at least about 300-fold higher than, at least about 400-fold higher than, at least about 500-fold higher than, at least about 600-fold higher than, at least about 700-fold higher than, at least about 800-fold higher than, at least about 900-fold higher than, or at least about 1000-fold higher than an IC₅₀ of WT IL-18's inhibition by IL-18BP. In some embodiments, the polymer modified IL-18 displays a half-maximal inhibitory concentration (IC₅₀) by IL-18BP which is at least about 100-fold higher than an IC₅₀ of WT IL-18's inhibition by IL-18BP. In some embodiments, the polymer modified IL-18 displays a half-maximal inhibitory concentration (IC₅₀) by IL-18BP which is at least about 500-fold higher than an IC₅₀ of WT IL-18's inhibition by IL-18BP. In some embodiments, the polymer modified IL-18 displays a half-maximal inhibitory concentration (IC₅₀) by IL-18BP which is at least about 1000-fold higher than an IC₅₀ of WT IL-18's inhibition by IL-18BP.

In some embodiments, the polymer modified IL-18 displays a half-maximal inhibitory concentration (IC₅₀) by IL-18BP which is at about 10 nM, at least about 50 nM, at least about 100 nM, at least about 200 nM, at least about 300 nM, at least about 400 nM, at least about 500 nM, at least about 600 nM, at least about 700 nM, at least about 800 nM, at least about 900 nM, or at least about 1000 nM. In some embodiments, the polymer modified IL-18 displays a half-maximal inhibitory concentration (IC₅₀) by IL-18BP which is at least about 10 nM. In some embodiments, the polymer modified IL-18 displays a half-maximal inhibitory concentration (IC₅₀) by IL-18BP which is at least about 100 nM. In some embodiments, the modified IL-18 displays a half-maximal inhibitory concentration (IC₅₀) by IL-18BP which is at least about 1000 nM.

Ratio of IC₅₀/EC₅₀

In some embodiments, the modified IL-18 polypeptide exhibits a favorable ratio of half-maximal inhibitory concentration (IC₅₀) by IL-18BP over a half-maximal effective concentration (EC₅₀) of IFNγ induction (IC₅₀/EC₅₀ ratio). In some embodiments, the IC₅₀/EC₅₀ ratio for the modified IL-18 polypeptide is increased compared to WT IL-18. In some embodiments, the IC₅₀/EC₅₀ ratio for the modified IL-18 polypeptide is increased by at least about 2-fold, at least about 5-fold, at least about 10-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, or at least about 1000-fold compared to WT IL-18. In some embodiments, the IC₅₀/EC₅₀ ratio for the modified IL-18 polypeptide is increased by at least about 10-fold compared to WT IL-18. In some embodiments, the IC₅₀/EC₅₀ ratio for the modified IL-18 polypeptide is increased by at least about 100-fold compared to WT IL-18. In some embodiments, the IC₅₀/EC₅₀ ratio for the modified IL-18 polypeptide is increased by at least about 500-fold compared to WT IL-18.

In certain embodiments, the ratio of half-maximal inhibitory concentration (IC50) by IL-18BP, to half-maximal effective concentration (EC50) to induce IFNγ production, for the modified IL-18BP is 150,000 to 3,000,000. In certain embodiments, the ratio of half-maximal inhibitory concentration (IC50) by IL-18BP, to half-maximal effective concentration (EC50) to induce IFNγ production, for the modified IL-18BP is 50,000 to 3,000,000. In certain embodiments, the ratio of IC50 by IL-18BP, to EC50 to induce IFNγ production, for the modified IL-18BP is 100,000 to 300,000, 100,000 to 400,000, 100,000 to 500,000, 100,000 to 725,000, 100,000 to 1,000,000, 100,000 to 1,250,000, 100,000 to 1,500,000, 100,000 to 1,750,000, 100,000 to 2,000,000, 100,000 to 3,000,000, 298,000 to 300,000, 298,000 to 400,000, 298,000 to 500,000, 298,000 to 725,000, 298,000 to 1,000,000, 298,000 to 1,250,000, 298,000 to 1,500,000, 298,000 to 1,750,000, 298,000 to 2,000,000, 298,000 to 3,000,000, 300,000 to 400,000, 300,000 to 500,000, 300,000 to 725,000, 300,000 to 1,000,000, 300,000 to 1,250,000, 300,000 to 1,500,000, 300,000 to 1,750,000, 300,000 to 2,000,000, 300,000 to 3,000,000, 400,000 to 500,000, 400,000 to 725,000, 400,000 to 1,000,000, 400,000 to 1,250,000, 400,000 to 1,500,000, 400,000 to 1,750,000, 400,000 to 2,000,000, 400,000 to 3,000,000, 500,000 to 725,000, 500,000 to 1,000,000, 500,000 to 1,250,000, 500,000 to 150,000, 500,000 to 1,750,000, 500,000 to 2,000,000, 500,000 to 3,000,000, 725,000 to 1,000,000, 725,000 to 1,250,000, 725,000 to 1,500,000, 725,000 to 1,750,000, 725,000 to 2,000,000, 725,000 to 3,000,000, 1,000,000 to 1,250,000, 1,000,000 to 1,500,000, 1,000,000 to 1,750,000, 1,000,000 to 2,000,000, 1,000,000 to 3,000,000, 1,250,000 to U.S. Pat. Nos. 1,500,000, 1,250,000 to U.S. Pat. Nos. 1,750,000, 1,250,000 to 2,000,000, 1,250,000 to 3,000,000, 1,500,000 to U.S. Pat. Nos. 1,750,000, 1,500,000 to 2,000,000, 1,500,000 to 3,000,000, 1,750,000 to 2,000,000, 1,750,000 to 3,000,000, or 2,000,000 to 3,000,000. In certain embodiments, the ratio of IC50 by IL-18BP, to EC50 to induce IFNγ production, for the modified IL-18BP is 298,000, 300,000, 400,000, 500,000, 725,000, 1,000,000, 1,250,000, 150,000, 1,750,000, 2,000,000, or 3,000,000. In certain embodiments, the ratio of IC50 by IL-18BP, to EC50 to induce IFNγ production, for the modified IL-18BP is at least 50,000, 100,000, 200,000, 298,000, 300,000, 400,000, 500,000, 725,000, 1,000,000, 1,250,000, 150,000, 1,750,000, 2,000,000 or 3,000,000. In certain embodiments, the ratio of IC50 by IL-18BP, to EC50 to induce IFNγ production, for the modified IL-18BP is at least 50,000. In certain embodiments, the ratio of IC50 by IL-18BP, to EC50 to induce IFNγ production, for the modified IL-18BP is at least 100,000. In certain embodiments, the ratio of IC50 by IL-18BP, to EC50 to induce IFNγ production, for the modified IL-18BP is at least 200,000. In certain embodiments, the ratio of IC50 by IL-18BP, to EC50 to induce IFNγ production, for the modified IL-18BP is at least 299,000. In certain embodiments, the ratio of IC50 by IL-18BP, to EC50 to induce IFNγ production, for the modified IL-18BP is at least 300,000. In certain embodiments, the ratio of IC50 by IL-18BP, to EC50 to induce IFNγ production, for the modified IL-18BP is at least 400,000. In certain embodiments, the ratio of IC50 by IL-18BP, to EC50 to induce IFNγ production, for the modified IL-18BP is at least 500,000. In certain embodiments, the ratio of IC50 by IL-18BP, to EC50 to induce IFNγ production, for the modified IL-18BP is at least 750,000. In certain embodiments, the ratio of IC50 by IL-18BP, to EC50 to induce IFNγ production, for the modified IL-18BP is at least 1,000,000. In certain embodiments, the ratio of IC50 by IL-18BP, to EC50 to induce IFNγ production, for the modified IL-18BP is at least 1,250,000. In certain embodiments, the ratio of IC50 by IL-18BP, to EC50 to induce IFNγ production, for the modified IL-18BP is at least 1,500,000. In certain embodiments, the ratio of IC50 by IL-18BP, to EC50 to induce IFNγ production, for the modified IL-18BP is at least 1,750,000. EC50 to induce IFNγ production can be measured using IFNγ Induction NK-92 Cellular Assay, and IC50 by IL-18BP can be measured by IL-18 Binding Protein-mediated Inhibition of IFNγ secretion in NK-92 Cellular Assay (e.g., as provided in the Examples herein).

In some embodiments, the polymer modified IL-18 polypeptide exhibits a favorable ratio of half-maximal inhibitory concentration (IC₅₀) by IL-18BP over a half-maximal effective concentration (EC₅₀) of IFNγ induction (IC₅₀/EC₅₀ ratio). In some embodiments, the IC₅₀/EC₅₀ ratio for the polymer modified IL-18 polypeptide is increased compared to WT IL-18. In some embodiments, the IC₅₀/EC₅₀ ratio for the polymer modified IL-18 polypeptide is increased by at least about 2-fold, at least about 5-fold, at least about 10-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, or at least about 1000-fold compared to WT IL-18. In some embodiments, the IC₅₀/EC₅₀ ratio for the polymer modified IL-18 polypeptide is increased by at least about 10-fold compared to WT IL-18. In some embodiments, the IC₅₀/EC₅₀ ratio for the polymer modified IL-18 polypeptide is increased by at least about 100-fold compared to WT IL-18. In some embodiments, the IC₅₀/EC₅₀ ratio for the polymer modified IL-18 polypeptide is increased by at least about 500-fold compared to WT IL-18. In some embodiments, the IC₅₀/EC₅₀ ratio of the modified IL-18 polypeptide is at least about 2, at least about 5, at least about 10, at least about 50, at least about 100, at least about 250, or at least about 500.

### V. Pharmaceutical Compositions

In one aspect, described herein is a pharmaceutical composition comprising: a modified IL-18 polypeptide described herein; and a pharmaceutically acceptable carrier or excipient. In some embodiments, the pharmaceutical composition comprises a plurality of the modified IL-18 polypeptides. In an aspect, described herein is a pharmaceutical composition comprising: a polymer modified IL-18 polypeptide described herein; and a pharmaceutically acceptable carrier or excipient. In some embodiments, the pharmaceutical composition comprises a plurality of the polymer modified IL-18 polypeptides. In an aspect, described herein is a pharmaceutical composition comprising: a modified IL-18 polypeptide described herein and/or a polymer modified IL-18 polypeptide described herein; and a pharmaceutically acceptable carrier or excipient. In some embodiments, the pharmaceutical composition comprises a plurality of the modified IL-18 polypeptides and/or a plurality of the polymer modified IL-18 polypeptides. In some embodiments, the pharmaceutical compositions further comprises one or more excipient selected from a carbohydrate, an inorganic salt, an antioxidant, a surfactant, or a buffer.

In some embodiments, the pharmaceutical composition further comprises a carbohydrate. In certain embodiments, the carbohydrate is selected from the group consisting of fructose, maltose, galactose, glucose, D-mannose, sorbose, lactose, sucrose, trehalose, cellobiose raffinose, melezitose, maltodextrins, dextrans, starches, mannitol, xylitol, maltitol, lactitol, xylitol, sorbitol (glucitol), pyranosyl sorbitol, myoinositol, cyclodextrins, and combinations thereof.

In some embodiments, the pharmaceutical composition comprises an inorganic salt. In certain embodiments, the inorganic salt is selected from the group consisting of sodium chloride, potassium chloride, magnesium chloride, calcium chloride, sodium phosphate, potassium phosphate, sodium sulfate, or combinations thereof.

In certain embodiments, the pharmaceutical composition comprises an antioxidant. In certain embodiments, the antioxidant is selected from the group consisting of ascorbyl palmitate, butylated hydroxyanisole, butylated hydroxytoluene, potassium metabisulfite, propyl gallate, sodium metabisulfite, sodium thio sulfate, vitamin E, 3,4-dihydroxybenzoic acid, and combinations thereof.

In certain embodiments, the pharmaceutical composition comprises a surfactant. In certain embodiments, the surfactant is selected from the group consisting of polysorbates, sorbitan esters, lipids, phospholipids, phosphatidylethanolamines, fatty acids, fatty acid esters, steroids, EDTA, zinc, and combinations thereof.

In certain embodiments, the pharmaceutical composition comprises a buffer. In certain embodiments, the buffer is selected from the group consisting of citric acid, sodium phosphate, potassium phosphate, acetic acid, ethanolamine, histidine, amino acids, tartaric acid, succinic acid, fumaric acid, lactic acid, tris, HEPES, CHAPS, or combinations thereof.

In some embodiments, the pharmaceutical composition is formulated for parenteral or enteral administration. In some embodiments, the pharmaceutical composition is formulated for intravenous or subcutaneous administration. In some embodiments, the pharmaceutical composition is in a lyophilized form.

In one aspect, described herein is a liquid or lyophilized composition that comprises a described modified IL-18 polypeptide. In some embodiments, the modified IL-18 polypeptide is a lyophilized powder. In an aspect, described herein is a liquid or lyophilized composition that comprises a described polymer modified IL-18 polypeptide. In some embodiments, the polymer modified IL-18 polypeptide is a lyophilized powder. In some embodiments, the lyophilized powder is resuspended in a buffer solution. In some embodiments, the buffer solution comprises a buffer, a sugar, a salt, a surfactant, or any combination thereof. In some embodiments, the buffer solution comprises a phosphate salt. In some embodiments, the phosphate salt is sodium Na₂HPO₄. In some embodiments, the salt is sodium chloride. In some embodiments, the buffer solution comprises phosphate buffered saline. In some embodiments, the buffer solution comprises mannitol. In some embodiments, the lyophilized powder is suspended in a solution comprising phosphate buffered saline solution (pH 7.4) with 50 mg/mL mannitol. In some embodiments, the pharmaceutical composition is a lyophilized composition which is reconstituted shortly before administration to a subject.

The modified IL-18 polypeptides described herein can be in a variety of dosage forms. In some embodiments, the modified IL-18 polypeptide is dosed as a lyophilized powder. In some embodiments, the modified IL-18 polypeptide is dosed as a suspension. In some embodiments, the modified IL-18 polypeptide is dosed as a solution. In some embodiments, the modified IL-18 polypeptide is dosed as an injectable solution. In some embodiments, the modified IL-18 polypeptide is dosed as an IV solution. The polymer modified IL-18 polypeptides described herein can be in a variety of dosage forms. In some embodiments, the polymer modified IL-18 polypeptide is dosed as a lyophilized powder. In some embodiments, the polymer modified IL-18 polypeptide is dosed as a suspension. In some embodiments, the polymer modified IL-18 polypeptide is dosed as a solution. In some embodiments, the polymer modified IL-18 polypeptide is dosed as an injectable solution. In some embodiments, the polymer modified IL-18 polypeptide is dosed as an IV solution.

### VI. Method of Treatment

In one aspect, described herein, is a method of treating cancer in a subject in need thereof, comprising: administering to the subject an effective amount of a modified IL-18 polypeptide as provided herein or a pharmaceutical composition as described herein.

In one aspect, described herein, is a method of treating cancer in a subject in need thereof, comprising: administering to the subject an effective amount of a polymer modified IL-18 polypeptide comprising a polymer as provided herein or a pharmaceutical composition as described herein.

In an aspect, described herein, is a modified IL-18 polypeptide as provided herein for use in treatment of cancer in a subject in need thereof. In another aspect, described herein, is a modified IL-18 polypeptide as provided herein for in the manufacture of a medicament for treatment of cancer in a subject in need thereof.

In another aspect, described herein, is a polymer modified IL-18 polypeptide comprising a polymer as provided herein for use in treatment of cancer in a subject in need thereof. In another aspect, described herein, is a polymer modified IL-18 polypeptide comprising a polymer as provided herein for in the manufacture of a medicament for treatment of cancer in a subject in need thereof.

In some embodiments, the cancer is a solid cancer. In some embodiments, the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer.

In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia.

A modified IL-18 polypeptide described herein can be administered to a subject in one or more doses. In some embodiments, the modified IL-18 polypeptide is administered in a single dose of the effective amount of the modified IL-18 polypeptide, including further embodiments in which (i) the modified IL-18 polypeptide is administered once a day; or (ii) the modified IL-18 polypeptide is administered to the subject multiple times over the span of one day. In some embodiments, the modified IL-18 polypeptide is administered daily, every other day, twice a week, 3 times a week, once a week, every 2 weeks, every 3 weeks, every 4 weeks, every 5 weeks, every 6 weeks, every 12 weeks, every 3 days, every 4 days, every 5 days, every 6 days, 2 times a week, 3 times a week, 4 times a week, 5 times a week, 6 times a week, once a month, twice a month, 3 times a month, 4 times a month, once every 2 months, once every 3 months, once every 4 months, once every 5 months, or once every 6 months. Administration includes, but is not limited to, injection by any suitable route (e.g., parenteral, enteral, intravenous, subcutaneous, etc.).

A polymer modified IL-18 polypeptide described herein can be administered to a subject in one or more doses. In some embodiments, the polymer modified IL-18 polypeptide is administered in a single dose of the effective amount of the IL-18 polypeptide, including further embodiments in which (i) the polymer modified IL-18 polypeptide is administered once a day; or (ii) the polymer modified IL-18 polypeptide is administered to the subject multiple times over the span of one day. In some embodiments, the polymer modified IL-18 is administered daily, every other day, twice a week, 3 times a week, once a week, every 2 weeks, every 3 weeks, every 4 weeks, every 5 weeks, every 6 weeks, every 12 weeks, every 3 days, every 4 days, every 5 days, every 6 days, 2 times a week, 3 times a week, 4 times a week, 5 times a week, 6 times a week, once a month, twice a month, 3 times a month, 4 times a month, once every 2 months, once every 3 months, once every 4 months, once every 5 months, or once every 6 months. Administration includes, but is not limited to, injection by any suitable route (e.g., parenteral, enteral, intravenous, subcutaneous, etc.).

An effective response is achieved when the subject experiences partial or total alleviation or reduction of signs or symptoms of illness, and specifically includes, without limitation, prolongation of survival. The expected progression-free survival times may be measured in months to years, depending on prognostic factors including the number of relapses, stage of disease, and other factors. Prolonging survival includes without limitation times of at least 1 month (mo), about at least 2 mos., about at least 3 mos., about at least 4 mos., about at least 6 mos., about at least 1 year, about at least 2 years, about at least 3 years, about at least 4 years, about at least 5 years, etc. Overall or progression-free survival can be also measured in months to years. Alternatively, an effective response may be that a subject's symptoms remain static and do not worsen. Further indications of treatment of indications are described in more detail below. In some instances, a cancer or tumor is reduced by at least 2%, 5%, 10%, 15%, 20%, 25%, 30%, 35%, 40%, 45%, 50%, 55%, 60%, 65%, 70%, 75%, 80%, 85%, 90%, 95%, or 100%.

In some embodiments, the method further comprises reconstituting a lyophilized form of the modified IL-18 polypeptide or the pharmaceutical composition. In some embodiments, the modified IL-18 polypeptide or the pharmaceutical composition is reconstituted before administration. In some embodiments, the composition is reconstituted immediately before administration, up to about 5 minutes before administration, up to about 20 minutes before administration, up to about 40 minutes before administration, up to an hour before administration, or up to about four hours before administration.

In some embodiments, the method further comprises reconstituting a lyophilized form of the polymer modified IL-18 polypeptide or the pharmaceutical composition. In some embodiments, the polymer modified IL-18 polypeptide or the pharmaceutical composition is reconstituted before administration. In some embodiments, the composition is reconstituted immediately before administration, up to about 5 minutes before administration, up to about 20 minutes before administration, up to about 40 minutes before administration, up to an hour before administration, or up to about four hours before administration.

### VII. Preparation of Recombinant IL-18 Polypeptides

In one aspect, described herein is a method of producing a modified IL-18 polypeptide, wherein the method comprises expressing the modified IL-18 polypeptide in a host cell.

In one aspect, described herein is a host cell comprising a modified IL-18 polypeptide.

In some embodiments, the host cell is a prokaryotic cell or a eukaryotic cell. In some embodiments, the host cell is a mammalian cell, an avian cell, or an insect cell. In some embodiments, the host cell is a mammalian cell, an avian cell, a fungal cell, or an insect cell. In some embodiments, the host cell is a CHO cell, a COS cell, or a yeast cell.

In some embodiments, producing the polymer modified IL-18 polypeptide comprises attaching a polymer to the modified IL-18 polypeptide. In some embodiments, the polymer is directly attached to a residue of the modified IL-18 polypeptide, optionally through a linker attached to the polymer. In some embodiments, a heterobifunctional linker is first attached to the modified IL-18 polypeptide, followed by attachment of the polymer to the heterobifunctional linker.

### VIII. Method of Manufacturing (Synthesis)

Also provided herein is a method synthesizing a modified IL-18 polypeptide provided herein. Also provided herein is a method synthesizing a polymer modified IL-18 polypeptide (e.g., a modified IL-18 polypeptide with a polymer attached as provided herein). In some cases, the modified IL-18 polypeptide is synthesized chemically rather than recombinantly expressed. In some instances, several fragment peptide precursors of the modified IL-18 polypeptide are synthesized and subsequently ligated together using a suitable ligation methodology (e.g., alpha-keto acid hydroxylamine (KAHA) ligation). In some cases, after ligation, the resulting modified IL-18 polypeptide is folded to produce a modified IL-18 polypeptide having a secondary and tertiary structure substantially identical to that of a recombinant or wild type IL-18 polypeptide. In some instances, several fragment peptide precursors of the polymer modified IL-18 polypeptide are synthesized and subsequently ligated together using a suitable ligation methodology (e.g., alpha-keto acid hydroxylamine (KAHA) ligation). In some cases, after ligation, the resulting polymer modified IL-18 polypeptide is folded to produce a modified IL-18 polypeptide having a secondary and tertiary structure substantially identical to that of a recombinant or wild type IL-18 polypeptide, and folded polypeptide is attached to a polymer to produce a polymer modified IL-18 polypeptide.

In some instances, methionine residues of the modified IL-18 polypeptide are substituted for stability purposes and/or to aid in the folding of the linear modified IL-18 polypeptide to produce the final modified IL-18 polypeptide. The side chain of methionine is prone to oxidation during the synthesis process (e.g., peptide synthesis and protein folding), thus resulting, in some cases, in a finalized IL-18 polypeptide of insufficient quality for certain uses due to a lack of uniformity.

In some cases, in order to combat these limitations, all methionine residues of the modified IL-18 polypeptide were replaced with norleucine residues. In some cases, synthesis of the linear peptide was successful, but the resulting polypeptide showed signs of instability, such as increased hydrophobicity and propensity to precipitate, and detuned biological activity, potentially because of misfolding resulting in altered secondary/tertiary structure of the modified IL-18 polypeptide relative to wild type or recombinant IL-18.

In some cases, modified IL-18 polypeptides were synthesized to directly incorporate oxidized methionine during the synthesis of the precursor peptides in an attempt to create a uniform linear protein without a complex mixture of partial methionine oxidation. In some cases, the modified linear IL-18 polypeptides were successfully synthesized, but difficulty was encountered in reducing the methionine back to the unoxidized form.

In order to combat these challenges, new modified IL-18 polypeptide variants were designed which replaced one or more methionine residues with O-methyl-L-homoserine (Omh) residues. Omh is a structural analog of natural methionine with the sulfur atom of methionine replaced with an oxygen. Due to the lack of the sulfur atom, Omh residues are less prone to oxidation and thus are predicted to give the modified IL-18 polypeptide greater stability and ease of synthesis/purification. Additionally, the increased hydrophilicity of the Omh residue compared to norleucine residues, along Omh's greater structural homology to the native methionine residues, is predicted to facilitate proper folding and greater stability of the modified IL-18 polypeptide as compared to a variant with norleucine residues in place of the methionines. Thus, it is predicted that a chemically synthesized modified IL-18 polypeptide which replaces methionine residues with Omh residues will provide several advantages over other synthesized modified IL-18 polypeptides.

In one aspect, described herein, is a method of making a modified IL-18 polypeptide. In another aspect, described herein, is a method of making a modified IL-18 polypeptide comprising synthesizing two or more fragments of the modified IL-18 polypeptide, and ligating the fragments.

In another aspect, described herein, is a method of making a polymer modified IL-18 polypeptide comprising a. synthesizing two or more fragments of the modified IL-18 polypeptide, b. ligating the fragments; c. folding the ligated fragments and d. attaching a polymer to the modified IL-18 polypeptide.

In another aspect, described herein, is a method of making a polymer modified IL-18 polypeptide comprising providing two or more fragments of the modified IL-18 polypeptide, ligating the fragments, and attaching a polymer to the modified IL-18 polypeptide.

In another aspect, described herein, is a method of making a modified IL-18 polypeptide comprising a. providing two or more fragments of the modified IL-18 polypeptide, b. ligating the fragments; and c. folding the ligated fragments.

In another aspect, described herein, is a method of making a modified IL-18 polypeptide comprising ligating two or more fragments of the modified IL-18 polypeptide, wherein at least one of the two or more fragments of the modified IL-18 polypeptide are synthesized, and folding the ligated fragments.

In some embodiments, the two or more fragments of the modified IL-18 polypeptide are synthesized chemically. In some embodiments, the two or more fragments of the modified IL-18 polypeptide are synthesized by solid phase peptide synthesis. In some embodiments, the two or more fragments of the modified IL-18 polypeptide are synthesized on an automated peptide synthesizer.

In some embodiments, the modified IL-18 polypeptide is ligated from 2, 3, 4, 5, 6, 7, 8, 9, 10, or more peptide fragments. In some embodiments, the modified peptide is ligated from 2 peptide fragments. In some embodiments, the modified IL-18 polypeptide is ligated from 3 peptide fragments. In some embodiments, the modified IL-18 polypeptide is ligated from 4 peptide fragments. In some embodiments, the modified IL-18 polypeptide is ligated from 5 peptide fragments. In some embodiments, the modified IL-18 polypeptide is ligated from 2 to 10 peptide fragments.

In some embodiments, the two or more fragments comprise an N-terminal fragment, a C-terminal fragment, and optionally one or more interior fragments, wherein the N-terminal fragment comprises the N-terminus of the modified IL-18 polypeptide and the C-terminal fragment comprises the C-terminus of the modified IL-18 polypeptide. In some embodiments, each of the N-terminal fragment and the one or more interior fragments comprise an alpha-keto amino acid as the C-terminal residue of each fragment. In some embodiments, each alpha-keto amino acid comprises a hydrophobic side chain. In some embodiments, each alpha-keto amino acid is selected from alpha-keto-phenylalanine, alpha-keto-tyrosine, alpha-keto-valine, alpha-keto-leucine, alpha-keto-isoleucine, alpha-keto-norleucine, and alpha-keto-O-methylhomoserine.

In some embodiments, each of the C-terminal fragment and the one or more interior fragments comprise a residue having a hydroxylamine or a cyclic hydroxylamine functionality as the N-terminal residue of each fragment. In some embodiments, each residue having the hydroxylamine or the cyclic hydroxylamine functionality is a 5-oxaproline residue.

In some embodiments, the two or more fragments of the modified IL-18 polypeptide are ligated together. In some embodiments, three or more fragments of the modified IL-18 polypeptide are ligated in a sequential fashion. In some embodiments, three or more fragments of the modified IL-18 polypeptide are ligated in a one-pot reaction.

### Four Fragment Ligation Strategy

In some embodiments, synthesizing two or more fragments of the modified IL-18 polypeptide comprises synthesizing four fragments. In some embodiments, providing two or more fragments of the modified IL-18 polypeptide comprises providing four fragments. In some embodiments, the four fragments include four fragments each having at least about 80% sequence identity to any sequence independently selected from those provided in Table 2. In some embodiments, the four fragments include four fragments having at least about 85% sequence identity to those provided in Table 2. In some embodiments, the four fragments include four fragments having at least about 90% sequence identity to those provided in Table 2. In some embodiments, the four fragments include four fragments having at least about 95% sequence identity to those provided in Table 2. In some embodiments, the four fragments include four fragments provided in Table 2.

TABLE 2
Exemplary Peptides used to synthesize IL-18

| SEQ | | | |
|---|---|---|---|
| ID | | | |
| NO | Comment | Sequence | Key |
| 301 | Peptide | YFGKLESKLS VIRNLNDQVL | Akf = alpha-keto- |
| | 1 | FIDQGNRPL(Akf) | phenylalanine |
| | (1-30) | | |
| 302 | Peptide | YFGKLKSKLS VIRNLNDQVL | Akf = alpha-keto- |
| | 1 E6K | FIDQGNRPL(Akf) | phenylalanine |
| 303 | Peptide | GFGKLESKLS VIRNLNDQVL | Akf = alpha-keto- |
| | 1 Y1G | FIDQGNRPL(Akf) | phenylalanine |
| 304 | Peptide | YAGKLESKLS VIRNLNDQVL | Akf = alpha-keto- |
| | 1 F2A | FIDQGNRPL(Akf) | phenylalanine |
| 305 | Peptide | GFGKLKSKLS VIRNLNDQVL | Akf = alpha-keto- |
| | 1 Y1G | FIDQGNRPL(Akf) | phenylalanine |
| | E6K | | |
| 306 | Peptide | YAGKLKSKLS VIRNLNDQVL | Akf = alpha-keto- |
| | 1 F2A | FIDQGNRPL(Akf) | phenylalanine |
| | E6K | | |
| 307 | Peptide | GAGKLKSKLS VIRNLNDQVL | Akf = alpha-keto- |
| | 1 Y1G | FIDQGNRPL(Akf) | phenylalanine |
| | F2A E6K | | |
| 308 | Peptide | GAGKLESKLS VIRNLNDQVL | Akf = alpha-keto- |
| | 1 Y1G | FIDQGNRPL(Akf) | phenylalanine |
| | F2A | | |
| 309 | Peptide | YFGKLKSKLS IIRNLNDQVL | Akf = alpha-keto- |
| | 1 E6K | FIDQGNRPL(Akf) | phenylalanine |
| | V111 | | |
| 310 | Peptide | (Opr) DMTDSDCRD NAPRTIFIIS | Opr = 5-oxa-proline; |
| | 2A | MYKDSQPRGM A(Akv) | Akv = alpha-keto-valine |
| | (31-62) | | |
| 311 | Peptide | (Opr) DXTDSDCRD NAPRTIFIIS | Opr = 5-oxa-proline; |
| | 2A | XYKDSQPRGX A(Akv) | Akv = alpha-keto-valine |
| | M33X, | | |
| | M51X, | | |
| | M60X | | |
| 312 | Peptide | (Opr) DJTDSDCRD NAPRTIFIIS | Opr = 5-oxa-proline; |
| | 2A | JYKDSQPRGJ A(Akv) | Akv = alpha-keto-valine |
| | M33J, | | |
| | M51J, | | |
| | M60J | | |
| 313 | Peptide | (Opr) DMTDSDCRD NAPRTIFIIS | Opr = 5-oxa-proline; |
| | 2A K53A | MYADSQPRGM A(Akv) | Akv = alpha-keto-valine |
| 314 | Peptide | (Opr) DXTDSDCRD NAPRTIFIIS | Opr = 5-oxa-proline; |
| | 2A | XYADSQPRGX A(Akv) | Akv = alpha-keto-valine |
| | M33X, | | |
| | M51X, | | |
| | K53A, | | |
| | M60X | | |
| 315 | Peptide | (Opr) DJTDSDCRD NAPRTIFIIS | Opr = 5-oxa-proline; |
| | 2A | JYADSQPRGJ A(Akv) | Akv = alpha-keto-valine |
| | M33J, | | |
| | M51J, | | |
| | K53A, | | |
| | M60J | | |
| 316 | Peptide | (Opr) DJTDSDSRD NAPRTIFIIS | Opr = 5-oxa-proline; |
| | 2A | JYADSQPRGJ A(Akv) | Akv = alpha-keto-valine |
| | M33J, | | |
| | C38S, | | |
| | M51J, | | |
| | K53A, | | |
| | M60J | | |
| 317 | Peptide | (Opr) DJTDSDSRD NAPRTIFIIS | Opr = 5-oxa-proline; |
| | 2A | JYKDSQPRGJ A(Akv) | Akv = alpha-keto-valine |
| | M33J, | | |
| | C38S, | | |
| | M51J, | | |
| | M60J | | |
| 318 | Peptide | (Opr) DMTDSDCRD NAPRTIFIIS | Opr = 5-oxa-proline; |
| | 2B | MYKDSQPRGM AVTISVKCEK IST(Akl) | Akl = alpha-keto-leucine |
| | (31-74) | | |
| 319 | Peptide | (Opr) DMTDSDCRD NAPRTIFIIS | Opr = 5-oxa-proline; |
| | 2B K53A | MYADSQPRGM AVTISVKCEK IST(Akl) | Akl = alpha-keto-leucine |
| 320 | Peptide | (Opr) DMTDSDCRD NAPRTIFIIS | Opr = 5-oxa-proline; |
| | 2B K53A, | MYADSQPRGM AVTISVKCEK IST(Akl) | Akl = alpha-keto-leucine |
| | T63A | | |
| 321 | Peptide | (Opr) DXTDSDCRD NAPRTIFIIS | Opr = 5-oxa-proline; |
| | 2B | XYKDSQPRGX AVTISVKCEK IST(Akl) | Akl = alpha-keto-leucine |
| | M33X, | | |
| | M51X, | | |
| | M60X | | |
| 322 | Peptide | (Opr) DJTDSDCRD NAPRTIFIIS | Opr = 5-oxa-proline; |
| | 2B | JYKDSQPRGJ AVTISVKCEK IST(Akl) | Akl = alpha-keto-leucine |
| | M33J, | | |
| | M51J, | | |
| | M60J | | |
| 323 | Peptide | (Opr) DJTDSDCRD NAPRTIFIIS | Opr = 5-oxa-proline; |
| | 2B | JYADSQPRGJ AVTISVKCEK IST(Akl) | Akl = alpha-keto-leucine |
| | M33J, | | |
| | M51J, | | |
| | K53A, | | |
| | M60J | | |
| 324 | Peptide | (Opr) DJTDSDCRD NAPRTIFIIS | Opr = 5-oxa-proline; |
| | 2B | JYADSQPRGJ AVAISVKCEK IST(Akl) | Akl = alpha-keto-leucine |
| | M33J, | | |
| | M51J, | | |
| | K53A, | | |
| | M60J, | | |
| | T63A | | |
| 325 | Peptide | (Opr) DJTDSDSRD NAPRTIFIIS | Opr = 5-oxa-proline; |
| | 2B | JYADSQPRGJ AVAISVKCEK IST(Akl) | Akl = alpha-keto-leucine |
| | M33J, | | |
| | C38S, | | |
| | M51J, | | |
| | K53A, | | |
| | M60J, | | |
| | T63A | | |
| 326 | Peptide | (Opr) DMTDSDCRD NAPRTIFIIS | Opr = 5-oxa-proline; |
| | 2B C68S | MYKDSQPRGM AVTISVKSEK IST(Akl) | Akl = alpha-keto-leucine |
| 327 | Peptide | (Opr) ISVKCEK ISTLSCENKI | Opr = 5-oxa-proline; |
| | 3A | ISFKEMNPPD NIKDTKSDII FFQRSVPGHD | Akf = alpha-keto- |
| | (63-115) | NKMQ(Akf) | phenylalanine |
| 328 | Peptide | (Opr) ISVKCEK ISTLSCENKI | Opr = 5-oxa-proline; |
| | 3A | ISFKEXNPPD NIKDTKSDII FFQRSVPGHD | Akf = alpha-keto- |
| | M86X, | NKXQ(Akf) | phenylalanine |
| | M113X | | |
| 329 | Peptide | (Opr) ISVKCEK ISTLSCENKI | Opr = 5-oxa-proline; |
| | 3A | ISFKEJNPPD NIKDTKSDII FFQRSVPGHD | Akf = alpha-keto- |
| | M86J, | NKJQ(Akf) | phenylalanine |
| | M113J | | |
| 330 | Peptide | (Opr) ISVKSEK ISTLSCENKI | Opr = 5-oxa-proline; |
| | 3A C68S | ISFKEMNPPD NIKDTKSDII FFQRSVPGHD | Akf = alpha-keto- |
| | | NKMQ(Akf) | phenylalanine |
| 331 | Peptide | (Opr) ISVKSEK ISTLSCENKI | Opr = 5-oxa-proline; |
| | 3A C68S, | ISFKEXNPPD NIKDTKSDII FFQRSVPGHD | Akf = alpha-keto- |
| | M86X, | NKXQ(Akf) | phenylalanine |
| | M113X | | |
| 332 | Peptide | (Opr) ISVKSEK ISTLSCENKI | Opr = 5-oxa-proline; |
| | 3A C68S, | ISFKEJNPPD NIKDTKSDII FFQRSVPGHD | Akf = alpha-keto- |
| | M86J, | NKJQ(Akf) | phenylalanine |
| | M113J | | |
| 333 | Peptide | (Opr) ISVKSEK ISTLSSENKI | Opr = 5-oxa-proline; |
| | 3A C68S, | ISFKEMNPPD NIKDTKSDII FFQRSVPGHD | Akf = alpha-keto- |
| | C76S | NKMQ(Akf) | phenylalanine |
| 334 | Peptide | (Opr) ISVKCEK ISTLSSENKI | Opr = 5-oxa-proline; |
| | 3A C76S | ISFKEMNPPD NIKDTKSDII FFQRSVPGHD | Akf = alpha-keto- |
| | | NKMQ(Akf) | phenylalanine |
| 335 | Peptide | (Opr) ISVKCEK ISTLSSENKI | Opr = 5-oxa-proline; |
| | 3A C76S, | ISFKEJNPPD NIKDTKSDII FFQRSVPGHD | Akf = alpha-keto- |
| | M86J, | NKJQ(Akf) | phenylalanine |
| | M113J | | |
| 336 | Peptide | (Opr) ISVKSEK ISTLSSENKI | Opr = 5-oxa-proline; |
| | 3A C68S, | ISFKEJNPPD NIKDTKSDII FFQRSVPGHD | Akf = alpha-keto- |
| | C76S, | NKJQ(Akf) | phenylalanine |
| | M86J, | | |
| | M113J | | |
| 337 | Peptide | (Opr) CENKI ISFKEMNPPD NIKDTKSDII | Opr = 5-oxa-proline; |
| | 3B | FFQRSVPGHD NKMQ(Akf) | Akf = alpha-keto- |
| | (75-115 | | phenylalanine |
| 338 | Peptide | (Opr) SENKI ISFKEMNPPD NIKDTKSDII | Opr = 5-oxa-proline; |
| | 3B C76S | FFQRSVPGHD NKMQ(Akf) | Akf = alpha-keto- |
| | | | phenylalanine |
| 339 | Peptide | (Opr) CENKI ISFKEXNPPD NIKDTKSDII | Opr = 5-oxa-proline; |
| | 3B | FFQRSVPGHD NKXQ(Akf) | Akf = alpha-keto- |
| | M86X, | | phenylalanine |
| | M113X | | |
| 340 | Peptide | (Opr) SENKI ISFKEXNPPD NIKDTKSDII | Opr = 5-oxa-proline; |
| | 3B C76S, | FFQRSVPGHD NKXQ(Akf) | Akf = alpha-keto- |
| | M86X, | | phenylalanine |
| | M113X | | |
| 341 | Peptide | (Opr) CENKI ISFKEJNPPD NIKDTKSDII | Opr = 5-oxa-proline; |
| | 3B | FFQRSVPGHD NKJQ(Akf) | Akf = alpha-keto- |
| | M86J, | | phenylalanine |
| | M113J | | |
| 342 | Peptide | (Opr) SENKI ISFKEJNPPD NIKDTKSDII | Opr = 5-oxa-proline; |
| | 3B C76S, | FFQRSVPGHD NKJQ(Akf) | Akf = alpha-keto- |
| | M86J, | | phenylalanine |
| | M113J | | |
| 343 | Peptide | (Opr) SSSY EGYFLACEKE RDLFKLILKK | Opr = 5-oxaproline |
| | 4 | EDELGDRSIM FTVQNED | |
| | (116-157) | | |
| 344 | Peptide | (Opr) SSSY EGYFLASEKE RDLFKLILKK | Opr = 5-oxaproline |
| | 4 C127S | EDELGDRSIM FTVQNED | |
| 345 | Peptide | (Opr) SSSY EGYFLACEKE RDLFKLILKK | Opr = 5-oxaproline |
| | 4 M150X | EDELGDRSIX FTVQNED | |
| 346 | Peptide | (Opr) SSSY EGYFLASEKE RDLFKLILKK | Opr = 5-oxaproline |
| | 4 C127S, | EDELGDRSIX FTVQNED | |
| | M150X | | |
| 347 | Peptide | (Opr) SSSY EGYFLACEKE RDLFKLILKK | Opr = 5-oxaproline |
| | 4 M150J | EDELGDRSIJ FTVQNED | |
| 348 | Peptide | (Opr) SSSY EGYFLASEKE RDLFKLILKK | Opr = 5-oxaproline |
| | 4 C127S, | EDELGDRSIJ FTVQNED | |
| | M150J | | |

X = Norleucine;
J = O-methyl-L-homoserine

In some embodiments, the four fragments comprise an N-terminal fragment, a first interior fragment, a second interior fragment, and a C-terminal fragment.

In some embodiments, the N-terminal fragment comprises residues which correspond to amino acids 1-30 of the modified IL-18 polypeptide, wherein residue position numbering of the modified IL-18 polypeptide is based on SEQ ID NO: 1 as a reference sequence, and further comprises amino acids of an N-terminal extension as provided herein In some embodiments, the N-terminal fragment comprises an amino acid sequence having at least 80% sequence identity with the amino acid sequence as set forth in SEQ ID NO: 301. In some embodiments, the N-terminal fragment comprises an amino acid sequence as set forth in any one of SEQ ID Nos: 301-309.

In some embodiments, the first interior fragment comprises residues which correspond to amino acids 31-62 of the modified IL-18 polypeptide, wherein residue position numbering of the modified IL-18 polypeptide is based on SEQ ID NO: 1 as a reference sequence. In some embodiments, the first interior fragment comprises an amino acid sequence having at least 80% sequence identity with the amino acid sequence as set forth in SEQ ID NO: 310. In some embodiments, the first interior fragment comprises an amino acid sequence as set forth in any one of SEQ ID Nos: 310-317.

In some embodiments, the second interior fragment comprises residues which correspond to amino acids 63-115 of the modified IL-18 polypeptide, wherein residue position numbering of the modified IL-18 polypeptide is based on SEQ ID NO: 1 as a reference sequence. In some embodiments, the second interior fragment comprises an amino acid sequence having at least 80% sequence identity with the amino acid sequence as set forth in SEQ ID NO: 327. In some embodiments, the second interior fragment comprises an amino acid sequence as set forth in any one of SEQ ID NOs: 327-336.

In some embodiments, the first interior fragment comprises residues which correspond to amino acids 31-74 of the modified IL-18 polypeptide, wherein residue position numbering of the modified IL-18 polypeptide is based on SEQ ID NO: 1 as a reference sequence. In some embodiments, the first interior fragment comprises an amino acid sequence having at least 80% sequence identity with the amino acid sequence as set forth in SEQ ID NO: 318. In some embodiments, the first interior fragment comprises an amino acid sequence as set forth in any one of SEQ ID NOs: 318-326.

In some embodiments, the second interior fragment comprises residues which correspond to amino acids 75-115 of the modified IL-18 polypeptide, wherein residue position numbering of the modified IL-18 polypeptide is based on SEQ ID NO: 1 as a reference sequence. In some embodiments, the second interior fragment comprises an amino acid sequence having at least 80% sequence identity with the amino acid sequence as set forth in SEQ ID NO: 337. In some embodiments, the second interior fragment comprises an amino acid sequence as set forth in any one of SEQ ID NOs: 337-342.

In some embodiments, the C-terminal fragment comprises residues which correspond to amino acids 116-157 of the modified IL-18 polypeptide, wherein residue position numbering of the modified IL-18 polypeptide is based on SEQ ID NO: 1 as a reference sequence. In some embodiments, the C-terminal fragment comprises an amino acid sequence having at least 80% sequence identity with the amino acid sequence as set forth in SEQ ID NO: 343. In some embodiments, the C-terminal fragment comprises an amino acid sequence as set forth in any one of SEQ ID NOs: 343-348.

In some embodiments, the second interior fragment and the C-terminal fragment are replaced with those shown in Table 3 (Peptide 4E and 5E), or with peptides having sequence having at least about 80%, at least about 85%, at least about 90%, or at least about 95% identical to the sequences of Peptide 4E and/or Peptide 5E.

In some embodiments, the N-terminal fragment, the first interior fragment, the second interior fragment, and the C-terminal fragment are arranged from the N-terminus to the C-terminus, respectively, in the modified IL-18 polypeptide.

### 5-Fragment Ligation Strategy

In some embodiments, the IL-18 polypeptide is prepared from 5 fragments. In some embodiments, the 5 fragments comprises an N-terminal fragment, a first interior fragment, a second interior fragment, a third interior fragment, and a C-terminal fragment. In some embodiments, synthesizing two or more fragments of the modified IL-18 polypeptide comprises synthesizing five fragments. In some embodiments, providing two or more fragments of the modified IL-18 polypeptide comprises providing five fragments. In some embodiments, the N-terminal fragment and the C-terminal fragment are as those in Table 2 (e.g., having a sequence at least about 80%, at least about 85%, at least about 90%, or at least about 95% identical to the relevant sequences). In some embodiments, the third interior fragment is one of those corresponding to residues 75-115 of the IL-18 polypeptide based on SEQ ID NO: 1 as a reference sequence shown in Table 2 (e.g., having a sequence at least about 70%, at least about 75%, at least about 80%, at least about 85%, at least about 90%, or at least about 95% identical to the relevant sequences). In some embodiments, the third interior fragment is one corresponding to residues 75-120 of the IL-18 polypeptide based on SEQ ID NO: 1 as a reference sequence and is selected from Table 3. In some embodiments, the first and second internal fragment each have at least about 80% sequence identity to any sequence independently selected from those provided in Table 3. In some embodiments, the three internal fragments each have at least about 85% sequence identity to those provided in Table 3. In some embodiments, the three internal fragments each have at least about 90% sequence identity to those provided in Table 3. In some embodiments, the three internal fragments each have at least about 95% sequence identity to those provided in Table 3. In some embodiments, the three internal fragments are each provided in Table 3. Any combination of peptides having at least about 70%, at least about 75%, at least about 80%, at least about 85%, at least about 90%, at least about 95%, or peptides identical to those shown in Table 2 and Table 3 can be combined to prepare a full IL-18 polypeptide, so long as every residue of the IL-18 polypeptide is present on the peptide fragments.

TABLE 3
Exemplary Peptides used to synthesize IL-18

| SEQ ID | | |
|---|---|---|
| NO | Comment | Sequence |
| 349 | Peptide 2C | (Opr) DMTDSDCRD NAPRTIFIIS MYKDSQPRGM |
| | (Residues 31-62) | A(Akv) |
| 350 | Peptide 3C | (Opr) ISVKCEK IST(Akl) |
| | (Residues 63-74) | |
| 351 | Peptide 2D | (Opr) DMTDSDCRD NAPRTIFIIS MYKDS(Akl) |
| | (Residues 31-56) | |
| 352 | Peptide 3D | (Opr) RGM AVTISVKCEK IST(Akl) |
| | (Residues 57-74) | |
| 353 | Peptide 2E | (Opr) DMTDSDCRD NAPRTIFI(Akl) |
| | (Residues 31-49) | |
| 354 | Peptide 3E | (Opr) MYKDSQPRGM AVTISVKCEK IST(Akl) |
| | (Residues 50-74) | |
| 355 | Peptide 2F | (Opr) DMTDSDCRD NAPRTIFIIS MYKDSQPRGM |
| | (Residues 31-66) | AVTIS(Akv) |
| 356 | Peptide 3F | (Opr) CEK IST(Akl) |
| | (Residues 67-74) | |
| 357 | Peptide 4E | (Opr) CENKI ISFKEMNPPD NIKDTKSDII |
| | (Residues 75-120) | FFQRSVPGHD NKMQFESSS(Akf) |
| 358 | Peptide 5E | (Opr) GYFLACEKE RDLFKLILKK EDELGDRSIM |
| | (Residues1 21-157) | FTVQNED |

*Opr = 5-oxa-proline;
Akl = alpha-keto leucine,
Akf = alpha-keto phenylalanine,
Akv = alpha-keto phenylalanine

In some embodiments, the N-terminal fragment comprises residues corresponding to residues 1-30 of SEQ ID NO: 1 (e.g., SEQ ID NO: 301, or a sequence having at least 70% identity therewith), the first interior fragment comprises residues corresponding to residues 31-62 of SEQ ID NO:1 (e.g., SEQ ID NO: 349, or a sequence having at least 70% identity therewith), the second interior fragment comprises residues corresponding to residues 63-74 of SEQ ID NO:1 (e.g., SEQ ID NO: 350, or a sequence having at least 70% identity therewith), the third interior fragment comprises residues corresponding to residues 75-115 of SEQ ID NO:1 (e.g., SEQ ID NO: 337, or a sequence having at least 70% identity therewith), and the C-terminal fragment comprises residues corresponding to residues 116-157 of SEQ ID NO:1 (e.g., SEQ ID NO: 343, or a sequence having at least 70% identity therewith). Exemplary peptides synthesized with this strategy can be found in SEQ ID NOs: 68-91. In some embodiments, In some embodiments, the method is used to make an IL-18 polypeptide having at least about 80%, at least about 85%, at least about 90%, or at least about 95% sequence identity to any one of SEQ ID NOs: 68-91.

In some embodiments, the N-terminal fragment comprises residues corresponding to residues 1-30 of SEQ ID NO: 1 (e.g., SEQ ID NO: 301, or a sequence having at least 70% identity therewith), the first interior fragment comprises residues corresponding to residues 31-56 of SEQ ID NO:1 (e.g., SEQ ID NO: 351, or a sequence having at least 70% identity therewith), the second interior fragment comprises residues corresponding to residues 57-74 of SEQ ID NO:1 (e.g., SEQ ID NO: 352, or a sequence having at least 70% identity therewith), the third interior fragment comprises residues corresponding to residues 75-115 of SEQ ID NO:1 (e.g., SEQ ID NO: 343, or a sequence having at least 70% identity therewith), and the C-terminal fragment comprises residues corresponding to residues 116-157 of SEQ ID NO:1 (e.g., SEQ ID NO: 343, or a sequence having at least 70% identity therewith). Exemplary peptides synthesized with this strategy can be found in SEQ ID NOs: 92-115. In some embodiments, In some embodiments, the method is used to make an IL-18 polypeptide having at least about 80%, at least about 85%, at least about 90%, or at least about 95% sequence identity to any one of SEQ ID NOs: 92-115.

In some embodiments, the N-terminal fragment comprises residues corresponding to residues 1-30 of SEQ ID NO: 1 (e.g., SEQ ID NO: 301, or a sequence having at least 70% identity therewith), the first interior fragment comprises residues corresponding to residues 31-49 of SEQ ID NO:1 (e.g., SEQ ID NO: 353, or a sequence having at least 70% identity therewith), the second interior fragment comprises residues corresponding to residues 50-74 of SEQ ID NO:1 (e.g., SEQ ID NO: 354, or a sequence having at least 70% identity therewith), the third interior fragment comprises residues corresponding to residues 75-120 of SEQ ID NO:1 (e.g., SEQ ID NO: 357, or a sequence having at least 70% identity therewith), and the C-terminal fragment comprises residues corresponding to residues 121-157 of SEQ ID NO:1 (e.g., SEQ ID NO: 358, or a sequence having at least 70% identity therewith). Exemplary peptides synthesized with this strategy can be found in SEQ ID NOs: 116-139. In some embodiments, In some embodiments, the method is used to make an IL-18 polypeptide having at least about 80%, at least about 85%, at least about 90%, or at least about 95% sequence identity to any one of SEQ ID NOs: 116-139.

In some embodiments, the N-terminal fragment comprises residues corresponding to residues 1-30 of SEQ ID NO: 1 (e.g., SEQ ID NO: 301, or a sequence having at least 70% identity therewith), the first interior fragment comprises residues corresponding to residues 31-66 of SEQ ID NO:1 (e.g., SEQ ID NO: 355, or a sequence having at least 70% identity therewith), the second interior fragment comprises residues corresponding to residues 67-74 of SEQ ID NO:1 (e.g., SEQ ID NO: 356, or a sequence having at least 70% identity therewith), the third interior fragment comprises residues corresponding to residues 75-115 of SEQ ID NO:1 (e.g., SEQ ID NO: 337, or a sequence having at least 70% identity therewith), and the C-terminal fragment comprises residues corresponding to residues 116-157 of SEQ ID NO:1 (e.g., SEQ ID NO: 343, or a sequence having at least 70% identity therewith). Exemplary peptides synthesized with this strategy can be found in SEQ ID NOs: 140-163. In some embodiments, In some embodiments, the method is used to make an IL-18 polypeptide having at least about 80%, at least about 85%, at least about 90%, or at least about 95% sequence identity to any one of SEQ ID NOs: 140-163.

In some embodiments, the method further comprises rearranging the ligated fragments. In some embodiments, rearranging the ligated fragments involves rearranging one or more depsipeptide bonds of the linear IL-18 polypeptide. In some embodiments, the one or more depsipeptide bonds are rearranged to form one or more amide bonds. In some embodiments, the depsipeptide bonds are formed as a result of the ligation of the fragments. In some embodiments, the depsipeptide bonds are between the hydroxyl moiety of a homoserine residue and an amino acid adjacent to the homoserine residue. In some embodiments, rearranging the ligated fragments occurs after each of the fragments have been ligated.

In some embodiments, ligated fragments are folded. In some embodiments, folding comprises forming one or more disulfide bonds within the modified IL-18 polypeptide. In some embodiments, the ligated fragments are subjected to a folding process. In some embodiments, the ligated fragments are folded using methods well known in the art. In some embodiments, the ligated polypeptide or the folded polypeptide are further modified by attaching one or more polymers thereto. In some embodiments, the ligated polypeptide or the folded polypeptide are further modified by PEGylation.

In some embodiments, the modified IL-18 polypeptide is synthetic.

An exemplary, non-limiting synthetic scheme of an IL-18 polypeptide modified with a polymer as provided herein is shown in FIG. 7. In general, in some embodiments, a first fragment (“Segment 1”) containing amino acids or amino acid precursors corresponding to residue numbers 1-30 of the modified IL-18 polypeptide is prepared (e.g., by solid phase peptide synthesis (SPPS)), as compared to the amino acid sequence set for in SEQ ID NO: 1. This is coupled to a second fragment (“Segment 2”) containing, in some embodiments, amino acids or amino precursors corresponding to either residue numbers 31-74 or residue numbers 31-62 of the modified IL-18 polypeptide to produce a single fragment (“Segment 12”). This second fragment is in some embodiments also prepared by SPPS. Similarly, a third fragment is prepared, in some embodiments by SPPS, having amino acids or amino acid precursors corresponding to either residue numbers 63-115 or 75-115 of the modified IL-18 polypeptide. This third fragment is coupled to a fourth fragment (“Segment 4”), in some embodiments prepared by SPPS, which contains amino acids or amino acid precursors corresponding to residue numbers 116-157 of the modified IL-18 polypeptide to produce a single fragment (“Segment 34”). Segment 12 and Segment 34 are then coupled to produce a full length fragment (“Segment 1234”). In embodiments where KAHA ligation is used to ligate the fragments, the site residues are then rearranged to produce amide bonds at the ligation points (e.g., depsipeptide homoserine rearrangement to amide bond). Finally, the full length linear fragment is then folded to produce a synthetic IL-18 polypeptide.

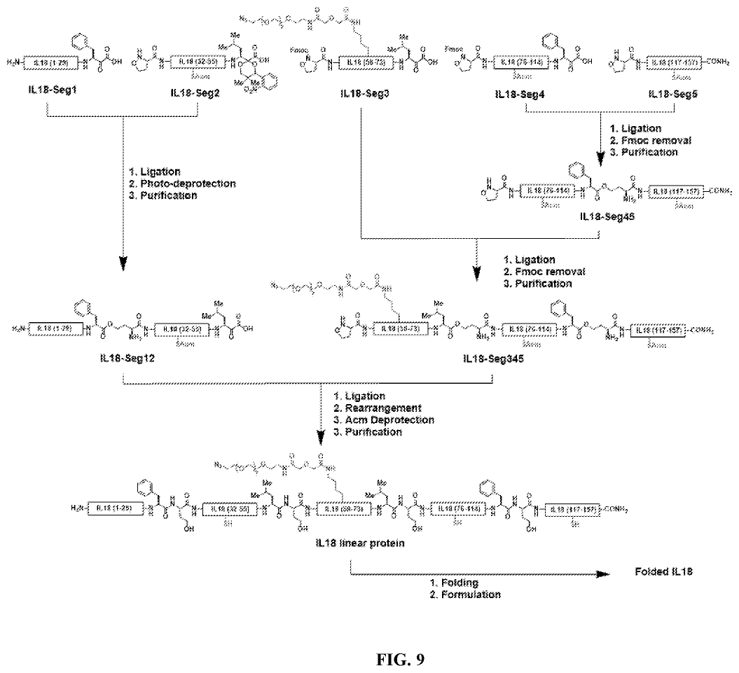

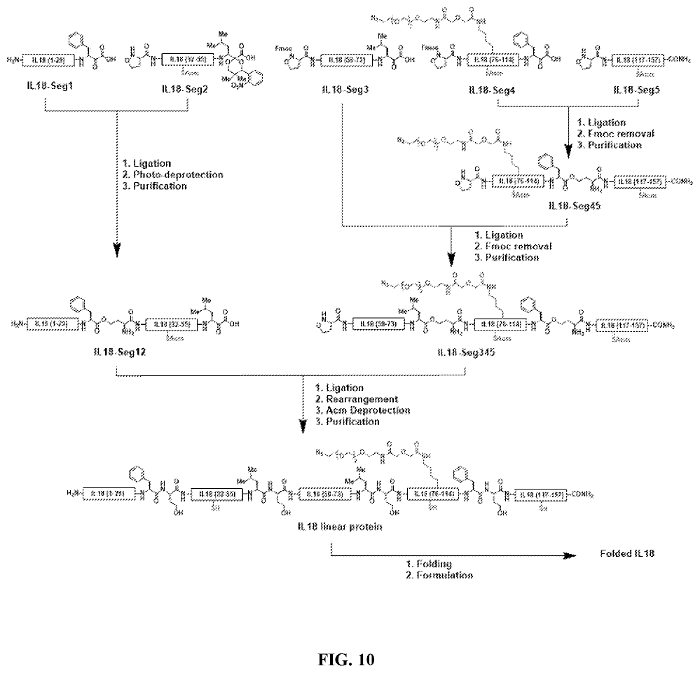

FIG. 8 shows an analogous protocol to that above, but the full length synthetic IL-18 is instead synthesized from 5 fragments. The IL-18 synthesis depicted therein incorporates an azide attached at residue position 68. The azide is attached to an aspartate residue through a PEG linker. FIG. 9 shows another synthesis of the full length synthetic IL-18 synthesized from 5 fragments. The IL-18 synthesis depicted therein incorporates an azide attached at residue position 68. The azide is attached to a lysine residue through a PEG linker. FIG. 10 shows an exemplary synthetic scheme for a synthesis of a modified IL-18 polypeptide comprising an azide attached to a lysine at residue position 86 from 5 fragments. In each of these exemplary syntheses, the azide is incorporated at the desired residue position during the synthesis of the IL-18 fragments. This azide functionality can later acts as a conjugation handle to attach a larger PEG group if desired, or an additional functionality (e.g., an additional polypeptide, such as an antibody or another cytokine).

### VIII. Synthetic IL-18

Also provided herein are chemically synthesized modified IL-18. Also provided herein are chemically synthesized IL-18s modified with polymers as provided herein. In some embodiments, the chemically synthesized IL-18s display a biological activity substantially identical to a recombinant IL-18 which contains the same functional modifications. In some embodiments, the chemically synthesized IL-18s contain modifications as provided herein for modified IL-18 polypeptides (e.g., any of the amino acid substitutions, addition of polymers, truncations, extensions, etc.). In some embodiments, the modifications provided herein modulate the biological activity of the synthetic IL-18 polypeptide as provided herein for modified IL-18 polypeptides.

Chemically synthesized IL-18 provide advantages over recombinant IL-18 because it can be synthesized to include any desired modification with ease in a site-specific manner, allowing ready modulation of the biological activity. Additionally, in some embodiments, synthetic IL-18 polypeptides incorporate non-canonical amino acids, allowing for more functional variation than is possible with recombinantly expressed. Additionally, the synthetic nature of the polypeptides allows for natural amino acid residues to be modified site specifically (e.g., during the synthesis process) to allow for addition of other groups to the synthetic IL-18 polypeptide (e.g., polymers or additional polypeptides) with, in some instances, minimal change to the polypeptide structure or function.

In one aspect provided herein is a synthetic IL-18 polypeptide comprising a polymer covalently attached to a residue as provided herein.

In some embodiments, the synthetic IL-18 polypeptide is prepared from one or more chemically synthesized fragments. In some embodiments, the synthetic IL-18 polypeptide is prepared from 1, 2, 3, 4, 5, 6, 7, 8, or more chemically synthesized fragments. In some embodiments, the synthetic IL-18 polypeptide is prepared from 4 chemically synthesized fragments. In some embodiments, the synthetic IL-18 polypeptide is prepared from 5 chemically synthesized fragments. In some embodiments, the synthetic IL-18 polypeptide is prepared form 4 or 5 chemically synthesized fragments.

In some embodiments, the synthetic IL-18 polypeptide, comprises a homoserine (Hse) residue at one or more positions within the synthetic polypeptide. In some embodiments, the synthetic IL-18 polypeptide comprises a homoserine residue at a position selected from the region of residues 21-41, residues 60-80, and residues 106-126, wherein residue position numbering of the synthetic IL-18 polypeptide is based on SEQ ID NO: 1 as a reference sequence. In some embodiments, the synthetic IL-18 comprises homoserine residues at positions selected from the region of residues 24-38, residues 60-66, residues 72-80, and residues 109-123 of the synthetic IL-18 polypeptide. In some embodiments, the synthetic IL-18 comprises homoserine residues at positions selected from the region of residues 27-35, residues 60-66, residues 72-80 and residues 112-120 of the synthetic IL-18 polypeptide. In some embodiments, the synthetic IL-18 comprises homoserine residues at positions selected from the region of residues 29-34, residues 60-66, residues 72-80, and residues 114-118 of the synthetic IL-18 polypeptide. In some embodiments, the synthetic IL-18 comprises homoserine residues at positions selected from the region of residues 30-33, residues 61-65, residues 73-79, and residues 115-117 of the synthetic IL-18 polypeptide. In some embodiments, the synthetic IL-18 polypeptide comprises a homoserine in one, two, or three of the regions provided herein.

In some embodiments, the synthetic IL-18 polypeptide comprises a Hse residue in one or more of the regions of residues 21-41, residues 53-73, and residues 106-126, wherein residue position numbering of the synthetic IL-18 polypeptide is based on SEQ ID NO: 1 as a reference sequence. In some embodiments, the synthetic IL-18 polypeptide comprises a Hse residue in one or more of the regions of residues 21-41, residues 65-85, and residues 106-126. In some embodiments, the synthetic IL-18 polypeptide comprises a Hse residue in two of the regions of residues 21-41, residues 53-73, and residues 106-126. In some embodiments, the synthetic IL-18 polypeptide comprises a Hse residue in two of the regions of residues 21-41, residues 65-85, and residues 106-126. In some embodiments, the synthetic IL-18 polypeptide comprises a Hse residue in each the regions of residues 21-41, residues 53-73, and residues 106-126. In some embodiments, the synthetic IL-18 polypeptide comprises a Hse residue in each the regions of residues 21-41, residues 65-85, and residues 106-126.

In some embodiments, the synthetic IL-18 polypeptide comprises a Hse residue in the regions of residues 21-41, a Hse residue in the region of residues 106-126, and two Hse residues in the region of residues 45-80, wherein residue position numbering of the synthetic IL-18 polypeptide is based on SEQ ID NO: 1 as a reference sequence. In some embodiments, the synthetic IL-18 polypeptide comprises a Hse residue in the regions of residues 21-41, a Hse residue in the region of residues 106-126, and two Hse residues selected from Hse 50, Hse57, Hse63, Hse 67, and Hse 75. In some embodiments, the synthetic IL-18 polypeptide comprises a Hse residue in the regions of residues 21-41, a Hse residue in the region of residues 106-126, a Hse residue in the region of residues 70-80, and a Hse residue in the region of residues 45-69. In some embodiments, the synthetic IL-18 polypeptide comprises a) Hse31, b) Hse 75, c) Hse 116 or Hse 121, and d) Hse50, Hse57, Hse63, or Hse 67.

In some embodiments, the synthetic IL-18 polypeptide comprises a Hse residue in two of the regions of residues 21-41, residues 53-73, and residues 106-126. In some embodiments, the synthetic IL-18 polypeptide comprises a Hse residue in two of the regions of residues 21-41, residues 65-85, and residues 106-126. In some embodiments, the synthetic IL-18 polypeptide comprises a Hse residue in each the regions of residues 21-41, residues 53-73, and residues 106-126. In some embodiments, the synthetic IL-18 polypeptide comprises a Hse residue in each the regions of residues 21-41, residues 65-85, and residues 106-126.

In some embodiments, the synthetic IL-18 polypeptide comprises a Hse residue at position 31. In some embodiments, the synthetic IL-18 polypeptide comprises a Hse residue at position 50. In some embodiments, the synthetic IL-18 polypeptide comprises a Hse residue at position 57. In some embodiments, the synthetic IL-18 polypeptide comprises a Hse residue at position 63. In some embodiments, the synthetic IL-18 polypeptide comprises a Hse residue at position 67. In some embodiments, the synthetic IL-18 polypeptide comprises a Hse residue at position 75. In some embodiments, the synthetic IL-18 polypeptide comprises a Hse residue at position 116. In some embodiments, the synthetic IL-18 polypeptide comprises a Hse residue at position 121. In some embodiments, the synthetic IL-18 polypeptide comprises Hse residues at one, two, three, or four of residues 31, 50, 57, 63, 67, 75, 116, and 121. In some embodiments, the synthetic IL-18 polypeptide comprises Hse residues at one, two, or three of residues 31, 75, and 116. In some embodiments, the synthetic IL-18 polypeptide comprises Hse residues at two or more of positions 31, 63, and 116. In some embodiments, the synthetic IL-18 polypeptide comprises Hse residues at two or more of positions 31, 75, and 116. In some embodiments, the synthetic IL-18 polypeptide comprises Hse residues at positions 31, 63, and 116. In some embodiments, the synthetic IL-18 polypeptide comprises Hse residues at positions 31, 75, and 116.

In some embodiments, the synthetic IL-18 polypeptide comprises an amino acid substitution of at least one methionine residue in SEQ ID NO: 1. In some embodiments, the amino acid substitution of at least one methionine residue comprises a substitution at M33, M51, M60, M86, M113, or M150. In some embodiments, the synthetic IL-18 polypeptide comprises substitutions of one, two, three, four, five or six methionine residues. In some embodiments, the synthetic IL-18 polypeptide comprises substitutions of at least two methionine residues. In some embodiments, the synthetic IL-18 polypeptide comprises substitutions of at least three methionine residues. In some embodiments, the synthetic IL-18 polypeptide comprises substitutions of at least four methionine residues. In some embodiments, the synthetic IL-18 polypeptide comprises substitutions of at least five methionine residues. In some embodiments, the synthetic IL-18 polypeptide comprises substitutions of six methionine residues.

In some embodiments, one or more methionine residues in the synthetic IL-18 polypeptide of SEQ ID NO: 1 are substituted for residues that do not contain sulfur atoms. In some embodiments, one or more methionine residues are each independently substituted for a methionine isostere. In some embodiments, one or more methionine residues are each independently substituted for norleucine (Nle) or O-methyl-homoserine (Omh). In some embodiments, at least one methionine residue is substituted for a Nle or Omh residue. In some embodiments, one methionine residue is substituted for Nle on Omh residue. In some embodiments, two methionine residues are each independently substituted for Nle or Omh residues. In some embodiments, three methionine residues are each independently substituted for Nle or Omh residues. In some embodiments, four methionine residues are each independently substituted for Nle or Omh residues. In some embodiments, five methionine residues are each independently substituted for Nle or Omh residues. In some embodiments, six methionine residues are each independently substituted for Nle or Omh residues. In some embodiments, each methionine is independently substituted for a Nle or Omh residue. In some embodiments, each methionine except for M86 is independently substituted for a Nle or Omh residue.

In some embodiments, at least one methionine residue is substituted for a Omh residue. In some embodiments, one methionine residue is substituted for Omh residue. In some embodiments, two methionine residues are substituted for Omh residues. In some embodiments, three methionine residues are substituted for Omh residues. In some embodiments, four methionine residues are substituted for Omh residues. In some embodiments, five methionine residues are substituted for Omh residues. In some embodiments, six methionine residues are substituted for Omh residues. In some embodiments, six methionine residues are substituted for Omh residues. In some embodiments, each methionine substitution is for Omh residues.

In some embodiments, the synthetic IL-18 polypeptide comprises an additional substitution to SEQ ID NO: 68, 92, 116, or 140. In some embodiments, the synthetic IL-18 polypeptide comprises an amino acid sequence at least about 75% identical to that of SEQ ID NO: 68, 92, 116, or 140. In some embodiments, the synthetic IL-18 polypeptide comprises an amino acid sequence at least about 80% identical to that of SEQ ID NO: 68, 92, 116, or 140. In some embodiments, the synthetic IL-18 polypeptide comprises an amino acid sequence at least about 85% identical to that of SEQ ID NO: 68, 92, 116, or 140. In some embodiments, the synthetic IL-18 polypeptide comprises an amino acid sequence at least about 90% identical to that of SEQ ID NO: 68, 92, 116, or 140. In some embodiments, the synthetic IL-18 polypeptide comprises an amino acid sequence at least about 95% identical to that of SEQ ID NO: 68, 92, 116, or 140. In some embodiments, the synthetic IL-18 polypeptide comprises an amino acid sequence identical to that of SEQ ID NO: 68, 92, 116, or 140.

The synthetic IL-18 polypeptide can comprise any of the modifications or substitutions of a modified IL-18 polypeptide provided herein. In some embodiments, the synthetic IL-18 polypeptide comprises an amino acid substitution at Y01, F02, E06, V11 E31, M33, C38, I49, S50, M51, K53, D54, S55, Q56, P57, M60, T63, C68, K70, S75, C76, E85, M86, T95, D98, M113, E116, E121, C127, or M150, wherein residue position numbering is based on SEQ ID NO: 1 as a reference sequence. In some embodiments, the synthetic IL-18 polypeptide comprises a substitution at residue E06. In some embodiments, the synthetic IL-18 polypeptide comprises an amino acid substitution at residue K53. In some embodiments, the synthetic IL-18 polypeptide comprises an amino acid substitution at residue T63. In some embodiments, the synthetic IL-18 polypeptide comprises an amino acid substitution at residue E85. In some embodiments, the synthetic IL-18 polypeptide comprises an amino acid substitution at residue M86. In some embodiments, the synthetic IL-18 polypeptide comprises an amino acid substitution at residue T95. In some embodiments, the synthetic IL-18 polypeptide comprises an amino acid substitution at residue D98. In some embodiments, the synthetic IL-18 polypeptide comprises a truncation or an extension polypeptide relative to the amino acid sequence set forth in SEQ ID NO: 1.

In some embodiments, the synthetic IL-18 polypeptide comprises a conjugation handle. In some embodiments, the conjugation handle is attached to a specified desired residue of the synthetic IL-18 polypeptide. In some embodiments, the conjugation handle is attached to a side chain of a residue of the modified IL-18 polypeptide. In some embodiments, the conjugation handle is attached to a side chain of a natural amino acid residue of the modified IL-18 polypeptide (e.g., the side chain of a C, D, E, K, N, Q, S, T, or Y residue). In some embodiments, the natural amino acid residue is an amino acid set for in SEQ ID NO: 1. In some embodiments, the natural amino acid with the conjugation handle attached to the side chain is substituted for an amino acid set forth in SEQ ID NO: 1. In some embodiments, the conjugation handle is attached to an unnatural amino acid (e.g., azidolysine). In some embodiments, the conjugation handle is attached to the N-terminal amine of the synthetic IL-18 polypeptide. In some embodiments, the conjugation handle is attached to a residue to the modified IL-18 polypeptide through a linker (e.g., a polymer as provided herein). In some embodiments, the conjugation handle is used to attach the polymer to the modified IL-18 polypeptide. In some embodiments, the conjugation handle is used to attach an additional group to the synthetic IL-18 polypeptide (e.g., a second polymer or an additional polypeptide).

In some embodiments, the synthetic IL-18 polypeptide comprises a polymer covalently attached to a residue of the synthetic IL-18 polypeptide (e.g., any one of residues 79-120 as provided herein). The polymer may be any of the polymers provided herein and may be attached at any residue as provided herein. In some embodiments, the polymer is water soluble polymer. In some embodiments, the polymer comprises a linker group attaching the polymer to the synthetic IL-18 polypeptide. In some embodiments, the synthetic IL-18 polypeptide comprises multiple polymers covalently attached to the synthetic IL-18 polypeptide.

In some embodiments, the synthetic IL-18 polypeptide is conjugated to an additional polypeptide. In some embodiments, the synthetic IL-18 polypeptide is covalently attached to the additional polypeptide. In some embodiments, the synthetic IL-18 polypeptide is covalently attached to the additional polypeptide through a linker. In some embodiments, the linker comprises a polymer. In some embodiments, the polymer comprises a water-soluble polymer (e.g., PEG). In some embodiments, the synthetic IL-18 polypeptide is attached to the additional polypeptide through a non-covalent interaction. In some embodiments, the non-covalent interaction is an interaction biotin with streptavidin or avidin. In some embodiments, the additional polypeptide is an antibody, antibody fragment, single chain variable fragments (ScFv), peptide aptamer, cyclic peptide, branched peptide, growth factor, peptide hormone, chemokine, or cytokine. In some embodiments, the additional polypeptide is an antibody or an antigen-binding fragment thereof. In some embodiments, the antibody comprises a humanized antibody, a murine antibody, a chimeric antibody, a bispecific antibody, any fragment thereof, or any combination thereof. In some embodiments, the antibody is a monoclonal antibody or a fragment thereof. In some embodiments, the additional polypeptide is a cytokine. In some embodiments, the additional polypeptide is a half-life extension polypeptide (e.g., albumin).

### NUMBERED EMBODIMENTS

1. A modified interleukin-18 (IL-18) polypeptide, comprising: a polymer attached to a residue of the modified IL-18 polypeptide, wherein the modified IL-18 polypeptide displays a lower binding affinity (resulting in a higher K_{D}) for an IL-18 receptor alpha/beta heterodimer (IL-18Rα/β) which is at most eight fold less than the binding affinity displayed by an identical modified IL-18 polypeptide with no polymer attached.
2. A modified interleukin-18 (IL-18) polypeptide, comprising: a polymer attached to any one of residues 79-120, wherein residue position numbering of the modified IL-18 polypeptide is based on SEQ ID NO: 1 as a reference sequence. 3. The modified IL-18 polypeptide of embodiment 1 or 2, wherein the polymer is covalently attached to a residue selected from residue 79, residue 80, residue 81, residue 82, residue 83, residue 84, residue 85, residue 86, residue 87, residue 88, residue 89, residue 90, residue 91, residue 92, residue 93, residue 94, residue 95, residue 96, residue 97, residue 98, residue 99, residue 100, residue 101, residue 102, residue 103, residue 104, residue 105, residue 106, residue 107, residue 108, residue 109, residue 110, residue 111, residue 112, residue 113, residue 114, residue 115, residue 116, residue 117, residue 118, residue 119, and residue 120. 4. The modified IL-18 polypeptide of embodiment 1 or 2, wherein the polymer is covalently attached at residue 85. 5. The modified IL-18 polypeptide of embodiment 1 or 2, wherein the polymer is covalently attached at residue 86. 6. The modified IL-18 polypeptide of embodiment 1 or 2, wherein the polymer is covalently attached at residue 98. 7. The modified IL-18 polypeptide of embodiment 1 or 2, wherein the polymer is covalently attached at residue E85, E85C, E85D, E85Q, E85K, E85N, or E85Y. 8. The modified IL-18 polypeptide of embodiment 1 or 2, wherein the polymer is covalently attached at residue M86C, M86D, M86Q, M86K, M86N, M86E, or M86Y. 9. The modified IL-18 polypeptide of embodiment 1 or 2, wherein the polymer is covalently attached at residue T95C, T95D, T95Q, T95K, T95N, T95E, T95Y, D98C, D98Q, D98K, D98N, D98E, or D98Y. 10. The modified IL-18 polypeptide of any one of embodiments 1-9, wherein the polymer is attached to a natural amino acid residue. 11. The modified IL-18 polypeptide of embodiment 10, wherein the natural amino acid residue is selected from asparagine, aspartic acid, cysteine, glutamic acid, glutamine, lysine, and tyrosine. 12. The modified IL-18 polypeptide of any one of embodiments 1-6, wherein the polymer is attached to an unnatural amino acid residue. 13. The modified IL-18 polypeptide of any one of embodiments 1-12, wherein one of residues 79-120 is substituted for a cysteine. 14. The modified IL-18 polypeptide of any one of embodiments 1-13, wherein the polymer comprises a conjugation handle or a reaction product of a conjugation handle with a complementary conjugation handle. 15. The modified IL-18 polypeptide of embodiment 14, wherein the conjugation handle or the reaction product of the conjugation handle with the complementary conjugation handle comprises an azide moiety, an alkyne moiety, or reaction product of an azide-alkyne cycloaddition reaction. 16. The modified IL-18 polypeptide of any one of embodiments 1-15, wherein the polymer has a weight average molecular weight of at most about 50,000 Daltons, at most about 25,000 Daltons, at most about 10,000 Daltons, at most about 6,000 Daltons, at most about 2,000 Daltons, at most about 1,000 Daltons, at most about 500 Daltons, at most about 250 Daltons, or at most about 100 Daltons. 17. The modified IL-18 polypeptide of any one of embodiments 1-16, wherein the polymer is a water-soluble polymer. 18. The modified IL-18 polypeptide of embodiment 17, wherein the water-soluble polymer comprises poly(alkylene oxide), polysaccharide, poly(vinyl pyrrolidone), poly(vinyl alcohol), polyoxazoline, poly(acryloylmorpholine), or a combination thereof. 19. The modified IL-18 polypeptide of embodiment 18, wherein the water-soluble polymer comprises poly(alkylene oxide). 20. The modified IL-18 polypeptide of embodiment 19, wherein the poly(alkylene oxide) is polyethylene glycol (PEG). 21. The modified IL-18 polypeptide of embodiment 20, wherein the polyethylene glycol has a weight average molecular weight of about 0.1 kDa, 0.25 kDa, 0.5 kDa, 1 kDa to about 50 kDa. 22. The modified IL-18 polypeptide of embodiment 21, wherein the polyethylene glycol has a weight average molecular weight of about 10 kDa, about 20 kDa, or about 30 kDa. 23. The modified IL-18 polypeptide of any one of embodiments 1-22, comprising one or more amino acid substitutions at residue Y1, F2, E6, V11, C38, K53, D54, S55, T63, C76, or C127, or any combination thereof. 24. The modified IL-18 polypeptide of any one of embodiments 1-23, comprising a Y01G, F02A, E06K, V11I, C38S, C38A, C38Q, D54A, S55A, T63A, C76S, C76A, C127S, or C127A amino acid substitution, or any combination thereof. 25. The modified IL-18 polypeptide of any one of embodiments 1-24, comprising E06K and K53A amino acid substitutions. 26. The modified IL-18 polypeptide of any one of embodiments 1-25, comprising a V11I amino acid substitution. The modified IL-18 polypeptide of any one of embodiments 1-26, comprising a T63A amino acid substitution. 28. The modified IL-18 polypeptide of any one of embodiments 1-27, wherein the modified IL-18 polypeptide comprises an N-terminal extension. 29. The modified IL-18 polypeptide of any one of embodiments 1-27, comprising an N-terminal truncation. 30. The modified IL-18 polypeptide of any one of embodiments 1-29, wherein the modified IL-18 polypeptide comprises a polypeptide sequence having at least about 80% sequence identity to any one of SEQ ID NOs: 2-203. 31. The modified IL-18 polypeptide of embodiment 30, wherein the polypeptide sequence is at least about 80% identical to SEQ ID NO: 4 or SEQ ID NO: 30. 32. The modified IL-18 polypeptide of embodiment 30, wherein the polypeptide sequence is at least about 80% identical to SEQ ID NO: 4. 33. The modified IL-18 polypeptide of embodiment 30, wherein the polypeptide sequence is at least about 90% identical to SEQ ID NO: 4. 34. The modified IL-18 polypeptide of embodiment 30, wherein the polypeptide sequence is at least about 95% identical to SEQ ID NO: 4. 35. The modified IL-18 polypeptide of embodiment 30, wherein the polypeptide sequence is at least about 80% identical to SEQ ID NO: 30. 36. The modified IL-18 polypeptide of embodiment 30, wherein the polypeptide sequence is at least about 90% identical to SEQ ID NO: 30. 37. The modified IL-18 polypeptide of embodiment 30, wherein the polypeptide sequence is at least about 95% identical to SEQ ID NO: 30. 38. The modified IL-18 polypeptide of any one of embodiments 1-37, wherein the modified IL-18 polypeptide is recombinant. 39. The modified IL-18 polypeptide of any one of embodiments 1-37, comprising one or more amino acid substitutions selected from: (i) a homoserine residue located at any one of residues 26-36; (ii) a homoserine residue located at any one of residues 45-67; (iii) a homoserine residue located at any one of residues 70-80; (iv) a homoserine residue located at any one of residues 100-130; (v) a norleucine or O-methyl-homoserine residue located at any one of residues 28-38; (vi) a norleucine or O-methyl-homoserine residue located at any one of residues 46-56; (vii) a norleucine or O-methyl-homoserine residue located at any one of residues 54-64; (viii) a norleucine or O-methyl-homoserine residue located at any one of residues 80-90; (ix) a norleucine or O-methyl-homoserine residue located at any one of residues 108-118; and (x) a norleucine or O-methyl-homoserine residue located at any one of residues 145-155; wherein residue position numbering of the modified IL-18 polypeptide is based on SEQ ID NO: 1 as a reference sequence. 40. The modified IL-18 polypeptide of embodiment 39, comprising one or more amino acid substitutions selected from homoserine (Hse) 31, norleucine (Nle) 33, O-methyl-homoserine (Omh) 33, Hse50, Nle51, Omh51, Hse57, Nle60, Hse63, Omh60, Hse75, Nle86, Omh86, Hse116, Nle113, Omh113, Hse 121, Nle150, and Omh150. 41. The modified IL-18 polypeptide of any one of embodiments 1-40, wherein the modified IL-18 polypeptide modulates IFNγ production, and wherein an EC50 (nM) of the modified IL-18 polypeptide's ability to induce IFNγ is less than 10-fold higher than, less than 5-fold higher than, or less than an EC50 (nM) of an IL-18 polypeptide of SEQ ID NO: 1. 42. The modified IL-18 polypeptide of embodiment 41, wherein the EC50 (nM) of the modified IL-18 polypeptide's ability to induce IFNγ is less than 5-fold greater than the EC50 (nM) of SEQ ID NO: 1. 43. The modified IL-18 polypeptide of embodiment 41, wherein the EC50 (nM) of the modified IL-18 polypeptide's ability to induce IFNγ is less than the EC50 (nM) an IL-18 polypeptide of SEQ ID NO: 1. 44. The modified IL-18 polypeptide of embodiment 41, wherein the EC50 (nM) of the modified IL-18 polypeptide's ability to induce IFNγ is at least about 10-fold less than the EC50 (nM) of SEQ ID NO: 1. 45. The modified IL-18 polypeptide of any one of embodiments 1-44, wherein the modified IL-18 polypeptide exhibits less than a 10-fold lower affinity, less than a 5-fold lower affinity, or a greater affinity to an IL-18 receptor alpha subunit (IL-18Rα) than to IL-18 binding protein (IL-18BP) as measured by K_{D}, and wherein [K_{D} IL-18Rα]/[K_{D} IL-18BP] is greater than 0.1. 46. The modified IL-18 polypeptide of any one of embodiments 1-45, wherein the modified IL-18 polypeptide binds to IL-18 receptor alpha (IL-18Rα). 47. The modified IL-18 polypeptide of embodiment 46, wherein the modified IL-18 polypeptide binds to IL-18Rα with a K_{D} of less than about 200 nM, less than about 100 nM, less than about 80 nM, less than about 70 nM, less than about 60 nM, or less than about 50 nM. 48. The modified IL-18 polypeptide of embodiment 47, wherein the modified IL-18 polypeptide binds to IL-18Rα with a K_{D} of less than about 50 nM. 49. The modified IL-18 polypeptide of any one of embodiments 1-48, wherein the modified IL-18 polypeptide binds to an IL-18 receptor alpha/beta (IL-18Rα/β) heterodimer. 50. The modified IL-18 polypeptide of embodiment 49, wherein the modified IL-18 polypeptide binds to the IL-18Rα/P heterodimer with a K_{D} of less than about 25 nM. 51. The modified IL-18 polypeptide of embodiment 49, wherein the modified IL-18 polypeptide binds to the IL-18Rα/P heterodimer with a K_{D} of less than about 10 nM. 52. The modified IL-18 polypeptide of any one of embodiments 1-51, wherein the modified IL-18 polypeptide is conjugated to an additional peptide.
53. A host cell comprising a modified IL-18 polypeptide of any one of embodiments 1-52.
54. A method of producing a modified IL-18 polypeptide of any one of embodiments 1-52, comprising expressing the modified IL-18 polypeptide in a host cell. 55. The host cell of embodiment 53, wherein the host cell is a prokaryotic cell or a eukaryotic cell. 56. The host cell of embodiment 53, wherein the host cell is a mammalian cell, an avian cell, a fungal cell, or an insect cell. 57. The host cell of embodiment 56, wherein the host cell is a CHO cell, a COS cell, or a yeast cell.
58. A pharmaceutical composition comprising: (a) a modified IL-18 polypeptide of any one of embodiments 1-52; and (b) a pharmaceutically acceptable carrier or excipient. 59. The pharmaceutical composition of embodiment 58, wherein the pharmaceutical composition is in a lyophilized form.
60. A method of treating cancer in a subject in need thereof, comprising: administering to the subject a pharmaceutically effective amount of a modified IL-18 polypeptide of any one of embodiments 1-52 or a pharmaceutical composition of embodiments 58 or 59. 61. The method of embodiment 60, wherein the cancer is a solid cancer. 62. The method of embodiment 61, wherein, the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer. 63. The method of embodiment 61, wherein the solid cancer is metastatic renal cell carcinoma or melanoma. 64. The method of embodiment 61, wherein the solid cancer is a carcinoma or a sarcoma. 65. The method of embodiment 60, wherein the cancer is a blood cancer. 66. The method of embodiment 65, wherein the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia. 67. The method of any one of embodiments 60-66, further comprising reconstituting a lyophilized form of the modified IL-18 polypeptide or the pharmaceutical composition.
68. A method of making a modified IL-18 polypeptide of any one of embodiments 1-51, comprising: (i) synthesizing two or more fragments of the modified IL-18 polypeptide; (ii) ligating the fragments; and (iii) folding the ligated fragments. 69. The method of embodiment 68, wherein at least one of the fragments of the IL-18 polypeptide comprises a conjugation handle. 70. The method of embodiment 69, further comprising attaching the polymer to the folded, ligated fragments by a reaction with the conjugation handle. 71. The method of embodiment 68, whereon one of the fragments comprises the polymer.
72. An modified IL-18 polypeptide having at least about 80%, at least about 85%, at least about 90%, at least about 95%, at least about 96%, at least about 97%, at least about 98%, or at least about 99% sequence identity to the sequence set forth in any one of SEQ ID NOs: 2-33.
73. An modified IL-18 polypeptide having at least about 80%, at least about 85%, at least about 90%, at least about 95%, at least about 96%, at least about 97%, at least about 98%, or at least about 99% sequence identity to the sequence set forth in any one of SEQ ID NOs: 24-33.
74. A modified IL-18 polypeptide having at least about 80%, at least about 85%, at least about 90%, at least about 95%, at least about 96%, at least about 97%, at least about 98%, or at least about 99% sequence identity to the sequence set forth in SEQ ID NO: 30. 75. The modified IL-18 polypeptide of any one of embodiments 72-74, further comprising a polymer covalently attached. 76. The modified IL-18 polypeptide of embodiment 75, wherein the polymer is covalently attached at residue 68, 69, or 70. 77. The modified IL-18 polypeptide of embodiment 76, wherein the polymer is covalently attached at residue 68.

The present disclosure is further illustrated in the following Examples which are given for illustration purposes only and are not intended to limit the disclosure in any way.

### EXAMPLES

### Example 1—Recombinant IL18 Expression and Purification

Recombinant IL-18 variants provided herein can be prepared according to the protocols provided below.

Soluble his-SUMO-IL18 Variants

Recombinant BL21 Star (DE3) containing plasmid encoding an IL-18 polypeptide provided herein (from glycerol stocks) is inoculated into LB medium containing 50 μg/mL kanamycin and cultured at 37° C. When the OD600 reaches about 0.8-1.0, cell culture is induced with 0.5 mM IPTG at 18° C./18 h. Cells are then harvested by centrifugation (4500×g, 45 min, 4° C.). Cells are pelleted and cell lysis is done by sonication in lysis buffer: PBS, pH 7.4. Soluble protein is purified via Ni-NTA beads 6FF (wash 1 with: PBS, 20 mM imidazole, pH7.4; wash 2 with PBS, 50 mM Imidazole, pH7.4; elution with PBS, 500 mM imidazole, pH7.4).

Cell pellets are resuspended with lysis buffer (50 mM NaH2PO4, 300 mM NaCl, 20 mM Imidazole, pH 7.4) followed by sonication on ice (20% amplitude, 5 seconds on/5 seconds off, 5 minutes total). The lysate is cleared by centrifugation (13500 RPM, 45 min, 4° C.) and the supernatant is kept for future purification. Target protein is obtained by three-step purification:
Step 1: Column: Histrap FF 5 mL (Cytiva)

Buffer A: 50 mM NaH2PO4, 300 mM NaCl, 20 mM Imidazole pH 7.4/Buffer B: 50 mM NaH2PO4, 300 mM NaCl, 500 mM Imidazole, pH 7.4. Elution with gradient 0-18% B over 5 CV (in waste), then constant 18% B over another 10 CV (3 ml Fractions).
Step 2: Column: Histrap FF 5 mL

The collected fractions are pooled into Dialysis tube (SnakeSkin, 10 K, 35 mm), then buffer-exchanged with 5 L dialysis buffer (50 mM NaH2PO4, 1 mM DTT, pH 7.4) over night at 4° C. to remove imidazole. SUMO protease is added into the tag-fused protein (amounts assessed by nanodrop) solution at a ratio of 1:50 (w/w), SUMO tag is fully cleaved after 1 h at 4° C. Reaction solution is then applied into Histrap FF 5 mL, flow through is collected, SUMO protease and His-SUMO tag are eluted with a gradient: 0% B over 10 CV (3 ml Fractions), 0-60% B over 5 CV (in waste), 60-100% B with 5 CV, 100% B with 5 CV.
Step 3: Column: HiLoad 16/600 Superdex 75 pg

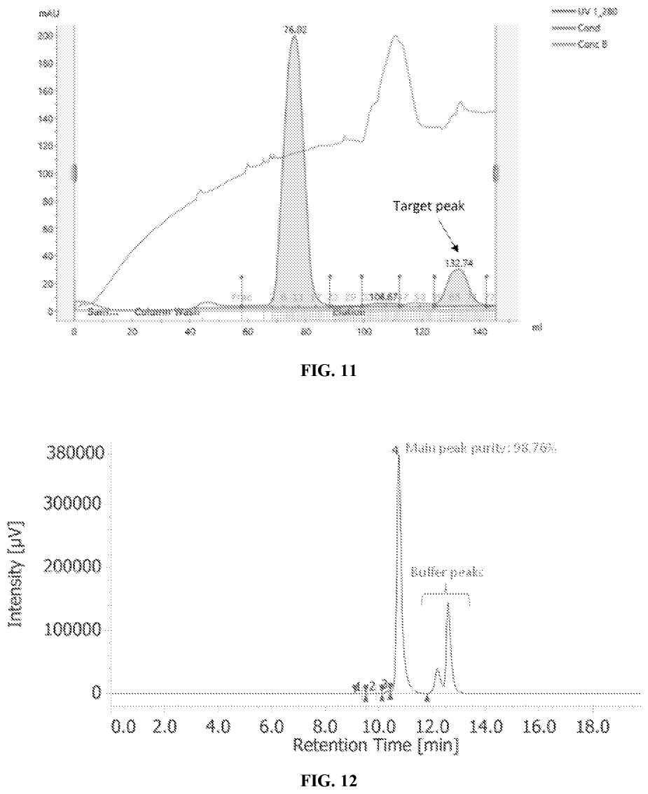

Pooled flow through is concentrated to 6-8 mg/mL, 5 mL concentrated protein solution is applied to the column, target protein was eluted with 1.2 CV elution buffer (1×PBS, 10% glycerol, pH 7.4). Target protein is pooled and sterilized by 0.22 m filter before stored in aliquots at −80° C. An exemplary purification trace of an IL-18 polypeptide of SEQ ID NO: 59 is shown in FIG. 11. The concentration is determined by nanodrop.

The protein purity and molecular weight are determined by standard SDS-PAGE, HR-MS, SEC- and RP-HPLC before undergoing endotoxin removal and filtration.

Soluble his-SUMO-IL18 Variants (Protocol 2)

IL18 candidates are produced as an N-terminal fusion to N-His-SUMO-IL18. The gene is synthesized and cloned into plasmids by a commercial service provider. Plasmids are transformed into E. coli BL21 (DE3). Transformed cells are inoculated into TB medium containing 50 μg/mL kanamycin and cultured at 37° C. When the OD600 reached about 1.2, the cell culture is induced with 0.1 mM IPTG at 18° C. for 20 h.

Cells are then harvested by centrifugation (4500×g, 45 min, 4° C.). Cells are pelleted and cell lysed with a homogenizer at 1000 bar.in lysis buffer: (20 mM Tris/HCl, pH 8.0, 0.15 M NaCl, 10 mM Imidazole, and one tablet of EDTA-free complete protease inhibitor (Roche, COEDTAF-RO) per liter original culture volume.

Lysates are clarified by centrifugation twice at 40,000 g for 45 minutes. Soluble lysates are then subsequent filtered through a 0.22 μm filter.

The soluble lysate is loaded on column containing Ni NTA resin (Cytiva, 17524802) that had been pre-equilibrated with 20 mM Tris/HCl, pH 8.0, 0.15 M NaCl, 10 mM Imidazole, at 5 m/min and washed with the same buffer for 5 CV. To remove endotoxins, the column is washed with 20 mM Tris/HCl, pH 8.0, 0.15 M NaCl, 10 mM Imidazole, 0.1% Triton X-114 at 10 mL/min for 30 CV. The column is washed with 20 mM Tris/HCl, pH 8.0, 0.15 M NaCl, 10 mM Imidazole, for 5 CV at 5 mL/min and the protein of interest eluted by linear increase of imidazole concentration.

To cleave the SUMO tag, SUMO protease is added to the elution pool at a w/w ratio of 1:250 (protein:SUMO enzyme) and incubated for 18 hours at 4° C. At the same time, the protein is dialysed versus 20 mM Tris, pH 8.0, 150 mM NaCl to reduce the imidazole concentration.

To remove the cleaved SUMO tag and the SUMO protease, the digested protein is passed through a Ni NTA resin column pre-equilibrated with 20 mM Tris/HCl, pH 8.0, 0.15 M NaCl, 10 mM Imidazole, at 5 mL/min. The unbound protein is collected.

The purified protein is concentrated to 2.6 mg/mL and buffer exchanged into either 20 mM HEPES, 150 mM NaCl, 0.5 mM TCEP, 10% glycerol, pH7.5 or PBS, 10% glycerol, pH7.4. Proteins are frozen in liquid nitrogen and stored at −70° C.

Insoluble his-SUMO-IL18 Variants

E. coli BL21 (DE3) harboring a plasmid encoding a N-His-SUMO tagged IL-18 variant fusion are inoculated into 10 L LB culture medium and induced with 0.4 mM IPTG at 30° C. for 6h. Cells are pelleted and cell lysis is done by sonication in lysis buffer: PBS, 8 M urea, pH 7.4. Protein is purified via Ni-NTA beads 6FF (wash 1 with: PBS, 8 M urea, 20 mM imidazole, pH7.4; wash 2 with PBS, 8 M urea, 50 mM Imidazole, pH7.4; elution with PBS, 8 M urea, 500 mM imidazole, pH7.4).

Fractions containing the protein are pooled, dialyzed into PBS pH 7.4 and followed by SUMO digestion. Then the protein is purified with Ni-NTA beads (equilibrate column with PBS, 8 M urea, pH 7.4, wash with PBS, 8 M urea, pH 7.4, elution with PBS, 8 M urea, pH 7.4). Fractions containing the protein are pooled, dialyzed into PBS pH 7.4 and QC is performed using analytical techniques, such as SDS-PAGE and analytical SEC.

### Insoluble Tagless IL18 Variants

E. coli BL21 (DE3) harboring a plasmid encoding mIL-18 is inoculated into 2 L LB culture medium and induced with 0.4 mM IPTG at 30° C. for 6h. Cells are pelleted and cell lysis was done by sonication in lysis buffer: 110 mM Tris, 1.1 M guanidine HCl, 5 mM DTT, pH 8.9. Protein as purified via Q Sepharose FF (balance buffer 20 mM MES, pH 7.0, elution with an increasing gradient from 0 to 1 M NaCl)

### Example 2—Chemical Synthesis of Modified IL-18 Polypeptides

Modified IL-18 polypeptides provide herein can also be prepared synthetically. A modified IL-18 polypeptide is prepared by ligating individual peptides synthesized using solid phase peptide synthesis (SPPS). Individual peptides are synthesized on an automated peptide synthesizer using the methods described below.

Commercially available reagents are purchased from Sigma-Aldrich, Acros, Merck or TCI Europe and used without further purification. Fluorenylmethoxycarbonyl (Fmoc) amino acids with suitable side-chain protecting groups for solid phase peptide synthesis are purchased from Novabiochem, Christof Senn Laboratories AG or PeptART and used as supplied. The polyethylene glycol derivatives used for peptide synthesis are purchased by Polypure. HPLC grade CH₃CN from Sigma Aldrich is used for analytical and preparative HPLC purification.

Peptides and proteins are characterized by high resolution Fourier-transform mass spectrometry (FTMS) using a Bruker solariX (9.4 T magnet) spectrometer equipped with a dual ESI/MALDI-FTICR source using 4-hydroxy-α-cyanocinnamic acid (HCCA) as matrix. CD spectra are recorded with a Jasco J-715 spectrometer with a 1.0 mm path length cell. CD spectra are collected at 25° C. in continuous scanning mode with standard sensitivity (100 mdeg), 0.5 nm data pitch, 50 nm/min scanning speed and 1 nm bandwidth. CD curves are obtained by averaging 5 scans and subtracting the background signal.

Peptide segments, ligated peptides, and linear proteins are analyzed and purified by reverse phase high performance liquid chromatography (RP-HPLC). The peptide analysis and reaction monitoring are performed on analytical Jasco instruments with dual pumps, mixer and in-line degasser, autosampler, a variable wavelength UV detector (simultaneous monitoring of the eluent at 220 nm and 254 nm), and an injector fitted with a 100 μL injection loop. The purification of the peptide segments is performed on a Gilson preparative instrument or Jasco semi-preparative instrument with 10-20 mL injection loop. The mobile phase is MilliQ-H₂O with 0.1% TFA (v/v) (Buffer A) and HPLC grade CH₃CN with 0.1% TFA (v/v) (Buffer B). Analytical HPLC is performed on bioZen™ Intact C4 column (3.6 μm, 150×4.6 mm) at room temperature or Aeris WIDEPORE XB-C18 column (3.6 μm, 150×4.6 mm) with a flow rate of 1 m/min at 60° C. Preparative HPLC is performed on a Gemini NX-C18 110 Å column (5 μm, 250×50 mm) or on a Shiseido capcell Pak UG80 C18 column (5 μm, 250×50 mm) at a flow rate of 40 m/min at 40° C. or 60° C. Semi-preparative HPLC was performed on a Shiseido capcell Pak C18 column (5 μm, 250×20 mm) at a flow rate of 10 mL/min at 60° C.

The peptide segments are synthesized on an automated peptide synthesizer using Fmoc-SPPS chemistry. The following Fmoc-amino acids with side-chain protecting groups are used: Fmoc-Ala-OH, Fmoc-Arg(Pbf)-OH, Fmoc-Asn(Trt)-OH, Fmoc-Asp(OtBu)-OH, Fmoc-Asp(OBno)-OH, Fmoc-Asp(OAll)-OH, Fmoc-Cys(Acm)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Lys(alloc)-OH, Fmoc-Met-OH, Fmoc-Met(O)—OH, Fmoc-Hse(Me)-OH, Fmoc-Nle-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH. Fmoc-pseudoproline dipeptides are incorporated in the synthesis if necessary. Fmoc deprotections are performed with 20% piperidine in DMF (2×8 min) or 25% piperidine in DMF containing 0.1 M Cl-HOBt (2×8 min) or 20% piperidine in DMF containing 0.1 M Cl-HOBt (2×8 min), and monitored by UV at 304 nm with a feedback loop to ensure complete Fmoc removal. Couplings are performed with Fmoc-amino acid (3.0-5.0 eq to resin substitution), HCTU or HATU (2.9-4.9 eq) as coupling reagents and DIPEA or NMM (6-10 eq) in DMF at room temperature or at 50° C. After pre-activating for 3 min, the solution is added to the resin and allowed to react for 15 min, 30 min or 2 h depending on the amino acid. In some cases, double couplings were required. In some cases, the resin is treated with 20% acetic anhydride in DMF for capping any unreacted free amine. LiCl washings are performed if required. The allyloxycarbonyl (Alloc) deprotection is performed under nitrogen using phenylsilane (24 eq) and tetrakis(triphenylphosphine)palladium(0) (0.5 eq) in nitrogen purged dichloromethane at room temperature for 30 min.

The synthesis of the peptide segments by SPPS is monitored by microcleavage using the following sample protocol: 10 mg of peptidyl resin are treated with a cleavage cocktail (200 μL) at room temperature for 1.5 h. The resin is filtered off and the filtrate was concentrated and treated with cold diethyl ether, triturated and centrifuged. The ether layer is carefully decanted, and the residue is suspended again in diethyl ether, triturated and centrifuged. Ether washings are repeated twice. The resulting paste is resolubilized in 1:1 CH₃CN/H₂O with 0.1% TFA (v/v) and analyzed by analytical HPLC using an Aeris WIDEPORE XB-C18 column (3.6 μm, 150×4.6 mm) at 60° C. and MALDI-TOF.

Once the peptide synthesis is completed, the peptide is cleaved from the resin using a cleavage cocktail at room temperature for 2 h. The resin is filtered off, and the filtrate is concentrated and treated with cold diethyl ether, triturated and centrifuged. The ether layer is carefully decanted, and the residue was suspended again in diethyl ether, triturated and centrifuged. Ether washings ae repeated twice. The resulting crude peptide is dried under vacuum and stored at −20° C.

A general synthesis scheme which can be used to produce modified IL-18 polypeptides provided herein is shown in FIG. 7. Briefly, linear peptide fragments (Fragments 1-4 as shown in FIG. 7) are prepared using SPPS, and any desired modification to the amino acid sequence of wild-type IL-18 (SEQ ID NO: 1) is incorporated during the syntheses. After purification of the individual segments, Segments 1 and 2 are ligated together, and Segments 3 and 4 are ligated together separately. Then, resulting Segments 1-2 and 3-4 are ligated together and universally deprotected to afford crude synthetic IL-18 polypeptide.

The Acm groups of IL18-Seg1234-Acm are then universally deprotected and purified to afford synthetic IL18 linear protein.

### 1.1 General Procedure for Synthesis of IL-18 Fragments

### 1.1.1. Segment 1: IL18(1-29)-Leu-α-Ketoacid.

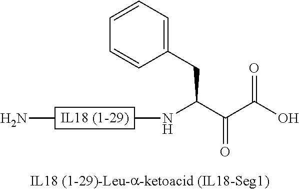

IL18(1-29)-Phe-α-ketoacid segment is synthesized on Rink Amide MBHA resin pre-loaded with protected Fmoc-α-Phe-ketoacid with a substitution capacity of 0.25 mmol/g. The synthesis is performed up to Tyr 1 by automated Fmoc-SPPS using the procedure described in the general methods section. Alternatively, for N-terminal truncation variants, synthesis is performed up to the desired residue.

Variants of segment 1: In some cases, Glu 6 is substituted with Lys.

The progress of the peptide synthesis is monitored by performing a microcleavage analysis as described in the general methods section. The cleavage cocktail is composed of a mixture of 95:2.5:2.5 TFA/DODT/H₂O.

Once the synthesis is complete, the peptide is cleaved from the resin by stirring the resin in a mixture of 95:2.5:2.5 TFA/DODT/H₂O (10 mL/g resin) at room temperature for 2 h, as described in the general methods. Purification of crude IL18(1-29)-Phe-α-ketoacid segment is performed by preparative HPLC using a Shiseido capcell Pak UG80 C18 column (5 μm, 250×50 mm) at a flow rate of 40 m/min at 60° C. with a gradient of 10 to 60% CH₃CN with 0.1% TFA (v/v) in 25 min. The fractions containing the purified product are pooled and lyophilized to obtain IL18(1-29)-Phe-α-ketoacid segment (IL18-Seg1). Analytical HPLC and ESI-HRMS are used to confirm the purity and mass of the product.

### 1.1.2 Segment 2: Opr-IL18(32-61)-Photoprotected-Val-α-Ketoacid and Opr-IL18(32-73)-Photoprotected-Leu-α-Ketoacid

### 1.1.2.1 Opr-IL18(32-61)-Photoprotected-Val-α-Ketoacid

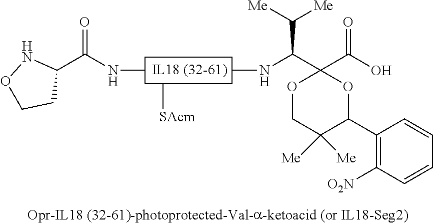

The Opr-IL18(32-61)-Val-photoprotected-α-ketoacid segment is synthesized on a 0.2 mmol scale on Rink Amide MBHA resin pre-loaded with Fmoc-Val-photoprotected-α-ketoacid with a substitution capacity of 0.24 mmol/g. The synthesis is performed up to Asp 32 by automated Fmoc-SPPS using the procedure described in the general methods section. Pseudoproline dipeptides are required for the synthesis of this segment and were manually coupled at positions 54-55, 49-50 and 35-36. Boc-5-(S)-oxaproline is manually coupled at the end of the sequence. Aspartic acid residues with non-conventional side-chain protecting groups are manually added at positions 32, 37 and 40. In some case, these protecting groups required an additional deprotection step after cleaving the peptide from the resin.

Variants of segment 2: In some cases, Lys 53 is substituted with Ala. In some cases, Cys(Acm) 38 is substituted with Ser. In some cases, Met 33, Met 51, and Met 60 are substituted with Nle or O-methyl-L-homoserine.

The progress of the peptide synthesis is monitored by performing a microcleavage described in the general methods section. The cleavage cocktail is composed of a mixture of 95:2.5:2.5 TFA/DODT/H₂O. Once the synthesis is complete, the peptide is cleaved from the resin using a mixture of 95:2.5:2.5 TFA/DODT/H₂O (15 mL/g resin) at room temperature for 2 h. The crude Opr-IL18(32-61)-photoprotected-Val-α-ketoacid segment is purified by preparative HPLC using Gemini NX-C18 110 Å column (5 μm, 250×50 mm) at a flow rate of 40 mL/min at 40° C. with a gradient of 10 to 60% CH₃CN with 0.1% TFA (v/v) in 30 min. The fractions with the purified product are pooled and lyophilized to obtain Opr-IL18(32-61)-photoprotected-Val-α-ketoacid (IL18-Seg2). Analytical HPLC and ESI-HRMS are used to confirm the purity and mass of the product. The fractions containing the purified product are pooled and lyophilized to obtain Opr-IL18(32-61)-photoprotected-Val-α-ketoacid (IL18-Seg2) as a white solid in >98% purity.

### 1.1.2.1 Opr-IL18(32-73)-Photoprotected-Leu-α-Ketoacid

Variations in segment 2 length: In some cases, the sequence of segment 2 of IL-18 is longer by a few amino acids and would comprise IL-18 sequence from position 31 to 74.

The segment Opr-IL18(32-73)-photoprotected-Phe-α-ketoacid segment is prepared on Rink Amide MBHA resin preloaded with Fmoc-Phe-photoprotected-α-ketoacid with a substitution capacity of 0.21 mmol/g. The synthesis is performed up to Asp 32 by automated Fmoc-SPPS using the procedure described in the general methods section. Boc-5-(S)-oxaproline is manually coupled to the sequence.

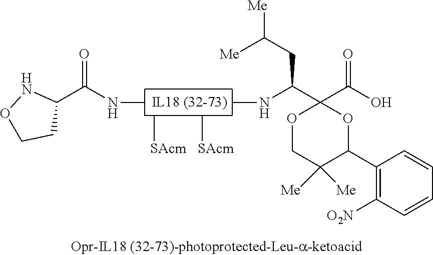

Variants of segment 2: In some cases, Lys 53 is substituted with Ala and Lys 70 is substituted with non-canonical N-α-(9-Fluorenylmethyloxycarbonyl)-ε-azido-L-lysine (Fmoc-Lys(N₃)—OH). In some cases, the side chain of Lys 70 is protected with an alloc group. The alloc group is then removed during an on-resin deprotection step, and the resulting free amine coupled with glutaric anhydride. The resulting free acid is then coupled to the corresponding desired group, for example a PEG group or PEG group bearing an azide functionality. In some cases, Cys(Acm) 38 and Cys(Acm) 68 are substituted with Ser or Ala. In some cases, Met 33, Met 51, and Met 60 are substituted with Nle or O-methyl-L-homoserine.

1.1.3 Segment 3: Fmoc-Opr-IL18(64-114)-Phe-α-ketoacid and Fmoc-Opr-IL18(76-114)-Phe-α-ketoacid.

### 1.1.3.1 Fmoc-Opr-IL18(64-114)-Phe-α-Ketoacid

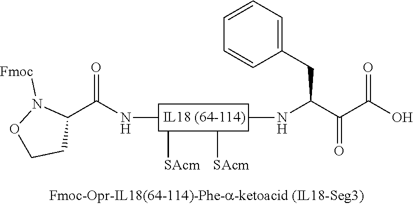

The Fmoc-Opr-IL18(64-114)-Phe-α-ketoacid segment is synthesized on a 0.1 mmol scale on Rink Amide ChemMatrix© resin pre-loaded with Fmoc-Phe-protected-α-ketoacid with a substitution capacity of 0.47 mmol/g. The synthesis is performed up to Ile 64 by automated Fmoc-SPPS using the procedure described in the general methods section. Pseudoproline dipeptides are required for the synthesis of this segment and are manually coupled at positions 81-82 and 71-72. Fmoc-5-(S)-oxaproline is manually coupled at the end of the sequence. In cases where a polymer is added to a residue as provided herein (e.g., a residue from 79-115), an amino acid residue capable of conjugating to the polymer is added at the desired location (e.g., a cysteine or a modified amino acid α as provided herein).

The progress of the peptide synthesis is monitored by performing a microcleavage described in the general methods section. The cleavage cocktail is composed of a mixture of 95:2.5:2.5 TFA/DODT/H₂O. Once the synthesis was complete, the peptide is cleaved from the resin using a mixture of 95:2.5:2.5 TFA/DODT/H₂O (15 mL/g resin) for 2 h. The crude Fmoc-Opr-IL18(76-114)-Phe-α-ketoacid segment is purified by preparative HPLC using a Gemini NX-C18 110 Å column (5 μm, 250×50 mm) at a flow rate of 40 mL/min at 40° C., with a gradient of 10 to 50% CH₃CN with 0.1% TFA (v/v) in 40 min. The fractions containing the purified product are pooled and lyophilized to obtain Fmoc-Opr-IL18(76-114)-Phe-α-ketoacid (IL18-Seg3). Analytical HPLC and ESI-HRMS are used to confirm the purity and mass of the product.

Variants of segment 3: In some cases, Cys(Acm) 68 and Cys(Acm) 76 are substituted with Ser. In some cases, Met86 and Met113 are substituted with Nle or O-methyl-L-homoserine. In some cases, Lys 70 is substituted with non-canonical N-α-(9-Fluorenylmethyloxycarbonyl)-ε-azido-L-lysine (Fmoc-Lys(N₃)—OH). In some cases, the side chain of Lys 70 is protected with an alloc group. The alloc group is then removed during an on-resin deprotection step, and the resulting free amine coupled with glutaric anhydride. The resulting free acid is then coupled to the corresponding desired group, for example a PEG group or PEG group bearing an azide functionality.

1.1.3.2 Fmoc-Opr-IL18(76-114)-Phe-α-ketoacid.

Variations in segment 3 length: In some cases, the sequence of segment 3 of IL-18 is shorter by a few amino acids and would comprise IL-18 sequence from position 75 to 115. The segment Fmoc-Opr-IL18(74-114)-Phe-α-ketoacid is then synthesized on Rink Amide ChemMatrix® resin pre-loaded with Fmoc-Phe-protected-α-ketoacid with a substitution capacity of 0.47 mmol/g. Automated Fmoc-SPPS is performed using the procedure described in the general methods section up to Cys(Acm) 76. Fmoc-5-(S)-oxaproline is manually coupled to the sequence.

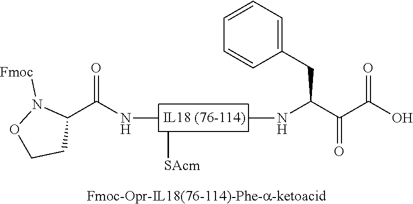

Variants of segment 3: In some cases, Cys(Acm) 76 is substituted with Ser. In some cases, Met86 and Met113 are substituted with Nle or O-methyl-L-homoserine. In cases where a polymer is added to a residue as provided herein (e.g., a residue from 79-115), an amino acid residue capable of conjugating to the polymer is added at the desired location (e.g., a cysteine or a modified amino acid α as provided herein).

### 1.1.4 Segment 4: Opr-IL18(117-157).

Preloading of Fmoc-Asp(OtBu)-OH is performed on a Fmoc-Rink-Amide MBHA resin. 4 g of resin (loading: 0.56 mmol/g, 2.24 mmol scale) is swollen in DMF for 15 min. The resin is treated with 20% in DMF (v/v) at r.t. for 20 min. The resin is washed several times with DMF. Fmoc-Asp(OtBu)-OH (691 mg, 1.68 mmol, 0.75 equiv) and HATU (638 mg, 1.68 mmol, 0.75 equiv) are dissolved in DMF (12 mL). Pre-activation is performed at r.t. for 3 min by addition of DIPEA (585 μL, 3.36 mmol, 1.5 equiv). The reaction mixture is added to the swollen resin. It is let to react overnight at r.t. under gentle agitation. The resin is rinsed thoroughly with DMF. Capping of unreacted amines on the resin is initiated by addition of a solution of acetic anhydride (1.17 mL) and DIPEA (2.34 mL) in DMF (12 mL). It is let to react at r.t. for 15 min under gentle agitation. The resin is rinsed thoroughly with DCM and dried. The loading of the resin is measured (0.34 mmol/g).

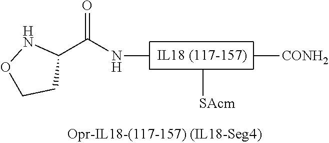

The Opr-IL18(117-157) segment is synthesized on Rink Amide MBHA resin pre-loaded with Fmoc-Asp(OtBu)-OH with a substitution capacity of 0.34 mmol/g. Automated Fmoc-SPPS is performed using the procedure described in the general methods section up to Ser 117. Boc-5-(S)-oxaproline is coupled to the sequence.

The progress of the peptide synthesis is monitored by performing a microcleavage described in the general methods section. The cleavage cocktail is composed of a mixture of 92.5:2.5:2.5:2.5 TFA/TIPS/DODT/H₂O. Once the synthesis is complete, the peptide is cleaved from the resin using a mixture of 92.5:2.5:2.5:2.5 TFA/TIPS/DODT/H₂O (10 mL/g resin) for 2 h. The crude Opr-IL18(117-157) segment is purified by preparative HPLC using a Gemini NX-C18 110 Å column (5 μm, 250×50 mm) at a flow rate of 40 mL/min at 40° C., with a gradient of 10 to 55% CH₃CN with 0.1% TFA (v/v) in 45 min. The fractions containing the purified product are pooled and lyophilized to obtain Opr-IL18(117-157) (IL18-Seg4). Analytical HPLC and ESI-HRMS are used to confirm the purity and mass of the product.

Variants of segment 4: In some cases, Cys(Acm) 127 is substituted with Ser. In some cases, Met 150 is substituted with Nle or O-methyl-L-homoserine. In cases where a polymer is added to a residue as provided herein (e.g., a residue from 116-120), an amino acid residue capable of conjugating to the polymer is added at the desired location (e.g., a cysteine or a modified amino acid α as provided herein).

### 1.2 KAHA Ligations for the Preparation of IL18 Linear Protein.

### 1.2.1. KAHA Ligation for the Synthesis of Segment 12 (IL18-Seg12)

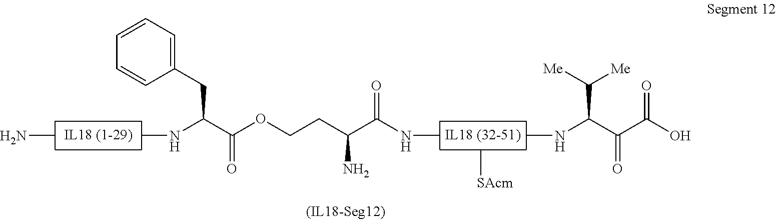

Ligation: IL18-Seg1 (1.2 eq) and IL18-Seg2 (1 eq) are dissolved in 9:1 DMSO/H₂O containing 0.1 M oxalic acid (20 mM peptide concentration for the limiting agent) and reacted at 60° C. for 15 h. The ligation vial is protected from light by wrapping the vial in aluminum foil. The progress of the KAHA ligation is monitored by HPLC using an Aeris WIDEPORE XB-C18 column (3.6 μm, 150×4.6 mm) at a flow rate of 1 mL/min at 60° C. with a gradient of 20 to 95% CH₃CN in 7 min.

Photodeprotection: After completion of the ligation, the mixture is diluted with 1:1 CH₃CN/H2O with 0.1% TFA (v/v) and irradiated at a wavelength of 365 nm for 1.5 h. Completion of the photolysis reaction was confirmed by HPLC and MALDI-TOF MS analysis.

Purification: The photo-deprotected sample is purified by preparative HPLC using a Shiseido capcell Pak UG80 C18 column (5 μm, 250×50 mm) kept at 60° C., with a 2-step gradient: 10 to 60% CH₃CN with 0.1% TFA (v/v) in 25 min, then hold 60% CH₃CN for 5 min, with a flow of 40 mL/min. The fractions containing the purified product are pooled and lyophilized to obtain IL18-Seg12. The purity and identity of the segment is confirmed by HPLC and ESI-HRMS analysis.

### 1.2.2 KAHA Ligation for the Synthesis of Segment 34 (IL18-Seg34)

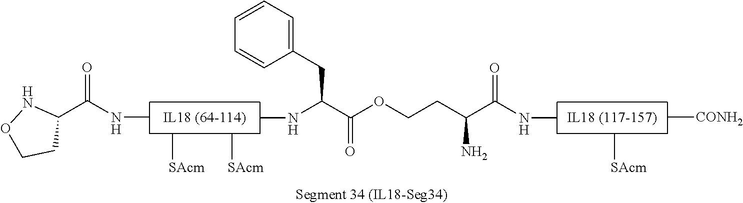

Ligation: IL18-Seg3 (1 eq) and IL18-Seg4 (1.2 eq) are dissolved in 97.5:2.5 DMSO/H₂O containing 0.1 M oxalic acid (20 mM peptide concentration for the limiting agent) and reacted for 16 h at 60° C. The progress of the KAHA ligation is monitored by HPLC using an Aeris WIDEPORE (3.6 μm, 150×4.6 mm) column with a flow rate of 1 mL/min at 60° C. with a gradient of 5 to 65% CH₃CN in 7 min.

Fmoc deprotection: After completion of ligation, the reaction mixture is diluted with DMSO (6.7 mM peptide concentration). Diethylamine is added (5%, v/v) and the reaction mixture is shaken at room temperature for 15 min. The reaction mixture is diluted a second time with DMSO (3.3 mM peptide concentration). Diethylamine is added (2.5%, v/v) and the reaction mixture is shaken at room temperature for another 15 min. The reaction mixture is then diluted with 1:1 CH₃CN/H₂O with 0.1% TFA (v/v).

Purification: The sample is purified by preparative HPLC on a Gemini NX-C18 110 Å column (5 μm, 250×50 mm) at a flow rate of 40 m/min at 40° C., with a gradient of 10 to 50% CH₃CN with 0.1% TFA (v/v) in 40 min. The fractions containing the purified product are pooled and lyophilized to obtain IL18-Seg34 (Seg34). Analytical HPLC and ESI-HRMS are used to confirm the purity and mass of the product.

### 1.2.3 KAHA Ligation for the Synthesis of IL18 Segment 1234 (IL18-Seg1234-Acm).

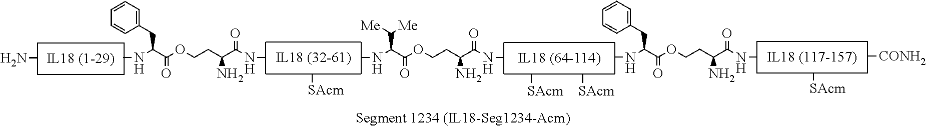

Ligation: IL18-Seg12 (1.2 eq) and IL18-Seg34 (1.0 eq) are dissolved in 9:1 DMSO/H₂O containing 0.1 M oxalic acid (15 mM peptide concentration), and the reaction is stirred for 24 h at 60° C. The progress of the KAHA ligation is monitored by analytical HPLC using an Aeris WIDEPORE (3.6 μm, 150×4.6 mm) column with a flow rate of 1 m/min at 60° C. using CH₃CN/H₂O with 0.1% TFA (v/v) as mobile phase, with a gradient of 20 to 95% CH₃CN in 7 min. After completion of ligation, the reaction mixture is diluted with DMSO followed by further dilution with a mixture of 1:1 CH₃CN/H₂O with 0.1% TFA (v/v).

Purification: The sample is purified by preparative HPLC on a Gemini NX-C18 110 Å column (5 μm, 250×50 mm) at a flow rate of 40 m/min at 60° C., with a gradient of 10 to 60% CH₃CN with 0.1% TFA (v/v) in 30 min. The fractions containing the purified product are pooled and lyophilized to obtain IL18-Seg1234 with cysteine residues protected with an Acm group (IL18-Seg1234-Acm). Analytical HPLC and ESI-HRMS are used to confirm the purity and mass of the product.

### 1.2.4 Rearrangement and Acm Deprotection for the Synthesis of IL18 Linear Protein

Rearrangement: IL18-Seg1234-Acm is dissolved in 6 M Gu·HCl containing 0.1 M Tris (pH 8.1) (1.5 mL, 0.13 mM protein concentration). The pH is adjusted to 8.0. It is let to react for 2 h at 50° C. After completion of reaction, the sample is diluted with 6 M Gu·HCl containing 0.1% TFA (v/v, 10 mL), and purified by preparative HPLC using a Proteonavi S5 column (250×20 mm) at a flow rate of 10 mL/min at 60° C. using CH₃CN/H₂O with 0.1% TFA (v/v) as mobile phase, with a gradient of 20 to 40% (in 19 min) and 40 to 50% (in 11 min) CH₃CN with 0.1% TFA (v/v). The fractions containing the product are pooled and lyophilized to obtain IL18 linear protein with Acm. Analytical HPLC and ESI-HRMS are used to confirm the purity and mass of the product.

Acm deprotection: IL18 linear protein with Acm is dissolved in 1:1 AcOH/H₂O (0.25 mM protein concentration), and silver acetate (1%, m/v) is added to the solution. The mixture is shaken for 2.5 h at 50° C. protected from light. The progress of the Acm deprotection reaction is monitored by analytical HPLC using an Aeris WIDEPORE (3.6 μm, 150×4.6 mm) column with a flow rate of 1 m/min at 60° C. using CH₃CN/H₂O with 0.1% TFA (v/v) as mobile phase, with a gradient of 20 to 95% CH₃CN in 7 min. After completion of the reaction, the sample is diluted with 1:1 CH₃CN/H₂O with 0.1% TFA (v/v).

Purification: The sample is purified by preparative HPLC on a Shiseido capcell Pak® UG80 C18 column (250×20 mm) at a flow rate of 10 m/min at room temperature using CH₃CN/H₂O with 0.1% TFA (v/v) as mobile phase, with a two-step gradient: 10 to 30% CH₃CN in 5 min and 30 to 95% CH₃CN in 20 min. The fractions containing the purified product are pooled and lyophilized to obtain the desired IL18 linear protein. Analytical HPLC and HRMS are used to confirm the purity and mass of the product.

### Modification to 5-Peptide Ligation Strategy.

The methods provided above can be readily modified in order to prepare an IL-18 polypeptide from 5 precursor fragments instead of the 4-peptide ligation strategy outlined above. Briefly, in one example, the N-terminal fragment synthesized above can be prepared and used as indicated (Seg1). Differing from above, the second fragment instead comprises residues corresponding to residues 31-62 (Seg2), the third fragment comprises residues corresponding to residues 63-74 (Seg3), and the remaining two fragments are as provided above (residues 75-115 (Seg4) and 116-157 (Seg5), respectively). The fragments are then ligated as indicated in the analogous FIGS. 8-10. Briefly, Seg1 and Seg2 are ligated in one ligation reaction. Seg 4 and Seg5 are ligated in one reaction. Seg3 is then ligated to Seg45 to produce Seg 345. Seg345 is then ligated with Seg12 to produce the full length linear IL-18. This protocol can be used to prepare IL-18s analogous to those of SEQ ID NOs: 68-91.

Alternatively to that discussed in the above paragraph, Seg2 comprises residues 31-56 and Seg3 comprises residues 57-74. The remaining fragments are the same, and are ligated in the same order. This protocol can be used to prepare IL-18s analogous to those of SEQ ID NOs: 92-115.

In another example, Seg2 comprises residues 31-66 and Seg3 comprises residues 67-75. The fragments are then ligated in the same order as indicated above. This protocol can be used to prepare IL-18s analogous to those of SEQ ID NOs: 140-163.

In another example, Seg2 comprises residues 31-49, Seg3 comprises residues 50-75, Seg 4 comprises residues 76-120, and Seg5 comprises residues 121-157. The fragments are then ligated in the same order. This protocol can be used to prepare IL-18s analogous to those of SEQ ID NOs: 116-139.

### Example 3—Folding of Modified IL-18 Polypeptides

The synthesized modified IL-18 polypeptides are dissolved in buffered solutions and subjected to specific buffer and pH conditions to promote folding of the polypeptides. The folded protein is confirmed using analytical techniques, such as HPLC, ESI-MS and/or MALDI-TOF. Several conditions are screened and tested by varying the composition of the folding buffers (Buffers A and B) and formulation buffers. Exemplary folding conditions and buffer compositions are shown below in Table 3. One or more conditions which result in the desired analytical and biochemical properties of the modified IL-18 polypeptide is selected for scale up folding protocols.
Step 1: The linear protein is dissolved in Buffer A (2 to 4 mg/mL protein concentration). The protein solution is gently shaken at 20° C. for up to 1 h.
Step 2: The solution of protein is slowly diluted in a dropwise fashion with Buffer B. A clear solution obtained at a concentration of 0.2 to 0.4 mg/mL is incubated at 4° C., 10° C. or 20° C. for 18 to 48 h.
Step 3: The solution is centrifuged at 10 000 RPM at 10° C. for 10 min. It is then dialyzed against PBS (pH 7.4) containing 0.02% Tween 80 and 5-6% sucrose at r.t. for 2 h. This step is repeated a second time. It is then dialyzed a third time at r.t. for 18 h with the same buffer.

The purity and identity of the pure folded protein is further confirmed by analytical HPLC and MALDI-TOF.

TABLE 3
Composition of Buffers A and B

| Buffer A | Buffer B |
|---|---|
| Tris buffer (50 mM, pH 8.0) | Tris buffer (50 mM, pH 7.8) containing 2 mM |
| containing 8M urea, 2 mM DTT | EDTA, 137 mM NaCl, 2.7 mM KCl, 400 mM |
| and 0.02% (m/v) Tween 80 | Arginine HCl, 2 mM DTT and 0.02% (m/v) |
| | Tween 80 |
| Tris buffer (50 mM, pH 8.0) | Tris buffer (50 mM, pH 7.8) containing 2 mM |
| containing 8M urea, 2 mM DTT | EDTA, 137 mM NaCl, 2.7 mM KCl, 400 mM |
| and 0.1% (m/v) Tween 80 | ArginineHCl, 2 mM DTT and 0.1% (m/v) |
| | Tween 80 |
| PBS (pH 7.4) containing 6M | PBS (pH 7.4) containing 0.02% Tween20 |
| urea, 0.02% Tween20 and 1 mM | |
| TCEP | |
| PBS (pH 7.4) containing 8M urea | PBS (pH 7.4) |
| Tris buffer (50 mM, pH 8.0) | Tris buffer (50 mM, pH 7.8) containing 2 mM |
| containing 8M urea | EDTA, 240 mM NaCl, 10 mM KCl, 200 mM |
| | ArginineHCl, 200 mM sucrose, 2 mM DTT |
| | and 0.02% (m/v) Tween 80 |
| HEPES buffer (50 mM, pH 8.0) | HEPES buffer (50 mM, pH 7.8) containing 2 |
| containing 8M urea, 2 mM DTT | mM EDTA, 137 mM NaCl, 2.7 mM KCl, 400 |
| and 0.02% (m/v) Tween 80 | mM ArginineHCl, 2 mM DTT and 0.02% |
| | (m/v) Tween 80 |
| HEPES buffer (100 mM, pH 7.5) | HEPES buffer (100 mM, pH 7.5) containing 1 |
| containing 8M urea, 1 mM | mM cysteine, 0.1 mM cystine, 1 mM EDTA |
| cysteine, 0.1 mM cystine, 1 | and 0.02% (m/v) Tween 80 |
| mM EDTA and 0.02% (m/v) | |
| Tween 80 | |

### Example 4—Synthesis of a Modified IL-18 Polypeptide of for SEQ ID NO: 164

A linear peptide of SEQ ID NO: 164 was prepared according to the protocol described below.

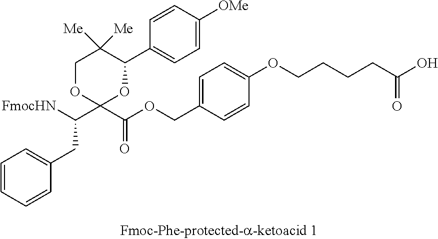

Segment 1 (IL-18 (1-29)-Phe-α-ketoacid): Preloading of Fmoc-Phe-protected-α-ketoacid 1 was performed on a Fmoc-Rink Amide MBHA resin. 5 g of resin (loading: 0.56 mmol/g, 1.8 mmol scale) was swollen in DMF for 20 min. The resin was treated twice with 20% piperidine in DMF (v/v) at room temperature for 10 min. and was washed several times with DMF. Ketoacid 1 (1.46 g, 1.8 mmol, 1.0 eq) and HATU (650 mg, 1.71 mmol, 0.95 eq) were dissolved in DMF (20 mL). Pre-activation was performed at room temperature for 3 min by adding NMM (396 μL, 3.6 mmol, 2 eq). The reaction mixture was added to the swollen resin and gently agitated at room temperature for 2.5 h. The resin was rinsed thoroughly with DMF. Capping of unreacted amines on the resin was initiated by adding a solution of acetic anhydride (1.17 mL) and DIPEA (2.34 mL) in DMF (20 mL) and gently agitating the reaction at room temperature for 15 min. The resin was rinsed thoroughly with DCM followed by diethyl ether and dried. The loading of the resin was measured (0.30 mmol/g).

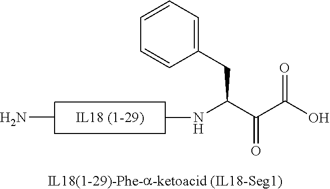

The IL18(1-29)-Phe-α-ketoacid segment was synthesized on a 0.45 mmol scale on Rink Amide MBHA resin pre-loaded with Fmoc-Phe-protected-α-ketoacid (1.5 g) with a substitution capacity of ˜0.30 mmol/g.

Automated Fmoc-SPPS of IL18(1-29)-Phe-α-ketoacid: The coupling reactions were performed at room temperature for 30 min by adding a solution of Fmoc-amino acids dissolved in DMF (10.0 mL, 0.4 M, 4 eq), HCTU in DMF (10.0 mL, 0.38 M, 3.8 eq) and NMM in DMF (10.0 mL, 0.8 M, 8 eq) to the resin. For position 14 to 1, double couplings were required. Washing with a solution of lithium chloride (0.8 M) in DMF was performed every five amino acids before the Fmoc deprotection reaction. When required, capping was performed at room temperature for 10 min by adding a 20% (v/v) acetic anhydride solution in DMF (10.0 mL) and NMM in DMF (0.8 M, 10.0 mL). The Fmoc deprotection reaction was performed using 20% (v/v) piperidine in DMF containing Cl-HOBt (0.1 M) at room temperature for 8 min.

The resin was washed with DCM and dried under vacuum. The mass of the dried peptidyl resin was 4.6 g. The peptide was cleaved from the resin using a mixture of 95:2.5:2.5 TFA/DODT/H₂O (10 mL/g resin) at room temperature for 2.0 h. The resin was filtered off from the cleavage cocktail, and the filtrate was concentrated and diluted 20-fold with cold diethyl ether (20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. The mass of crude peptide was 1.8 g. Purification of crude IL18(1-29)-Phe-α-ketoacid segment was performed by preparative HPLC using Shiseido Capcell Pak UG80 C18 column (5 μm, 250×50 mm) at a flow rate of 40 mL/min at 60° C. using CH₃CN/H₂O with 0.1% TFA (v/v) as mobile phase, with a gradient of 10 to 60% CH₃CN with 0.1% TFA (v/v) in 25 min. The fractions containing the purified product were pooled and lyophilized to obtain IL18(1-29)-Phe-α-ketoacid (IL18-Seg1) as a white solid in >98% purity. The isolated yield based on the resin loading was 472 mg (28%). MS (ESI): C₂₃₃H₃₄₈N₅₈O₆₉S; Average isotope calculated 3550.8936 Da [M]. found: 3550.8948 Da.

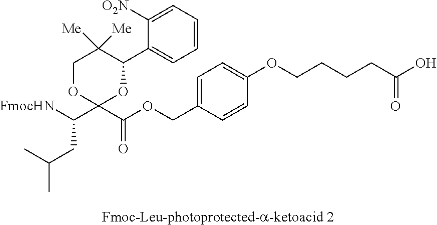

Segment 2 (Opr-IL18(32-73)-Leu-photoprotected-α-ketoacid): Preloading of Fmoc-Leu-photoprotected-α-ketoacid 2 was performed on a Fmoc-Rink-Amide MBHA resin. 5 g of resin (loading: 0.56 mmol/g, 2.25 mmol scale) was swollen in DMF for 20 min. Ketoacid 2 (1.79 g, 2.25 mmol, 1 eq) and HATU (813 mg, 2.14 mmol, 0.95 eq) were dissolved in DMF (25 mL). Pre-activation was performed at room temperature for 2 min by adding NMM (495 μL, 4.5 mmol, 2 eq). The reaction mixture was added to the swollen resin and gently agitated for 6 h at room temperature. The resin was rinsed thoroughly with DMF. Capping of unreacted amines on the resin was initiated by adding a solution of acetic anhydride (2.0 mL) and DIPEA (2.0 mL) in DMF (20 mL) and gently agitating the mixture at room temperature for 15 min. The resin was rinsed thoroughly with DCM and diethyl ether and dried. The loading of the resin was measured (0.34 mmol/g).

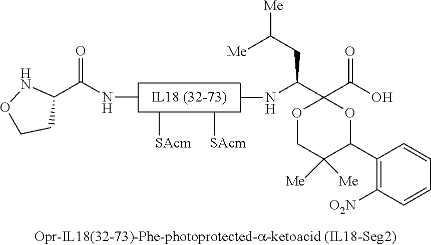

Opr-IL18(32-73)-Phe-photoprotected-α-ketoacid segment was synthesized on a 0.2 mmol scale on Rink Amide MBHA resin pre-loaded with Fmoc-Phe-Leu-photoprotected-α-ketoacid with a substitution capacity of ˜0.34 mmol/g.

Automated Fmoc-SPPS from position 73 to 66: The coupling reactions were performed at room temperature for 30 min by adding the Fmoc-amino acids dissolved in DMF (2.0 mL, 0.4 M, 4 eq), HCTU in DMF (2.0 mL, 0.38 M, 3.8 eq) and NMM in DMF (2.0 mL, 0.8 M, 8 eq) to the resin. The Fmoc deprotection reaction was performed twice for each coupling cycle using 20% (v/v) piperidine in DMF containing Cl-HOBt (0.1 M) at room temperature for 7 min.

Manual coupling of Fmoc-L-Ile-L-Ser[Ψ(Me,Me)Pro]-OH was then performed. A solution of Fmoc-L-Ile-L-Ser[Ψ(Me,Me)Pro]-OH (384 mg, 0.8 mmol, 4 eq), HATU (290 mg, 0.76 mmol, 3.8 eq) and NMM (176 μL, 1.6 mmol, 8 eq) in 3 mL of DMF was prepared (3 min of pre-activation at room temperature) and added to the resin. The reaction was gently agitated at room temperature for 1 h.

Automated Fmoc-SPPS for position 63: The coupling reactions were performed using the conditions described above. Triple coupling was required for position 63.

Automated Fmoc-SPPS from position 64 to 56: The coupling reactions were performed using the conditions described above.

Manual coupling of Fmoc-L-Asp(tBu)-L-Ser[Ψ(Me,Me)Pro]-OH was then performed. A solution of Fmoc-L-Asp(tBu)-L-Ser[Ψ(Me,Me)Pro]-OH (430 mg, 0.8 mmol, 4 eq), HATU (290 mg, 0.76 mmol, 3.8 eq) and NMM (176 μL, 1.6 mmol, 8 eq) in 3 mL of DMF was prepared (3 min of pre-activation at room temperature) and added to the resin. The reaction was gently agitated at room temperature for 1 h.

Automated Fmoc-SPPS from position 53 to 51: The coupling reactions were performed using the same conditions as previously mentioned for the beginning of the sequence.

Manual coupling of Fmoc-L-Ile-L-Ser[Ψ(Me,Me)Pro]-OH was then performed. A solution of Fmoc-L-Ile-L-Ser[Ψ(Me,Me)Pro]-OH (384 mg, 0.8 mmol, 4 eq), HATU (290 mg, 0.76 mmol, 3.8 eq) and NMM (176 μL, 1.6 mmol, 8 eq) in 3 mL of DMF was prepared (3 min of pre-activation at room temperature) and added to the resin. The reaction was gently agitated at room temperature for 1 h.

Automated SPPS from position 48 to 37: The coupling reactions were performed using the conditions described above. Double couplings were required for each position.

Manual coupling of Fmoc-L-Asp(tBu)-L-Ser[Ψ(Me,Me)Pro]-OH was then performed. A solution of Fmoc-L-Asp(tBu)-L-Ser[Ψ(Me,Me)Pro]-OH (430 mg, 0.8 mmol, 4 eq), HATU (290 mg, 0.76 mmol, 3.8 eq) and NMM (176 μL, 1.6 mmol, 8 eq) in 3 mL of DMF was prepared (3 min of pre-activation at room temperature) and added to the resin. The reaction was gently agitated at room temperature for 1 h.

Automated Fmoc-SPPS from position 34 to 32: The coupling reactions were performed using the conditions described above. Double couplings were required for each position. Capping was performed at room temperature for 10 min at each position by adding 20% (v/v) acetic anhydride in DMF (2 mL) and NMM in DMF (0.8 M, 2 mL). Fmoc deprotection was performed using 20% (v/v) piperidine in DMF containing Cl-HOBt (0.1 M) at room temperature for 7 min.

Manual coupling of Boc-5-(S)-oxaproline was then performed. A solution of Boc-5-(S)-oxaproline (217 mg, 1.0 mmol, 5 eq), HATU (361 mg, 0.95 mmol, 4.8 eq) and NMM (220 μL, 2.0 mmol, 10 eq) in 7 mL of DMF was prepared (3 min of pre-activation at room temperature) and added to the resin. The reaction was gently agitated at room temperature for 2.5 h. The resin was washed with DCM and diethyl ether and dried under vacuum. The mass of the dried peptidyl resin was 1.8 g.

The peptide was cleaved from the resin using a mixture of 95:2.5:2.5 TFA/DODT/H₂O (15 mL/g resin) and gently agitating the mixture at room temperature for 2.0 h. The resin was filtered off from the cleavage cocktail, and the filtrate was concentrated and diluted 20-fold with cold diethyl ether (20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. The mass of crude peptide was 1.2 g. Purification of the crude Opr-IL18(32-73)-Phe-photoprotected-α-ketoacid segment was performed by preparative HPLC using Gemini NX-C18 110 Å column (5 μm, 250×50 mm) at a flow rate of 40 m/min at 40° C. using CH₃CN/H₂O with 0.1% TFA (v/v) as mobile phase, with a gradient of 10 to 60% CH₃CN with 0.1% TFA (v/v) in 30 min. The fractions containing the purified product were pooled and lyophilized to obtain Opr-IL18(32-73)-Phe-photoprotected-α-ketoacid (IL18-Seg2) as a white solid in >98% purity. The isolated yield based on the resin loading was 148 mg (14%). LC-MS (ESI): 4.88 min; C₂₃₃H₃₄₈N₅₈O₆₉S; m/z calculated: 1315.4233 Da [M+4H]. found: 1315.4231 Da [M+4H].

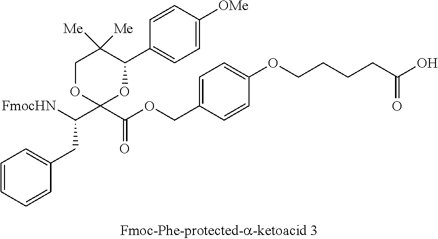

Segment 3 (Fmoc-Opr-IL18(76-114)-Phe-α-ketoacid): 222 mg of resin (loading: 0.47 mmol/g, 0.1 mmol scale) was swollen in DMF for 15 min. Ketoacid 3 (163 mg, 0.2 mmol, 2 eq) and HATU (76 mg, 0.2 mmol, 2 eq) were dissolved in DMF (2 mL). Pre-activation was performed at room temperature for 2 min by adding DIPEA (100 μL, 0.6 mmol, 6 eq). The reaction mixture was added to the swollen resin. The reaction was gently agitated overnight at room temperature. The resin was rinsed thoroughly with DMF. Capping of unreacted amines on the resin was initiated by adding a solution of acetic anhydride (100 μL) and DIPEA (100 μL) in DMF (2 mL). The reaction was gently agitated at room temperature for 15 min. The resin was rinsed thoroughly with DMF. The final loading of the resin was not calculated and was estimated to be unchanged (0.47 mmol/g).

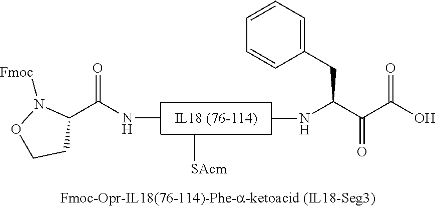

The Fmoc-Opr-IL18(76-114)-Phe-α-ketoacid segment was synthesized on a 0.1 mmol scale on Rink Amide ChemMatrix© resin pre-loaded with Fmoc-Phe-protected-α-ketoacid with a substitution capacity of ˜0.47 mmol/g.

Automated Fmoc-SPPS from position 96 to 114: The coupling reactions were performed at room temperature for 30 min by adding Fmoc-amino acids dissolved in DMF (1.0 mL, 0.5 M, 5 eq), HCTU in DMF (1.0 mL, 0.48 M, 4.8 eq) and DIPEA in NMP (0.4 mL, 0.2 M, 8 eq) to the resin. Fmoc deprotection was performed using 20% (v/v) piperidine in DMF at room temperature for 15 min.

Manual coupling of Fmoc-L-Asp(tBu)-L-Thr[Ψ(Me,Me)Pro]-OH was then performed. A solution of Fmoc-L-Asp(tBu)-L-Thr[Ψ(Me,Me)Pro]-OH (166 mg, 0.3 mmol, 3 eq), HATU (114 mg, 0.3 mmol, 3 eq) and DIPEA (100 μL, 0.6 mmol, 6 eq) in 3 mL of DMF was prepared (3 min of pre-activation at room temperature) and added to the resin. The reaction was gently agitated at room temperature for 2 h.

Automated Fmoc-SPPS from position 76 to 93: The coupling reactions were performed using the conditions described above. Double couplings were required for each position. Capping was performed at room temperature for 10 min at each position by adding 20% (v/v) acetic anhydride in DMF (1 mL) and DIPEA in DMF (0.2 M, 1 mL). Fmoc deprotection reactions were performed using 25% (v/v) piperidine in DMF containing Cl-HOBt (0.1 M) at room temperature for 15 min.

Manual coupling of Fmoc-5-(S)-oxaproline was then performed. A solution of Fmoc-5-(S)-oxaproline (102 mg, 0.3 mmol, 3 eq), HATU (114 mg, 0.3 mmol, 3 eq) and DIPEA (100 μL, 0.6 mmol, 6 eq) in 3 mL of DMF was prepared (3 min of pre-activation at room temperature) and added to the resin. The reaction was gently agitated at room temperature for 2 h. The resin was washed with DCM and dried under vacuum. The mass of the dried peptidyl resin was 1.0 g.

The peptide was cleaved from the resin by stirring the resin in a mixture of 95:2.5:2.5 TFA/DODT/H₂O (15 mL/g resin) at room temperature for 2.0 h. The resin was filtered off from the cleavage cocktail, and the filtrate was concentrated and diluted 20-fold with cold diethyl ether (20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. Mass of crude peptide was 540 mg. Purification of crude Fmoc-Opr-IL18(76-114)-Phe-α-ketoacid segment was performed by preparative HPLC using Gemini NX-C18 110 Å column (5 μm, 250×250 mm) at a flow rate of 40 m/min at 40° C. using CH₃CN/H₂O with 0.1% TFA (v/v) as mobile phase, with a gradient of 10 to 50% CH₃CN with 0.1% TFA (v/v) in 40 min. The fractions containing the purified product were pooled and lyophilized to obtain the Fmoc-Opr-IL18(76-114)-Phe-α-ketoacid (IL18-Seg3) as a white solid. The isolated yield based on the resin loading was 128 mg (25%). MS (ESI): C₂₃₃H₃₄₈N₅₈O₆₉S; Average isotope calculated 5094.5226 Da [M]. found: 5094.5224 Da.

Segment 4 (Opr-IL18 (117-157)): Preloading of Fmoc-Asp(OtBu)-OH was performed on a Fmoc-Rink-Amide MBHA resin. 4 g of resin (loading: 0.56 mmol/g, 2.24 mmol scale) was swollen in DMF for 15 min. The resin was treated with 20% in DMF (v/v) at room temperature for 20 min. The resin was washed several times with DMF. Fmoc-Asp(OtBu)-OH (691 mg, 1.68 mmol, 0.75 eq) and HATU (638 mg, 1.68 mmol, 0.75 eq) were dissolved in DMF (12 mL). Pre-activation was performed at room temperature for 3 min by adding DIPEA (585 μL, 4.48 mmol, 2 eq). The reaction mixture was added to the swollen resin and gently agitated overnight at room temperature. The resin was rinsed thoroughly with DMF. Capping of unreacted amines on the resin was initiated by adding a solution of acetic anhydride (1.17 mL) and DIPEA (2.34 mL) in DMF (12 mL) and gently agitating the mixture at room temperature for 15 min. The resin was rinsed thoroughly with DCM and dried. The loading of the resin was measured (0.34 mmol/g).

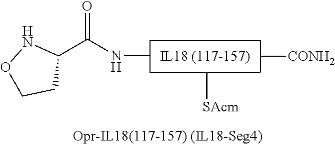

Opr-IL18(117-157) segment was synthesized on a 0.1 mmol scale on Rink Amide MBHA resin pre-loaded with Fmoc-Asp(OtBu)-OH with a substitution capacity of ˜0.34 mmol/g. 294 mg of resin was swollen in DMF for 15 min.

Automated Fmoc-SPPS from position 147 to 157: The coupling reactions were performed at room temperature for 30 min by adding Fmoc-amino acids dissolved in DMF (1.0 mL, 0.5 M, 5 eq), HCTU in DMF (1.0 mL, 0.48 M, 4.8 eq) and DIPEA in NMP (0.4 mL, 0.2 M, 8 eq) to the resin. Fmoc deprotection was performed using 20% (v/v) piperidine in DMF at room temperature for 15 min. Double coupling was required from position 117 to 146 as well as capping steps. Capping was performed at room temperature for 10 min at each position by adding 20% (v/v) acetic anhydride in DMF (1 mL) and DIPEA in DMF (0.2 M, 1 mL).

Manual coupling of Boc-5-(S)-oxaproline was then performed. A solution of Boc-5-(S)-oxaproline (65 mg, 0.3 mmol, 3 eq), HATU (114 mg, 0.3 mmol, 3 eq) and DIPEA (100 μL, 0.6 mmol, 6 eq) in 3 mL of DMF was prepared (3 min of pre-activation at room temperature) and added to the resin. The mixture was reacted at room temperature for 2 h.

The resin was washed with DCM and dried under vacuum. The mass of the dried peptidyl resin was 1.2 g. The peptide was cleaved from the resin using a mixture of 92.5:2.5:2.5:2.5 TFA/TIPS/DODT/H₂O (10 mL/g resin) at room temperature for 2 h. The resin was filtered off from the cleavage cocktail, and the filtrate was concentrated and diluted 20-fold with cold diethyl ether (20° C.), allowing the peptide to precipitate. After centrifugation, the ether layer was carefully decanted, and the peptide precipitate was resuspended in diethyl ether, triturated and centrifuged. Ether washings were repeated twice, and the resulting peptide precipitate was dried. Mass of crude peptide was 770 mg. Purification of crude Opr-IL18(117-157) segment was performed by preparative HPLC using Gemini NX-C18 110 Å column (5 μm, 250×50 mm) at a flow rate of 40 m/min at 40° C. using CH₃CN/H₂O with 0.1% TFA (v/v) as mobile phase, with a gradient of 10 to 50% CH₃CN with 0.1% TFA (v/v) in 40 min. The fractions containing the purified product were pooled and lyophilized to obtain Opr-IL18(117-157) (IL18-Seg4) as a white solid. The isolated yield based on the resin loading was 106 mg (21%). MS (ESI): C₂₂₂H₃₄₆N₅₆O₇₃S; Average isotope calculated 1250.9051 Da [M+4H⁺]. found: 1250.6293 Da.

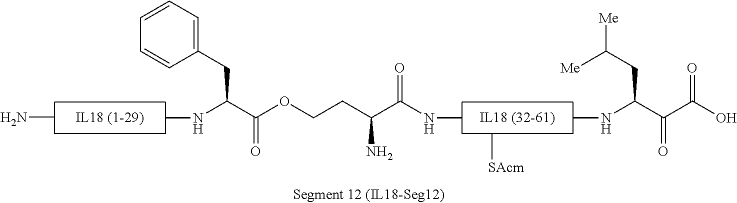

Peptide photoprotected ketoacid IL18-Seg12 (28.4 mg; 7.98 μmol; 1.2 equiv) and hydroxylamine peptide IL18-Seg2 (25.9 mg; 6.65 μmol; 1.0 equiv) were in dissolved in 9:1 DMSO/H₂O containing 0.1 M oxalic acid (333 μL). A homogeneous liquid solution was obtained. The ligation vial was protected from light by wrapping the vial in aluminum foil, and the reaction was left overnight at 60° C. After completion of the ligation the mixture was diluted with 1:1 CH₃CN/H₂O with 0.1% TFA (v/v) (1780 μL) and irradiated at a wavelength of 365 nm for 1.5 h to allow photodeprotection of the C-terminal ketoacid. The reaction mixture was further diluted with 1:1 CH₃CN/H₂O (q.s. 10 mL) with TFA (0.1%, v/v). The diluted mixture was filtered and injected into preparative HPLC. Crude ligated peptide was purified by preparative HPLC using Gemini NX-C18 110 Å column (5 μm, 250×250 mm) at a flow rate of 40 m/min at 60° C., with a gradient of 10 to 60% CH₃CN with 0.1% TFA (v/v) in 30 min. The fractions containing the purified product were pooled and lyophilized to obtain IL18-Seg12 as a white solid in >98% purity.

The isolated yield was 23.9 mg (50%).

LC-MS (ESI): 4.63 min; C₃₂₂H₅₁₅N₈₉O₉₆S; m/z calculated: 7196.7874 Da [M]. found: 7196.7476 Da [M].

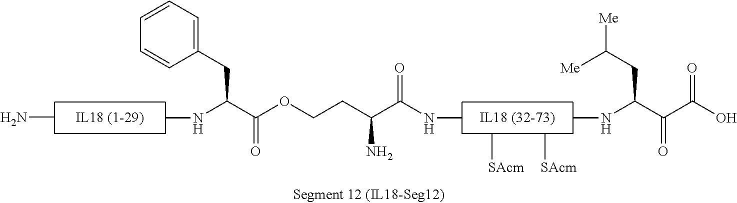

IL18-Seg12 preparation: Peptide photo-protected ketoacid IL18-Seg1 (18.1 mg; 5.09 mol; 1.2 eq) and hydroxylamine peptide IL18-Seg2 (22.3 mg; 4.24 μmol; 1.0 eq) were in dissolved in a 9:1 DMSO/H₂O solution containing 0.1 M oxalic acid (220 μL). A very homogeneous liquid solution was obtained. The ligation vial was protected from light by wrapping the vial in aluminum foil and gently agitated overnight at 60° C. After completion of the ligation, the mixture was diluted with 1:1 CH₃CN/H₂O with 0.1% TFA (v/v) (1780 μL) and irradiated at a wavelength of 365 nm for 1.5 h to allow photo deprotection of the C-terminal ketoacid. The mixture was further diluted with 1:1 CH₃CN/H₂O (q.s. 10 mL) with TFA (0.1%, v/v). The diluted mixture was filtered and injected into preparative HPLC. Crude ligated peptide was purified by preparative HPLC using Gemini NX-C18 110 Å column (5 μm, 250×250 mm) at a flow rate of 40 mL/min at 60° C., with a gradient of 10% to 60% CH₃CN with 0.1% TFA (v/v) in 25 min. The fractions containing the purified product were pooled and lyophilized to obtain IL18-Seg12 as a white solid in >98% purity. The isolated yield was 16.9 mg (47%).

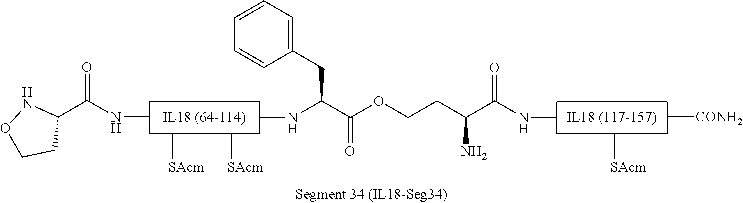

IL18-Seg34 preparation: Peptide ketoacid IL18-Seg3 (54.6 mg; 10.9 μmol; 1.0 eq) and hydroxylamine peptide IL18-Seg4 (66.8 mg; 13.1 μmol; 1.2 eq) were in dissolved in 9:1 DMSO/H₂O containing 0.1 M oxalic acid (546 μL). A very homogeneous liquid solution was obtained, which was gently agitated overnight at 60° C. Upon completion of the ligation reaction, the mixture was diluted with DMSO (1092 μL). Fmoc deprotection was initiated by the adding diethylamine (82 μL, 5%, v/v) and gently agitated at room temperature for 15 min. A second solution of diethylamine (82 μL) in DMSO (1638 μL) was added to the reaction mixture and gently agitated at room temperature for another 15 min. Gel formation was expected. Trifluoroacetic acid (200 μL) was added to neutralize the reaction mixture. A homogeneous and colorless liquid solution was obtained, which was further diluted with 1:1 CH₃CN/H₂O (q.s. 17 mL) with TFA (0.1%, v/v). The diluted mixture was directly injected into preparative HPLC. The crude ligated peptide solution was filtered and purified by preparative HPLC using Gemini NX-C18 110 Å column (5 μm, 250×50 mm) at a flow rate of 40 mL/min at 40° C. using CH₃CN/H₂O with 0.1% TFA (v/v) as mobile phase, with a gradient of 10% to 50% CH₃CN with 0.1% TFA (v/v) in 40 min. The diluted mixture was directly injected into preparative HPLC. The fractions containing the purified product were pooled and lyophilized to obtain IL18-Seg34 as a white solid. The isolated yield was 60.1 mg (56%). MS (ESI): C₄₃₉H₆₈₄N₁₁₄O₁₃₈S₂; Average isotope calculated 9831.0810 Da [M]. found: 9830.9439 Da.

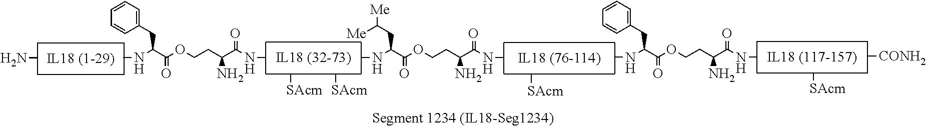

IL18-Seg1234 with Acm preparation: Peptide ketoacid IL18-Seg12 (16.1 mg; 1.97 μmol; 1.2 eq) and hydroxylamine peptide IL18-Seg34 (16.1 mg; 1.64 μmol; 1.0 eq) were in dissolved in 9:1 DMSO/H₂O containing 0.1 M oxalic acid (110 μL). A homogeneous liquid solution was obtained, which was reacted overnight at 60° C. After completion of the ligation reaction, the mixture was diluted first with DMSO (1890 μL). The mixture was further diluted with 1:1 H₂O/CH₃CN (q.s. 8 mL) containing TFA (0.1%, v/v). The diluted mixture was filtered and injected into preparative HPLC. Crude ligated peptide was purified by preparative HPLC using Gemini NX-C18 110 Å column (5 μm, 250×50 mm) at a flow rate of 40 mL/min at 60° C. using CH₃CN/H₂O with 0.1% TFA (v/v) as mobile phase, with a gradient of 10 to 60% CH₃CN with 0.1% TFA (v/v) in 30 min. The fractions containing the purified product were pooled and lyophilized to obtain IL18-Seg12 as a white solid in >98% purity. The isolated yield was 10.0 mg (33%).

Table 4 shows modified IL-18 polypeptides which may be prepared according to the methods provided herein.

| SEQ | | |
|---|---|---|
| ID | Sequence | |
| NO: | modifications* | Sequence |
| 1 | Native sequence | YFGKLESKLS VIRNLNDQVL FIDQGNRPLF |
| | | EDMTDSDCRD NAPRTIFIIS MYKDSQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 2 | E6K, C38A, K53A, | YFGKLKSKLS VIRNLNDQVL FIDQGNRPLF |
| | C68A, E85C | EDMTDSDARD NAPRTIFIIS MYADSQPRGM |
| | | AVTISVKAEK ISTLSCENKI ISFKCMNPPD NIKDTKSDII |
| | | FFQRSVPGHD NKMQFESSSY EGYFLACEKE |
| | | RDLFKLILKK EDELGDRSIM FTVQNED |
| 3 | E6K, V11I, C38A, | YFGKLKSKLS IIRNLNDQVL FIDQGNRPLF |
| | K53A, T63A, | EDMTDSDARD NAPRTIFIIS MYADSQPRGM |
| | C68A, C76A, | AVAISVKAEK ISTLSAENKI ISFKCMNPPD NIKDTKSDII |
| | E85C, C127A | FFQRSVPGHD NKMQFESSSY EGYFLAAEKE |
| | | RDLFKLILKK EDELGDRSIM FTVQNED |
| 4 | E6K, C38A, K53A, | YFGKLKSKLS VIRNLNDQVL FIDQGNRPLF |
| | C68A, M86C | EDMTDSDARD NAPRTIFIIS MYADSQPRGM |
| | | AVTISVKAEK ISTLSCENKI ISFKECNPPD NIKDTKSDII |
| | | FFQRSVPGHD NKMQFESSSY EGYFLACEKE |
| | | RDLFKLILKK EDELGDRSIM FTVQNED |
| 5 | E6K, V11I, C38A, | YFGKLKSKLS IIRNLNDQVL FIDQGNRPLF |
| | K53A, T63A, | EDMTDSDARD NAPRTIFIIS MYADSQPRGM |
| | C68A, C76A, | AVAISVKAEK ISTLSAENKI ISFKECNPPD NIKDTKSDII |
| | M86C, C127A | FFQRSVPGHD NKMQFESSSY EGYFLAAEKE |
| | | RDLFKLILKK EDELGDRSIM FTVQNED |
| 6 | E6K, V11I, C38A, | YFGKLKSKLS VIRNLNDQVL FIDQGNRPLF |
| | K53A, T63A, | EDMTDSDARD NAPRTIFIIS MYADSQPRGM |
| | C68A, C76A, | AVAISVKAEK ISTLSAENKI ISFKEMNPPD NIKDTKSCII |
| | D98C, C127A | |
| | | FFQRSVPGHD NKMQFESSSY EGYFLAAEKE |
| | | RDLFKLILKK EDELGDRSIM FTVQNED |
| 7 | E6K, C38A, K53A, | YFGKLKSKLS VIRNLNDQVL FIDQGNRPLF |
| | C68A, C76A, | EDMTDSDARD NAPRTIFIIS MYADSQPRGM |
| | M86C, C127A | AVTISVKAEK ISTLSAENKI ISFKECNPPD NIKDTKSDII |
| | | FFQRSVPGHD NKMQFESSSY EGYFLAAEKE |
| | | RDLFKLILKK EDELGDRSIM FTVQNED |
| 8 | E6K, V11I, C38A, | YFGKLKSKLS IIRNLNDQVL FIDQGNRPLF |
| | K53A, T63A, | EDMTDSDARD NAPRTIFIIS MYADSQPRGM |
| | C68A, C76A, | AVAISVKAEK ISTLSAENKI ISFKEMNPPD NIKDCKSDII |
| | T95C, C127A | FFQRSVPGHD NKMQFESSSY EGYFLAAEKE |
| | | RDLFKLILKK EDELGDRSIM FTVQNED |
| 9 | E6K, C38A, K53A, | YFGKLKSKLS VIRNLNDQVL FIDQGNRPLF |
| | C68A, D98C | EDMTDSDARD NAPRTIFIIS MYADSQPRGM |
| | | AVTISVKAEK ISTLSCENKI ISFKEMNPPD NIKDTKSCII |
| | | FFQRSVPGHD NKMQFESSSY EGYFLACEKE |
| | | RDLFKLILKK EDELGDRSIM FTVQNED |
| 10 | E6K, C38A, K53A, | YFGKLKSKLS VIRNLNDQVL FIDQGNRPLF |
| | C68A, C76A, | EDMTDSDARD NAPRTIFIIS MYADSQPRGM |
| | D98C, C127A | AVTISVKAEK ISTLSAENKI ISFKEMNPPD NIKDTKSCII |
| | | FFQRSVPGHD NKMQFESSSY EGYFLAAEKE |
| | | RDLFKLILKK EDELGDRSIM FTVQNED |
| 11 | E6K, V11I, C38A, | YFGKLKSKLS IIRNLNDQVL FIDQGNRPLF |
| | K53A, C76A, | EDMTDSDARD NAPRTIFIIS MYADSQPRGM |
| | C127A | AVTISVKCEK ISTLSAENKI ISFKEMNPPD NIKDTKSDII |
| | | FFQRSVPGHD NKMQFESSSY EGYFLAAEKE |
| | | RDLFKLILKK EDELGDRSIM FTVQNED |
| 12 | E6K, C38A, K53A, | YFGKLKSKLS VIRNLNDQVL FIDQGNRPLF |
| | T63A, C76A, | EDMTDSDARD NAPRTIFIIS MYADSQPRGM |
| | C127A | AVAISVKCEK ISTLSAENKI ISFKEMNPPD NIKDTKSDII |
| | | FFQRSVPGHD NKMQFESSSY EGYFLAAEKE |
| | | RDLFKLILKK EDELGDRSIM FTVQNED |
| 13 | E6K, K53A, T63N | YFGKLKSKLS VIRNLNDQVL FIDQGNRPLF |
| | | EDMTDSDCRD NAPRTIFIIS MYADSQPRGM |
| | | AVNISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 14 | E6K, K53A, S50A, | YFGKLKSKLS VIRNLNDQVL FIDQGNRPLF |
| | T63N | EDMTDSDCRD NAPRTIFIIA MYADSQPRGM |
| | | AVNISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 15 | E6K, K53A, S50H, | YFGKLKSKLS VIRNLNDQVL FIDQGNRPLF |
| | T63N | EDMTDSDCRD NAPRTIFIIH MYADSQPRGM |
| | | AVNISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 16 | E6K, K53A, T63N, | YFGKLKSKLS VIRNLNDQVL FIDQGNRPLF |
| | S65A | EDMTDSDCRD NAPRTIFIIS MYADSQPRGM |
| | | AVNIAVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 17 | E6K, K53A, S50H | YFGKLKSKLS VIRNLNDQVL FIDQGNRPLF |
| | | EDMTDSDCRD NAPRTIFIIH MYADSQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 18 | E6K, C38A, K53A, | YFGKLKSKLS VIRNLNDQVL FIDQGNRPLF |
| | C68A | EDMTDSDARD NAPRTIFIIS MYADSQPRGM |
| | | AVTISVKAEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 19 | E6K, K53A, K79A | YFGKLKSKLS VIRNLNDQVL FIDQGNRPLF |
| | | EDMTDSDCRD NAPRTIFIIS MYADSQPRGM |
| | | AVTISVKCEK ISTLSCENAI ISFKEMNPPD NIKDTKSDII |
| | | FFQRSVPGHD NKMQFESSSY EGYFLACEKE |
| | | RDLFKLILKK EDELGDRSIM FTVQNED |
| 20 | E6K, K53A, | YFGKLKSKLS VIRNLNDQVL FIDQGNRPLF |
| | R104A | EDMTDSDCRD NAPRTIFIIS MYADSQPRGM |
| | | AVTISVKCEK ISTLSCENAI ISFKEMNPPD NIKDTKSDII |
| | | FFQRSVPGHD NKMQFESSSY EGYFLACEKE |
| | | RDLFKLILKK EDELGDRSIM FTVQNED |
| 21 | E6K, K53A, | YFGKLKSKLS VIRNLNDQVL FIDQGNRPLF |
| | G108A | EDMTDSDCRD NAPRTIFIIS MYADSQPRGM |
| | | AVTISVKCEK ISTLSCENKI ISFKEMNPPD NIKDTKSDII |
| | | FFQRSVPAHD NKMQFESSSY EGYFLACEKE |
| | | RDLFKLILKK EDELGDRSIM FTVQNED |
| 22 | E6K, K53A, | YFGKLKSKLS VIRNLNDQVL FIDQGNRPLF |
| | H109A | EDMTDSDCRD NAPRTIFIIS MYADSQPRGM |
| | | AVTISVKCEK ISTLSCENKI ISFKEMNPPD NIKDTKSDII |
| | | FFQRSVPGAD NKMQFESSSY EGYFLACEKE |
| | | RDLFKLILKK EDELGDRSIM FTVQNED |
| 23 | E6K, K53A, | YFGKLKSKLS VIRNLNDQVL FIDQGNRPLF |
| | K112A | EDMTDSDCRD NAPRTIFIIS MYADSQPRGM |
| | | AVTISVKCEK ISTLSCENKI ISFKEMNPPD NIKDTKSDII |
| | | FFQRSVPGHD NAMQFESSSY EGYFLACEKE |
| | | RDLFKLILKK EDELGDRSIM FTVQNED |
| 24 | E6K, C38A, K53A, | YFGKLKSKLS VIRNLNDQVL FIDQGNRPLF |
| | T63A, C76A | EDMTDSDARD NAPRTIFIIS MYADSQPRGM |
| | | AVAISVKCEK ISTLSAENKI ISFKEMNPPD NIKDTKSDII |
| | | FFQRSVPGHD NKMQFESSSY EGYFLACEKE |
| | | RDLFKLILKK EDELGDRSIM FTVQNED |
| 25 | E6K, C38Q, K53A, | YFGKLKSKLS VIRNLNDQVL FIDQGNRPLF |
| | T63A, C76A | EDMTDSDQRD NAPRTIFIIS MYADSQPRGM |
| | | AVAISVKCEK ISTLSAENKI ISFKEMNPPD NIKDTKSDII |
| | | FFQRSVPGHD NKMQFESSSY EGYFLACEKE |
| | | RDLFKLILKK EDELGDRSIM FTVQNED |
| 26 | E6K, C38A, K53A, | YFGKLKSKLS VIRNLNDQVL FIDQGNRPLF |
| | T63A, C127A | EDMTDSDARD NAPRTIFIIS MYADSQPRGM |
| | | AVAISVKCEK ISTLSCENKI ISFKEMNPPD NIKDTKSDII |
| | | FFQRSVPGHD NKMQFESSSY EGYFLAAEKE |
| | | RDLFKLILKK EDELGDRSIM FTVQNED |
| 27 | E6K, C38Q, K53A, | YFGKLKSKLS VIRNLNDQVL FIDQGNRPLF |
| | T63A, C127A | EDMTDSDQRD NAPRTIFIIS MYADSQPRGM |
| | | AVAISVKCEK ISTLSCENKI ISFKEMNPPD NIKDTKSDII |
| | | FFQRSVPGHD NKMQFESSSY EGYFLAAEKE |
| | | RDLFKLILKK EDELGDRSIM FTVQNED |
| 28 | E6K, C38A, K53A, | YFGKLKSKLS VIRNLNDQVL FIDQGNRPLF |
| | T63A, C76A, | EDMTDSDARD NAPRTIFIIS MYADSQPRGM |
| | C127A | AVAISVKCEK ISTLSAENKI ISFKEMNPPD NIKDTKSDII |
| | | FFQRSVPGHD NKMQFESSSY EGYFLAAEKE |
| | | RDLFKLILKK EDELGDRSIM FTVQNED |
| 29 | E6K, V11I, C38A, | YFGKLKSKLS IIRNLNDQVL FIDQGNRPLF |
| | K53A, T63A | EDMTDSDARD NAPRTIFIIS MYADSQPRGM |
| | | AVAISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 30 | E6K, V11I, C38A, | YFGKLKSKLS IIRNLNDQVL FIDQGNRPLF |
| | K53A, T63A, | EDMTDSDARD NAPRTIFIIS MYADSQPRGM |
| | C76A, C127A | AVAISVKCEK ISTLSAENKI ISFKEMNPPD |
| | | NIKDTKSDII FFQRSVPGHD NKMQFESSSY |
| | | EGYFLAAEKE RDLFKLILKK EDELGDRSIM |
| | | FTVQNED |
| 31 | C38A, C76A, | YFGKLESKLS VIRNLNDQVL FIDQGNRPLF |
| | C127A | EDMTDSDARD NAPRTIFIIS MYKDSQPRGM |
| | | AVTISVKCEK ISTLSAENKI ISFKEMNPPD NIKDTKSDII |
| | | FFQRSVPGHD NKMQFESSSY EGYFLAAEKE |
| | | RDLFKLILKK EDELGDRSIM FTVQNED |
| 32 | C38A | YFGKLESKLS VIRNLNDQVL FIDQGNRPLF |
| | | EDMTDSDARD NAPRTIFIIS MYKDSQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 33 | E6K, C38A, K53A, | YFGKLKSKLS VIRNLNDQVL FIDQGNRPLF |
| | T63A | EDMTDSDARD NAPRTIFIIS MYADSQPRGM |
| | | AVAISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 34 | E06K, K53A, | YFGKLKSKLS VIRNLNDQVL FIDQGNRPLF |
| | S55A | EDMTDSDCRD NAPRTIFIIS MYADAQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 35 | Y01G, F02A, | GAGKLKSKLS VIRNLNDQVL FIDQGNRPLF |
| | E06K, M51G, | EDMTDSDCRD NAPRTIFIIS GYAAAQPRGM |
| | K53A, D54A, | AVAISVKCEK ISTLSCENKI ISFKEMNPPD NIKDTKSDII |
| | S55A, T63A | FFQRSVPGHD NKMQFESSSY EGYFLACEKE |
| | | RDLFKLILKK EDELGDRSIM FTVQNED |
| 36 | K53A | YFGKLESKLS VIRNLNDQVL FIDQGNRPLF |
| | | EDMTDSDCRD NAPRTIFIIS MYADSQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 37 | S55A | YFGKLESKLS VIRNLNDQVL FIDQGNRPLF |
| | | EDMTDSDCRD NAPRTIFIIS MYKDAQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 38 | E06K | YFGKLKSKLS VIRNLNDQVL FIDQGNRPLF |
| | | EDMTDSDCRD NAPRTIFIIS MYKDSQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 39 | E06K, K53A | YFGKLKSKLS VIRNLNDQVL FIDQGNRPLF |
| | | EDMTDSDCRD NAPRTIFIIS MYADSQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 40 | E06K, S55A | YFGKLKSKLS VIRNLNDQVL FIDQGNRPLF |
| | | EDMTDSDCRD NAPRTIFIIS MYKDAQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 41 | K53A, S55A | YFGKLESKLS VIRNLNDQVL FIDQGNRPLF |
| | | EDMTDSDCRD NAPRTIFIIS MYADAQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 42 | E06K, K53A, | YFGKLKSKLS VIRNLNDQVL FIDQGNRPLF |
| | S55A, T63A | EDMTDSDCRD NAPRTIFIIS MYADAQPRGM |
| | | AVAISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 43 | E06K, K53A, | GFGKLKSKLS VIRNLNDQVL FIDQGNRPLF |
| | S55A, Y01G | EDMTDSDCRD NAPRTIFIIS MYADAQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 44 | E06K, K53A, | YAGKLKSKLS VIRNLNDQVL FIDQGNRPLF |
| | S55A, F02A | EDMTDSDCRD NAPRTIFIIS MYADAQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 45 | E06K, K53A, | YFGKLKSKLS VIRNLNDQVL FIDQGNRPLF |
| | S55A, D54A | EDMTDSDCRD NAPRTIFIIS MYAAAQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 46 | E06K, K53A, | YFGKLKSKLS VIRNLNDQVL FIDQGNRPLF |
| | S55A, M51G | EDMTDSDCRD NAPRTIFIIS GYADAQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 47 | C38S, C68S, C76S, | YFGKLESKLS VIRNLNDQVL FIDQGNRPLF |
| | C127S | EDMTDSDSRD NAPRTIFIIS MYKDSQPRGM |
| | | AVTISVKSEK ISTLSSENKI ISFKEMNPPD NIKDTKSDII |
| | | FFQRSVPGHD NKMQFESSSY EGYFLASEKE |
| | | RDLFKLILKK EDELGDRSIM FTVQNED |
| 48 | C38S, C68S, C76S, | YFGKLESKLS VIRNLNDQVL FIDQGNRPLF |
| | C127S, K70C | EDMTDSDSRD NAPRTIFIIS MYKDSQPRGM |
| | | AVTISVKSEC ISTLSSENKI ISFKEMNPPD NIKDTKSDII |
| | | FFQRSVPGHD NKMQFESSSY EGYFLASEKE |
| | | RDLFKLILKK EDELGDRSIM FTVQNED |
| 49 | E06K, K53A, | YFGKLKSKLS VIRNLNDQVL FIDQGNRPLF |
| | S55A, C38S, C68S, | EDMTDSDSRD NAPRTIFIIS MYADAQPRGM |
| | C76S, C127S, | AVTISVKSEC ISTLSSENKI ISFKEMNPPD NIKDTKSDII |
| | K70C | FFQRSVPGHD NKMQFESSSY EGYFLASEKE |
| | | RDLFKLILKK EDELGDRSIM FTVQNED |
| 50 | E06K, K53A, | YFGKLKSKLS VIRNLNDQVL FIDQGNRPLF |
| | T63A | EDMTDSDCRD NAPRTIFIIS MYADSQPRGM |
| | | AVAISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 51 | T63A | YFGKLESKLS VIRNLNDQVL FIDQGNRPLF |
| | | EDMTDSDCRD NAPRTIFIIS MYKDSQPRGM |
| | | AVAISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 52 | E06K, T63A | YFGKLKSKLS VIRNLNDQVL FIDQGNRPLF |
| | | EDMTDSDCRD NAPRTIFIIS MYKDSQPRGM |
| | | AVAISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 53 | K53A, T63A | YFGKLESKLS VIRNLNDQVL FIDQGNRPLF |
| | | EDMTDSDCRD NAPRTIFIIS MYADSQPRGM |
| | | AVAISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 54 | E06K, K53A, | YFGKLKSKLS VIRNLNDQVL FIDQGNRPLF |
| | C38S, C68S, C76S, | EDMTDSDSRD NAPRTIFIIS MYADSQPRGM |
| | C127S, K70C | AVTISVKSEC ISTLSSENKI ISFKEMNPPD NIKDTKSDII |
| | | FFQRSVPGHD NKMQFESSSY EGYFLASEKE |
| | | RDLFKLILKK EDELGDRSIM FTVQNED |
| 55 | K53A, T63A, | YFGKLESKLS VIRNLNDQVL FIDQGNRPLF |
| | C38S, C68S, C76S, | EDMTDSDSRD NAPRTIFIIS MYADSQPRGM |
| | C127S, K70C | AVAISVKSEC ISTLSSENKI ISFKEMNPPD NIKDTKSDII |
| | | FFQRSVPGHD NKMQFESSSY EGYFLASEKE |
| | | RDLFKLILKK EDELGDRSIM FTVQNED |
| 56 | E6K, K53A, C38S, | YFGKLKSKLS VIRNLNDQVL FIDQGNRPLF |
| | C76S, C127S | EDMTDSDSRD NAPRTIFIIS MYADSQPRGM |
| | | AVTISVKCEK ISTLSSENKI ISFKEMNPPD NIKDTKSDII |
| | | FFQRSVPGHD NKMQFESSSY EGYFLASEKE |
| | | RDLFKLILKK EDELGDRSIM FTVQNED |
| 57 | E6K, C38S, K53A | YFGKLKSKLS VIRNLNDQVL FIDQGNRPLF |
| | | EDMTDSDSRD NAPRTIFIIS MYADSQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 58 | E6K, K53A, C38S, | YFGKLKSKLS VIRNLNDQVL FIDQGNRPLF |
| | C68S, C76S, | EDMTDSDSRD NAPRTIFIIS MYADSQPRGM |
| | C127S, K70C | AVTISVKSEC ISTLSSENKI ISFKEMNPPD NIKDTKSDII |
| | | FFQRSVPGHD NKMQFESSSY EGYFLASEKE |
| | | RDLFKLILKK EDELGDRSIM FTVQNED |
| 59 | E6K, C38A, K53A | YFGKLKSKLS VIRNLNDQVL FIDQGNRPLF |
| | | EDMTDSDARD NAPRTIFIIS MYADSQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 60 | E6K, C38Q, K53A | YFGKLKSKLS VIRNLNDQVL FIDQGNRPLF |
| | | EDMTDSDQRD NAPRTIFIIS MYADSQPRGM |
| | | AVTISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 61 | E6K, C38A, K53A, | YFGKLKSKLS VIRNLNDQVL FIDQGNRPLF |
| | C76A | EDMTDSDARD NAPRTIFIIS MYADSQPRGM |
| | | AVTISVKCEK ISTLSAENKI ISFKEMNPPD NIKDTKSDII |
| | | FFQRSVPGHD NKMQFESSSY EGYFLACEKE |
| | | RDLFKLILKK EDELGDRSIM FTVQNED |
| 62 | E6K, C38A, K53A, | YFGKLKSKLS VIRNLNDQVL FIDQGNRPLF |
| | C127A | EDMTDSDARD NAPRTIFIIS MYADSQPRGM |
| | | AVTISVKCEK ISTLSCENKI ISFKEMNPPD NIKDTKSDII |
| | | FFQRSVPGHD NKMQFESSSY EGYFLAAEKE |
| | | RDLFKLILKK EDELGDRSIM FTVQNED |
| 63 | E6K, C38A, K53A, | YFGKLKSKLS VIRNLNDQVL FIDQGNRPLF |
| | C76A, C127A | EDMTDSDARD NAPRTIFIIS MYADSQPRGM |
| | | AVTISVKCEK ISTLSAENKI ISFKEMNPPD NIKDTKSDII |
| | | FFQRSVPGHD NKMQFESSSY EGYFLAAEKE |
| | | RDLFKLILKK EDELGDRSIM FTVQNED |
| 64 | E6K, K53A, C38A, | YFGKLKSKLS VIRNLNDQVL FIDQGNRPLF |
| | S55A, T63A | EDMTDSDARD NAPRTIFIIS MYADAQPRGM |
| | | AVAISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 65 | E6K, C38Q, K53A, | YFGKLKSKLS VIRNLNDQVL FIDQGNRPLF |
| | S55A, T63A | EDMTDSDQRD NAPRTIFIIS MYADAQPRGM |
| | | AVAISVKCEK ISTLSCENKI ISFKEMNPPD NIKDTKSDII |
| | | FFQRSVPGHD NKMQFESSSY EGYFLACEKE |
| | | RDLFKLILKK EDELGDRSIM FTVQNED |
| 66 | E6K, K53A, K84A | YFGKLKSKLS VIRNLNDQVL FIDQGNRPLF |
| | | EDMTDSDCRD NAPRTIFIIS MYADSQPRGM |
| | | AVTISVKCEK ISTLSCENKI ISFAEMNPPD NIKDTKSDII |
| | | FFQRSVPGHD NKMQFESSSY EGYFLACEKE |
| | | RDLFKLILKK EDELGDRSIM FTVQNED |
| 67 | E6K, K53A, D98A | YFGKLKSKLS VIRNLNDQVL FIDQGNRPLF |
| | | EDMTDSDCRD NAPRTIFIIS MYADSQPRGM |
| | | AVTISVKCEK ISTLSCENKI ISFKEMNPPD NIKDTKSAII |
| | | FFQRSVPGHD NKMQFESSSY EGYFLACEKE |
| | | RDLFKLILKK EDELGDRSIM FTVQNED |
| 68 | E31Hse, T63Hse, | YFGKLESKLS VIRNLNDQVL FIDQGNRPLF |
| | S75Hse, E116Hse | ZDMTDSDCRD NAPRTIFIIS MYKDSQPRGM |
| | | AVZISVKCEK ISTLZCENKI ISFKEMNPPD NIKDTKSDII |
| | | FFQRSVPGHD NKMQFZSSSY EGYFLACEKE |
| | | RDLFKLILKK EDELGDRSIM FTVQNED |
| 74 | E6K, E31Hse, | YFGKLKSKLS VIRNLNDQVL FIDQGNRPLF |
| | K53A, T63Hse, | ZDMTDSDCRD NAPRTIFIIS MYADSQPRGM |
| | S75Hse, E116Hse | AVZISVKCEK ISTLZCENKI ISFKEMNPPD NIKDTKSDII |
| | | FFQRSVPGHD NKMQFZSSSY EGYFLACEKE |
| | | RDLFKLILKK EDELGDRSIM FTVQNED |
| 80 | E6K, E31Hse, | YFGKLKSKLS VIRNLNDQVL FIDQGNRPLF |
| | K53A, T63Hse, | ZDMTDSDCRD NAPRTIFIIS MYADSQPRGM |
| | S65A, S75Hse, | AVZIAVKCEK ISTLZCENKI ISFKEMNPPD NIKDTKSDII |
| | E116Hse | FFQRSVPGHD NKMQFZSSSY EGYFLACEKE |
| | | RDLFKLILKK EDELGDRSIM FTVQNED |
| 86 | E6K, E31Hse, | YFGKLKSKLS VIRNLNDQVL FIDQGNRPLF |
| | C38A, K53A, | ZDMTDSDARD NAPRTIFIIS MYADSQPRGM |
| | T63Hse, C68A, | AVZISVKAEK ISTLZAENKI ISFKEMNPPD NIKDTKSDII |
| | S75Hse, C76A, | FFQRSVPGHD NKMQFZSSSY EGYFLAAEKE |
| | E116Hse, C127A | RDLFKLILKK EDELGDRSIM FTVQNED |
| 92 | E31Hse, Q56L, | YFGKLESKLS VIRNLNDQVL FIDQGNRPLF |
| | P57Hse, S75Hse, | ZDMTDSDCRD NAPRTIFIIS MYKDSLZRGM |
| | E116Hse | AVTISVKCEK ISTLZCENKI ISFKEMNPPD NIKDTKSDII |
| | | FFQRSVPGHD NKMQFZSSSY EGYFLACEKE |
| | | RDLFKLILKK EDELGDRSIM FTVQNED |
| 98 | E6K, E31Hse, | YFGKLKSKLS VIRNLNDQVL FIDQGNRPLF |
| | K53A, Q56L, | ZDMTDSDCRD NAPRTIFIIS MYADSLZRGM |
| | P57Hse, S75Hse, | AVTISVKCEK ISTLZCENKI ISFKEMNPPD NIKDTKSDII |
| | E116Hse | FFQRSVPGHD NKMQFZSSSY EGYFLACEKE |
| | | RDLFKLILKK EDELGDRSIM FTVQNED |
| 104 | E6K, V11I, | YFGKLKSKLS IIRNLNDQVL FIDQGNRPLF |
| | E31Hse, K53A, | ZDMTDSDCRD NAPRTIFIIS MYADSLZRGM |
| | Q56L, P57Hse, | AVAISVKCEK ISTLZCENKI ISFKEMNPPD NIKDTKSDII |
| | T63A, S75Hse, | FFQRSVPGHD NKMQFZSSSY EGYFLACEKE |
| | E116Hse | RDLFKLILKK EDELGDRSIM FTVQNED |
| 110 | E6K, E31Hse, | YFGKLKSKLS VIRNLNDQVL FIDQGNRPLF |
| | C38A, K53A, | ZDMTDSDARD NAPRTIFIIS MYADSLZRGM |
| | Q56L, P57Hse, | AVTISVKAEK ISTLZAENKI ISFKEMNPPD NIKDTKSDII |
| | C68A, S75Hse, | FFQRSVPGHD NKMQFZSSSY EGYFLAAEKE |
| | C76A, E116Hse | RDLFKLILKK EDELGDRSIM FTVQNED |
| | C127A | |
| 116 | E31Hse, 149L, | YFGKLESKLS VIRNLNDQVL FIDQGNRPLF |
| | S50Hse, S75Hse, | ZDMTDSDCRD NAPRTIFILZ MYKDSQPRGM |
| | Y120F, E121Hse | AVTISVKCEK ISTLZCENKI ISFKEMNPPD NIKDTKSDII |
| | | FFQRSVPGHD NKMQFESSSF ZGYFLACEKE |
| | | RDLFKLILKK EDELGDRSIM FTVQNED |
| 122 | E6K, E31Hse, | YFGKLKSKLS VIRNLNDQVL FIDQGNRPLF |
| | I49L, S50Hse, | ZDMTDSDCRD NAPRTIFILZ MYADSQPRGM |
| | K53A, S75Hse, | AVTISVKCEK ISTLZCENKI ISFKEMNPPD NIKDTKSDII |
| | Y120F, E121Hse | FFQRSVPGHD NKMQFESSSF ZGYFLACEKE |
| | | RDLFKLILKK EDELGDRSIM FTVQNED |
| 128 | E6K, V11I, | YFGKLKSKLS IIRNLNDQVL FIDQGNRPLF |
| | E31Hse, 149L, | ZDMTDSDCRD NAPRTIFILZ MYADSQPRGM |
| | S50Hse, K53A, | AVAISVKCEK ISTLZCENKI ISFKEMNPPD NIKDTKSDII |
| | T63A, S75Hse, | FFQRSVPGHD NKMQFESSSF ZGYFLACEKE |
| | Y120F, E121Hse | RDLFKLILKK EDELGDRSIM FTVQNED |
| 134 | E6K, E31Hse, | YFGKLKSKLS VIRNLNDQVL FIDQGNRPLF |
| | C38A, 149L, | ZDMTDSDARD NAPRTIFILZ MYADSQPRGM |
| | S50Hse, K53A, | AVTISVKAEK ISTLZAENKI ISFKEMNPPD NIKDTKSDII |
| | C68A, S75Hse, | FFQRSVPGHD NKMQFESSSF ZGYFLAAEKE |
| | C76A, Y120F, | RDLFKLILKK EDELGDRSIM FTVQNED |
| | E121Hse, C127A | |
| 136 | E6K, E31Hse, | YFGKLKSKLS VIRNLNDQVL FIDQGNRPLF |
| | C38A, 149L, | ZDMTDSDARD NAPRTIFILZ MYADSQPRGM |
| | S50Hse, K53A, | AVTISVKAaK ISTLZAENKI ISFKEMNPPD NIKDTKSDII |
| | C68A, E69a, | FFQRSVPGHD NKMQFESSSF ZGYFLAAEKE |
| | S75Hse, Y120F, | RDLFKLILKK EDELGDRSIM FTVQNED |
| | E121Hse, C127A | |
| 140 | E31Hse, K67Hse, | YFGKLESKLS VIRNLNDQVL FIDQGNRPLF |
| | S75Hse, E116Hse | ZDMTDSDCRD NAPRTIFIIS MYKDSQPRGM |
| | | AVTISVZCEK ISTLZCENKI ISFKEMNPPD NIKDTKSDII |
| | | FFQRSVPGHD NKMQFZSSSY EGYFLACEKE |
| | | RDLFKLILKK EDELGDRSIM FTVQNED |
| 146 | E6K, E31Hse, | YFGKLKSKLS VIRNLNDQVL FIDQGNRPLF |
| | K53A, K67Hse, | ZDMTDSDCRD NAPRTIFIIS MYADSQPRGM |
| | S75Hse, E116Hse | AVTISVZCEK ISTLZCENKI ISFKEMNPPD NIKDTKSDII |
| | | FFQRSVPGHD NKMQFZSSSY EGYFLACEKE |
| | | RDLFKLILKK EDELGDRSIM FTVQNED |
| 152 | E6K, V11I, | YFGKLKSKLS IIRNLNDQVL FIDQGNRPLF |
| | E31Hse, K53A, | ZDMTDSDCRD NAPRTIFIIS MYADSQPRGM |
| | T63A, K67Hse, | AVAISVZCEK ISTLZCENKI ISFKEMNPPD NIKDTKSDII |
| | S75Hse, E116Hse | FFQRSVPGHD NKMQFZSSSY EGYFLACEKE |
| | | RDLFKLILKK EDELGDRSIM FTVQNED |
| 158 | E6K, E31Hse, | YFGKLKSKLS VIRNLNDQVL FIDQGNRPLF |
| | C38A, K53A, | ZDMTDSDARD NAPRTIFIIS MYADSQPRGM |
| | C68A, K67Hse, | AVTISVZAEK ISTLZAENKI ISFKEMNPPD NIKDTKSDII |
| | S75Hse, C76A, | FFQRSVPGHD NKMQFZSSSY EGYFLAAEKE |
| | E116Hse C127A | RDLFKLILKK EDELGDRSIM FTVQNED |
| 170 | E31Hse, T63Hse, | YFGKLESKLS VIRNLNDQVL FIDQGNRPLF |
| | E116Hse | ZDMTDSDCRD NAPRTIFIIS MYKDSQPRGM |
| | | AVZISVKCEK ISTLSCENKI ISFKEMNPPD NIKDTKSDII |
| | | FFQRSVPGHD NKMQFZSSSY EGYFLACEKE |
| | | RDLFKLILKK EDELGDRSIM FTVQNED |
| 171 | E6K, E31Hse, | YFGKLKSKLS VIRNLNDQVL FIDQGNRPLF |
| | K53A, T63Hse, | ZDMTDSDCRD NAPRTIFIIS MYADSQPRGM |
| | K70PEG, E116Hse | AVZISVKCEB ISTLSCENKI ISFKEMNPPD NIKDTKSDII |
| | | FFQRSVPGHD NKMQFZSSSY EGYFLACEKE |
| | | RDLFKLILKK EDELGDRSIM FTVQNED |
| 172 | E31Hse, C38S, | YFGKLESKLS VIRNLNDQVL FIDQGNRPLF |
| | T63Hse, C68S, | ZDMTDSDSRD NAPRTIFIIS MYKDSQPRGM |
| | C76S, E116Hse, | AVZISVKSEK ISTLSSENKI ISFKEMNPPD NIKDTKSDII |
| | C127S | FFQRSVPGHD NKMQFZSSSY EGYFLASEKE |
| | | RDLFKLILKK EDELGDRSIM FTVQNED |
| 173 | E6K, E31Hse, | YFGKLKSKLS VIRNLNDQVL FIDQGNRPLF |
| | C38S, K53A, | ZDMTDSDSRD NAPRTIFIIS MYADSQPRGM |
| | T63Hse, C68S, | AVZISVKSEB ISTLSSENKI ISFKEMNPPD NIKDTKSDII |
| | K70PEG, C76S, | FFQRSVPGHD NKMQFZSSSY EGYFLASEKE |
| | E116Hse, C127S | RDLFKLILKK EDELGDRSIM FTVQNED |
| 174 | E31Hse, M33J, | YFGKLESKLS VIRNLNDQVL FIDQGNRPLF |
| | M51J, M60J, | ZDJTDSDCRD NAPRTIFIIS JYKDSQPRGJ AVZISVKCEK |
| | T63Hse, M86J, | ISTLSCENKI ISFKEJNPPD NIKDTKSDII FFQRSVPGHD |
| | M113J, E116Hse, | NKJQFZSSSY EGYFLACEKE RDLFKLILKK |
| | M150J | EDELGDRSIJ FTVQNED |
| 175 | E6K, E31Hse, | YFGKLKSKLS VIRNLNDQVL FIDQGNRPLF |
| | M33J, M51J, | ZDJTDSDCRD NAPRTIFIIS JYADSQPRGJ AVZISVKCEB |
| | K53A, M60J, | ISTLSCENKI ISFKEJNPPD NIKDTKSDII FFQRSVPGHD |
| | T63Hse, K70PEG, | NKJQFZSSSY EGYFLACEKE RDLFKLILKK |
| | M86J, M113J, | EDELGDRSIJ FTVQNED |
| | E116Hse, M150J | |
| 176 | E31Hse, M33J, | YFGKLESKLS VIRNLNDQVL FIDQGNRPLF |
| | C38S, M51J, | ZDJTDSDSRD NAPRTIFIIS JYKDSQPRGJ AVZISVKSEK |
| | M60J, T63Hse, | ISTLSSENKI ISFKEJNPPD NIKDTKSDII FFQRSVPGHD |
| | C68S, C76S, M86J, | NKJQFZSSSY EGYFLASEKE RDLFKLILKK |
| | M113J, E116Hse, | EDELGDRSIJ FTVQNED |
| | C127S, M150J | |
| 177 | E6K, E31Hse, | YFGKLKSKLS VIRNLNDQVL FIDQGNRPLF |
| | M33J, C38S, | ZDJTDSDSRD NAPRTIFIIS JYADSQPRGJ AVZISVKSEB |
| | M51J, K53A, | ISTLSSENKI ISFKEJNPPD NIKDTKSDII FFQRSVPGHD |
| | M60J, T63Hse, | NKJQFZSSSY EGYFLASEKE RDLFKLILKK |
| | C68S, K70PEG, | EDELGDRSIJ FTVQNED |
| | C76S, M86J, | |
| | M113J, E116Hse, | |
| | C127S, M150J | |
| 179 | E6K, E31Hse, | YFGKLKSKLS VIRNLNDQVL FIDQGNRPLF |
| | K53A, T63Hse, | ZDMTDSDCRD NAPRTIFIIS MYADSQPRGM |
| | E116Hse | AVZISVKCEK ISTLSCENKI ISFKEMNPPD NIKDTKSDII |
| | | FFQRSVPGHD NKMQFZSSSY EGYFLACEKE |
| | | RDLFKLILKK EDELGDRSIM FTVQNED |
| 180 | E6K, E31Hse, | YFGKLKSKLS VIRNLNDQVL FIDQGNRPLF |
| | C38S, K53A, | ZDMTDSDSRD NAPRTIFIIS MYADSQPRGM |
| | T63Hse, C68S, | AVZISVKSEK ISTLSSENKI ISFKEMNPPD NIKDTKSDII |
| | C76S, E116Hse, | FFQRSVPGHD NKMQFZSSSY EGYFLASEKE |
| | C127S | RDLFKLILKK EDELGDRSIM FTVQNED |
| 183 | E6K, E31Hse, | YFGKLKSKLS VIRNLNDQVL FIDQGNRPLF |
| | M33J, M51J, | ZDJTDSDCRD NAPRTIFIIS JYADSQPRGJ AVZISVKCEK |
| | K53A, M60J, | ISTLSCENKI ISFKEJNPPD NIKDTKSDII FFQRSVPGHD |
| | T63Hse, M86J, | NKJQFZSSSY EGYFLACEKE RDLFKLILKK |
| | M113J, E116Hse, | EDELGDRSIJ FTVQNED |
| | M150J | |
| 184 | E6K, E31Hse, | YFGKLKSKLS VIRNLNDQVL FIDQGNRPLF |
| | M33J, C38S, | ZDJTDSDSRD NAPRTIFIIS JYADSQPRGJ AVZISVKSEK |
| | M51J, K53A, | ISTLSSENKI ISFKEJNPPD NIKDTKSDII FFQRSVPGHD |
| | M60J, T63Hse, | NKJQFZSSSY EGYFLASEKE RDLFKLILKK |
| | C68S, C76S, M86J, | EDELGDRSIJ FTVQNED |
| | M113J, E116Hse, | |
| | C127S, M150J | |
| 186 | E31Hse, T63Hse, | YFGKLESKLS VIRNLNDQVL FIDQGNRPLF |
| | K70PEG, E116Hse | ZDMTDSDCRD NAPRTIFIIS MYKDSQPRGM |
| | | AVZISVKCEB ISTLSCENKI ISFKEMNPPD NIKDTKSDII |
| | | FFQRSVPGHD NKMQFZSSSY EGYFLACEKE |
| | | RDLFKLILKK EDELGDRSIM FTVQNED |
| 187 | E31Hse, C38S, | YFGKLESKLS VIRNLNDQVL FIDQGNRPLF |
| | T63Hse, C68S, | ZDMTDSDSRD NAPRTIFIIS MYKDSQPRGM |
| | K70PEG, C76S, | AVZISVKSEB ISTLSSENKI ISFKEMNPPD NIKDTKSDII |
| | E116Hse, C127S | FFQRSVPGHD NKMQFZSSSY EGYFLASEKE |
| | | RDLFKLILKK EDELGDRSIM FTVQNED |
| 190 | E31Hse, M33J, | YFGKLESKLS VIRNLNDQVL FIDQGNRPLF |
| | M51J, M60J, | ZDJTDSDCRD NAPRTIFIIS JYKDSQPRGJ AVZISVKCEB |
| | T63Hse, K70PEG, | ISTLSCENKI ISFKEJNPPD NIKDTKSDII FFQRSVPGHD |
| | M86J, M113J, | NKJQFZSSSY EGYFLACEKE RDLFKLILKK |
| | E116Hse, M150J | EDELGDRSIJ FTVQNED |
| 191 | E31Hse, M33J, | YFGKLESKLS VIRNLNDQVL FIDQGNRPLF |
| | C38S, M51J, | ZDJTDSDSRD NAPRTIFIIS JYKDSQPRGJ AVZISVKSEB |
| | M60J, T63Hse, | ISTLSSENKI ISFKEJNPPD NIKDTKSDII FFQRSVPGHD |
| | C68S, K70PEG, | NKJQFZSSSY EGYFLASEKE RDLFKLILKK |
| | C76S, M86J, | EDELGDRSIJ FTVQNED |
| | M113J, E116Hse, | |
| | C127S, M150J | |
| 192 | E6K, E31Hse, | YFGKLKSKLS VIRNLNDQVL FIDQGNRPLF |
| | K53A, T63A, | ZDMTDSDCRD NAPRTIFIIS MYADSQPRGM |
| | S75Hse, E116Hse | AVAISVKCEK ISTLZCENKI ISFKEMNPPD NIKDTKSDII |
| | | FFQRSVPGHD NKMQFZSSSY EGYFLACEKE |
| | | RDLFKLILKK EDELGDRSIM FTVQNED |
| 193 | E6K, E31Hse, | YFGKLKSKLS VIRNLNDQVL FIDQGNRPLF |
| | C38S, K53A, | ZDMTDSDSRD NAPRTIFIIS MYADSQPRGM |
| | T63A, C68S, | AVAISVKSEK ISTLZSENKI ISFKEMNPPD NIKDTKSDII |
| | S75Hse, C76S, | FFQRSVPGHD NKMQFZSSSY EGYFLASEKE |
| | E116Hse, C127S | RDLFKLILKK EDELGDRSIM FTVQNED |
| 196 | E6K, E31Hse, | YFGKLKSKLS VIRNLNDQVL FIDQGNRPLF |
| | M33J, M51J, | ZDJTDSDCRD NAPRTIFIIS JYADSQPRGJ AVAISVKCEK |
| | K53A, M60J, | ISTLZCENKI ISFKEJNPPD NIKDTKSDII FFQRSVPGHD |
| | T63A, S75Hse, | NKJQFZSSSY EGYFLACEKE RDLFKLILKK |
| | M86J, M113J, | EDELGDRSIJ FTVQNED |
| | E116Hse, M150J | |
| 197 | E6K, E31Hse, | YFGKLKSKLS VIRNLNDQVL FIDQGNRPLF |
| | M33J, C38S, | ZDJTDSDSRD NAPRTIFIIS JYADSQPRGJ AVAISVKCEK |
| | M51J, K53A, | ISTLZSENKI ISFKEJNPPD NIKDTKSDII FFQRSVPGHD |
| | M60J, T63A, | NKJQFZSSSY EGYFLASEKE RDLFKLILKK |
| | C68S, S75Hse, | EDELGDRSIJ FTVQNED |
| | C76S, M86J, | |
| | M113J, E116Hse, | |
| | C127S, M150J | |
| 200 | E6K, E31Hse, | YFGKLKSKLS VIRNLNDQVL FIDQGNRPLF |
| | M33J, C38S, | ZDJTDSDSRD NAPRTIFIIS JYADSQPRGJ AVZISVKCEK |
| | M51J, K53A, | ISTLSCENKI ISFKEJNPPD NIKDTKSDII FFQRSVPGHD |
| | M60J, T63Hse, | NKJQFZSSSY EGYFLACEKE RDLFKLILKK |
| | M86J, M113J, | EDELGDRSIJ FTVQNED |
| | E116Hse, M150J | |
| 201 | E6K, E31Hse, | YFGKLKSKLS VIRNLNDQVL FIDQGNRPLF |
| | M33J, C38A, | ZDJTDSDARD NAPRTIFIIS JYADSQPRGJ AVZISVKCEK |
| | M51J, K53A, | ISTLSCENKI ISFKEJNPPD NIKDTKSDII FFQRSVPGHD |
| | M60J, T63Hse, | NKJQFZSSSY EGYFLACEKE RDLFKLILKK |
| | M86J, M113J, | EDELGDRSIJ FTVQNED |
| | E116Hse, M150J | |
| 202 | E6K, E31Hse, | YFGKLKSKLS VIRNLNDQVL FIDQGNRPLF |
| | C38S, K53A, | ZDMTDSDSRD NAPRTIFIIS MYADSQPRGM |
| | T63Hse, E116Hse | AVZISVKCEK ISTLSCENKI ISFKEMNPPD NIKDTKSDII |
| | | FFQRSVPGHD NKMQFZSSSY EGYFLACEKE |
| | | RDLFKLILKK EDELGDRSIM FTVQNED |
| 203 | E6K, E31Hse, | YFGKLKSKLS VIRNLNDQVL FIDQGNRPLF |
| | C38A, K53A, | ZDMTDSDARD NAPRTIFIIS MYADSQPRGM |
| | T63Hse, E116Hse | AVZISVKCEK ISTLSCENKI ISFKEMNPPD NIKDTKSDII |
| | | FFQRSVPGHD NKMQFZSSSY EGYFLACEKE |
| | | RDLFKLILKK EDELGDRSIM FTVQNED |
| 204 | V11I, C38A, | YFGKLESKLSIIRNLNDQVLFIDQGNRPLFEDMTDSDARD |
| | K53A, C76A, | NAPRTIFIISMYADSQPRGMAVTISVKCEKISTLSAENKIIS |
| | C127A | FKEMNPPDNIKDTKSDIIFFQRSVPGHDNKMQFESSSYEG |
| | | YFLAAEKERDLFKLILKKEDELGDRSIMFTVQNED |
| 205 | V11I, C38A, | YFGKLESKLSIIRNLNDQVLFIDQGNRPLFEDMTDSDARD |
| | K53A, T63A, | NAPRTIFIISMYADSQPRGMAVAISVKCEKISTLSAENKIIS |
| | C76A, C127A | FKEMNPPDNIKDTKSDIIFFQRSVPGHDNKMQFESSSYEG |
| | | YFLAAEKERDLFKLILKKEDELGDRSIMFTVQNED |
| 206 | V11I, C38A, | YFGKLESKLSIIRNLNDQVLFIDQGNRPLFEDMTDSDARDN |
| | K53A, S55A, | APRTIFIISMYADAQPRGMAVTISVKCEKISTLSAENKIIS |
| | C76A, C127A | FKEMNPPDNIKDTKSDIIFFQRSVPGHDNKMQFESSSYEG |
| | | YFLAAEKERDLFKLILKKEDELGDRSIMFTVQNED |
| 207 | V11I, C38A, | YFGKLESKLSIIRNLNDQVLFIDQGNRPLFEDMTDSDARDN |
| | M51G, K53A, | APRTIFIISGYADSQPRGMAVTISVKCEKISTLSAENKIIS |
| | C76A, C127A | FKEMNPPDNIKDTKSDIIFFQRSVPGHDNKMQFESSSYEG |
| | | YFLAAEKERDLFKLILKKEDELGDRSIMFTVQNED |
| 208 | V11I, C38A, | YFGKLESKLSIIRNLNDQVLFIDQGNRPLFEDMTDSDARDN |
| | K53A, D54A, | APRTIFIISMYAASQPRGMAVTISVKCEKISTLSAENKIIS |
| | C76A, C127A | FKEMNPPDNIKDTKSDIIFFQRSVPGHDNKMQFESSSYEG |
| | | YFLAAEKERDLFKLILKKEDELGDRSIMFTVQNED |
| 209 | F2A, V11I, C38A, | YAGKLESKLSIIRNLNDQVLFIDQGNRPLFEDMTDSDARDN |
| | K53A, C76A, | APRTIFIISMYADSQPRGMAVTISVKCEKISTLSAENKIIS |
| | C127A | FKEMNPPDNIKDTKSDIIFFQRSVPGHDNKMQFESSSYEG |
| | | YFLAAEKERDLFKLILKKEDELGDRSIMFTVQNED |
| 210 | V11I, E31A, | YFGKLESKLSIIRNLNDQVLFIDQGNRPLFADMTDSDARDN |
| | C38A, K53A, | APRTIFIISMYADSQPRGMAVTISVKCEKISTLSAENKIIS |
| | C76A, C127A | FKEMNPPDNIKDTKSDIIFFQRSVPGHDNKMQFESSSYEG |
| | | YFLAAEKERDLFKLILKKEDELGDRSIMFTVQNED |
| 211 | V11I, T34A, | YFGKLESKLSIIRNLNDQVLFIDQGNRPLFEDMADSDARDN |
| | C38A, K53A, | APRTIFIISMYADSQPRGMAVTISVKCEKISTLSAENKIIS |
| | C76A, C127A | FKEMNPPDNIKDTKSDIIFFQRSVPGHDNKMQFESSSYEG |
| | | YFLAAEKERDLFKLILKKEDELGDRSIMFTVQNED |
| 212 | V11I, D35A, | YFGKLESKLSIIRNLNDQVLFIDQGNRPLFEDMTASDARDN |
| | C38A, K53A, | APRTIFIISMYADSQPRGMAVTISVKCEKISTLSAENKIIS |
| | C76A, C127A | FKEMNPPDNIKDTKSDIIFFQRSVPGHDNKMQFESSSYEG |
| | | YFLAAEKERDLFKLILKKEDELGDRSIMFTVQNED |
| 213 | V11I, S36A, C38A, | YFGKLESKLSIIRNLNDQVLFIDQGNRPLFEDMTDADARDN |
| | K53A, C76A, | APRTIFIISMYADSQPRGMAVTISVKCEKISTLSAENKIIS |
| | C127A | FKEMNPPDNIKDTKSDIIFFQRSVPGHDNKMQFESSSYEG |
| | | YFLAAEKERDLFKLILKKEDELGDRSIMFTVQNED |
| 214 | V11I, D37A, | YFGKLESKLSIIRNLNDQVLFIDQGNRPLFEDMTDSAARDN |
| | C38A, K53A, | APRTIFIISMYADSQPRGMAVTISVKCEKISTLSAENKIIS |
| | C76A, C127A | FKEMNPPDNIKDTKSDIIFFQRSVPGHDNKMQFESSSYEG |
| | | YFLAAEKERDLFKLILKKEDELGDRSIMFTVQNED |
| 215 | V11I, E31A, | YFGKLESKLSIIRNLNDQVLFIDQGNRPLFADMTDSAARDN |
| | D37A, C38A, | APRTIFIISMYADSQPRGMAVTISVKCEKISTLSAENKIIS |
| | K53A, C76A, | FKEMNPPDNIKDTKSDIIFFQRSVPGHDNKMQFESSSYEG |
| | C127A | YFLAAEKERDLFKLILKKEDELGDRSIMFTVQNED |
| 216 | V11I, C38A, | YFGKLESKLSIIRNLNDQVLFIDQGNRPLFEDMTDSDARAN |
| | D40A, K53A, | APRTIFIISMYADSQPRGMAVTISVKCEKISTLSAENKIIS |
| | C76A, C127A | FKEMNPPDNIKDTKSDIIFFQRSVPGHDNKMQFESSSYEG |
| | | YFLAAEKERDLFKLILKKEDELGDRSIMFTVQNED |
| 217 | V11I, C38A, | YFGKLESKLSIIRNLNDQVLFIDQGNRPLFEDMTDSDARDA |
| | N41A, K53A, | APRTIFIISMYADSQPRGMAVTISVKCEKISTLSAENKIIS |
| | C76A, C127A | FKEMNPPDNIKDTKSDIIFFQRSVPGHDNKMQFESSSYEG |
| | | YFLAAEKERDLFKLILKKEDELGDRSIMFTVQNED |
| 218 | V11I, C38A, | YFGKLESKLSIIRNLNDQVLFIDQGNRPLFEDMTDSDARDN |
| | K53A, C76A, | APRTIFIISMYADSQPRGMAVTISVKCEKISTLSAENKIIS |
| | C127A, D132A | FKEMNPPDNIKDTKSDIIFFQRSVPGHDNKMQFESSSYEG |
| | | YFLAAEKERALFKLILKKEDELGDRSIMFTVQNED |
| 219 | V11I, C38A, | YFGKLESKLSIIRNLNDQVLFIDQGNRPLFEDMTDSDARDN |
| | K53A, C76A, | APRTIFIISMYADSQPRGMAVTISVKCEKISTLSAENKIIS |
| | G108A, C127A | FKEMNPPDNIKDTKSDIIFFQRSVPAHDNKMQFESSSYEG |
| | | YFLAAEKERDLFKLILKKEDELGDRSIMFTVQNED |
| 220 | V11I, C38A, | YFGKLESKLSIIRNLNDQVLFIDQGNRPLFEDMTDSDARD |
| | K53A, C76A, | NAPRTIFIISMYADSQPRGMAVTISVKCEKISTLSAENKIIS |
| | H109A, C127A | FKEMNPPDNIKDTKSDIIFFQRSVPGADNKMQFESSSYEG |
| | | YFLAAEKERDLFKLILKKEDELGDRSIMFTVQNED |
| 221 | V11I, C38A, | YFGKLESKLSIIRNLNDQVLFIDQGNRPLFEDMTDSDARD |
| | K53A, C76A, | NAPRTIFIISMYADSQPRGMAVTISVKCEKISTLSAENKIIS |
| | D110A, C127A | FKEMNPPDNIKDTKSDIIFFQRSVPGHANKMQFESSSYEG |
| | | YFLAAEKERDLFKLILKKEDELGDRSIMFTVQNED |
| 222 | K8R, V11I, C38A, | YFGKLESRLSIIRNLNDQVLFIDQGNRPLFEDMTDSDARD |
| | C76A, Q103E, | NAPRTIFIISMYKDSQPRGMAVTISVKCEKISTLSAENKIIS |
| | C127A | FKEMNPPDNIKDTKSDIIFFERSVPGHDNKMQFESSSYEG |
| | | YFLAAEKERDLFKLILKKEDELGDRSIMFTVQNED |
| 223 | K8E, V11I, C38A, | YFGKLESELSIIRNLNDQVLFIDQGNRPLFEDMTDSDARD |
| | C76A, Q103R, | NAPRTIFIISMYKDSQPRGMAVTISVKCEKISTLSAENKIIS |
| | C127A | FKEMNPPDNIKDTKSDIIFFRRSVPGHDNKMQFESSSYEG |
| | | YFLAAEKERDLFKLILKKEDELGDRSIMFTVQNED |
| 224 | V11I, C38A, | YFGKLESKLSIIRNLNDQVLFIDQGNRPLFEDMTDSDARD |
| | C76A, Q103K, | NAPRTIFIISMYKDSQPRGMAVTISVKCEKISTLSAENKIIS |
| | C127A | FKEMNPPDNIKDTKSDIIFFKRSVPGHDNKMQFESSSYEG |
| | | YFLAAEKERDLFKLILKKEDELGDRSIMFTVQNED |
| 225 | V11I, C38A, S55H, | YFGKLESKLSIIRNLNDQVLFIDQGNRPLFEDMTDSDARD |
| | C76A, C127A | NAPRTIFIISMYKDHQPRGMAVTISVKCEKISTLSAENKIIS |
| | | FKEMNPPDNIKDTKSDIIFFQRSVPGHDNKMQFESSSYEG |
| | | YFLAAEKERDLFKLILKKEDELGDRSIMFTVQNED |
| 226 | V11I, C38A, S55R, | YFGKLESKLSIIRNLNDQVLFIDQGNRPLFEDMTDSDARD |
| | C76A, C127A | NAPRTIFIISMYKDRQPRGMAVTISVKCEKISTLSAENKIIS |
| | | FKEMNPPDNIKDTKSDIIFFQRSVPGHDNKMQFESSSYEG |
| | | YFLAAEKERDLFKLILKKEDELGDRSIMFTVQNED |
| 227 | V11I, C38A, S55T, | YFGKLESKLSIIRNLNDQVLFIDQGNRPLFEDMTDSDARD |
| | C76A, C127A | NAPRTIFIISMYKDTQPRGMAVTISVKCEKISTLSAENKIIS |
| | | FKEMNPPDNIKDTKSDIIFFQRSVPGHDNKMQFESSSYEG |
| | | YFLAAEKERDLFKLILKKEDELGDRSIMFTVQNED |
| 228 | V11I, C38A, | YFGKLESKLSIIRNLNDQVLFIDQGNRPLFEDMTDSDARD |
| | C76A, S105I, | NAPRTIFIISMYKDSQPRGMAVTISVKCEKISTLSAENKIIS |
| | C127A | FKEMNPPDNIKDTKSDIIFFQRIVPGHDNKMQFESSSYEG |
| | | YFLAAEKERDLFKLILKKEDELGDRSIMFTVQNED |
| 229 | V11I, C38A, | YFGKLESKLSIIRNLNDQVLFIDQGNRPLFEDMTDSDARD |
| | C76A, S105K, | NAPRTIFIISMYKDSQPRGMAVTISVKCEKISTLSAENKIIS |
| | C127A | FKEMNPPDNIKDTKSDIIFFQRKVPGHDNKMQFESSSYEG |
| | | YFLAAEKERDLFKLILKKEDELGDRSIMFTVQNED |
| 230 | K8L, E6K, V11I, | YFGKLKSLLSIIRNLNDQVLFIDQGNRPLFEDMTDSDARD |
| | C38A, K53A, | NAPRTIFIISMYADSQPRGMAVAISVKCEKISTLSAENKIIS |
| | T63A, C76A, | FKEMNPPDNIKDTKSDIIFFQRSVPGHDNKMQFESSSYEG |
| | C127A | YFLAAEKERDLFKLILKKEDELGDRSIMFTVQNED |
| 231 | E6K, V11I, C38A, | YFGKLKSKLSIIRNLNDQVLFIDQGNRPLFEDMTDSDARD |
| | I49E, K53A, T63A, | NAPRTIFIESMYADSQPRGMAVAISVKCEKISTLSAENKII |
| | C76A, C127A | SFKEMNPPDNIKDTKSDIIFFQRSVPGHDNKMQFESSSYE |
| | | GYFLAAEKERDLFKLILKKEDELGDRSIMFTVQNED |
| 232 | E6K, V11I, C38A, | YFGKLKSKLSIIRNLNDQVLFIDQGNRPLFEDMTDSDARD |
| | I49M, K53A, | NAPRTIFIMSMYADSQPRGMAVAISVKCEKISTLSAENKII |
| | T63A, C76A, | SFKEMNPPDNIKDTKSDIIFFQRSVPGHDNKMQFESSSYE |
| | C127A | GYFLAAEKERDLFKLILKKEDELGDRSIMFTVQNED |
| 233 | E6K, V11I, C38A, | YFGKLKSKLSIIRNLNDQVLFIDQGNRPLFEDMTDSDARD |
| | I49R, K53A, | NAPRTIFIRSMYADSQPRGMAVAISVKCEKISTLSAENKII |
| | T63A, C76A, | SFKEMNPPDNIKDTKSDIIFFQRSVPGHDNKMQFESSSYE |
| | C127A | GYFLAAEKERDLFKLILKKEDELGDRSIMFTVQNED |
| 234 | E6K, V11I, C38A, | YFGKLKSKLSIIRNLNDQVLFIDQGNRPLFEDMTDSDARD |
| | K53A, T63A, | NAPRTIFIISMYADSQPRGMAVAISVKCEKISTLSAENKIIS |
| | C76A, Q103R, | FKEMNPPDNIKDTKSDIIFFRRSVPGHDNKMQFESSSYEG |
| | C127A | YFLAAEKERDLFKLILKKEDELGDRSIMFTVQNED |
| 235 | E6K, K8E, V11I, | YFGKLKSELSIIRNLNDQVLFIDQGNRPLFEDMTDSDARD |
| | C38A, K53A, | NAPRTIFIISMYADSQPRGMAVAISVKCEKISTLSAENKIIS |
| | T63A, C76A, | FKEMNPPDNIKDTKSDIIFFRRSVPGHDNKMQFESSSYEG |
| | Q103R, C127A | YFLAAEKERDLFKLILKKEDELGDRSIMFTVQNED |
| 236 | E6K, V11I, C38A, | YFGKLKSKLSIIRNLNDQVLFIDQGNRPLFEDMTDSDARD |
| | K53A, T63A, | NAPRTIFIISMYADSQPRGMAVAISVKCEKISTLSAENKIIS |
| | C76A, C127A, | FKEMNPPDNIKDTKSDIIFFQRSVPGHDNKMQFESSSYEG |
| | V153R | YFLAAEKERDLFKLILKKEDELGDRSIMFTRQNED |
| 237 | E6K, V11I, C38A, | YFGKLKSKLSIIRNLNDQVLFIDQGNRPLFEDMTDSDARD |
| | K53A, T63A, | NAPRTIFIISMYADSQPRGMAVAISVKCEKISTLSAENKIIS |
| | C76A, C127A, | FKEMNPPDNIKDTKSDIIFFQRSVPGHDNKMQFESSSYEG |
| | V153E | YFLAAEKERDLFKLILKKEDELGDRSIMFTEQNED |
| 238 | E6K, V11I, C38A, | YFGKLKSKLSIIRNLNDQVLFIDQGNRPLFEDMTDSDARD |
| | K53A, T63A, | NAPRTIFIISMYADSQPRGMAVAISVKCEKISTLSAENKIIS |
| | C76A, C127A, | FKEMNPPDNIKDTKSDIIFFQRSVPGHDNKMQFESSSYEG |
| | V153Y | YFLAAEKERDLFKLILKKEDELGDRSIMFTYQNED |
| 239 | E6K, V11I, C38A, | YFGKLKSKLSIIRNLNDQVLFIDQGNRPLFEDMTDSDARD |
| | M51G, K53A, | NAPRTIFIISGYADSQPRGMAVAISVKCEKISTLSAENKIIS |
| | T63A, C76A, | FKEMNPPDNIKDTKSDIIFFQRSVPGHDNKMQFESSSYEG |
| | C127A | YFLAAEKERDLFKLILKKEDELGDRSIMFTVQNED |
| 240 | E6R, V11I, C38A, | YFGKLRSKLSIIRNLNDQVLFIDQGNRPLFEDMTDSDARD |
| | K53A, T63A, | NAPRTIFIISMYADSQPRGMAVAISVKCEKISTLSAENKIIS |
| | C76A, C127A | FKEMNPPDNIKDTKSDIIFFQRSVPGHDNKMQFESSSYEG |
| | | YFLAAEKERDLFKLILKKEDELGDRSIMFTVQNED |
| 241 | N-terminal 4xG, | GGGGYFGKLKSKLSIIRNLNDQVLFIDQGNRPLFEDMTDS |
| | E6K, V11I, C38A, | DARDNAPRTIFIISMYADSQPRGMAVAISVKCEKISTLSAE |
| | K53A, T63A, | NKIISFKEMNPPDNIKDTKSDIIFFQRSVPGHDNKMQFESS |
| | C76A, C127A | SYEGYFLAAEKERDLFKLILKKEDELGDRSIMFTVQNED |
| 242 | N-terminal G, E6K, | GYFGKLKSKLSIIRNLNDQVLFIDQGNRPLFEDMTDSDAR |
| | V11I, C38A, | DNAPRTIFIISMYADSQPRGMAVAISVKCEKISTLSAENKII |
| | K53A, T63A, | SFKEMNPPDNIKDTKSDIIFFQRSVPGHDNKMQFESSSYE |
| | C76A, C127A | GYFLAAEKERDLFKLILKKEDELGDRSIMFTVQNED |
| 243 | Y1M, E6K, V11I, | MFGKLKSKLSIIRNLNDQVLFIDQGNRPLFEDMTDSDARD |
| | C38A, K53A, | NAPRTIFIISMYADSQPRGMAVAISVKCEKISTLSAENKIIS |
| | T63A, C76A, | FKEMNPPDNIKDTKSDIIFFQRSVPGHDNKMQFESSSYEG |
| | C127A | YFLAAEKERDLFKLILKKEDELGDRSIMFTVQNED |
| 244 | N-terminal G, E6K, | GYFGKLKSKLSIIRNLNDQVLFIDQGNRPLFEDMTDSDAR |
| | V11I, C38A, | DNAPRTIFIISGYADSQPRGMAVAISVKCEKISTLSAENKII |
| | M51G, K53A, | SFKEMNPPDNIKDTKSDIIFFQRSVPGHDNKMQFESSSYE |
| | T63A, C76A, | GYFLAAEKERDLFKLILKKEDELGDRSIMFTVQNED |
| | C127A | |

*Residue position numbering based on SEQ ID NO: 1 as a reference sequence

n the table above, Z=homoserine (Hse), J=O-methyl-homoserine; α has a structure selected from:

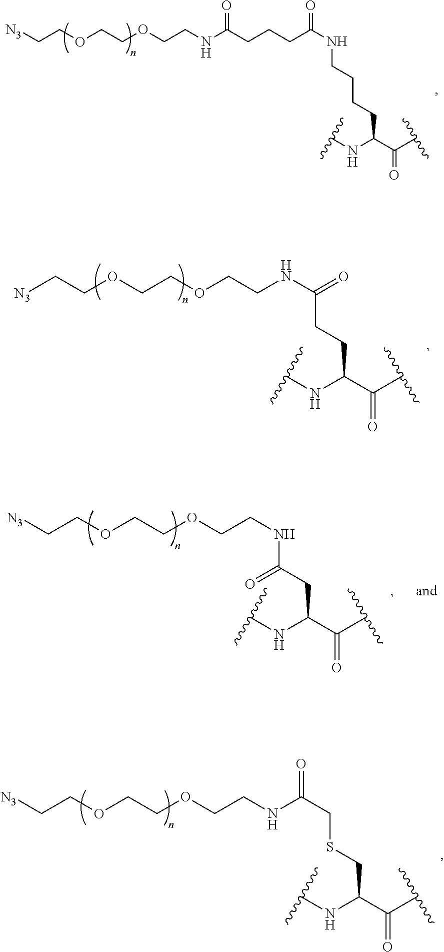

wherein each n is independently an integer from 1-30; and B has a structure

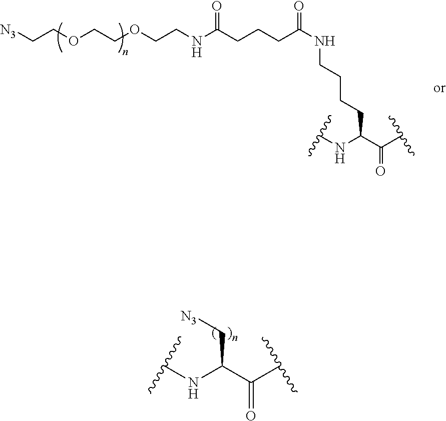

wherein each n is independently an integer from 1-30.

The methods provided herein can also be used to prepare any of the constructs provided in the table below:

### Additional IL-18 Polypeptide

| Composition ID/ | | Composition ID/ | | Composition ID/ | |
|---|---|---|---|---|---|
| Substitutions to SEQ ID NO: 1 | | Substitutions to SEQ ID NO: 1 | | Substitutions to SEQ ID NO: 1 | |
| S69 | E31Hse, T63Hse, | S78 | E6K, E31Hse, K53A, | S88 | E6K, E31Hse, C38A |
| | C68α, S75Hse, | | T63Hse, S75Hse, | | K53A, T63Hse, C68A, |
| | E116Hse | | M86α, E116Hse | | E69α, C76A, S75Hse, |
| | | | | | E116Hse, C127A |
| S70 | E31Hse, T63Hse, | S79 | E6K, E31Hse, K53A, | S89 | E6K, E31Hse, C38A, |
| | E69α, S75Hse, | | T63Hse, S75Hse, | | K53A, T63Hse, C68A, |
| | E116Hse | | D98α, E116Hse | | E69α, C76A, S75Hse, |
| | | | | | E116Hse, C127A |
| S71 | E31Hse, T63Hse, | S81 | E6K, E31Hse, K53A, | S90 | E6K, E31Hse, C38A, |
| | E69α, S75Hse, | | T63Hse, S65A, C68α, | | K53A, T63Hse, C68A, |
| | E116Hse | | S75Hse, E116Hse | | C76A, S75Hse, M86α, |
| | | | | | E116Hse, C127A |
| S72 | E31Hse, T63Hse, | S82 | E6K, E31Hse, K53A, | S91 | E6K, E31Hse, C38A, |
| | S75Hse, M86α, | | T63Hse, S65A, E69α, | | K53A, T63Hse, C68A, |
| | E116Hse | | S75Hse, E116Hse | | C76A, S75Hse, M86α, |
| | | | | | E116Hse, C127A |
| S73 | E31Hse, T63Hse, | S83 | E6K, E31Hse, K53A, | S93 | E31Hse, Q56L, P57Hse, |
| | S75Hse, D98α, | | T63Hse, S65A, E69α, | | C68α, S75Hse, |
| | E116Hse | | S75Hse, E116Hse | | E116Hse |
| S75 | E6K, E31Hse, K53A, | S84 | E6K, E31Hse, K53A, | S94 | E31Hse, Q56L, P57Hse, |
| | T63Hse, C68α, | | T63Hse, S65A, | | E69α, S75Hse, E116Hse |
| | S75Hse, E116Hse | | S75Hse, M86α, | | |
| | | | E116Hse | | |
| S76 | E6K, E31Hse, K53A, | S85 | E6K, E31Hse, K53A, | S95 | E31Hse, Q56L, P57Hse, |
| | T63Hse, E69α, | | T63Hse, S65A, | | K70α, S75Hse, E116Hse |
| | S75Hse, E116Hse | | S75Hse, D98α, | | |
| | | | E116Hse | | |
| S77 | E6K, E31Hse, K53A, | S87 | E6K, E31Hse, C38A, | S96 | E31Hse, Q56L, P57Hse, |
| | T63Hse, E69α, | | K53A, T63Hse, | | S75Hse, M86α E116Hse |
| | S75Hse, E116Hse | | C68α, S75Hse, C76A, | | |
| | | | E116Hse, C127A | | |
| S97 | E31Hse, Q56L, | S101 | E6K, E31Hse, K53A, | S105 | E6K, V11I, E31Hse, |
| | P57Hse, S75Hse, | | Q56L, P57Hse, K70α, | | K53A, Q56L, P57Hse, |
| | D98α, E116Hse | | S75Hse, E116Hse | | T63A, C68α, S75Hse, |
| | | | | | E116Hse |
| S99 | E6K, E31Hse, K53A, | S102 | E6K, E31Hse, K53A, | S107 | E6K, V11I, E31Hse, |
| | Q56L, P57Hse, | | Q56L, P57Hse, | | K53A, Q56L, P57Hse, |
| | C68α, S75Hse, | | S75Hse, M86α | | T63A, K70α, S75Hse, |
| | E116Hse | | E116Hse | | E116Hse |
| S100 | E6K, E31Hse, K53A, | S103 | E6K, E31Hse, K53A, | S108 | E6K, V11I, E31Hse, |
| | Q56L, P57Hse, | | Q56L, P57Hse, | | K53A, Q56L, P57Hse, |
| | E69α, S75Hse, | | S75Hse, D98α, | | T63A, S75Hse, M86α |
| | E116Hse | | E116Hse | | E116Hse |
| S109 | E6K, V11I, E31Hse, | S123 | E6K, E31Hse, I49L, | S136 | E6K, E31Hse, C38A, |
| | K53A, Q56L, | | S50Hse, K53A, C68α, | | I49L, S50Hse, K53A, |
| | P57Hse, T63A, | | S75Hse, Y120F, | | C68A, E69α, S75Hse, |
| | S75Hse, D98α, | | E121Hse | | Y120F, E121Hse, |
| | E116Hse | | | | C127A |
| S111 | E6K, E31Hse, C38A, | S124 | E6K, E31Hse, I49L, | S137 | E6K, E31Hse, C38A, |
| | K53A, Q56L, | | S50Hse, K53A, E69α, | | I49L, S50Hse, K53A, |
| | P57Hse, C68α, | | S75Hse, Y120F, | | C68A, K70α, S75Hse, |
| | S75Hse, C76A, | | E121Hse | | Y120F, E121Hse, |
| | E116Hse, C127A | | | | C127A |
| S112 | E6K, E31Hse, C38A, | S125 | E6K, E31Hse, I49L, | S138 | E6K, E31Hse, C38A, |
| | K53A, Q56L, | | S50Hse, K53A, K70α, | | I49L, S50Hse, K53A, |
| | P57Hse, C68A, | | S75Hse, Y120F, | | C68A, S75Hse, M86α, |
| | E69α, S75Hse, | | E121Hse | | Y120F, E121Hse, |
| | C76A, E116Hse, | | | | C127A |
| | C127A | | | | |
| S113 | E6K, E31Hse, C38A, | S126 | E6K, E31Hse, I49L, | S139 | E6K, E31Hse, C38A, |
| | K53A, Q56L, | | S50Hse, K53A, | | I49L, S50Hse, K53A, |
| | P57Hse, C68A, | | S75Hse, M86α, | | C68α, S75Hse, D98α, |
| | K70α, S75Hse, | | Y120F, E121Hse | | Y120F, E121Hse, |
| | C76A, E116Hse, | | | | C127A |
| | C127A | | | | |
| S114 | E6K, E31Hse, C38S, | S127 | E6K, E31Hse, I49L, | S141 | E31Hse, K67Hse, C68α, |
| | K53A, Q56L, | | S50Hse, K53A, | | S75Hse, E116Hse |
| | P57Hse, C68A, | | S75Hse, D98α, | | |
| | S75Hse, C76A, | | Y120F, E121Hse | | |
| | M86α E116Hse, | | | | |
| | C127A | | | | |
| S115 | E6K, E31Hse, C38S, | S129 | E6K, V11I, E31Hse, | S142 | E31Hse, K67Hse, E69α, |
| | K53A, Q56L, | | I49L, S50Hse, K53A, | | S75Hse, E116Hse |
| | P57Hse, C68A, | | C68α, T63A, S75Hse, | | |
| | S75Hse, C76A, | | Y120F, E121Hse | | |
| | D98α, E116Hse, | | | | |
| | C127A | | | | |
| S117 | E31Hse, I49L, | S130 | E6K, V11I, E31Hse, | S143 | E31Hse, K67Hse, K70α, |
| | S50Hse, C68α, | | I49L, S50Hse, K53A, | | S75Hse, E116Hse |
| | S75Hse, Y120F, | | T63A, E69α, S75Hse, | | |
| | E121Hse | | Y120F, E121Hse | | |
| S118 | E31Hse, I49L, | S131 | E6K, V11I, E31Hse, | S144 | E31Hse, K67Hse, |
| | S50Hse, E69α, | | I49L, S50Hse, K53A, | | S75Hse, M86α E116Hse |
| | S75Hse, Y120F, | | T63A, K70α, S75Hse, | | |
| | E121Hse | | Y120F, E121Hse | | |
| S119 | E31Hse, I49L, | S132 | E6K, V11I, E31Hse, | S145 | E31Hse, K67Hse, |
| | S50Hse, K70α, | | I49L, S50Hse, K53A, | | S75Hse, D98α, E116Hse |
| | S75Hse, Y120F, | | T63A, S75Hse, M86α, | | |
| | E121Hse | | Y120F, E121Hse | | |
| S120 | E31Hse, I49L, | S133 | E6K, V11I, E31Hse, | S147 | E6K, E31Hse, K53A, |
| | S50Hse, S75Hse, | | I49L, S50Hse, K53A, | | K67Hse, C68α, S75Hse, |
| | M86α, Y120F, | | T63A, S75Hse, D98α, | | E116Hse |
| | E121Hse | | Y120F, E121Hse | | |
| S121 | E31Hse, I49L, | S135 | E6K, E31Hse, C38A, | S148 | E6K, E31Hse, K53A, |
| | S50Hse, S75Hse, | | I49L, S50Hse, K53A, | | K67Hse, E69α, S75Hse, |
| | D98α, Y120F, | | C68α, S75Hse, C76A, | | E116Hse |
| | E121Hse | | Y120F, E121Hse, | | |
| | | | C127A | | |
| S150 | E6K, E31Hse, K53A, | S151 | E6K, E31Hse, K53A, | S149 | E6K, E31Hse, K53A, |
| | K67Hse, S75Hse, | | K67Hse, S75Hse, | | K67Hse, K70α, S75Hse, |
| | M86α E116Hse | | D98α, E116Hse | | E116Hse |
| S153 | E6K, V11I, E31Hse, | S178 | E6K, E31Hse, | S164 | E31Hse, M33Nle, |
| | K53A, T63A, | | M33Nle, M51Nle, | | M51Nle, M60Nle, |
| | K67Hse, C68α, | | K53A, M60Nle, | | S75Hse, M86Nle, |
| | S75Hse, E116Hse | | S75Hse, M86Nle, | | M113Nle, E116Hse, |
| | | | M113Nle, E116Hse, | | M150Nle |
| | | | M150Nle | | |
| S154 | E6K, V11I, E31Hse, | S181 | E6K, E31Hse, | S165 | E31Hse, M33Nle, C38S, |
| | K53A, T63A, | | M33Nle, M51Nle, | | M51Nle, M60Nle, C68S, |
| | K67Hse, E69α, | | K53A, M60Nle, | | S75Hse, C76S, M86Nle, |
| | S75Hse, E116Hse | | T63Hse, M86Nle, | | M113Nle, E116Hse, |
| | | | M113Nle, E116Hse, | | C127S, M150Nle |
| | | | M150Nle | | |
| S155 | E6K, V11I, E31Hse, | S182 | E6K, E31Hse, | S166 | E31Hse M33Nle, |
| | K53A, T63A, | | M33Nle, C38S, | | M51Nle, M60Nle, |
| | K67Hse, K70α, | | M51Nle, K53A, | | T63Hse, M86Nle, |
| | S75Hse, E116Hse | | M60Nle, T63Hse, | | M113Nle, E116Hse, |
| | | | C68S, C76S, M86Nle, | | M150Nle |
| | | | M113Nle, E116Hse, | | |
| | | | C127S, M150Nle | | |
| S156 | E6K, V11I, E31Hse, | S185 | E31Hse, M33Nle, | S167 | E6K, E31Hse, M33Nle, |
| | K53A, T63A, | | M51Nle, M60Nle, | | M51Nle, K53A, |
| | K67Hse, S75Hse, | | K70PEG, S75Hse, | | M60Nle, T63Hse, |
| | M86α E116Hse | | M86Nle, M113Nle, | | K70PEG, M86Nle, |
| | | | E116Hse, M150Nle | | M113Nle, E116Hse, |
| | | | | | M150Nle |
| S157 | E6K, V11I, E31Hse, | S188 | E31Hse, M33Nle, | S168 | E31Hse, M33Nle, C38S, |
| | K53A, T63A, | | M51Nle, M60Nle, | | M51Nle, M60Nle, |
| | K67Hse, S75Hse, | | T63Hse, K70PEG, | | T63Hse, C68S, C76S, |
| | D98α, E116Hse | | M86Nle, M113Nle, | | M86Nle, M113Nle, |
| | | | E116Hse, M150Nle | | E116Hse, C127S, |
| | | | | | M150Nle |
| S159 | E6K, E31Hse, C38A, | S189 | E31Hse, M33Nle, | S169 | E6K, E31Hse, M33Nle, |
| | K53A, K67Hse, | | C38S, M51Nle, | | C38S, M51Nle, K53A, |
| | C68α, S75Hse, | | M60Nle, T63Hse, | | M60Nle, T63Hse, C68S, |
| | C76A, E116Hse, | | C68S, K70PEG, | | K70PEG, C76S, |
| | C127A | | C76S, M86Nle, | | M86Nle, M113Nle, |
| | | | M113Nle, E116Hse, | | E116Hse, C127S, |
| | | | C127S, M150Nle | | M150Nle |
| S160 | E6K, E31Hse, C38A, | S194 | E6K, E31Hse, | S106 | E6K, V11I, E31Hse, |
| | K53A, K67Hse, | | M33Nle, M51Nle, | | K53A, Q56L, P57Hse, |
| | C68A, E69α, | | K53A, M60Nle, | | T63A, E69α, S75Hse, |
| | S75Hse, C76A, | | T63A, S75Hse, | | E116Hse |
| | E116Hse, C127A | | M86Nle, M113Nle, | | |
| | | | E116Hse, M150Nle | | |
| S161 | E6K, E31Hse, C38A, | S195 | E6K, E31Hse, | S163 | E6K, E31Hse, C38S, |
| | K53A, K67Hse, | | M33Nle, C38S, | | K53A, K67Hse, C68A, |
| | C68A, K70α, | | M51Nle, K53A, | | S75Hse, C76A, D98α, |
| | S75Hse, C76A, | | M60Nle, T63A, C68S, | | E116Hse, C127A |
| | E116Hse, C127A | | S75Hse, C76S, | | |
| | | | M86Nle, M113Nle, | | |
| | | | E116Hse, C127S, | | |
| | | | M150Nle | | |
| S162 | E6K, E31Hse, C38S, | S198 | E6K, E31Hse, | S199 | E6K, E31Hse, M33Nle, |
| | K53A, K67Hse, | | M33Nle, M51Nle, | | M51Nle, K53A, |
| | C68A, S75Hse, | | K53A, M60Nle, | | M60Nle, T63Hse, |
| | C76A, M86α | | T63Hse, C68PEG, | | E69PEG, M86Nle, |
| | E116Hse, C127A | | M86Nle, M113Nle, | | M113Nle, E116Hse, |
| | | | E116Hse, M150Nle | | M150Nle |

wherein the indicated substitutions are relative to SEQ ID NO: 1.

FIG. 12 shows an exemplary Analytical SEC-HPLC chromatogram of purified SEQ ID NO: 207 (detection: 220 nm).

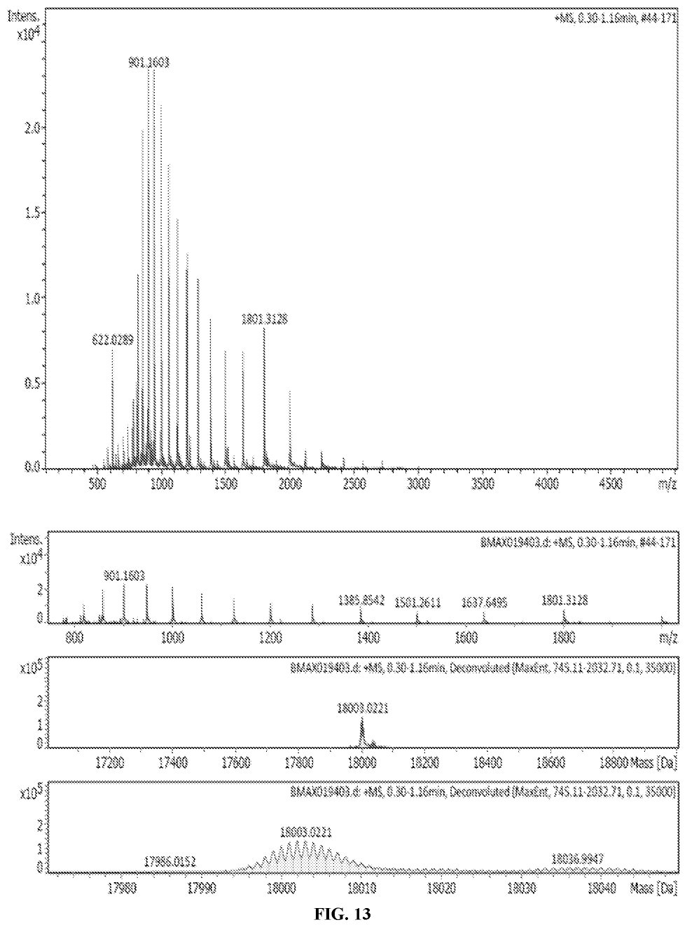

FIG. 13 shows an exemplary ESI-Q-TOF-HRMS spectra of purified SEQ ID NO: 207.

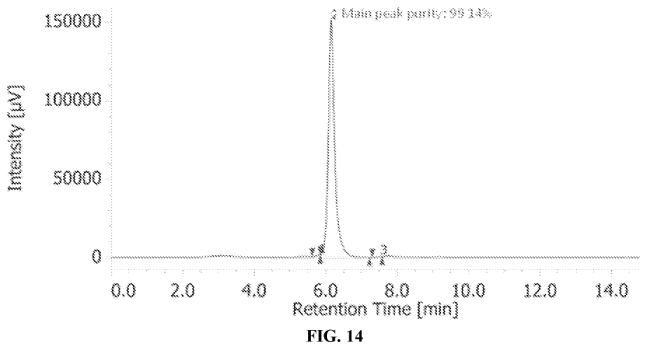

FIG. 14 shows an exemplary Analytical SEC-HPLC chromatogram of purified SEQ ID NO: 239 (detection: 220 nm).

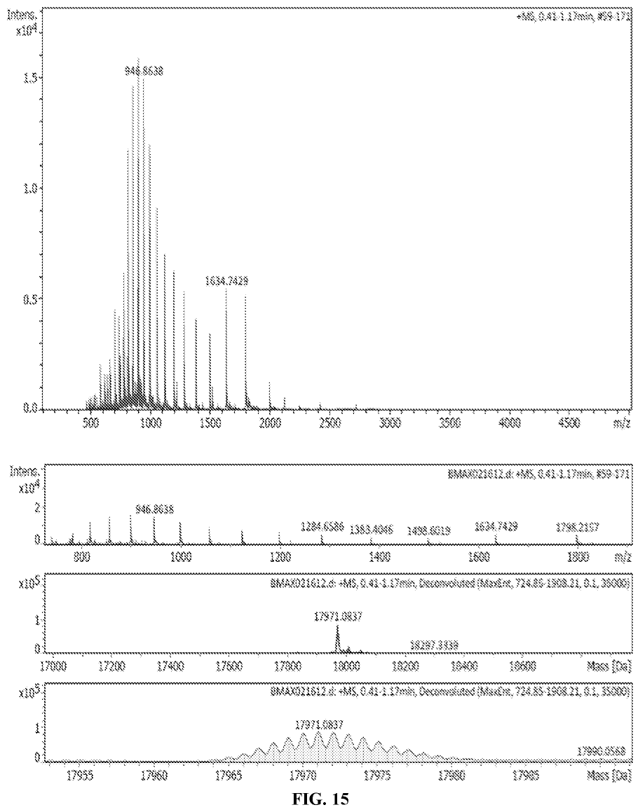

FIG. 15 shows an exemplary ESI-Q-TOF-HRMS spectra of purified SEQ ID NO: 239.

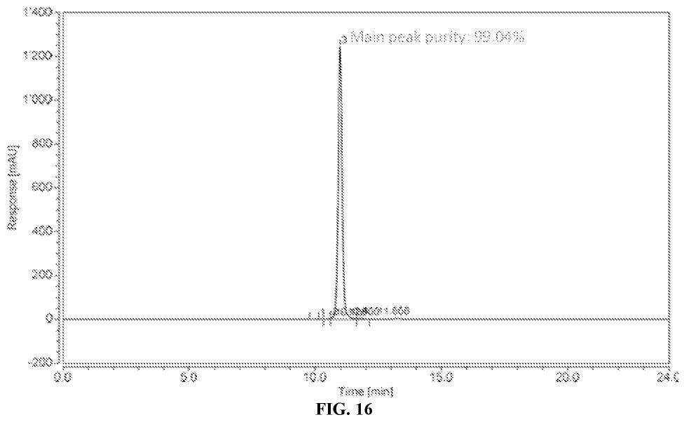

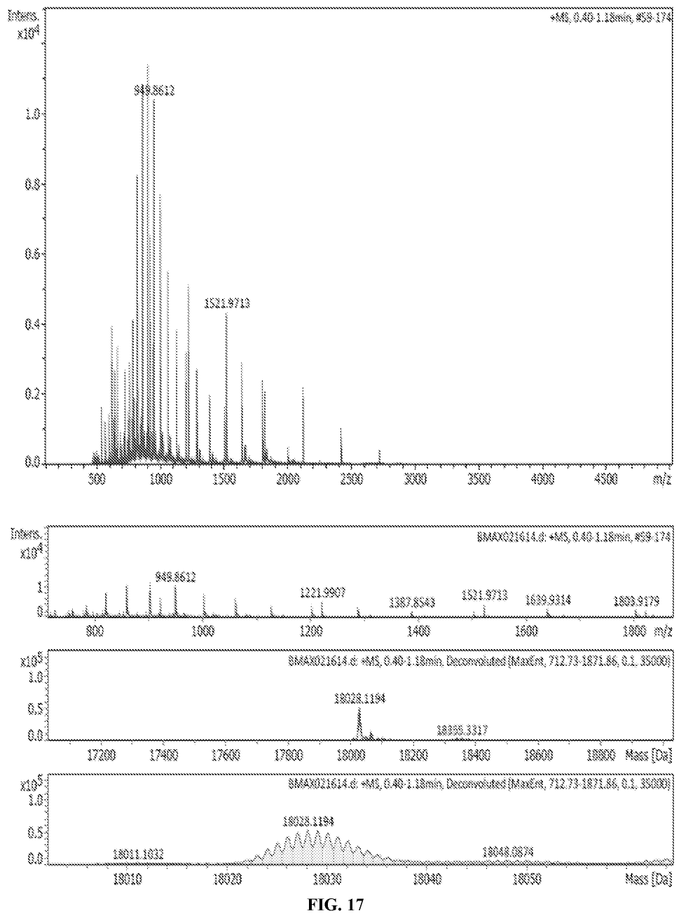

FIG. 16 shows an exemplary Analytical SEC-HPLC chromatogram of purified SEQ ID NO: 244 (detection: 220 nm).

FIG. 17 shows an exemplary ESI-Q-TOF-HRMS spectra of purified SEQ ID NO: 244.

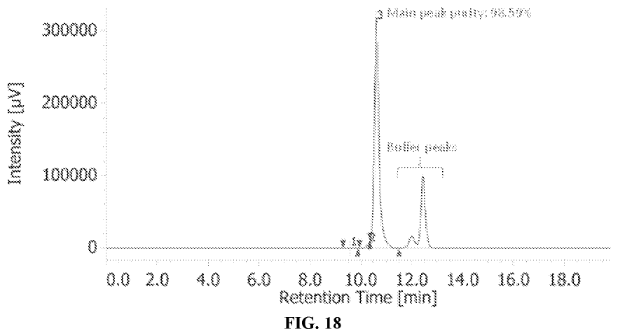

FIG. 18 shows an exemplary Analytical SEC-HPLC chromatogram of purified SEQ ID NO: 242 (detection: 220 nm).

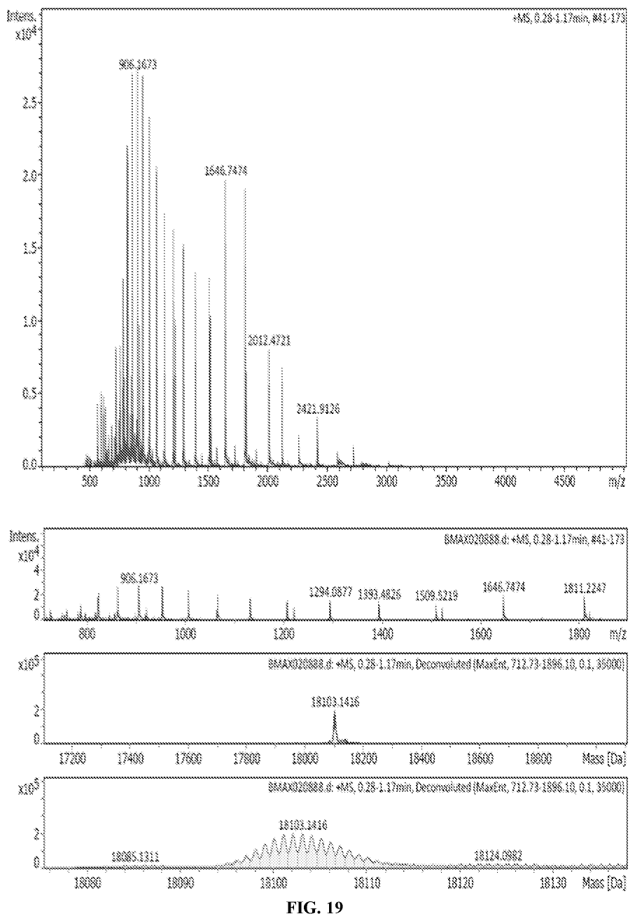

FIG. 19 shows an exemplary ESI-Q-TOF-HRMS spectra of purified SEQ ID NO: 242.

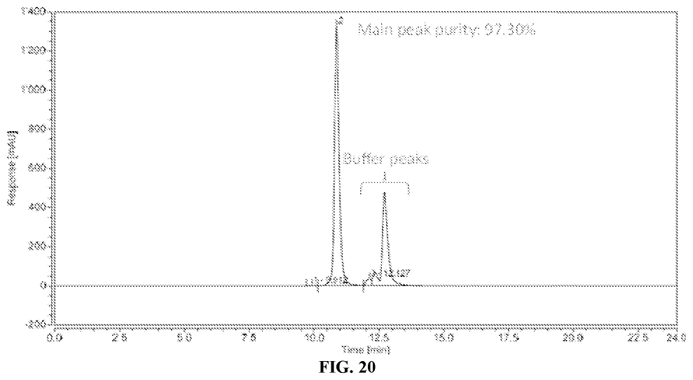

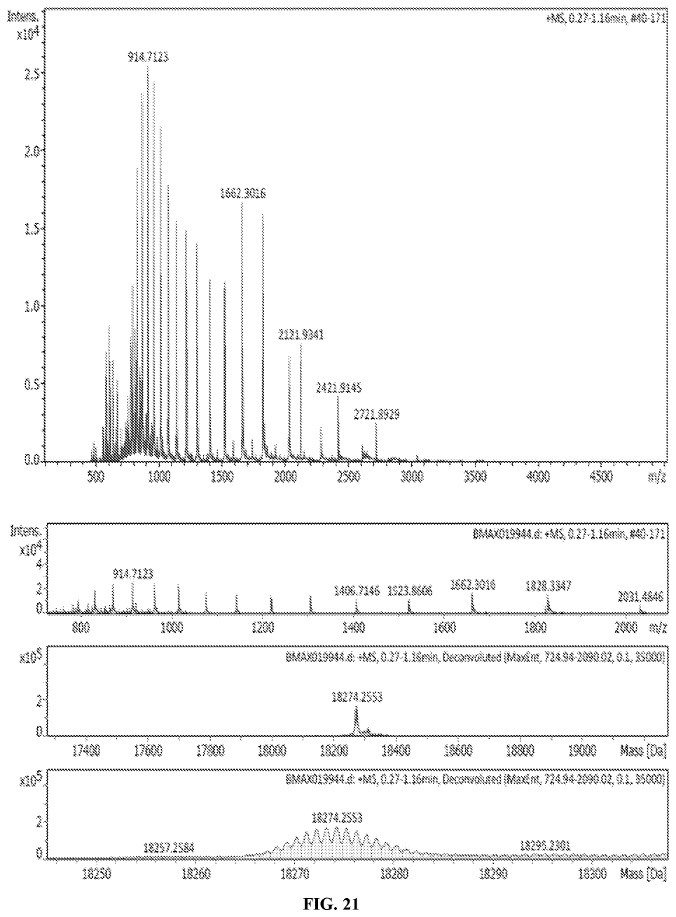

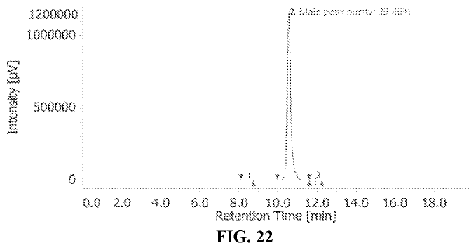

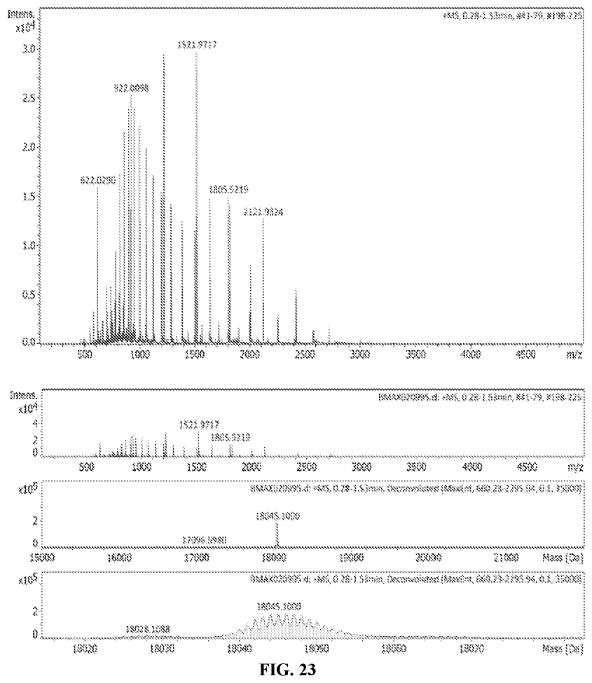

FIG. 20 shows an exemplary Analytical SEC-HPLC chromatogram of purified SEQ ID NO: 241 (detection: 220 nm).

### Example 5—Conjugation of Modified IL-18 Polypeptides

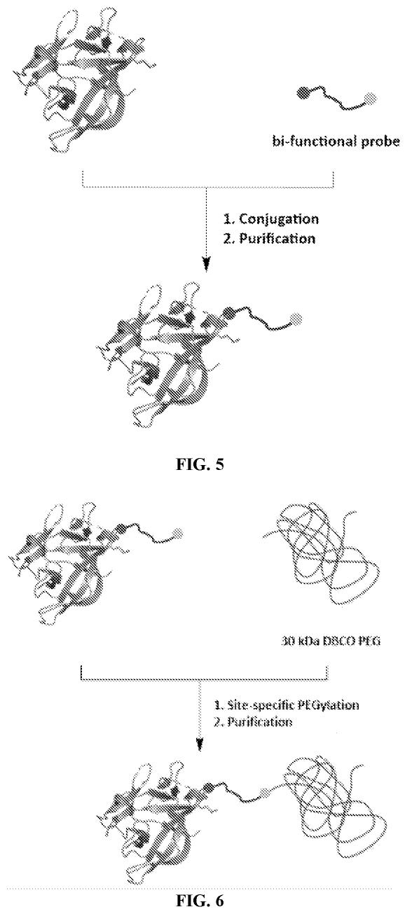

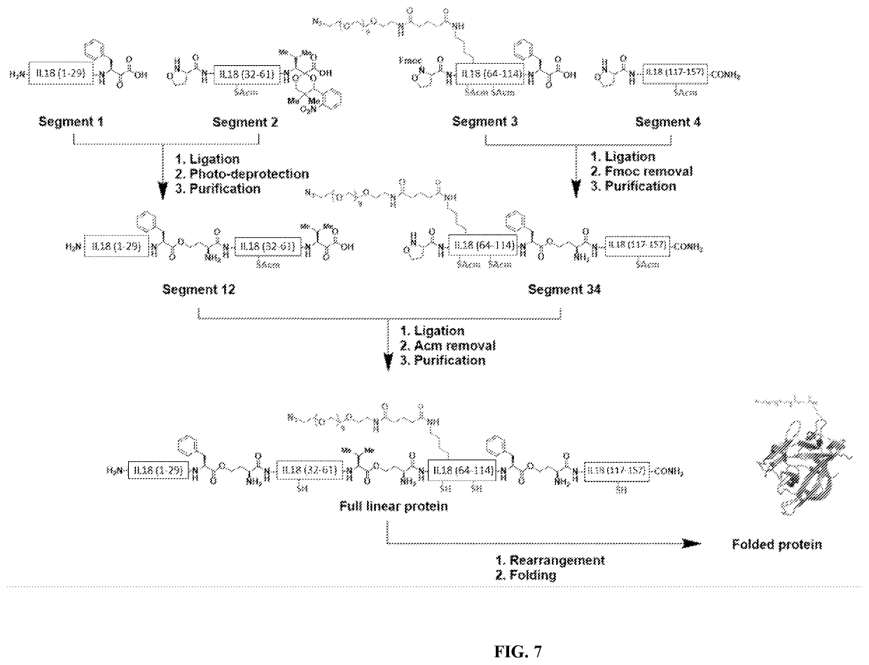

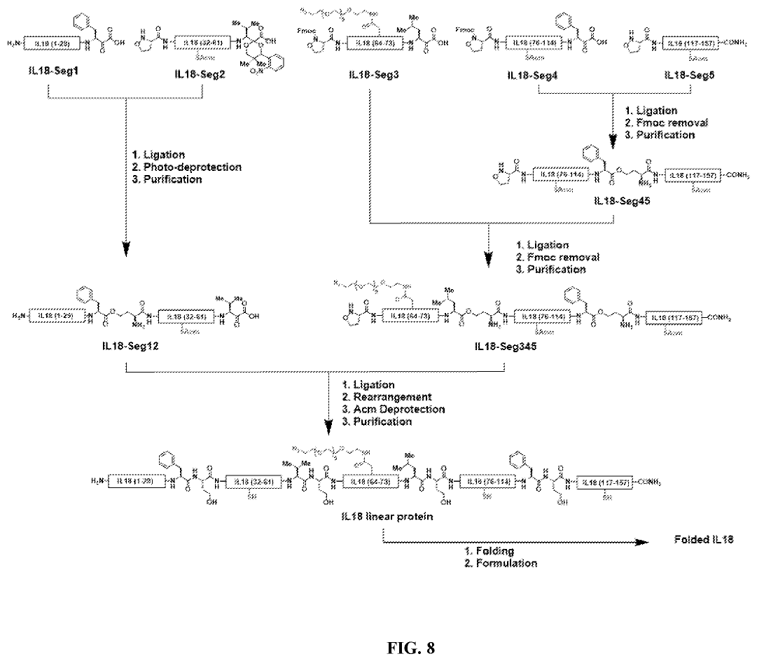

A modified IL-18 polypeptide is conjugated to a PEG functionality. In some cases, the PEG is attached via a bifunctional linker which first attaches to a desired residue of the modified IL-18 polypeptide (e.g., E85C or suitable naturally occurring cysteine or a cysteine residue which has been incorporated at a desired site, such as residue 86 or 98). Once attached to the IL-18 polypeptide, the second functionality of the bifunctional linker is used to attach the PEG moiety. An exemplary schematic of such a process is shown in FIGS. 5 and 6. An exemplary protocol on a recombinant IL-18 variant provided herein is described below.

Protocol for Conjugation with Bromoacetamido-PEG5-azide linker
Protein is stored in PBS with 10% glycerol at pH 7.4 at −70 C and thawed on ice. Stock protein solution is diluted as necessary with PBS pH 7.4. Bromoacetamido-PEG5-azide is dissolved in water at a concentration of 40 mM. Protein stock solution at 0.2 mg/mL and the linker stock solution at (40 mM, 100 equivalents) is mixed together at 100 equivalents and the reaction is gently mixed. The reaction is allowed to proceed at 20° C. for 3h with very gentle mixing. Purification is performed immediately after 3 h to stop the reaction. Protein is separated from excess linker on ÄKTA with HiTrap Desalting FF columns. The column buffer is: 25 mM Tris, pH 7.4. Fractions are analyzed with RP-HPLC and fractions containing the protein are pooled and stored at 4° C. Concentration is assessed with nanodrop based on the theoretical MW and extinction coefficient (5960 M−1 cm-1).
   PEGylation reaction
Ten equivalents of 30 kDa DBCO-mPEG is added directly to purified protein solution. The reaction is incubated overnight at 20° C. with protection from light and with very slow mixing. After completion, the PEGylated protein is purified via anion exchange chromatography on an ÄKTA FPLC using a 5 ml HiTrap Capto Q ImpRes column with buffer A: 25 mM Tris, pH 7.4; and Buffer B: 25 mM Tris, 350 mM NaCl, pH 7.4. Protein is eluted from the column using a two part linear gradient: Part 1 is 0-10 CV, 0-50% buffer B; then part 2 is 10-15 CV, 50-100% buffer B. Using this protocol, the PEGylated IL-18 protein readily separates from excess PEG and pure PEGylated protein is obtained.

Characterization—The purity and identity of the recombinant protein from commercial source and the conjugated protein is confirmed by aSEC, HPLC and MALDI-TOF MS.

### Example 6—PEGylation of Modified IL-18 Polypeptides

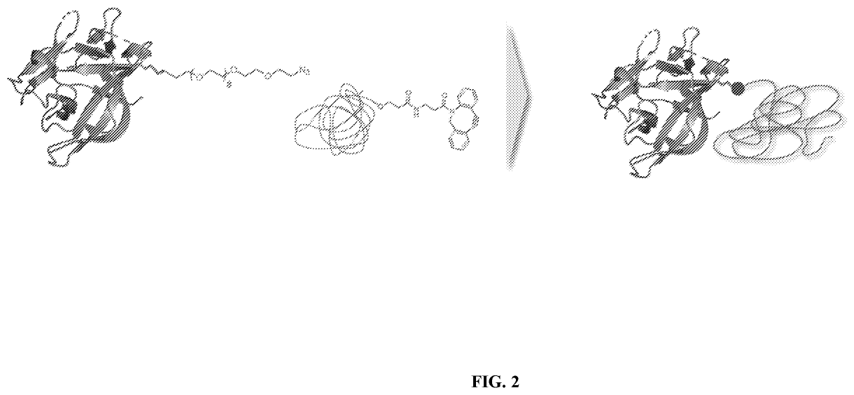

After conjugation of the bifunctional linker as described in Example 19 and as shown in FIG. 5, the modified IL-18 polypeptide can be covalently linked with a PEG group. An exemplary schematic of this process is shown in FIG. 6. An exemplary protocol of the conjugation reaction between a PEG and a suitably activated IL-18 polypeptide is provided below. Additionally, the protocol below can be used to covalently link a desired PEG group to a modified IL-18 polypeptide which incorporates a conjugation handle directly during the preparation of the modified IL-18 polypeptide (e.g., during the synthesis of a synthetic IL-18 polypeptide). An exemplary schematic of such a process is shown in FIG. 2.

Conjugation—Recombinant modified IL-18 polypeptide of SEQ ID NO: 71 is stored at −80° C. in PBS (pH 7.4) containing 75 mM NaCl and 5% (v/v) glycerol. Prior to PEGylation reaction, the sample is thawed on ice yielding a clear solution. 200 μL of the protein solution (0.4 mg/mL) are mixed with 2.0 mg of 30 kDa DBCO-polyethylene glycol polymer. It is let to react overnight at 20° C.

The progress of the synthesis is monitored by reverse-phase HPLC using a gradient of 5 to 30% (2.5 min) and 30 to 75% (7.5 min) CH₃CN with 0.1% TFA (v/v) on a Aeris WIDEPORE C4 200 Å column (3.6 μm, 150×4.6 mm) at a flow rate of 1 mL/min at 40° C. and by MALDI-TOF MS.

Purification—To remove the excess of PEG, the reaction mixture is diluted with Tris buffer (25 mM, pH 7.4) and flowed through a Hi-Trap-Q-FF column using 25 mM Tris (pH 7.4) as the buffer. The column is eluted with a linear gradient of 0-0.35 M NaCl in the same buffer. The fractions containing the target protein are gathered, buffer exchanged (25 mM Tris, pH 7.4, 75 mM NaCl, 5% glycerol) and concentrated at 0.04 mg/mL. The concentration of purified protein is determined by BCA protein assay. The protein solution is kept at −80° C.

Characterization—The purity and identity of the conjugated protein is confirmed by HPLC and MALDI-TOF MS.

### Example 7—Characterization of Modified IL-18 Polypeptides

Modified IL-18 polypeptides provided herein are subject to a series of analytical experiments to characterize the compositions. The modified IL-18 polypeptides are analyzed by HPLC to determine the degree of uniformity in the compositions. The modified IL-18 polypeptide compositions are also analyzed by MALDI-MS to determine the MW and distribution of molecular weights of the compositions. The modified IL-18 polypeptide compositions are further analyzed by circular dichroism to compare the folding of the modified IL-18 polypeptide compositions compared to wild type IL-18.

### Example 8—Formulation of Modified IL-18 Polypeptides

Lyophilized modified IL-18 polypeptides are suspended in a solution comprising PBS buffer (pH 7.4) with 50 mg/mL mannitol.

### Example 9—IL-18 SPR Measurements

The interaction of the wild type and of modified IL-18 polypeptides with human IL-18 receptor subunits are measured with Surface Plasmon Resonance (SPR) technology. Anti-human IgG antibodies are bound by amine coupling onto a CM5 chip to capture 6 μg/mL of Fc fused human IL-18Rα, 6 μg/mL of Fc fused human IL-18Rβ, or 2 μg/mL of Fc fused human IL-18BP isoform a (IL-18BPa) for 30 min before capture. In other settings, 6 μg/mL of alpha and beta IL-18 receptors are mixed and pre-incubated for 30 min before capture of the alpha/beta heterodimer IL-18 receptor.

The kinetic binding of the IL-18 analytes are measured with a Biacore 8K instrument in two fold serial dilutions starting at 1 μM down to 0.98 nM. Regeneration of the surface back to amine coupled anti IgG antibody is done after every concentration of analyte. To measure the protein association to the receptors, the samples are injected with a flow rate of 50 μL/min for 60 s, followed by 300 s buffer only to detect the dissociation. The used running buffer is 1×PBS with 0.05% Tween20. The relative response units (RU, Y-axis) are plotted against time (s, X-axis) and analyzed in a kinetic 1:1 binding model for the monomer receptor binding and for the binding to the IL-18BP. A kinetic heterogenous ligand fit model is applied for the alpha/beta heterodimer binding.

### Example 10—IL-18BP Binding alphaLISA Assay

A human IL-18BP AlphaLISA Assay Kit is used to determine the binding affinity of each IL-18 variant for IL-18BP, which detected the presence of free form IL-18BP.

Sixteen three fold serial dilutions of IL-18 analytes are prepared in alpha-MEM medium supplemented with 20% FCS, Glutamax™, and 25 μM β-mercaptoethanol in the presence of 5 ng/mL of His-tagged human IL-18BP. Final IL-18 analytes concentration range from 2778 nM to 0.2 pM.

After 1 hr incubation at room temperature, free IL-18BP levels are measured using a Human IFNγ AlphaLISA Assay Kit. In a 384 well OPTIplate, 5 μL of 5× Anti-IL-18BP acceptor beads are added to 7.5 μL of an IL-18/IL-8BP mix. After 30 min incubation at room temperature with shaking, 5 μL of biotinylated Anti-IL-18B3P antibodies are added to each well. The plate is incubated further for 1 hr at room temperature. Under subdued light, 12.5 μL of 2× streptavidin (SA) donor beads are pipetted into each well, and the wells are incubated with shaking for an additional 30 min at room temperature. The AlphaLisa signal is then measured on an Enspire plate reader with 680 and 615 nm as excitation and emission wavelengths, respectively. The dissociation constant (K_{D}) is calculated based on a variable slope, four parameter analysis using GraphPad PRISM software.

### Example 11—Binding of IL-18 Variants to IL-18Rα Monomer

Table 5 shows results of the dissociation constants (K_{D}) observed for the IL-18 variants described to IL-18Rα using the protocol as set forth in Example 9.

TABLE 5
Binding of IL-18Rα monomer

| SEQ ID | | Kₒₙ | K_{off} | K_{D} |
|---|---|---|---|---|
| NO: | Sequence modifications | (1/Ms) | (1/s) | (nM) |
| 1 | Native sequence | 669000 | 0.00484 | 7.24 |
| 57 | E6K, C38S, K53A | 208000 | 0.0151 | 72.4 |
| 59 | E6K, C38A, K53A | 1.77E+06 | 1.65E−02 | 17.8 |
| 30 | E6K, V11I, C38A, K53A, | 1.77E+06 | 5.36E−02 | 30.3 |
| | T63A, C76A, C127A | | | |
| 62 | E6K, C38A, K53A, C127A | 6.64E+05 | 4.24E−02 | 63.9 |
| 61 | E6K, C38A, K53A, C76A | 2.22E+06 | 6.74E−02 | 30.4 |
| 60 | E6K, C38Q, K53A | 1.03E+06 | 1.02E−02 | 9.85 |
| 63 | E6K, C38A, K53A, C76A, | 2.31E+07 | 9.76E−01 | 42.2 |
| | C127A | | | |

### Example 12—Binding of IL-18 Variants to IL-18Rα/β Heterodimer

Table 6 shows results of the dissociation constants (K_{D}) observed for the IL-18 variants described to IL-18Rα/β heterodimer using the experimental as described in Example 9.

TABLE 6
Binding of IL-18Rα/β heterodimer

| SEQ ID | | Kₒₙ | K_{off} | K_{D} |
|---|---|---|---|---|
| NO: | Sequence modifications | (1/Ms) | (1/s) | (nM) |
| 1 | Native sequence | 5800000 | 0.010900 | 1.88 |
| 57 | E6K, C38S, K53A | 279000 | 0.0008 | 2.92 |
| 59 | E6K, C38A, K53A | 1.24E+06 | 8.15E−04 | 0.66 |
| 30 | E6K, V11I, C38A, K53A, | 8.66E+05 | 3.11E−04 | 0.49 |
| | T63A, C76A, C127A | | | |
| 62 | E6K, C38A, K53A, C127A | 1.21E+06 | 1.49E−03 | 1.24 |
| 61 | E6K, C38A, K53A, C76A | 1.09E+06 | 1.10E−03 | 1.01 |
| 60 | E6K, C38Q, K53A | 1.21E+06 | 5.89E−04 | 0.490 |
| 63 | E6K, C38A, K53A, C76A, | 1.15E+06 | 4.67E−04 | 0.41 |
| | C127A | | | |

### Example 13—Binding Assay to IL-18BP Monomer

Table 7 shows results of the dissociation constants (K_{D}) observed for the IL-18 variants described to IL-18BP using an analogous protocol to that described in Example 9.

TABLE 7
Binding of IL-18BP monomer determined by SPR

| SEQ | | Kₒₙ | K_{off} | K_{D} |
|---|---|---|---|---|
| ID NO: | Sequence modifications | (1/Ms) | (1/s) | (nM) |
| 1 | Native sequence | 1.49E+06 | 3.83E−04 | 0.270 |
| 57 | E6K, C38S, K53A | 1140000 | 0.0031 | 2.72 |
| 59 | E6K, C38A, K53A | 1.47E+06 | 3.01E−03 | 2.05 |
| 30 | E6K, V11I, C38A, K53A, | 1.05E+06 | 3.45E−03 | 3.05 |
| | T63A, C76A, C127A | | | |
| 62 | E6K, C38A, K53A, C127A | 1.61E+06 | 3.15E−03 | 1.96 |
| 61 | E6K, C38A, K53A, C76A | 1.74E+06 | 3.60E−03 | 2.1 |
| 60 | E6K, C38Q, K53A | 1.87E+06 | 3.08E−03 | 1.700 |
| 63 | E6K, C38A, K53A, C76A, | 2.82E+06 | 4.24E−03 | 1.5 |
| | C127A | | | |

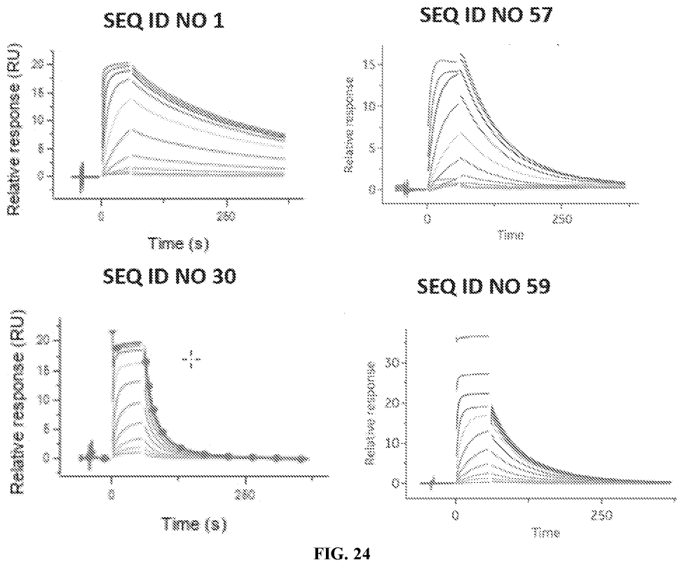

FIG. 24 shows plots measuring the binding activity of wild type IL-18 and of modified IL-18 polypeptides to human IL-18 receptor alpha (CD218a) in surface plasmon resonance experiments, where the x-axis is time, and the y-axis is relative response. The modified IL-18 polypeptides tested in these experiments are native IL-18 wild-type (SEQ ID NO: 01), SEQ ID NO: 57, SEQ ID NO: 59, and SEQ ID NO: 30.

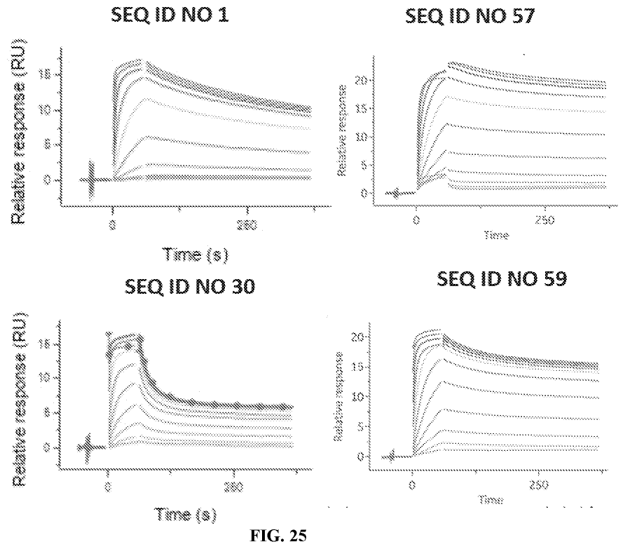

FIG. 25 shows plots measuring the binding activity of wild type IL-18 and of modified IL-18 polypeptides to heterodimeric human IL-18 receptor alpha (CD218a) and IL-18R accessory protein (IL-18RAP/CDw218b) in surface plasmon resonance experiments, where the x-axis is time, and the y-axis is relative response. The modified IL-18 polypeptides tested in these experiments are native IL-18 wild-type (SEQ ID NO: 01), SEQ ID NO: 57, SEQ ID NO: 59, and SEQ ID NO: 30.

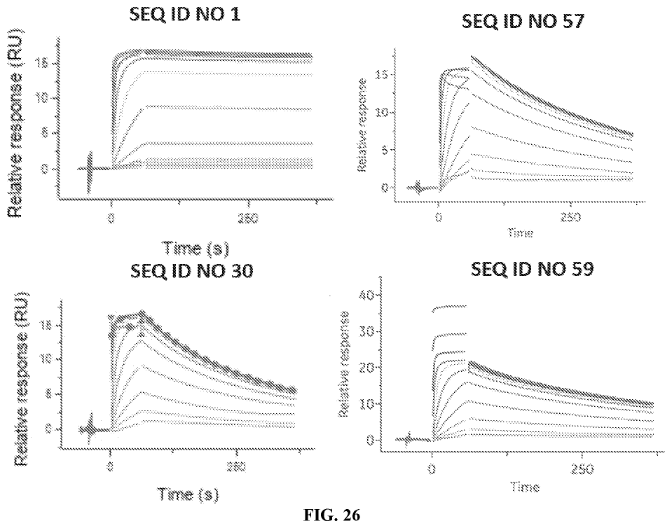

FIG. 26 shows plots measuring the binding activity of wild type IL-18 and of modified IL-18 polypeptides to human IL-18 binding protein (IL-18BP) in surface plasmon resonance experiments, where the x-axis is time, and the y-axis is relative response. The modified IL-18 polypeptides tested in these experiments are native IL-18 wild-type (SEQ ID NO: 01), SEQ ID NO: 57, SEQ ID NO: 59, and SEQ ID NO: 30.

Table 8 shows results of the dissociation constants (K_{D}) observed for the IL-18 variants described to IL-18BP as measured using the protocol described in Example 10.

TABLE 8
Binding of IL-18BP monomer determined by alphaLISA

| SEQ ID | | KD |
|---|---|---|
| NO: | Sequence modifications | (nM) |
| 1 | Native Sequence | 0 . . . 414 |
| 57 | E6K, K53A, C38S | 48.4 |
| 8 | E6K, V11I, C38A, K53A, T63A, C68A, C76A, | 61.055 |
| | T95C, C127A | |
| 11 | E6K, V11I, C38A, K53A, C76A, C127A | 36.800 |
| 6 + PEG | E6K, V11I, C38A, K53A, T63A, C68A, C76A, | 20.180 |
| | C127A, D98C-30kPEG | |
| 6 | E6K, V11I, C38A, K53A, T63A, C68A, C76A, | 630.900 |
| | C127A, D98C | |
| 5 + PEG | E6K, V11I, C38A, K53A, T63A, C68A, C76A, | 31.410 |
| | C127A, M86C-30kPEG | |
| 5 | E6K, V11I, C38A, K53A, T63A, C68A, C76A, | 180.680 |
| | C127A, M86C | |
| 9 + PEG | E6K, C38A, K53A, C68A, D98C-30kPEG | 150.600 |
| 9 | E6K, C38A, K53A, C68A, D98C | 82.650 |
| 4 + PEG | E6K, C38A, K53A, C68A, M86C-30kPEG | 128.600 |
| 4 | E6K, C38A, K53A, C68A, M86C | 55.600 |
| 28 | E6K, C38A, K53A, T63A, C76A, C127A | 44.410 |
| 30 | E6K, V11I, C38A, K53A, T63A, C76A, C127A | 24.600 |
| 62 | E6K, C38A, K53A, C127A | 69.620 |
| 60 | E6K, C38Q, K53A | 24.800 |
| 59 | E6K, C38A, K53A | 35.950 |
| 63 | E6K, C38A, K53A, C76A, C127A | 20.257 |
| 204 | V11I, C38A, K53A, C76A, C127A | 8.86 |
| 205 | V11I, C38A, K53A, T63A, C76A, C127A | 0.66 |
| 206 | V11I, C38A, K53A, S55A, C76A, C127A | 9.74 |
| 207 | V11I, C38A, M51G, K53A, C76A, C127A | 373.30 |
| 208 | V11I, C38A, K53A, D54A, C76A, C127A | 25.77 |
| 209 | F2A, V11I, C38A, K53A, C76A, C127A | 57.21 |
| 210 | V11I, E31A, C38A, K53A, C76A, C127A | 0.64 |
| 211 | V11I, T34A, C38A, K53A, C76A, C127A | 1.24 |
| 212 | V11I, D35A, C38A, K53A, C76A, C127A | 2.88 |
| 213 | V11I, S36A, C38A, K53A, C76A, C127A | 1.12 |
| 214 | V11I, D37A, C38A, K53A, C76A, C127A | 4.55 |
| 215 | V11I, E31A, D37A, C38A, K53A, C76A, C127A | 2.12 |
| 216 | V11I, C38A, D40A, K53A, C76A, C127A | 0.74 |
| 217 | V11I, C38A, N41A, K53A, C76A, C127A | 18.47 |
| 218 | V11I, C38A, K53A, C76A, C127A, D132A | 13.70 |
| 219 | V11I, C38A, K53A, C76A, G108A, C127A | 1.24 |
| 220 | V11I, C38A, K53A, C76A, H109A, C127A | 0.55 |
| 221 | V11I, C38A, K53A, C76A, D110A, C127A | 0.71 |
| 222 | K8R, V11I, C38A, C76A, Q103E, C127A | 0.06 |
| 223 | K8E, V11I, C38A, C76A, Q103R, C127A | 0.85 |
| 224 | V11I, C38A, C76A, Q103K, C127A | 0.05 |
| 225 | V11I, C38A, S55H, C76A, C127A | 0.08 |
| 226 | V11I, C38A, S55R, C76A, C127A | 0.15 |
| 227 | V11I, C38A, S55T, C76A, C127A | 0.02 |
| 228 | V11I, C38A, C76A, S105I, C127A | 0.04 |
| 229 | V11I, C38A, C76A, S105K, C127A | 0.05 |
| 230 | K8L, E6K, V11I, C38A, K53A, T63A, C76A, | 0.14 |
| | C127A | |
| 231 | E6K, V11I, C38A, I49E, K53A, T63A, C76A, | |
| | C127A | |
| 232 | E6K, V11I, C38A, I49M, K53A, T63A, C76A, | 25.84 |
| | C127A | |
| 233 | E6K, V11I, C38A, I49R, K53A, T63A, C76A, | >2800 |
| | C127A | |
| 234 | E6K, V11I, C38A, K53A, T63A, C76A, Q103R, | >2800 |
| | C127A | |
| 235 | E6K, K8E, V11I, C38A, K53A, T63A, C76A, | >2800 |
| | Q103R, C127A | |
| 236 | E6K, V11I, C38A, K53A, T63A, C76A, C127A, | |
| | V153R | |
| 237 | E6K, V11I, C38A, K53A, T63A, C76A, C127A, | >2800 |
| | V153E | |
| 238 | E6K, V11I, C38A, K53A, T63A, C76A, C127A, | >2800 |
| | V153Y | |
| 239 | E6K, V11I, C38A, M51G, K53A, T63A, C76A, | >2800 |
| | C127A | |
| 240 | E6R, V11I, C38A, K53A, T63A, C76A, C127A | 5.46 |
| 241 | N-terminal 4xG, E6K, V11I, C38A, K53A, | >2800 |
| | T63A, C76A, C127A | |
| 242 | N-terminal G, E6K, V11I, C38A, K53A, T63A, | >2800 |
| | C76A, C127A | |
| 243 | Y1M, E6K, V11I, C38A, K53A, T63A, C76A, | 2.25 |
| | C127A | |
| 244 | N-terminal G, E6K, V11I, C38A, M51G, K53A, | >2800 |
| | T63A, C76A, C127A | |

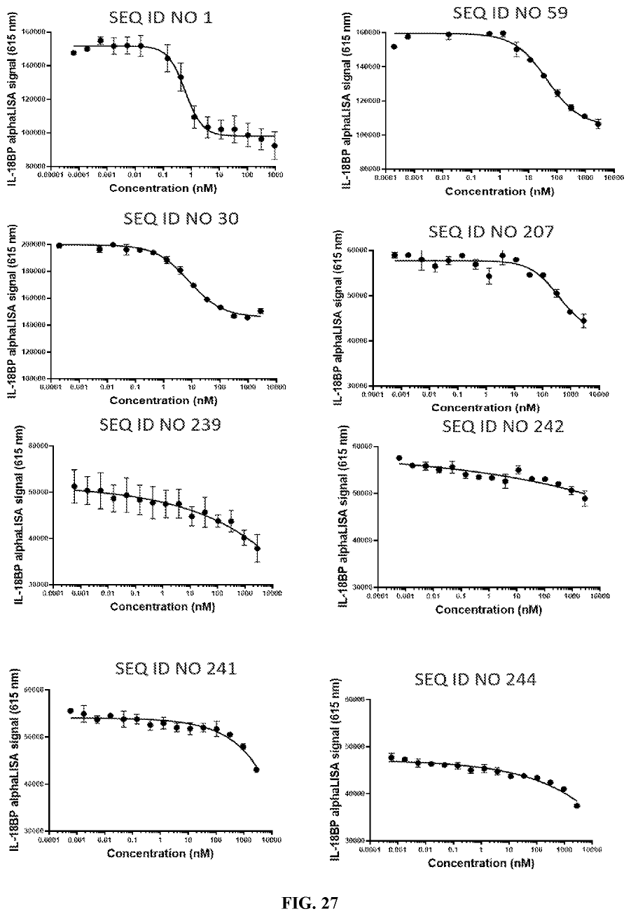

FIG. 27 shows plots measuring the ability of wild type IL-18 and of modified IL-18 polypeptides to bind to the human IL-18 Binding Protein (IL-18BP). The figure shows mean free IL-18BP AlphaLISA signal on the y-axis, and dosage of of wild type IL-18 and of modified IL-18 polypeptides on the x-axis. The unconjugated IL-18 variants are native IL-18 wild-type (SEQ ID NO: 01), SEQ ID NO: 57, SEQ ID NO: 59, SEQ ID NO: 30, SEQ ID NO: 207, SEQ ID NO: 239, SEQ ID NO: 241, SEQ ID NO: 242, and SEQ ID NO: 244.

### Example 14—IFNγ Induction NK-92 Cellular Assay

The ability of IL-18 polypeptides provided herein are assessed for ability to induce IFNγ in a cellular assay according to the protocol below.

The NK cell line NK-92 derived from a patient with lymphoma (ATCC® CRL-2407™) is cultured in aMEM medium supplemented with 20% FCS, Glutamax™, 25 μM B-mercaptoethanol, and 100 IU/mL of recombinant human IL-2.

On the day of experiment, cells are harvested and washed with aMEM medium without IL-2 and containing 1 ng/mL of recombinant human IL-12. After counting, cells are seeded at 100,000 cells/well in a 384 well titer plate and incubated at 37° C./5% CO₂. Sixteen 4-fold serial dilutions of IL-18 analytes are prepared in aMEM medium, and 1 ng/mL of IL-12 were added to the NK-92 cells. Final IL-18 analyte concentrations range from 56 nM to 5×10⁻⁵ pM.

After incubating the cells for 16-20 hr at 37° C./5% CO₂, 5 μL of supernatant is carefully transferred to a 384 microwell OptiPlate. IFNγ levels are measured using a human IFNγ AlphaLISA Assay Kit. Briefly, 10 μL of 2.5× AlphaLISA Anti-IFNγ acceptor beads and biotinylated antibody anti-IFNγ mix are added to the 5 L of NK-92 supernatants. The mixtures are incubated for 1 hr at room temperature with shaking. Under subdued light, 2.5 μL of 2X streptavidin (SA) donor beads are pipetted into each well, and the wells are incubated for 30 min at room temperature with shaking. AlphaLISA signals are then measured on an EnSpire™ plate reader using 680 nm and 615 nm as excitation and emission wavelengths, respectively. Half maximal effective concentrations (EC₅₀) are calculated based on a variable slope and four parameter analysis using GraphPad PRISM software.

Results of this experiment for various IL-18 polypeptides is shown below in Table 9 (EC₅₀ data).

### Example 15—IL-18 Binding Protein-Mediated Inhibition of IFNγ Secretion in NK-92 Cellular Assay

The NK cell line NK-92 derived from a patient with lymphoma (ATCC® CRL-2407™) is cultured in aMEM medium supplemented with 20% FCS-Glutamax™, 25 μM B-mercaptoethanol, and 100 IU/mL of recombinant human IL-2.

On the day of experiment, cells are harvested and washed with aMEM medium without IL-2 and containing 1 ng/mL of recombinant human IL-12. After counting, the cells are seeded at 100,000 cells/well in a 384 well titer plate and incubated at 37° C./5% CO₂. Sixteen 2-fold serial dilutions of Fc-fused human IL-18 binding protein isoform a (IL-18BPa) are prepared in aMEM medium. 1 ng/mL of IL-12 containing 2 nM of each modified IL-18 polypeptide variant is added to the NK-92 cells. The final IL-18 analyte concentration is 1 nM, and the final IL-18BPa concentration ranged from 566 nM to 17 pM.

After incubating the cells for 16-20 hr at 37° C./5% CO₂, 5 μL of the supernatant is carefully transferred to a 384 microwell OptiPlate. IFNγ levels are measured using a human IFNγ AlphaLISA Assay Kit. Briefly, 10 μL of 2.5× AlphaLISA anti-IFNγ acceptor beads and biotinylated antibody anti-IFNγ mix are added to 5 μL of NK-92 supernatants. The mixtures are incubated for 1 hr at room temperature with shaking. Under subdued light, 2.5 μL of 2× SA donor beads are pipetted in each well and incubated for 30 min at room temperature with shaking. AlphaLISA signals are then measured on an EnSpire™ plate reader using 680 nm and 615 nm as excitation and emission wavelengths, respectively. Half maximal inhibitory concentrations (IC₅₀) are calculated based on a variable slope and four parameter analysis using GraphPad PRISM software.

Modified IL-18 variants of the disclosure are active and able to induce IFNγ secretion in vitro. Table 9 shows the ability of many of the tested IL-18 variants to induce IFNγ production while some IL-18 variants are significantly less sensitive to inhibition by IL-18BP, as measured by EC₅₀ and IC₅₀, respectively.

TABLE 9
IC₅₀/EC₅₀ in NK92 IFNγ Release Assay Data

| SEQ | | | |
|---|---|---|---|
| ID | Sequence | EC₅₀ | IC₅₀ |
| NO: | modifications | (nM) | (nM) |
| 1 | Native sequence | 0.533 | 0.787 |
| 8 | E6K, V11I, C38A, | 0.022 | 522 |
| | K53A, T63A, C68A, | | |
| | C76A, T95C, C127A | | |
| 11 | E6K, V11I, C38A, | 0.008 | 532 |
| | K53A, C76A, C127A | | |
| 6 + PEG | E6K, V11I, C38A, | 0.342 | 14.31 |
| | K53A, T63A, C68A, | | |
| | C76A, C127A, D98C- | | |
| | 30kPEG | | |
| 6 | E6K, V11I, C38A, | 1.366 | 5.847 |
| | K53A, T63A, C68A, | | |
| | C76A, C127A, D98C | | |
| 5 + PEG | E6K, V11I, C38A, | 1.862 | 11.96 |
| | K53A, T63A, C68A, | | |
| | C76A, C127A, M86C- | | |
| | 30kPEG | | |
| 5 | E6K, V11I, C38A, | 0.075 | 62.37 |
| | K53A, T63A, C68A, | | |
| | C76A, C127A, M86C | | |
| 9 + PEG | E6K, C38A, K53A, | 0.64 | 27.2 |
| | C68A, D98C-30kPEG | | |
| 9 | E6K, C38A, K53A, | 0.069 | 960.8 |
| | C68A, D98C | | |
| 4 + PEG | E6K, C38A, K53A, | 0.425 | 34 |
| | C68A, M86C-30kPEG | | |
| 4 | E6K, C38A, K53A, | 0.022 | 396.3 |
| | C68A, M86C | | |
| 28 | E6K, C38A, K53A, | 0.005 | 233.050 |
| | T63A, C76A, C127A | | |
| 30 | E6K, V11I, C38A, | 0.0014 | 487.800 |
| | K53A, T63A, C76A, | | |
| | C127A | | |
| 62 | E6K, C38A, K53A, | 0.026 | 283.6 |
| | C127A | | |
| 61 | E6K, C38A, K53A, | 0.007 | 778.900 |
| | C76A | | |
| 60 | E6K, C38Q, K53A | 0.006 | 780.5 |
| 59 | E6K, C38A, K53A | 0.015 | 653.5 |
| 57 | E6K, C38S, K53A | 0.045 | 146.2 |
| 63 | E6K, C38A, K53A, | 0.007 | 286.000 |
| | C76A, C127A | | |
| 204 | V11I, C38A, K53A, | 0.138 | 1.625 |
| | C76A, C127A | | |
| 205 | V11I, C38A, K53A, | 0.012 | 7.522 |
| | T63A, C76A, C127A | | |
| 206 | V11I, C38A, K53A, | 0.087 | 10.24 |
| | S55A, C76A, C127A | | |
| 207 | V11I, C38A, M51G, | 0.037 | 732.9 |
| | K53A, C76A, C127A | | |
| 208 | V11I, C38A, K53A, | 0.079 | 47.63 |
| | D54A, C76A, C127A | | |
| 209 | F2A, V11I, C38A, | 0.256 | 5.055 |
| | K53A, C76A, C127A | | |
| 210 | V11I, E31A, C38A, | 0.187 | 1.167 |
| | K53A, C76A, C127A | | |
| 211 | V11I, T34A, C38A, | 0.015 | 21.27 |
| | K53A, C76A, C127A | | |
| 212 | V11I, D35A, C38A, | 0.061 | 3.622 |
| | K53A, C76A, C127A | | |
| 213 | V11I, S36A, C38A, | 0.033 | 7.85 |
| | K53A, C76A, C127A | | |
| 214 | V11I, D37A, C38A, | 0.175 | 2.222 |
| | K53A, C76A, C127A | | |
| 215 | V11I, E31A, D37A, | 0.062 | 3.709 |
| | C38A, K53A, C76A, | | |
| | C127A | | |
| 216 | V11I, C38A, D40A, | 0.067 | 3.233 |
| | K53A, C76A, C127A | | |
| 217 | V11I, C38A, N41A, | 0.558 | 0.681 |
| | K53A, C76A, C127A | | |
| 218 | V11I, C38A, K53A, | 0.056 | 6.082 |
| | C76A, C127A, D132A | | |
| 219 | V11I, C38A, K53A, | 0.073 | 3.981 |
| | C76A, G108A, C127A | | |
| 220 | V11I, C38A, K53A, | 0.123 | 1.807 |
| | C76A, H109A, C127A | | |
| 221 | V11I, C38A, K53A, | 0.028 | 3.181 |
| | C76A, D110A, C127A | | |
| 222 | K8R, V11I, C38A, | 0.057 | 1.073 |
| | C76A, Q103E, C127A | | |
| 223 | K8E, V11I, C38A, | 0.061 | 7.292 |
| | C76A, Q103R, C127A | | |
| 224 | V11I, C38A, C76A, | 0.093 | 0.823 |
| | Q103K, C127A | | |
| 225 | V11I, C38A, S55H, | 0.414 | 0.456 |
| | C76A, C127A | | |
| 226 | V11I, C38A, S55R, | 0.176 | 0.885 |
| | C76A, C127A | | |
| 227 | V11I, C38A, S55T, | 0.098 | 0.44 |
| | C76A, C127A | | |
| 228 | V11I, C38A, C76A, | 0.103 | 0.809 |
| | S105I, C127A | | |
| 229 | V11I, C38A, C76A, | 0.098 | 0.176 |
| | S105K, C127A | | |

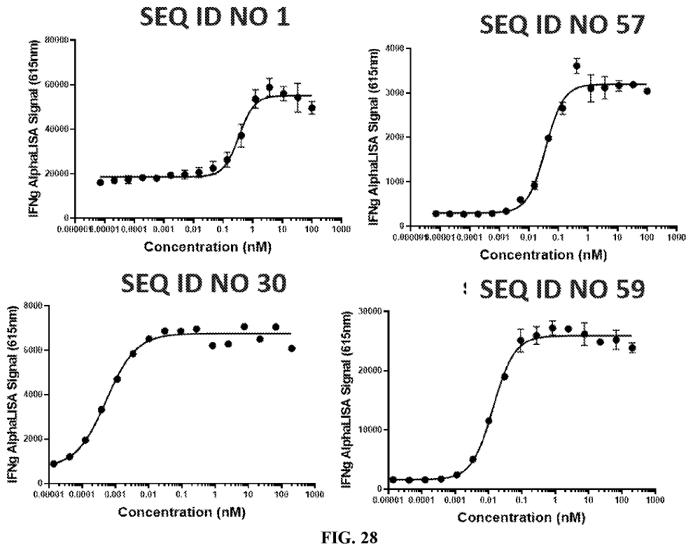

FIG. 28 shows plots measuring ability of wild type IL-18 and of modified IL-18 polypeptides to stimulate the secretion of IFNgamma by NK-92 cells. The figure shows mean IFNg alphaLISA signal on the y-axis and dosage of the IL-18 polypeptides on the x-axis. The IL-18 polypeptides are native IL-18 wild-type (SEQ ID NO: 01), SEQ ID NO: 57, SEQ ID NO: 59, and SEQ ID NO: 30.

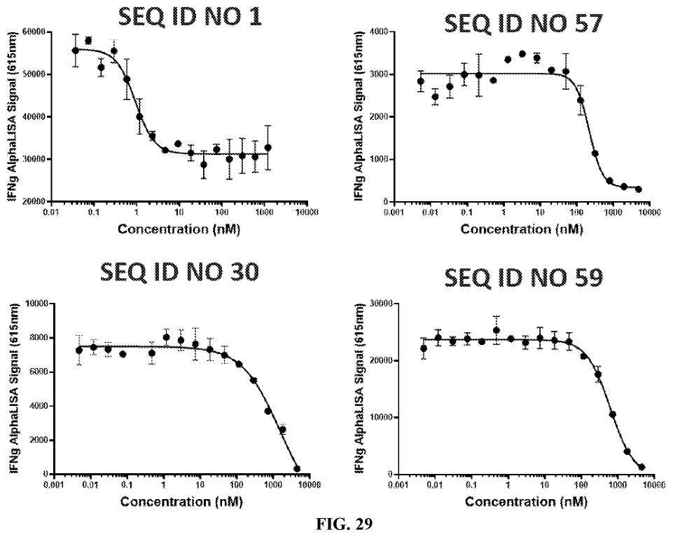

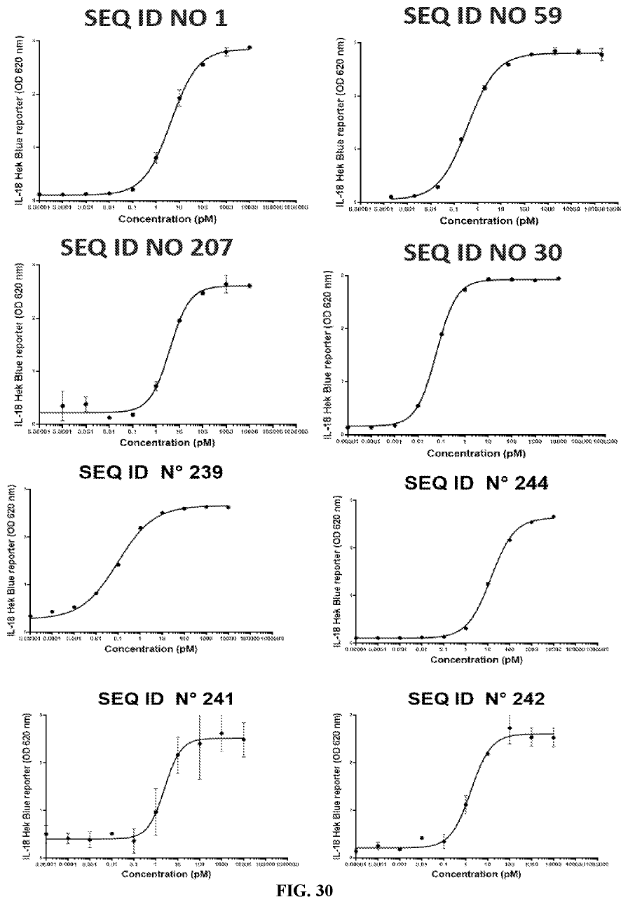

FIG. 29 shows plots measuring the ability of the human IL-18 Binding Protein to inhibit the secretion of IFNgamma by NK92 cells stimulated with 2 nM of wild type IL-18 and of modified IL-18 polypeptides. The figure shows mean IFNg alphaLISA signal on the y-axis, and dosage of the human IL-18 Binding Protein on the x-axis. The IL-18 polypeptides are native IL-18 wild-type (SEQ ID NO: 01), SEQ ID NO: 57, SEQ ID NO: 59, and SEQ ID NO: 30.

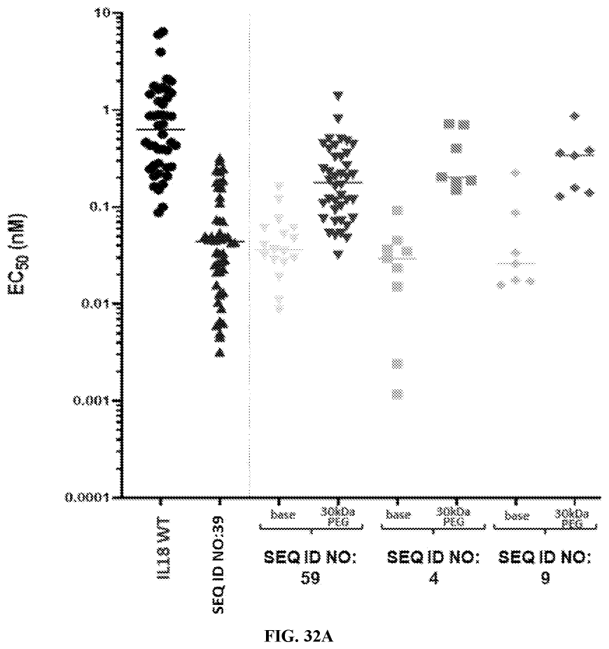

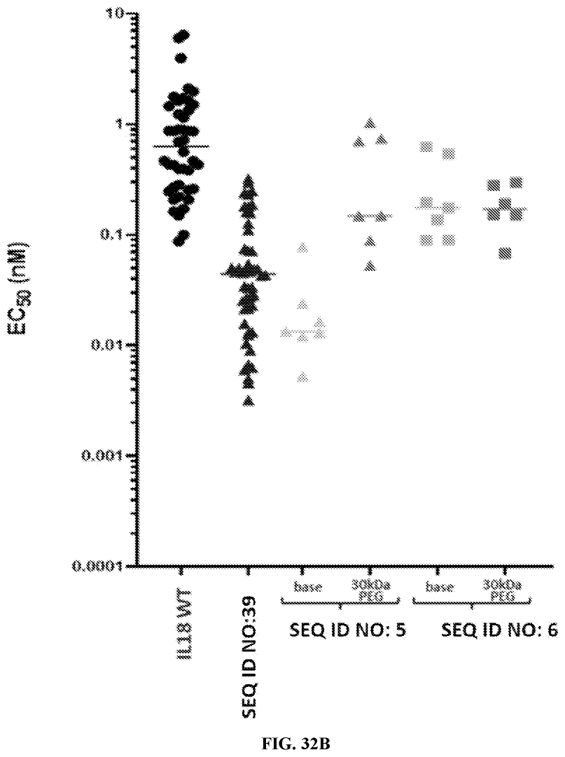

FIGS. 32A-B shows effect of attaching 30 kDa PEG to IL-18 variants on IL-18 mediated IFNγ secretion in human peripheral blood mononuclear cells. EC50 for IL-18 mediated IFNγ secretion for IL-18 of SEQ ID NO: 59, 4 and 9 is shown in FIG. 32A, and of SEQ ID NO: 5 and 6 is shown in FIG. 32B.

### Example 16—HEK-Blue IL18R Reporter Assay

An IL-18R positive HEK-Blue reporter cell line is used to determine binding of IL-18 variants to IL-18R and subsequent downstream signaling. The general protocol is outlined below.

5×10⁴ cells HEK-Blue IL18R reporter cells (InvivoGen, #hkb-hmi118) are seeded into each well of a 96 well plate and stimulated with 0-100 nM of IL-18 polypeptide variants at 37° C. and 5% CO₂. After 20h incubation, 20 μL of cell culture supernatant is then taken from each well and mixed with 180 μL QUANTI-Blue media in a 96 well plate, incubated for 1 hour at 37° C. and 5% CO₂. The absorbance signal at 620 nm is then measured on an Enspire plate reader with 680 and 615 nm as excitation and emission wavelengths, respectively. Half Maximal Effective dose (EC50) is calculated based on a variable slope, four parameter analysis using GraphPad PRISM software.

Results of this experiment for select variants are shown below in Table 10.

TABLE 10
EC₅₀ in HEK-Blue IL18R Reporter Assay Data

| SEQ | | |
|---|---|---|
| ID | | EC₅₀ |
| NO: | Sequence modifications | (nM) |
| 1 | Native sequence | 2.75 |
| 8 | E6K, V11I, C38A, K53A, T63A, C68A, | |
| | C76A, T95C, C127A | |
| 11 | E6K, V11I, C38A, K53A, C76A, C127A | |
| 6 + PEG | E6K, V11I, C38A, K53A, T63A, C68A, | |
| | C76A, C127A, D98C-30kPEG | |
| 6 | E6K, V11I, C38A, K53A, T63A, C68A, | |
| | C76A, C127A, D98C | |
| 5 + PEG | E6K, V11I, C38A, K53A, T63A, C68A, | |
| | C76A, C127A, M86C-30kPEG | |
| 5 | E6K, V11I, C38A, K53A, T63A, C68A, | |
| | C76A, C127A, M86C | |
| 9 + PEG | E6K, C38A, K53A, C68A, D98C-30kPEG | |
| 9 | E6K, C38A, K53A, C68A, D98C | |
| 4 + PEG | E6K, C38A, K53A, C68A, M86C-30kPEG | |
| 4 | E6K, C38A, K53A, C68A, M86C | |
| 28 | E6K, C38A, K53A, T63A, C76A, C127A | |
| 30 | E6K, V11I, C38A, K53A, T63A, C76A, C127A | 0.053 |
| 63 | E6K, C38A, K53A, C76A, C127A | 0.01389 |
| 62 | E6K, C38A, K53A, C127A | 0.17 |
| 61 | E6K, C38A, K53A, C76A | 0.084 |
| 60 | E6K, C38Q, K53A | 0.203 |
| 59 | E6K, C38A, K53A | 0.268 |
| 57 | E6K, C38S, K53A | 0.53 |
| 204 | V11I, C38A, K53A, C76A, C127A | 0.98 |
| 205 | V11I, C38A, K53A, T63A, C76A, C127A | 0.17 |
| 206 | V11I, C38A, K53A, S55A, C76A, C127A | 3.63 |
| 207 | V11I, C38A, M51G, K53A, C76A, C127A | 0.8 |
| 208 | V11I, C38A, K53A, D54A, C76A, C127A | 1 |
| 209 | F2A, V11I, C38A, K53A, C76A, C127A | 7.28 |
| 210 | V11I, E31A, C38A, K53A, C76A, C127A | 6.6 |
| 211 | V11I, T34A, C38A, K53A, C76A, C127A | 0.7 |
| 212 | V11I, D35A, C38A, K53A, C76A, C127A | 13.12 |
| 213 | V11I, S36A, C38A, K53A, C76A, C127A | 0.25 |
| 214 | V11I, D37A, C38A, K53A, C76A, C127A | 14.12 |
| 215 | V11I, E31A, D37A, C38A, K53A, C76A, C127A | 11.95 |
| 216 | V11I, C38A, D40A, K53A, C76A, C127A | 0.52 |
| 217 | V11I, C38A, N41A, K53A, C76A, C127A | 11.7 |
| 218 | V11I, C38A, K53A, C76A, C127A, D132A | 1.95 |
| 219 | V11I, C38A, K53A, C76A, G108A, C127A | 15.56 |
| 220 | V11I, C38A, K53A, C76A, H109A, C127A | 19.5 |
| 221 | V11I, C38A, K53A, C76A, D110A, C127A | 2.02 |
| 222 | K8R, V11I, C38A, C76A, Q103E, C127A | 2.01 |
| 223 | K8E, V11I, C38A, C76A, Q103R, C127A | 2.3 |
| 224 | V11I, C38A, C76A, Q103K, C127A | 1.5 |
| 225 | V11I, C38A, S55H, C76A, C127A | 3.14 |
| 226 | V11I, C38A, S55R, C76A, C127A | 1.91 |
| 227 | V11I, C38A, S55T, C76A, C127A | 4.73 |
| 228 | V11I, C38A, C76A, S105I, C127A | 5.37 |
| 229 | V11I, C38A, C76A, S105K, C127A | 7.73 |
| 230 | K8L, E6K, V11I, C38A, K53A, T63A, C76A, C127A | 0.29 |
| 231 | E6K, V11I, C38A, I49E, K53A, T63A, C76A, C127A | |
| 232 | E6K, V11I, C38A, I49M, K53A, T63A, C76A, C127A | 0.07 |
| 233 | E6K, V11I, C38A, I49R, K53A, T63A, C76A, C127A | 0.04 |
| 234 | E6K, V11I, C38A, K53A, T63A, C76A, Q103R, C127A | 0.26 |
| 235 | E6K, K8E, V11I, C38A, K53A, T63A, C76A, Q103R, | 0.4 |
| | C127A | |
| 236 | E6K, V11I, C38A, K53A, T63A, C76A, C127A, V153R | |
| 237 | E6K, V11I, C38A, K53A, T63A, C76A, C127A, V153E | 0.1 |
| 238 | E6K, V11I, C38A, K53A, T63A, C76A, C127A, | 0.08 |
| | V153Y | |
| 239 | E6K, V11I, C38A, M51G, K53A, T63A, C76A, C127A | 0.1 |
| 240 | E6R, V11I, C38A, K53A, T63A, C76A, C127A | 0.04 |
| 241 | E6K, V11I, C38A, K53A, T63A, C76A, C127A | 2.5 |
| 242 | E6K, V11I, C38A, K53A, T63A, C76A, C127A | 1.68 |
| 243 | Y1M, E6K, V11I, C38A, K53A, T63A, C76A, C127A | 0.02 |
| 244 | E6K, V11I, C38A, M51G, K53A, T63A, C76A, C127A | 13.99 |

FIG. 30 shows plots measuring ability of wild type IL-18 and of modified IL-18 polypeptides to induce the NF-κB/AP-1-inducible secreted embryonic alkaline phosphatase (SEAP) reporter gene in Hek Blue cells expressing the IL-18 receptor. The figure shows mean SEAP reporter signal (GD 620 nm) on the y-axis, and dosage of the IL-18 polypeptides on the x-axis. The IL-18 polypeptides are native IL-18 wild-type (SEQ ID NO: 01), SEQ ID NO: 57, SEQ ID NO: 59, SEQ ID NO: 207, SEQ ID NO: 239, SEQ ID NO: 241, SEQ ID NO: 242, and SEQ ID NO: 244.

### Example 17—Pharmacokinetic and Pharmacodynamic Properties of Modified IL-18 Polypeptide Variants

The pharmacokinetic (PK) and pharmacodynamic (PD) properties of select IL-18 polypeptide variants are measured. Three C57BL/6 mice are tested per group and per time point. IL-18 variants are applied via single intravenous injections. Mice are divided into four dose groups: 0.5 mg/kg, 0.1 mg/kg, 0.02 mg/kg, 0.004 mg/kg; and four time point groups: 5 min, 6 hr, 24 hr, 48 hr.

Immune-related PD effects are determined by analyzing cytokine levels in plasma. The following plasma cytokines ae measured: IFNγ, CXCL9, CXCL10, GM-CSF, IL-la, FasL, and IL-18BP. The activation status of leukocytes is determined by monitoring surface markers: ICOS, PD-1, CD25, CD69, and Fas. Bioanalysis is conducted by detecting the total amount of IL-18 variants (free and IL-18BP-complexed). Corning high-binding half-area plates (Fisher Scientific, Reinach, Switzerland) are coated overnight at 4° C. with 25 μl of anti-IL18 monoclonal antibody (MBL, cat #D043-3, Clone 25-2G) at 2 μg/ml in PBS. Plates are then washed four times with 100 μl of PBS-0.02% Tween20. Plates surfaces are blocked with 25 μl of PBS-0.02% Tween20-1% BSA at 37° C. during 1 h. Plates are then washed four times with 100 μl of PBS-0.02% Tween20. Twenty-five microliters of IL-18 variants (or of mouse plasma) are added in eight fold serial dilutions starting at 50 nM down to 0.02 nM into PBS-0.02% Tween20-0.1% BSA and incubated at 37° C. during 2h. Plates are then washed four times with 100 μl of PBS-0.02% Tween20 and 25 μl of of biotinylated anti-IL18 monoclonal antibody (MBL, cat #D045-6, Clone 159-12B) at 2 μg/ml in PBS. Plates are incubated during 2h at 37° C. and ae then washed four times with 100 l of PBS-0.02% Tween20. Twenty-five microliters of Streptavidin-Horseradish peroxidase (#RABHRP3, Merck, Buchs, Switzerland) diluted at 1:500 into PBS-0.02% Tween20-0.1% BSA are added to each well and incubated at Room Temperature during 30 min. Plates are then washed four times with 100 μl of PBS-0.02% Tween20. Fifty microliters of TMB substrate reagent (#CL07, Merck, Buchs, Switzerland) are added to each well and incubated at 37° C. during 5 min. After 5 min at 37° C., Horseradish peroxidase reaction is stopped by adding 50 μl/well of 0.5M H2SO4 stop solution. ELISA signal is then measured at 450 nm on an EnSpire plate reader from Perkin Elmer (Schwerzenbach, Switzerland)

### Example 18—PBMC Stimulation Assay

Ability of IL-18 variants to stimulate peripheral blood mononuclear cells (PBMCs) is assessed according to the following protocol.

Isolation of lymphocytes: Blood from Buffy Coats of healthy volunteers is diluted with equal volume of PBS and slowly poured on top of SepMate tube prefilled with 15 mL Histopaque-1077. Tubes are centrifuged for 10 minutes at 1200 g, the top layer is collected and washed 3 times with PBS containing 2% of Fetal Bovine Serum. PBMCs are counted and cryopreserved as aliquots of 20×10⁶ cells.

Cryopreserved PBMCs are thawed and stimulated with gradient of human IL-18 variants ranging from 0.2 pM to 1 μM in RPMI containing 10% Fetal Bovine Serum.

Cytokine production after 24 hr stimulation is measured using Legendplex bead-based cytokine assay (Biolegend #740930) following manufacturer instructions. Half maximal effective concentrations (EC₅₀) of IFNγ released in culture supernatant are calculated based on a variable slope and four parameter analysis using GraphPad PRISM software.

Surface expression of FcγRIII on NK cells is measured by flow cytometry (Mouse IgG1 clone 3G8) after 72 hr stimulation.

TABLE 11
EC₅₀ in IFNγ secretion assay in PBMCs

| SEQ ID | | EC₅₀ |
|---|---|---|
| NO: | Sequence modifications | (nM) |
| 1 | Native sequence | 0.429 |
| 8 | E6K, V11I, C38A, K53A, T63A, C68A, C76A, | 0.0086 |
| | T95C, C127A | |
| 11 | E6K, V11I, C38A, K53A, C76A, C127A | 0.0052 |
| 6 + PEG | E6K, V11I, C38A, K53A, T63A, C68A, C76A, | 0.177 |
| | C127A, D98C-30kPEG | |
| 6 | E6K, V11I, C38A, K53A, T63A, C68A, C76A, | 0.196 |
| | C127A, D98C | |
| 5 + PEG | E6K, V11I, C38A, K53A, T63A, C68A, C76A, | 0.149 |
| | C127A, M86C-30kPEG | |
| 5 | E6K, V11I, C38A, K53A, T63A, C68A, C76A, | 0.013 |
| | C127A, M86C | |
| 9 + PEG | E6K, C38A, K53A, C68A, D98C-30kPEG | 0.340 |
| 9 | E6K, C38A, K53A, C68A, D98C | 0.027 |
| 4 + PEG | E6K, C38A, K53A, C68A, M86C-30kPEG | 0.203 |
| 4 | E6K, C38A, K53A, C68A, M86C | 0.035 |
| 30 | E6K, V11I, C38A, K53A, T63A, C76A, C127A | 0.0021 |
| 63 | E6K, C38A, K53A, C76A, C127A | 0.034 |
| 28 | E6K, C38A, K53A, T63A, C76A, C127A | 0.0017 |
| 62 | E6K, C38A, K53A, C127A | 0.074 |
| 61 | E6K, C38A, K53A, C76A | 0.004 |
| 60 | E6K, C38Q, K53A | 0.033 |
| 59 | E6K, C38A, K53A | 0.035 |
| 57 | E6K, C38S, K53A | 2.580 |

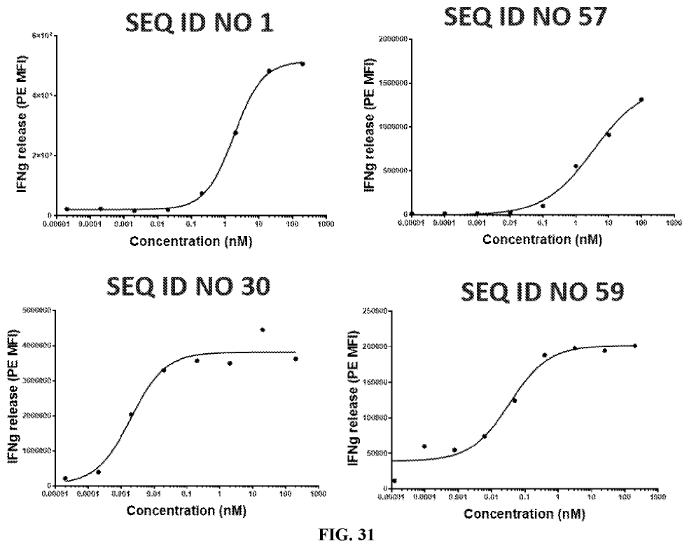

FIG. 31 shows plots measuring ability of wild type IL-18 and of modified IL-18 polypeptides to stimulate the secretion of IFNgamma by human Peripheral Blood Mononuclear Cells (PBMCs). The figure shows mean IFNg signal on the y-axis and dosage of the IL-18 polypeptides on the x-axis. The IL-18 polypeptides are native IL-18 wild-type (SEQ ID NO: 01), SEQ ID NO: 57, SEQ ID NO: 59, and SEQ ID NO: 30.

Although the present disclosure and its advantages have been described in detail, it should be understood that various changes, substitutions and alterations can be made herein without departing from the spirit and scope of the disclosure as defined in the appended claims.

1-4. (canceled)

5. A modified interleukin 18 (IL-18) polypeptide, wherein the modified IL-18 polypeptide comprises a substitution at residue V11 and at least one additional substitution at a residue selected from Y1, F2, E6, K8, S10, D17, T34, D35, S36, D37, D40, N41, I49, K53, M51, D54, 555, Q103, S105, G108, H109, D110, and D132, wherein residue position numbering is based on SEQ ID NO: 1 as a reference sequence.

6. (canceled)

7. (canceled)

8. The modified IL-18 polypeptide of claim 5, wherein the modified IL-18 polypeptide exhibits a to half-maximal effective concentration (EC50) to induce IFNγ production in of a cell which is at least 30-fold, 40-fold, 50-fold, 60-fold, 70-fold, or 80-fold lower than the EC50 for wild type IL-18 (WT IL-18).

9. The modified IL-18 polypeptide of claim 5, wherein the modified IL-18 polypeptide exhibits half maximal effective concentration (EC50) for signaling through the IL-18 receptor which is at least 5-fold, 10-fold, 20-fold, 30-fold, 40-fold, or 50-fold lower than the EC50 for wild type IL-18 (WT IL-18).

10. The modified IL-18 polypeptide of claim 5, wherein the modified IL-18 polypeptide exhibits a binding affinity (KD) with the IL-18 receptor alpha subunit which is at least 60-fold, 70-fold, 80-fold, 90-fold, or 100-fold lower than wild type IL-18.

11-15. (canceled)

16. The modified IL-18 polypeptide of claim 5, wherein the modified IL-18 polypeptide comprises a substitutions at residue T63, K53, E6, and/or M51.

17. The modified IL-18 polypeptide of claim 5, wherein the modified IL-18 polypeptide comprises a T63A substitution.

18. (canceled)

19. The modified IL-18 polypeptide of claim 5, wherein the modified IL-18 polypeptide comprises a K53A substitution.

20. (canceled)

21. The modified IL-18 polypeptide of claim 5, wherein the modified IL-18 polypeptide comprises an E6K substitution.

22. (canceled)

23. (canceled)

24. The modified IL-18 polypeptide of claim 5, wherein the modified IL-18 polypeptide comprises a V11I substitution.

25. The modified IL-18 polypeptide of claim 5, wherein the modified IL-18 polypeptide comprises E6K, V11I, K53A, and T63A substitutions.

26. The modified IL-18 polypeptide of claim 5, wherein the modified IL-18 polypeptide comprises V11I, M51G, and K53A substitution.

27. The modified IL-18 polypeptide of any one of claim 5, wherein the modified IL-18 polypeptide comprises E6K, V11I, M51G, K53A, and T63A substitutions.

28. The modified Il-18 polypeptide of claim 5, wherein the modified IL-18 polypeptide comprises E6K, V11I, C38A, K53A, T63A, C76A, and C127A substitutions.

29. The modified Il-18 polypeptide of claim 5, wherein the modified IL-18 polypeptide comprises V11I, C38A, K53A, C76A, C127A and M51G substitutions.

30. The modified Il-18 polypeptide of claim 5, wherein the modified IL-18 polypeptide comprises E6K, V11I, C38A, K53A, T63A, C76A, C127A and M51G substitutions.

31. The modified IL-18 polypeptide of claim 5, further comprising at least one glycine residue attached to the N-terminus of the IL-18 polypeptide.

32. (canceled)

33. (canceled)

34. The modified IL-18 polypeptide of claim 5, wherein the modified IL-18 polypeptide comprises an amino acid sequence having at least 80%, 85%, 90%, 95%, 98%, 99%, or 100% sequence identity to the sequence set forth in SEQ ID NO: 30.

35. The modified IL-18 polypeptide of claim 5, wherein the modified IL-18 polypeptide comprises the sequence set forth in SEQ ID NO: 30.

36-46. (canceled)

47. A modified IL-18 polypeptide comprising the sequence set forth in SEQ ID NO: 30.

48-72. (canceled)

73. A modified IL-18 polypeptide comprising E6K, V11I, C38A, K53A, T63A, C76A, and C127A amino acid substitutions, wherein residue position numbering is based on SEQ ID NO: 1 as a reference sequence, wherein the modified IL-18 polypeptide comprises an amino acid sequence having at least 80%, at least 85%, at least 90%, at least 95%, at least 96%, at least 97%, at least 98%, or at least 99% sequence identity to SEQ ID NO: 30.

74-77. (canceled)


SEQUENCE LISTING (428 sequences)

SEQ ID NO: 1 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKCMNPPDNIKDTKSDIIFFQRSVPGHDNKMQFESSSYEGYFLACEKERDLFKLILKKEDELGDRSIMFTVQNED

SEQ ID NO: 3 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKCMNPPDNIKDTKSDIIFFQRSVPGHDNKMQFESSSYEGYFLAAEKERDLFKLILKKEDELGDRSIMFTVQNED

SEQ ID NO: 4 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKECNPPDNIKDTKSDIIFFQRSVPGHDNKMQFESSSYEGYFLACEKERDLFKLILKKEDELGDRSIMFTVQNED

SEQ ID NO: 5 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKECNPPDNIKDTKSDIIFFQRSVPGHDNKMQFESSSYEGYFLAAEKERDLFKLILKKEDELGDRSIMFTVQNED

SEQ ID NO: 6 - protein, 157 aa, synthetic construct
YFGKLKSKLSVIRNLNDQVLFIDQGNRPLFEDMTDSDARDNAPRTIFIISMYADSQPRGMAVAISVKAEKISTLSAENKIISFKEMNPPDNIKDTKSCIIFFQRSVPGHDNKMQFESSSYEGYFLAAEKERDLFKLILKKEDELGDRSIMFTVQNED

SEQ ID NO: 7 - protein, 157 aa, synthetic construct
YFGKLKSKLSVIRNLNDQVLFIDQGNRPLFEDMTDSDARDNAPRTIFIISMYADSQPRGMAVTISVKAEKISTLSAENKIISFKECNPPDNIKDTKSDIIFFQRSVPGHDNKMQFESSSYEGYFLAAEKERDLFKLILKKEDELGDRSIMFTVQNED

SEQ ID NO: 8 - protein, 157 aa, synthetic construct
YFGKLKSKLSIIRNLNDQVLFIDQGNRPLFEDMTDSDARDNAPRTIFIISMYADSQPRGMAVAISVKAEKISTLSAENKIISFKEMNPPDNIKDCKSDIIFFQRSVPGHDNKMQFESSSYEGYFLAAEKERDLFKLILKKEDELGDRSIMFTVQNED

SEQ ID NO: 9 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKEMNPPDNIKDTKSCIIFFQRSVPGHDNKMQFESSSYEGYFLACEKERDLFKLILKKEDELGDRSIMFTVQNED

SEQ ID NO: 10 - protein, 157 aa, synthetic construct
YFGKLKSKLSVIRNLNDQVLFIDQGNRPLFEDMTDSDARDNAPRTIFIISMYADSQPRGMAVTISVKAEKISTLSAENKIISFKEMNPPDNIKDTKSCIIFFQRSVPGHDNKMQFESSSYEGYFLAAEKERDLFKLILKKEDELGDRSIMFTVQNED

SEQ ID NO: 11 - protein, 157 aa, synthetic construct
YFGKLKSKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 12 - protein, 157 aa, synthetic construct
YFGKLKSKLSVIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 13 - protein, 157 aa, synthetic construct
YFGKLKSKLSVIRNLNDQVLFIDQGNRPLFEDMTDSDCRDNAPRTIFIISMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 14 - protein, 157 aa, synthetic construct
YFGKLKSKLSVIRNLNDQVLFIDQGNRPLFEDMTDSDCRDNAPRTIFIIAMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 15 - protein, 157 aa, synthetic construct
YFGKLKSKLSVIRNLNDQVLFIDQGNRPLFEDMTDSDCRDNAPRTIFIIHMYADSQPRGMAVNISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 16 - protein, 157 aa, synthetic construct
YFGKLKSKLSVIRNLNDQVLFIDQGNRPLFEDMTDSDCRDNAPRTIFIISMYADSQPRGMAVNIAVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 17 - protein, 157 aa, synthetic construct
YFGKLKSKLSVIRNLNDQVLFIDQGNRPLFEDMTDSDCRDNAPRTIFIIHMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 18 - protein, 157 aa, synthetic construct
YFGKLKSKLSVIRNLNDQVLFIDQGNRPLFEDMTDSDARDNAPRTIFIISMYADSQPRGMAVTISVKAEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 19 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENAIISFKEMNPPDNIKDTKSDIIFFQRSVPGHDNKMQFESSSYEGYFLACEKERDLFKLILKKEDELGDRSIMFTVQNED

SEQ ID NO: 20 - protein, 157 aa, synthetic construct; identical to SEQ ID NO: 19

SEQ ID NO: 21 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPAHDNKMQFESSSYEGYFLACEKERDLFKLILKKEDELGDRSIMFTVQNED

SEQ ID NO: 22 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGADNKMQFESSSYEGYFLACEKERDLFKLILKKEDELGDRSIMFTVQNED

SEQ ID NO: 23 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNAMQFESSSYEGYFLACEKERDLFKLILKKEDELGDRSIMFTVQNED

SEQ ID NO: 24 - protein, 157 aa, synthetic construct
YFGKLKSKLSVIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 25 - protein, 157 aa, synthetic construct
YFGKLKSKLSVIRNLNDQVLFIDQGNRPLFEDMTDSDQRDNAPRTIFIISMYADSQPRGMAVAISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 26 - protein, 157 aa, synthetic construct
YFGKLKSKLSVIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 27 - protein, 157 aa, synthetic construct
YFGKLKSKLSVIRNLNDQVLFIDQGNRPLFEDMTDSDQRDNAPRTIFIISMYADSQPRGMAVAISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 28 - protein, 157 aa, synthetic construct; identical to SEQ ID NO: 12

SEQ ID NO: 29 - protein, 157 aa, synthetic construct
YFGKLKSKLSIIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 30 - protein, 157 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 31 - protein, 157 aa, synthetic construct
YFGKLESKLSVIRNLNDQVLFIDQGNRPLFEDMTDSDARDNAPRTIFIISMYKDSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 32 - protein, 157 aa, synthetic construct
YFGKLESKLSVIRNLNDQVLFIDQGNRPLFEDMTDSDARDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 33 - protein, 157 aa, synthetic construct
YFGKLKSKLSVIRNLNDQVLFIDQGNRPLFEDMTDSDARDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 34 - protein, 157 aa, synthetic construct
YFGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 35 - protein, 157 aa, synthetic construct
GAGKLKSKLSVIRNLNDQVLFIDQGNRPLFEDMTDSDCRDNAPRTIFIISGYAAAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 36 - protein, 157 aa, synthetic construct
YFGKLESKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 37 - protein, 157 aa, synthetic construct
YFGKLESKLSVIRNLNDQVLFIDQGNRPLFEDMTDSDCRDNAPRTIFIISMYKDAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 38 - protein, 157 aa, synthetic construct
YFGKLKSKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 39 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 40 - protein, 157 aa, synthetic construct
YFGKLKSKLSVIRNLNDQVLFIDQGNRPLFEDMTDSDCRDNAPRTIFIISMYKDAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 41 - protein, 157 aa, synthetic construct
YFGKLESKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 42 - protein, 157 aa, synthetic construct
YFGKLKSKLSVIRNLNDQVLFIDQGNRPLFEDMTDSDCRDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 43 - protein, 157 aa, synthetic construct
GFGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 44 - protein, 157 aa, synthetic construct
YAGKLKSKLSVIRNLNDQVLFIDQGNRPLFEDMTDSDCRDNAPRTIFIISMYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 45 - protein, 157 aa, synthetic construct
YFGKLKSKLSVIRNLNDQVLFIDQGNRPLFEDMTDSDCRDNAPRTIFIISMYAAAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 46 - protein, 157 aa, synthetic construct
YFGKLKSKLSVIRNLNDQVLFIDQGNRPLFEDMTDSDCRDNAPRTIFIISGYADAQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 47 - protein, 157 aa, synthetic construct
YFGKLESKLSVIRNLNDQVLFIDQGNRPLFEDMTDSDSRDNAPRTIFIISMYKDSQPRGMAVTISVKSEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 48 - protein, 157 aa, synthetic construct
YFGKLESKLSVIRNLNDQVLFIDQGNRPLFEDMTDSDSRDNAPRTIFIISMYKDSQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 49 - protein, 157 aa, synthetic construct
YFGKLKSKLSVIRNLNDQVLFIDQGNRPLFEDMTDSDSRDNAPRTIFIISMYADAQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 50 - protein, 157 aa, synthetic construct
YFGKLKSKLSVIRNLNDQVLFIDQGNRPLFEDMTDSDCRDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 51 - protein, 157 aa, synthetic construct
YFGKLESKLSVIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 52 - protein, 157 aa, synthetic construct
YFGKLKSKLSVIRNLNDQVLFIDQGNRPLFEDMTDSDCRDNAPRTIFIISMYKDSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 53 - protein, 157 aa, synthetic construct
YFGKLESKLSVIRNLNDQVLFIDQGNRPLFEDMTDSDCRDNAPRTIFIISMYADSQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 54 - protein, 157 aa, synthetic construct
YFGKLKSKLSVIRNLNDQVLFIDQGNRPLFEDMTDSDSRDNAPRTIFIISMYADSQPRGMAVTISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 55 - protein, 157 aa, synthetic construct
YFGKLESKLSVIRNLNDQVLFIDQGNRPLFEDMTDSDSRDNAPRTIFIISMYADSQPRGMAVAISVKSECISTLSSENKIISFKEMNPPDNIKDTKSDIIFFQRSVPGHDNKMQFESSSYEGYFLASEKERDLFKLILKKEDELGDRSIMFTVQNED

SEQ ID NO: 56 - protein, 157 aa, synthetic construct
YFGKLKSKLSVIRNLNDQVLFIDQGNRPLFEDMTDSDSRDNAPRTIFIISMYADSQPRGMAVTISVKCEKISTLSSENKIISFKEMNPPDNIKDTKSDIIFFQRSVPGHDNKMQFESSSYEGYFLASEKERDLFKLILKKEDELGDRSIMFTVQNED

SEQ ID NO: 57 - protein, 157 aa, synthetic construct
YFGKLKSKLSVIRNLNDQVLFIDQGNRPLFEDMTDSDSRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 58 - protein, 157 aa, synthetic construct; identical to SEQ ID NO: 54

SEQ ID NO: 59 - protein, 157 aa, synthetic construct
YFGKLKSKLSVIRNLNDQVLFIDQGNRPLFEDMTDSDARDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 60 - protein, 157 aa, synthetic construct
YFGKLKSKLSVIRNLNDQVLFIDQGNRPLFEDMTDSDQRDNAPRTIFIISMYADSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 61 - protein, 157 aa, synthetic construct
YFGKLKSKLSVIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLACEKERDLFKLILKKEDELGDRSIMFTVQNED

SEQ ID NO: 62 - protein, 157 aa, synthetic construct
YFGKLKSKLSVIRNLNDQVLFIDQGNRPLFEDMTDSDARDNAPRTIFIISMYADSQPRGMAVTISVKCEKISTLSCENKIISFKEMNPPDNIKDTKSDIIFFQRSVPGHDNKMQFESSSYEGYFLAAEKERDLFKLILKKEDELGDRSIMFTVQNED

SEQ ID NO: 63 - protein, 157 aa, synthetic construct
YFGKLKSKLSVIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 64 - protein, 157 aa, synthetic construct
YFGKLKSKLSVIRNLNDQVLFIDQGNRPLFEDMTDSDARDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 65 - protein, 157 aa, synthetic construct
YFGKLKSKLSVIRNLNDQVLFIDQGNRPLFEDMTDSDQRDNAPRTIFIISMYADAQPRGMAVAISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 66 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFAEMNPPDNIKDTKSDIIFFQRSVPGHDNKMQFESSSYEGYFLACEKERDLFKLILKKEDELGDRSIMFTVQNED

SEQ ID NO: 67 - protein, 157 aa, synthetic construct
YFGKLKSKLSVIRNLNDQVLFIDQGNRPLFEDMTDSDCRDNAPRTIFIISMYADSQPRGMAVTISVKCEKISTLSCENKIISFKEMNPPDNIKDTKSAIIFFQRSVPGHDNKMQFESSSYEGYFLACEKERDLFKLILKKEDELGDRSIMFTVQNED

SEQ ID NO: 68 - protein, 157 aa, synthetic construct
  SITE 31: note = Homoserine
  SITE 63: note = Homoserine
  SITE 75: note = Homoserine
  SITE 116: note = Homoserine
YFGKLESKLSVIRNLNDQVLFIDQGNRPLFXDMTDSDCRDNAPRTIFIISMYKDSQPRGMAVXISVKCEKISTLXCENKIISFKEMNPPDNIKDTKSDIIFFQRSVPGHDNKMQFXSSSYEGYFLACEKERDLFKLILKKEDELGDRSIMFTVQNED

SEQ ID NO: 69-73 - skipped (5 entries)

SEQ ID NO: 74 - protein, 157 aa, synthetic construct
  SITE 31: note = Homoserine
  SITE 63: note = Homoserine
  SITE 75: note = Homoserine
  SITE 116: note = Homoserine
YFGKLKSKLSVIRNLNDQVLFIDQGNRPLFXDMTDSDCRDNAPRTIFIISMYADSQPRGMAVXISVKCEKISTLXCENKIISFKEMNPPDNIKDTKSDIIFFQRSVPGHDNKMQFXSSSYEGYFLACEKERDLFKLILKKEDELGDRSIMFTVQNED

SEQ ID NO: 75-79 - skipped (5 entries)

SEQ ID NO: 80 - protein, 157 aa, synthetic construct
  SITE 31: note = Homoserine
  SITE 63: note = Homoserine
  SITE 75: note = Homoserine
  SITE 116: note = Homoserine
YFGKLKSKLSVIRNLNDQVLFIDQGNRPLFXDMTDSDCRDNAPRTIFIISMYADSQPRGMAVXIAVKCEKISTLXCENKIISFKEMNPPDNIKDTKSDIIFFQRSVPGHDNKMQFXSSSYEGYFLACEKERDLFKLILKKEDELGDRSIMFTVQNED

SEQ ID NO: 81-85 - skipped (5 entries)

SEQ ID NO: 86 - protein, 157 aa, synthetic construct
  SITE 31: note = Homoserine
  SITE 63: note = Homoserine
  SITE 75: note = Homoserine
  SITE 116: note = Homoserine
YFGKLKSKLSVIRNLNDQVLFIDQGNRPLFXDMTDSDARDNAPRTIFIISMYADSQPRGMAVXISVKAEKISTLXAENKIISFKEMNPPDNIKDTKSDIIFFQRSVPGHDNKMQFXSSSYEGYFLAAEKERDLFKLILKKEDELGDRSIMFTVQNED

SEQ ID NO: 87-91 - skipped (5 entries)

SEQ ID NO: 92 - protein, 157 aa, synthetic construct
  SITE 31: note = Homoserine
  SITE 57: note = Homoserine
  SITE 75: note = Homoserine
  SITE 116: note = Homoserine
YFGKLESKLSVIRNLNDQVLFIDQGNRPLFXDMTDSDCRDNAPRTIFIISMYKDSLXRGMAVTISVKCEKISTLXCENKIISFKEMNPPDNIKDTKSDIIFFQRSVPGHDNKMQFXSSSYEGYFLACEKERDLFKLILKKEDELGDRSIMFTVQNED

SEQ ID NO: 93-97 - skipped (5 entries)

SEQ ID NO: 98 - protein, 157 aa, synthetic construct
  SITE 31: note = Homoserine
  SITE 57: note = Homoserine
  SITE 75: note = Homoserine
  SITE 116: note = Homoserine
YFGKLKSKLSVIRNLNDQVLFIDQGNRPLFXDMTDSDCRDNAPRTIFIISMYADSLXRGMAVTISVKCEKISTLXCENKIISFKEMNPPDNIKDTKSDIIFFQRSVPGHDNKMQFXSSSYEGYFLACEKERDLFKLILKKEDELGDRSIMFTVQNED

SEQ ID NO: 99-103 - skipped (5 entries)

SEQ ID NO: 104 - protein, 157 aa, synthetic construct
  SITE 31: note = Homoserine
  SITE 57: note = Homoserine
  SITE 75: note = Homoserine
  SITE 116: note = Homoserine
YFGKLKSKLSIIRNLNDQVLFIDQGNRPLFXDMTDSDCRDNAPRTIFIISMYADSLXRGMAVAISVKCEKISTLXCENKIISFKEMNPPDNIKDTKSDIIFFQRSVPGHDNKMQFXSSSYEGYFLACEKERDLFKLILKKEDELGDRSIMFTVQNED

SEQ ID NO: 105-109 - skipped (5 entries)

SEQ ID NO: 110 - protein, 157 aa, synthetic construct
  SITE 31: note = Homoserine
  SITE 57: note = Homoserine
  SITE 75: note = Homoserine
  SITE 116: note = Homoserine
YFGKLKSKLSVIRNLNDQVLFIDQGNRPLFXDMTDSDARDNAPRTIFIISMYADSLXRGMAVTISVKAEKISTLXAENKIISFKEMNPPDNIKDTKSDIIFFQRSVPGHDNKMQFXSSSYEGYFLAAEKERDLFKLILKKEDELGDRSIMFTVQNED

SEQ ID NO: 111-115 - skipped (5 entries)

SEQ ID NO: 116 - protein, 157 aa, synthetic construct
  SITE 31: note = Homoserine
  SITE 50: note = Homoserine
  SITE 75: note = Homoserine
  SITE 121: note = Homoserine
YFGKLESKLSVIRNLNDQVLFIDQGNRPLFXDMTDSDCRDNAPRTIFILXMYKDSQPRGMAVTISVKCEKISTLXCENKIISFKEMNPPDNIKDTKSDIIFFQRSVPGHDNKMQFESSSFXGYFLACEKERDLFKLILKKEDELGDRSIMFTVQNED

SEQ ID NO: 117-121 - skipped (5 entries)

SEQ ID NO: 122 - protein, 157 aa, synthetic construct
  SITE 31: note = Homoserine
  SITE 50: note = Homoserine
  SITE 75: note = Homoserine
  SITE 121: note = Homoserine
YFGKLKSKLSVIRNLNDQVLFIDQGNRPLFXDMTDSDCRDNAPRTIFILXMYADSQPRGMAVTISVKCEKISTLXCENKIISFKEMNPPDNIKDTKSDIIFFQRSVPGHDNKMQFESSSFXGYFLACEKERDLFKLILKKEDELGDRSIMFTVQNED

SEQ ID NO: 123-127 - skipped (5 entries)

SEQ ID NO: 128 - protein, 157 aa, synthetic construct
  SITE 31: note = Homoserine
  SITE 50: note = Homoserine
  SITE 75: note = Homoserine
  SITE 121: note = Homoserine
YFGKLKSKLSIIRNLNDQVLFIDQGNRPLFXDMTDSDCRDNAPRTIFILXMYADSQPRGMAVAISVKCEKISTLXCENKIISFKEMNPPDNIKDTKSDIIFFQRSVPGHDNKMQFESSSFXGYFLACEKERDLFKLILKKEDELGDRSIMFTVQNED

SEQ ID NO: 129-133 - skipped (5 entries)

SEQ ID NO: 134 - protein, 157 aa, synthetic construct
  SITE 31: note = Homoserine
  SITE 50: note = Homoserine
  SITE 75: note = Homoserine
  SITE 121: note = Homoserine
YFGKLKSKLSVIRNLNDQVLFIDQGNRPLFXDMTDSDARDNAPRTIFILXMYADSQPRGMAVTISVKAEKISTLXAENKIISFKEMNPPDNIKDTKSDIIFFQRSVPGHDNKMQFESSSFXGYFLAAEKERDLFKLILKKEDELGDRSIMFTVQNED

SEQ ID NO: 135 - skipped

SEQ ID NO: 136 - protein, 157 aa, synthetic construct
  SITE 31: note = Homoserine
  SITE 50: note = Homoserine
  VARIANT 69: note = N-(glutaryl-(Polyethylene glycol)x-azide)-L-lysine, N-((Polyethylene glycol)x-azide)-L-glutamine, N-((Polyethylene glycol)x-azide)-L-asparagine, or S-((N-((Polyethylene glycol)x-azide))amidyl-methyl)-L-cysteine, wherein each x is independently an integer from 1-30
  SITE 75: note = Homoserine
  SITE 121: note = Homoserine
YFGKLKSKLSVIRNLNDQVLFIDQGNRPLFXDMTDSDARDNAPRTIFILXMYADSQPRGMAVTISVKAXKISTLXAENKIISFKEMNPPDNIKDTKSDIIFFQRSVPGHDNKMQFESSSFXGYFLAAEKERDLFKLILKKEDELGDRSIMFTVQNED

SEQ ID NO: 137-139 - skipped (3 entries)

SEQ ID NO: 140 - protein, 157 aa, synthetic construct
  SITE 31: note = Homoserine
  SITE 67: note = Homoserine
  SITE 75: note = Homoserine
  SITE 116: note = Homoserine
YFGKLESKLSVIRNLNDQVLFIDQGNRPLFXDMTDSDCRDNAPRTIFIISMYKDSQPRGMAVTISVXCEKISTLXCENKIISFKEMNPPDNIKDTKSDIIFFQRSVPGHDNKMQFXSSSYEGYFLACEKERDLFKLILKKEDELGDRSIMFTVQNED

SEQ ID NO: 141-145 - skipped (5 entries)

SEQ ID NO: 146 - protein, 157 aa, synthetic construct
  SITE 31: note = Homoserine
  SITE 67: note = Homoserine
  SITE 75: note = Homoserine
  SITE 116: note = Homoserine
YFGKLKSKLSVIRNLNDQVLFIDQGNRPLFXDMTDSDCRDNAPRTIFIISMYADSQPRGMAVTISVXCEKISTLXCENKIISFKEMNPPDNIKDTKSDIIFFQRSVPGHDNKMQFXSSSYEGYFLACEKERDLFKLILKKEDELGDRSIMFTVQNED

SEQ ID NO: 147-151 - skipped (5 entries)

SEQ ID NO: 152 - protein, 157 aa, synthetic construct
  SITE 31: note = Homoserine
  SITE 67: note = Homoserine
  SITE 75: note = Homoserine
  SITE 116: note = Homoserine
YFGKLKSKLSIIRNLNDQVLFIDQGNRPLFXDMTDSDCRDNAPRTIFIISMYADSQPRGMAVAISVXCEKISTLXCENKIISFKEMNPPDNIKDTKSDIIFFQRSVPGHDNKMQFXSSSYEGYFLACEKERDLFKLILKKEDELGDRSIMFTVQNED

SEQ ID NO: 153-157 - skipped (5 entries)

SEQ ID NO: 158 - protein, 157 aa, synthetic construct
  SITE 31: note = Homoserine
  SITE 67: note = Homoserine
  SITE 75: note = Homoserine
  SITE 116: note = Homoserine
YFGKLKSKLSVIRNLNDQVLFIDQGNRPLFXDMTDSDARDNAPRTIFIISMYADSQPRGMAVTISVXAEKISTLXAENKIISFKEMNPPDNIKDTKSDIIFFQRSVPGHDNKMQFXSSSYEGYFLAAEKERDLFKLILKKEDELGDRSIMFTVQNED

SEQ ID NO: 159-169 - skipped (11 entries)

SEQ ID NO: 170 - protein, 157 aa, synthetic construct
  SITE 31: note = Homoserine
  SITE 63: note = Homoserine
  SITE 116: note = Homoserine
YFGKLESKLSVIRNLNDQVLFIDQGNRPLFXDMTDSDCRDNAPRTIFIISMYKDSQPRGMAVXISVKCEKISTLSCENKIISFKEMNPPDNIKDTKSDIIFFQRSVPGHDNKMQFXSSSYEGYFLACEKERDLFKLILKKEDELGDRSIMFTVQNED

SEQ ID NO: 171 - protein, 157 aa, synthetic construct
  SITE 31: note = Homoserine
  SITE 63: note = Homoserine
  VARIANT 70: note = N-(glutaryl-(Polyethylene glycol)x-azide)-L-lysine or C-(methylenylx-azide)-L-glycine, wherein each x is independently an integer from 1-30
  SITE 116: note = Homoserine
YFGKLKSKLSVIRNLNDQVLFIDQGNRPLFXDMTDSDCRDNAPRTIFIISMYADSQPRGMAVXISVKCEXISTLSCENKIISFKEMNPPDNIKDTKSDIIFFQRSVPGHDNKMQFXSSSYEGYFLACEKERDLFKLILKKEDELGDRSIMFTVQNED

SEQ ID NO: 172 - protein, 157 aa, synthetic construct
  SITE 31: note = Homoserine
  SITE 63: note = Homoserine
  SITE 116: note = Homoserine
YFGKLESKLSVIRNLNDQVLFIDQGNRPLFXDMTDSDSRDNAPRTIFIISMYKDSQPRGMAVXISVKSEKISTLSSENKIISFKEMNPPDNIKDTKSDIIFFQRSVPGHDNKMQFXSSSYEGYFLASEKERDLFKLILKKEDELGDRSIMFTVQNED

SEQ ID NO: 173 - protein, 157 aa, synthetic construct
  SITE 31: note = Homoserine
  SITE 63: note = Homoserine
  VARIANT 70: note = N-(glutaryl-(Polyethylene glycol)x-azide)-L-lysine or C-(methylenylx-azide)-L-glycine, wherein each x is independently an integer from 1-30
  SITE 116: note = Homoserine
YFGKLKSKLSVIRNLNDQVLFIDQGNRPLFXDMTDSDSRDNAPRTIFIISMYADSQPRGMAVXISVKSEXISTLSSENKIISFKEMNPPDNIKDTKSDIIFFQRSVPGHDNKMQFXSSSYEGYFLASEKERDLFKLILKKEDELGDRSIMFTVQNED

SEQ ID NO: 174 - protein, 157 aa, synthetic construct
  SITE 31: note = Homoserine
  SITE 33: note = O-methyl-homoserine
  SITE 51: note = O-methyl-homoserine
  SITE 60: note = O-methyl-homoserine
  SITE 63: note = Homoserine
  SITE 86: note = O-methyl-homoserine
  SITE 113: note = O-methyl-homoserine
  SITE 116: note = Homoserine
  SITE 150: note = O-methyl-homoserine
YFGKLESKLSVIRNLNDQVLFIDQGNRPLFXDXTDSDCRDNAPRTIFIISXYKDSQPRGXAVXISVKCEKISTLSCENKIISFKEXNPPDNIKDTKSDIIFFQRSVPGHDNKXQFXSSSYEGYFLACEKERDLFKLILKKEDELGDRSIXFTVQNED

SEQ ID NO: 175 - protein, 157 aa, synthetic construct
  SITE 31: note = Homoserine
  SITE 33: note = O-methyl-homoserine
  SITE 51: note = O-methyl-homoserine
  SITE 60: note = O-methyl-homoserine
  SITE 63: note = Homoserine
  VARIANT 70: note = N-(glutaryl-(Polyethylene glycol)x-azide)-L-lysine or C-(methylenylx-azide)-L-glycine, wherein each x is independently an integer from 1-30
  SITE 86: note = O-methyl-homoserine
  SITE 113: note = O-methyl-homoserine
  SITE 116: note = Homoserine
  SITE 150: note = O-methyl-homoserine
YFGKLKSKLSVIRNLNDQVLFIDQGNRPLFXDXTDSDCRDNAPRTIFIISXYADSQPRGXAVXISVKCEXISTLSCENKIISFKEXNPPDNIKDTKSDIIFFQRSVPGHDNKXQFXSSSYEGYFLACEKERDLFKLILKKEDELGDRSIXFTVQNED

SEQ ID NO: 176 - protein, 157 aa, synthetic construct
  SITE 31: note = Homoserine
  SITE 33: note = O-methyl-homoserine
  SITE 51: note = O-methyl-homoserine
  SITE 60: note = O-methyl-homoserine
  SITE 63: note = Homoserine
  SITE 86: note = O-methyl-homoserine
  SITE 113: note = O-methyl-homoserine
  SITE 116: note = Homoserine
  SITE 150: note = O-methyl-homoserine
YFGKLESKLSVIRNLNDQVLFIDQGNRPLFXDXTDSDSRDNAPRTIFIISXYKDSQPRGXAVXISVKSEKISTLSSENKIISFKEXNPPDNIKDTKSDIIFFQRSVPGHDNKXQFXSSSYEGYFLASEKERDLFKLILKKEDELGDRSIXFTVQNED

SEQ ID NO: 177 - protein, 157 aa, synthetic construct
  SITE 31: note = Homoserine
  SITE 33: note = O-methyl-homoserine
  SITE 51: note = O-methyl-homoserine
  SITE 60: note = O-methyl-homoserine
  SITE 63: note = Homoserine
  VARIANT 70: note = N-(glutaryl-(Polyethylene glycol)x-azide)-L-lysine or C-(methylenylx-azide)-L-glycine, wherein each x is independently an integer from 1-30
  SITE 86: note = O-methyl-homoserine
  SITE 113: note = O-methyl-homoserine
  SITE 116: note = Homoserine
  SITE 150: note = O-methyl-homoserine
YFGKLKSKLSVIRNLNDQVLFIDQGNRPLFXDXTDSDSRDNAPRTIFIISXYADSQPRGXAVXISVKSEXISTLSSENKIISFKEXNPPDNIKDTKSDIIFFQRSVPGHDNKXQFXSSSYEGYFLASEKERDLFKLILKKEDELGDRSIXFTVQNED

SEQ ID NO: 178 - skipped

SEQ ID NO: 179 - protein, 157 aa, synthetic construct
  SITE 31: note = Homoserine
  SITE 63: note = Homoserine
  SITE 116: note = Homoserine
YFGKLKSKLSVIRNLNDQVLFIDQGNRPLFXDMTDSDCRDNAPRTIFIISMYADSQPRGMAVXISVKCEKISTLSCENKIISFKEMNPPDNIKDTKSDIIFFQRSVPGHDNKMQFXSSSYEGYFLACEKERDLFKLILKKEDELGDRSIMFTVQNED

SEQ ID NO: 180 - protein, 157 aa, synthetic construct
  SITE 31: note = Homoserine
  SITE 63: note = Homoserine
  SITE 116: note = Homoserine
YFGKLKSKLSVIRNLNDQVLFIDQGNRPLFXDMTDSDSRDNAPRTIFIISMYADSQPRGMAVXISVKSEKISTLSSENKIISFKEMNPPDNIKDTKSDIIFFQRSVPGHDNKMQFXSSSYEGYFLASEKERDLFKLILKKEDELGDRSIMFTVQNED

SEQ ID NO: 181-182 - skipped (2 entries)

SEQ ID NO: 183 - protein, 157 aa, synthetic construct
  SITE 31: note = Homoserine
  SITE 33: note = O-methyl-homoserine
  SITE 51: note = O-methyl-homoserine
  SITE 60: note = O-methyl-homoserine
  SITE 63: note = Homoserine
  SITE 86: note = O-methyl-homoserine
  SITE 113: note = O-methyl-homoserine
  SITE 116: note = Homoserine
  SITE 150: note = O-methyl-homoserine
YFGKLKSKLSVIRNLNDQVLFIDQGNRPLFXDXTDSDCRDNAPRTIFIISXYADSQPRGXAVXISVKCEKISTLSCENKIISFKEXNPPDNIKDTKSDIIFFQRSVPGHDNKXQFXSSSYEGYFLACEKERDLFKLILKKEDELGDRSIXFTVQNED

SEQ ID NO: 184 - protein, 157 aa, synthetic construct
  SITE 31: note = Homoserine
  SITE 33: note = O-methyl-homoserine
  SITE 51: note = O-methyl-homoserine
  SITE 60: note = O-methyl-homoserine
  SITE 63: note = Homoserine
  SITE 86: note = O-methyl-homoserine
  SITE 113: note = O-methyl-homoserine
  SITE 116: note = Homoserine
  SITE 150: note = O-methyl-homoserine
YFGKLKSKLSVIRNLNDQVLFIDQGNRPLFXDXTDSDSRDNAPRTIFIISXYADSQPRGXAVXISVKSEKISTLSSENKIISFKEXNPPDNIKDTKSDIIFFQRSVPGHDNKXQFXSSSYEGYFLASEKERDLFKLILKKEDELGDRSIXFTVQNED

SEQ ID NO: 185 - skipped

SEQ ID NO: 186 - protein, 157 aa, synthetic construct
  SITE 31: note = Homoserine
  SITE 63: note = Homoserine
  VARIANT 70: note = N-(glutaryl-(Polyethylene glycol)x-azide)-L-lysine or C-(methylenylx-azide)-L-glycine, wherein each x is independently an integer from 1-30
  SITE 116: note = Homoserine
YFGKLESKLSVIRNLNDQVLFIDQGNRPLFXDMTDSDCRDNAPRTIFIISMYKDSQPRGMAVXISVKCEXISTLSCENKIISFKEMNPPDNIKDTKSDIIFFQRSVPGHDNKMQFXSSSYEGYFLACEKERDLFKLILKKEDELGDRSIMFTVQNED

SEQ ID NO: 187 - protein, 157 aa, synthetic construct
  SITE 31: note = Homoserine
  SITE 63: note = Homoserine
  VARIANT 70: note = N-(glutaryl-(Polyethylene glycol)x-azide)-L-lysine or C-(methylenylx-azide)-L-glycine, wherein each x is independently an integer from 1-30
  SITE 116: note = Homoserine
YFGKLESKLSVIRNLNDQVLFIDQGNRPLFXDMTDSDSRDNAPRTIFIISMYKDSQPRGMAVXISVKSEXISTLSSENKIISFKEMNPPDNIKDTKSDIIFFQRSVPGHDNKMQFXSSSYEGYFLASEKERDLFKLILKKEDELGDRSIMFTVQNED

SEQ ID NO: 188-189 - skipped (2 entries)

SEQ ID NO: 190 - protein, 157 aa, synthetic construct
  SITE 31: note = Homoserine
  SITE 33: note = O-methyl-homoserine
  SITE 51: note = O-methyl-homoserine
  SITE 60: note = O-methyl-homoserine
  SITE 63: note = Homoserine
  VARIANT 70: note = N-(glutaryl-(Polyethylene glycol)x-azide)-L-lysine or C-(methylenylx-azide)-L-glycine, wherein each x is independently an integer from 1-30
  SITE 86: note = O-methyl-homoserine
  SITE 113: note = O-methyl-homoserine
  SITE 116: note = Homoserine
  SITE 150: note = O-methyl-homoserine
YFGKLESKLSVIRNLNDQVLFIDQGNRPLFXDXTDSDCRDNAPRTIFIISXYKDSQPRGXAVXISVKCEXISTLSCENKIISFKEXNPPDNIKDTKSDIIFFQRSVPGHDNKXQFXSSSYEGYFLACEKERDLFKLILKKEDELGDRSIXFTVQNED

SEQ ID NO: 191 - protein, 157 aa, synthetic construct
  SITE 31: note = Homoserine
  SITE 33: note = O-methyl-homoserine
  SITE 51: note = O-methyl-homoserine
  SITE 60: note = O-methyl-homoserine
  SITE 63: note = Homoserine
  VARIANT 70: note = N-(glutaryl-(Polyethylene glycol)x-azide)-L-lysine or C-(methylenylx-azide)-L-glycine, wherein each x is independently an integer from 1-30
  SITE 86: note = O-methyl-homoserine
  SITE 113: note = O-methyl-homoserine
  SITE 116: note = Homoserine
  SITE 150: note = O-methyl-homoserine
YFGKLESKLSVIRNLNDQVLFIDQGNRPLFXDXTDSDSRDNAPRTIFIISXYKDSQPRGXAVXISVKSEXISTLSSENKIISFKEXNPPDNIKDTKSDIIFFQRSVPGHDNKXQFXSSSYEGYFLASEKERDLFKLILKKEDELGDRSIXFTVQNED

SEQ ID NO: 192 - protein, 157 aa, synthetic construct
  SITE 31: note = Homoserine
  SITE 75: note = Homoserine
  SITE 116: note = Homoserine
YFGKLKSKLSVIRNLNDQVLFIDQGNRPLFXDMTDSDCRDNAPRTIFIISMYADSQPRGMAVAISVKCEKISTLXCENKIISFKEMNPPDNIKDTKSDIIFFQRSVPGHDNKMQFXSSSYEGYFLACEKERDLFKLILKKEDELGDRSIMFTVQNED

SEQ ID NO: 193 - protein, 157 aa, synthetic construct
  SITE 31: note = Homoserine
  SITE 75: note = Homoserine
  SITE 116: note = Homoserine
YFGKLKSKLSVIRNLNDQVLFIDQGNRPLFXDMTDSDSRDNAPRTIFIISMYADSQPRGMAVAISVKSEKISTLXSENKIISFKEMNPPDNIKDTKSDIIFFQRSVPGHDNKMQFXSSSYEGYFLASEKERDLFKLILKKEDELGDRSIMFTVQNED

SEQ ID NO: 194-195 - skipped (2 entries)

SEQ ID NO: 196 - protein, 157 aa, synthetic construct
  SITE 31: note = Homoserine
  SITE 33: note = O-methyl-homoserine
  SITE 51: note = O-methyl-homoserine
  SITE 60: note = O-methyl-homoserine
  SITE 75: note = Homoserine
  SITE 86: note = O-methyl-homoserine
  SITE 113: note = O-methyl-homoserine
  SITE 116: note = Homoserine
  SITE 150: note = O-methyl-homoserine
YFGKLKSKLSVIRNLNDQVLFIDQGNRPLFXDXTDSDCRDNAPRTIFIISXYADSQPRGXAVAISVKCEKISTLXCENKIISFKEXNPPDNIKDTKSDIIFFQRSVPGHDNKXQFXSSSYEGYFLACEKERDLFKLILKKEDELGDRSIXFTVQNED

SEQ ID NO: 197 - protein, 157 aa, synthetic construct
  SITE 31: note = Homoserine
  SITE 33: note = O-methyl-homoserine
  SITE 51: note = O-methyl-homoserine
  SITE 60: note = O-methyl-homoserine
  SITE 75: note = Homoserine
  SITE 86: note = O-methyl-homoserine
  SITE 113: note = O-methyl-homoserine
  SITE 116: note = Homoserine
  SITE 150: note = O-methyl-homoserine
YFGKLKSKLSVIRNLNDQVLFIDQGNRPLFXDXTDSDSRDNAPRTIFIISXYADSQPRGXAVAISVKCEKISTLXSENKIISFKEXNPPDNIKDTKSDIIFFQRSVPGHDNKXQFXSSSYEGYFLASEKERDLFKLILKKEDELGDRSIXFTVQNED

SEQ ID NO: 198-199 - skipped (2 entries)

SEQ ID NO: 200 - protein, 157 aa, synthetic construct
  SITE 31: note = Homoserine
  SITE 33: note = O-methyl-homoserine
  SITE 51: note = O-methyl-homoserine
  SITE 60: note = O-methyl-homoserine
  SITE 63: note = Homoserine
  SITE 86: note = O-methyl-homoserine
  SITE 113: note = O-methyl-homoserine
  SITE 116: note = Homoserine
  SITE 150: note = O-methyl-homoserine
YFGKLKSKLSVIRNLNDQVLFIDQGNRPLFXDXTDSDSRDNAPRTIFIISXYADSQPRGXAVXISVKCEKISTLSCENKIISFKEXNPPDNIKDTKSDIIFFQRSVPGHDNKXQFXSSSYEGYFLACEKERDLFKLILKKEDELGDRSIXFTVQNED

SEQ ID NO: 201 - protein, 157 aa, synthetic construct
  SITE 31: note = Homoserine
  SITE 33: note = O-methyl-homoserine
  SITE 51: note = O-methyl-homoserine
  SITE 60: note = O-methyl-homoserine
  SITE 63: note = Homoserine
  SITE 86: note = O-methyl-homoserine
  SITE 113: note = O-methyl-homoserine
  SITE 116: note = Homoserine
  SITE 150: note = O-methyl-homoserine
YFGKLKSKLSVIRNLNDQVLFIDQGNRPLFXDXTDSDARDNAPRTIFIISXYADSQPRGXAVXISVKCEKISTLSCENKIISFKEXNPPDNIKDTKSDIIFFQRSVPGHDNKXQFXSSSYEGYFLACEKERDLFKLILKKEDELGDRSIXFTVQNED

SEQ ID NO: 202 - protein, 157 aa, synthetic construct
  SITE 31: note = Homoserine
  SITE 63: note = Homoserine
  SITE 116: note = Homoserine
YFGKLKSKLSVIRNLNDQVLFIDQGNRPLFXDMTDSDSRDNAPRTIFIISMYADSQPRGMAVXISVKCEKISTLSCENKIISFKEMNPPDNIKDTKSDIIFFQRSVPGHDNKMQFXSSSYEGYFLACEKERDLFKLILKKEDELGDRSIMFTVQNED

SEQ ID NO: 203 - protein, 157 aa, synthetic construct
  SITE 31: note = Homoserine
  SITE 63: note = Homoserine
  SITE 116: note = Homoserine
YFGKLKSKLSVIRNLNDQVLFIDQGNRPLFXDMTDSDARDNAPRTIFIISMYADSQPRGMAVXISVKCEKISTLSCENKIISFKEMNPPDNIKDTKSDIIFFQRSVPGHDNKMQFXSSSYEGYFLACEKERDLFKLILKKEDELGDRSIMFTVQNED

SEQ ID NO: 204 - protein, 157 aa, synthetic construct
YFGKLESKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 205 - protein, 157 aa, synthetic construct
YFGKLESKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 206 - protein, 157 aa, synthetic construct
YFGKLESKLSIIRNLNDQVLFIDQGNRPLFEDMTDSDARDNAPRTIFIISMYADAQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 207 - protein, 157 aa, synthetic construct
YFGKLESKLSIIRNLNDQVLFIDQGNRPLFEDMTDSDARDNAPRTIFIISGYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 208 - protein, 157 aa, synthetic construct
YFGKLESKLSIIRNLNDQVLFIDQGNRPLFEDMTDSDARDNAPRTIFIISMYAASQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 209 - protein, 157 aa, synthetic construct
YAGKLESKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 210 - protein, 157 aa, synthetic construct
YFGKLESKLSIIRNLNDQVLFIDQGNRPLFADMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 211 - protein, 157 aa, synthetic construct
YFGKLESKLSIIRNLNDQVLFIDQGNRPLFEDMADSDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 212 - protein, 157 aa, synthetic construct
YFGKLESKLSIIRNLNDQVLFIDQGNRPLFEDMTASDARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 213 - protein, 157 aa, synthetic construct
YFGKLESKLSIIRNLNDQVLFIDQGNRPLFEDMTDADARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 214 - protein, 157 aa, synthetic construct
YFGKLESKLSIIRNLNDQVLFIDQGNRPLFEDMTDSAARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 215 - protein, 157 aa, synthetic construct
YFGKLESKLSIIRNLNDQVLFIDQGNRPLFADMTDSAARDNAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 216 - protein, 157 aa, synthetic construct
YFGKLESKLSIIRNLNDQVLFIDQGNRPLFEDMTDSDARANAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 217 - protein, 157 aa, synthetic construct
YFGKLESKLSIIRNLNDQVLFIDQGNRPLFEDMTDSDARDAAPRTIFIISMYADSQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 218 - protein, 157 aa, synthetic construct
YFGKLESKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHDNKMQFESSSYEGYFLAAEKERALFKLILKKEDELGDRSIMFTVQNED

SEQ ID NO: 219 - protein, 157 aa, synthetic construct
YFGKLESKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPAHDNKMQFESSSYEGYFLAAEKERDLFKLILKKEDELGDRSIMFTVQNED

SEQ ID NO: 220 - protein, 157 aa, synthetic construct
YFGKLESKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGADNKMQFESSSYEGYFLAAEKERDLFKLILKKEDELGDRSIMFTVQNED

SEQ ID NO: 221 - protein, 157 aa, synthetic construct
YFGKLESKLSIIRNLNDQVLFIDQGNRPLFEDMTDSDARDNAPRTIFIISMYADSQPRGMAVTISVKCEKISTLSAENKIISFKEMNPPDNIKDTKSDIIFFQRSVPGHANKMQFESSSYEGYFLAAEKERDLFKLILKKEDELGDRSIMFTVQNED

SEQ ID NO: 222 - protein, 157 aa, synthetic construct
YFGKLESRLSIIRNLNDQVLFIDQGNRPLFEDMTDSDARDNAPRTIFIISMYKDSQPRGMAVTISVKCEKISTLSAENKIISFKEMNPPDNIKDTKSDIIFFERSVPGHDNKMQFESSSYEGYFLAAEKERDLFKLILKKEDELGDRSIMFTVQNED

SEQ ID NO: 223 - protein, 157 aa, synthetic construct
YFGKLESELSIIRNLNDQVLFIDQGNRPLFEDMTDSDARDNAPRTIFIISMYKDSQPRGMAVTISVKCEKISTLSAENKIISFKEMNPPDNIKDTKSDIIFFRRSVPGHDNKMQFESSSYEGYFLAAEKERDLFKLILKKEDELGDRSIMFTVQNED

SEQ ID NO: 224 - protein, 157 aa, synthetic construct
YFGKLESKLSIIRNLNDQVLFIDQGNRPLFEDMTDSDARDNAPRTIFIISMYKDSQPRGMAVTISVKCEKISTLSAENKIISFKEMNPPDNIKDTKSDIIFFKRSVPGHDNKMQFESSSYEGYFLAAEKERDLFKLILKKEDELGDRSIMFTVQNED

SEQ ID NO: 225 - protein, 157 aa, synthetic construct
YFGKLESKLSIIRNLNDQVLFIDQGNRPLFEDMTDSDARDNAPRTIFIISMYKDHQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 226 - protein, 157 aa, synthetic construct
YFGKLESKLSIIRNLNDQVLFIDQGNRPLFEDMTDSDARDNAPRTIFIISMYKDRQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 227 - protein, 157 aa, synthetic construct
YFGKLESKLSIIRNLNDQVLFIDQGNRPLFEDMTDSDARDNAPRTIFIISMYKDTQPRGMAVTISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 228 - protein, 157 aa, synthetic construct
YFGKLESKLSIIRNLNDQVLFIDQGNRPLFEDMTDSDARDNAPRTIFIISMYKDSQPRGMAVTISVKCEKISTLSAENKIISFKEMNPPDNIKDTKSDIIFFQRIVPGHDNKMQFESSSYEGYFLAAEKERDLFKLILKKEDELGDRSIMFTVQNED

SEQ ID NO: 229 - protein, 157 aa, synthetic construct
YFGKLESKLSIIRNLNDQVLFIDQGNRPLFEDMTDSDARDNAPRTIFIISMYKDSQPRGMAVTISVKCEKISTLSAENKIISFKEMNPPDNIKDTKSDIIFFQRKVPGHDNKMQFESSSYEGYFLAAEKERDLFKLILKKEDELGDRSIMFTVQNED

SEQ ID NO: 230 - protein, 157 aa, synthetic construct
YFGKLKSLLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 231 - protein, 157 aa, synthetic construct
YFGKLKSKLSIIRNLNDQVLFIDQGNRPLFEDMTDSDARDNAPRTIFIESMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 232 - protein, 157 aa, synthetic construct
YFGKLKSKLSIIRNLNDQVLFIDQGNRPLFEDMTDSDARDNAPRTIFIMSMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 233 - protein, 157 aa, synthetic construct
YFGKLKSKLSIIRNLNDQVLFIDQGNRPLFEDMTDSDARDNAPRTIFIRSMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 234 - protein, 157 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFRRSVPGHDNKMQFESSSYEGYFLAAEKERDLFKLILKKEDELGDRSIMFTVQNED

SEQ ID NO: 235 - protein, 157 aa, synthetic construct
YFGKLKSELSIIRNLNDQVLFIDQGNRPLFEDMTDSDARDNAPRTIFIISMYADSQPRGMAVAISVKCEKISTLSAENKIISFKEMNPPDNIKDTKSDIIFFRRSVPGHDNKMQFESSSYEGYFLAAEKERDLFKLILKKEDELGDRSIMFTVQNED

SEQ ID NO: 236 - protein, 157 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTRQNED

SEQ ID NO: 237 - protein, 157 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTEQNED

SEQ ID NO: 238 - protein, 157 aa, synthetic construct
YFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTYQNED

SEQ ID NO: 239 - protein, 157 aa, synthetic construct
YFGKLKSKLSIIRNLNDQVLFIDQGNRPLFEDMTDSDARDNAPRTIFIISGYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 240 - protein, 157 aa, synthetic construct
YFGKLRSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 241 - protein, 161 aa, synthetic construct
GGGGYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 242 - protein, 158 aa, synthetic construct
GYFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 243 - protein, 157 aa, synthetic construct
MFGKLKSKLSIIRNLNDQVLFIDQGNRPLFEDMTDSDARDNAPRTIFIISMYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 244 - protein, 158 aa, synthetic construct
GYFGKLKSKLSIIRNLNDQVLFIDQGNRPLFEDMTDSDARDNAPRTIFIISGYADSQPRGMAVAISVKCEKISTLSAENKIISFKEMNPPDNIKDTKSDIIFFQRSVPGHDNKMQFESSSYEGYFLAAEKERDLFKLILKKEDELGDRSIMFTVQNED

SEQ ID NO: 245-300 - skipped (56 entries)

SEQ ID NO: 301 - protein, 30 aa, synthetic construct
  SITE 30: note = Alpha-keto-phenylalanine
YFGKLESKLSVIRNLNDQVLFIDQGNRPLX

SEQ ID NO: 302 - protein, 30 aa, synthetic construct
  SITE 30: note = alpha-keto-phenylalanine
YFGKLKSKLSVIRNLNDQVLFIDQGNRPLX

SEQ ID NO: 303 - protein, 30 aa, synthetic construct
  SITE 30: note = alpha-keto-phenylalanine
GFGKLESKLSVIRNLNDQVLFIDQGNRPLX

SEQ ID NO: 304 - protein, 30 aa, synthetic construct
  SITE 30: note = alpha-keto-phenylalanine
YAGKLESKLSVIRNLNDQVLFIDQGNRPLX

SEQ ID NO: 305 - protein, 30 aa, synthetic construct
  SITE 30: note = alpha-keto-phenylalanine
GFGKLKSKLSVIRNLNDQVLFIDQGNRPLX

SEQ ID NO: 306 - protein, 30 aa, synthetic construct
  SITE 30: note = alpha-keto-phenylalanine
YAGKLKSKLSVIRNLNDQVLFIDQGNRPLX

SEQ ID NO: 307 - protein, 30 aa, synthetic construct
  SITE 30: note = alpha-keto-phenylalanine
GAGKLKSKLSVIRNLNDQVLFIDQGNRPLX

SEQ ID NO: 308 - protein, 30 aa, synthetic construct
  SITE 30: note = alpha-keto-phenylalanine
GAGKLESKLSVIRNLNDQVLFIDQGNRPLX

SEQ ID NO: 309 - protein, 30 aa, synthetic construct
  SITE 30: note = alpha-keto-phenylalanine
YFGKLKSKLSIIRNLNDQVLFIDQGNRPLX

SEQ ID NO: 310 - protein, 32 aa, synthetic construct
  SITE 1: note = 5-oxa-proline
  SITE 32: note = alpha-keto-valine
XDMTDSDCRDNAPRTIFIISMYKDSQPRGMAX

SEQ ID NO: 311 - protein, 32 aa, synthetic construct
  SITE 1: note = 5-oxa-proline
  SITE 3: note = Norleucine
  SITE 21: note = Norleucine
  SITE 30: note = Norleucine
  SITE 32: note = alpha-keto-valine
XDXTDSDCRDNAPRTIFIISXYKDSQPRGXAX

SEQ ID NO: 312 - protein, 32 aa, synthetic construct; identical to SEQ ID NO: 311
  SITE 1: note = 5-oxa-proline
  SITE 3: note = O-methyl-homoserine
  SITE 21: note = O-methyl-homoserine
  SITE 30: note = O-methyl-homoserine
  SITE 32: note = alpha-keto-valine

SEQ ID NO: 313 - protein, 32 aa, synthetic construct
  SITE 1: note = 5-oxa-proline
  SITE 32: note = alpha-keto-valine
XDMTDSDCRDNAPRTIFIISMYADSQPRGMAX

SEQ ID NO: 314 - protein, 32 aa, synthetic construct
  SITE 1: note = 5-oxa-proline
  SITE 3: note = Norleucine
  SITE 21: note = Norleucine
  SITE 30: note = Norleucine
  SITE 32: note = alpha-keto-valine
XDXTDSDCRDNAPRTIFIISXYADSQPRGXAX

SEQ ID NO: 315 - protein, 32 aa, synthetic construct; identical to SEQ ID NO: 314
  SITE 1: note = 5-oxa-proline
  SITE 3: note = O-methyl-homoserine
  SITE 21: note = O-methyl-homoserine
  SITE 30: note = O-methyl-homoserine
  SITE 32: note = alpha-keto-valine

SEQ ID NO: 316 - protein, 32 aa, synthetic construct
  SITE 1: note = 5-oxa-proline
  SITE 3: note = O-methyl-homoserine
  SITE 21: note = O-methyl-homoserine
  SITE 30: note = O-methyl-homoserine
  SITE 32: note = alpha-keto-valine
XDXTDSDSRDNAPRTIFIISXYADSQPRGXAX

SEQ ID NO: 317 - protein, 32 aa, synthetic construct
  SITE 1: note = 5-oxa-proline
  SITE 3: note = O-methyl-homoserine
  SITE 21: note = O-methyl-homoserine
  SITE 30: note = O-methyl-homoserine
  SITE 32: note = alpha-keto-valine
XDXTDSDSRDNAPRTIFIISXYKDSQPRGXAX

SEQ ID NO: 318 - protein, 44 aa, synthetic construct
  SITE 1: note = 5-oxa-proline
  SITE 44: note = alpha-keto-leucine
XDMTDSDCRDNAPRTIFIISMYKDSQPRGMAVTISVKCEKISTX

SEQ ID NO: 319 - protein, 44 aa, synthetic construct
  SITE 1: note = 5-oxa-proline
  SITE 44: note = alpha-keto-leucine
XDMTDSDCRDNAPRTIFIISMYADSQPRGMAVTISVKCEKISTX

SEQ ID NO: 320 - protein, 44 aa, synthetic construct; identical to SEQ ID NO: 319
  SITE 1: note = 5-oxa-proline
  SITE 44: note = alpha-keto-leucine

SEQ ID NO: 321 - protein, 44 aa, synthetic construct
  SITE 1: note = 5-oxa-proline
  SITE 3: note = Norleucine
  SITE 21: note = Norleucine
  SITE 30: note = Norleucine
  SITE 44: note = alpha-keto-leucine
XDXTDSDCRDNAPRTIFIISXYKDSQPRGXAVTISVKCEKISTX

SEQ ID NO: 322 - protein, 44 aa, synthetic construct; identical to SEQ ID NO: 321
  SITE 1: note = 5-oxa-proline
  SITE 3: note = O-methyl-homoserine
  SITE 21: note = O-methyl-homoserine
  SITE 30: note = O-methyl-homoserine
  SITE 44: note = alpha-keto-leucine

SEQ ID NO: 323 - protein, 44 aa, synthetic construct
  SITE 1: note = 5-oxa-proline
  SITE 3: note = O-methyl-homoserine
  SITE 21: note = O-methyl-homoserine
  SITE 30: note = O-methyl-homoserine
  SITE 44: note = alpha-keto-leucine
XDXTDSDCRDNAPRTIFIISXYADSQPRGXAVTISVKCEKISTX

SEQ ID NO: 324 - protein, 44 aa, synthetic construct
  SITE 1: note = 5-oxa-proline
  SITE 3: note = O-methyl-homoserine
  SITE 21: note = O-methyl-homoserine
  SITE 30: note = O-methyl-homoserine
  SITE 44: note = alpha-keto-leucine
XDXTDSDCRDNAPRTIFIISXYADSQPRGXAVAISVKCEKISTX

SEQ ID NO: 325 - protein, 44 aa, synthetic construct
  SITE 1: note = 5-oxa-proline
  SITE 3: note = O-methyl-homoserine
  SITE 21: note = O-methyl-homoserine
  SITE 30: note = O-methyl-homoserine
  SITE 44: note = alpha-keto-leucine
XDXTDSDSRDNAPRTIFIISXYADSQPRGXAVAISVKCEKISTX

SEQ ID NO: 326 - protein, 44 aa, synthetic construct
  SITE 1: note = 5-oxa-proline
  SITE 44: note = alpha-keto-leucine
XDMTDSDCRDNAPRTIFIISMYKDSQPRGMAVTISVKSEKISTX

SEQ ID NO: 327 - protein, 53 aa, synthetic construct
  SITE 1: note = 5-oxa-proline
  SITE 53: note = alpha-keto-phenylalanine
XISVKCEKISTLSCENKIISFKEMNPPDNIKDTKSDIIFFQRSVPGHDNKMQX

SEQ ID NO: 328 - protein, 53 aa, synthetic construct
  SITE 1: note = 5-oxa-proline
  SITE 24: note = Norleucine
  SITE 51: note = Norleucine
  SITE 53: note = alpha-keto-phenylalanine
XISVKCEKISTLSCENKIISFKEXNPPDNIKDTKSDIIFFQRSVPGHDNKXQX

SEQ ID NO: 329 - protein, 53 aa, synthetic construct; identical to SEQ ID NO: 328
  SITE 1: note = 5-oxa-proline
  SITE 24: note = O-methyl-homoserine
  SITE 51: note = O-methyl-homoserine
  SITE 53: note = alpha-keto-phenylalanine

SEQ ID NO: 330 - protein, 53 aa, synthetic construct
  SITE 1: note = 5-oxa-proline
  SITE 53: note = alpha-keto-phenylalanine
XISVKSEKISTLSCENKIISFKEMNPPDNIKDTKSDIIFFQRSVPGHDNKMQX

SEQ ID NO: 331 - protein, 53 aa, synthetic construct
  SITE 1: note = 5-oxa-proline
  SITE 24: note = Norleucine
  SITE 51: note = Norleucine
  SITE 53: note = alpha-keto-phenylalanine
XISVKSEKISTLSCENKIISFKEXNPPDNIKDTKSDIIFFQRSVPGHDNKXQX

SEQ ID NO: 332 - protein, 53 aa, synthetic construct; identical to SEQ ID NO: 331
  SITE 1: note = 5-oxa-proline
  SITE 24: note = O-methyl-homoserine
  SITE 51: note = O-methyl-homoserine
  SITE 53: note = alpha-keto-phenylalanine

SEQ ID NO: 333 - protein, 53 aa, synthetic construct
  SITE 1: note = 5-oxa-proline
  SITE 53: note = alpha-keto-phenylalanine
XISVKSEKISTLSSENKIISFKEMNPPDNIKDTKSDIIFFQRSVPGHDNKMQX

SEQ ID NO: 334 - protein, 53 aa, synthetic construct
  SITE 1: note = 5-oxa-proline
  SITE 53: note = alpha-keto-phenylalanine
XISVKCEKISTLSSENKIISFKEMNPPDNIKDTKSDIIFFQRSVPGHDNKMQX

SEQ ID NO: 335 - protein, 53 aa, synthetic construct
  SITE 1: note = 5-oxa-proline
  SITE 24: note = O-methyl-homoserine
  SITE 51: note = O-methyl-homoserine
  SITE 53: note = alpha-keto-phenylalanine
XISVKCEKISTLSSENKIISFKEXNPPDNIKDTKSDIIFFQRSVPGHDNKXQX

SEQ ID NO: 336 - protein, 53 aa, synthetic construct
  SITE 1: note = 5-oxa-proline
  SITE 24: note = O-methyl-homoserine
  SITE 51: note = O-methyl-homoserine
  SITE 53: note = alpha-keto-phenylalanine
XISVKSEKISTLSSENKIISFKEXNPPDNIKDTKSDIIFFQRSVPGHDNKXQX

SEQ ID NO: 337 - protein, 41 aa, synthetic construct
  SITE 1: note = 5-oxa-proline
  SITE 41: note = alpha-keto-phenylalanine
XCENKIISFKEMNPPDNIKDTKSDIIFFQRSVPGHDNKMQX

SEQ ID NO: 338 - protein, 41 aa, synthetic construct
  SITE 1: note = 5-oxa-proline
  SITE 41: note = alpha-keto-phenylalanine
XSENKIISFKEMNPPDNIKDTKSDIIFFQRSVPGHDNKMQX

SEQ ID NO: 339 - protein, 41 aa, synthetic construct
  SITE 1: note = 5-oxa-proline
  SITE 12: note = Norleucine
  SITE 39: note = Norleucine
  SITE 41: note = alpha-keto-phenylalanine
XCENKIISFKEXNPPDNIKDTKSDIIFFQRSVPGHDNKXQX

SEQ ID NO: 340 - protein, 41 aa, synthetic construct
  SITE 1: note = 5-oxa-proline
  SITE 12: note = Norleucine
  SITE 39: note = Norleucine
  SITE 41: note = alpha-keto-phenylalanine
XSENKIISFKEXNPPDNIKDTKSDIIFFQRSVPGHDNKXQX

SEQ ID NO: 341 - protein, 41 aa, synthetic construct; identical to SEQ ID NO: 339
  SITE 1: note = 5-oxa-proline
  SITE 12: note = O-methyl-homoserine
  SITE 39: note = O-methyl-homoserine
  SITE 41: note = alpha-keto-phenylalanine

SEQ ID NO: 342 - protein, 41 aa, synthetic construct; identical to SEQ ID NO: 340
  SITE 1: note = 5-oxa-proline
  SITE 12: note = O-methyl-homoserine
  SITE 39: note = O-methyl-homoserine
  SITE 41: note = alpha-keto-phenylalanine

SEQ ID NO: 343 - protein, 42 aa, synthetic construct
  SITE 1: note = 5-oxa-proline
XSSSYEGYFLACEKERDLFKLILKKEDELGDRSIMFTVQNED

SEQ ID NO: 344 - protein, 42 aa, synthetic construct
  SITE 1: note = 5-oxa-proline
XSSSYEGYFLASEKERDLFKLILKKEDELGDRSIMFTVQNED

SEQ ID NO: 345 - protein, 42 aa, synthetic construct
  SITE 1: note = 5-oxa-proline
  SITE 35: note = Norleucine
XSSSYEGYFLACEKERDLFKLILKKEDELGDRSIXFTVQNED

SEQ ID NO: 346 - protein, 42 aa, synthetic construct
  SITE 1: note = 5-oxa-proline
  SITE 35: note = Norleucine
XSSSYEGYFLASEKERDLFKLILKKEDELGDRSIXFTVQNED

SEQ ID NO: 347 - protein, 42 aa, synthetic construct; identical to SEQ ID NO: 345
  SITE 1: note = 5-oxa-proline
  SITE 35: note = O-methyl-homoserine

SEQ ID NO: 348 - protein, 42 aa, synthetic construct; identical to SEQ ID NO: 346
  SITE 1: note = 5-oxa-proline
  SITE 35: note = O-methyl-homoserine

SEQ ID NO: 349 - protein, 32 aa, synthetic construct; identical to SEQ ID NO: 310
  SITE 1: note = 5-oxa-proline
  SITE 32: note = alpha-keto-valine

SEQ ID NO: 350 - protein, 12 aa, synthetic construct
  SITE 1: note = 5-oxa-proline
  SITE 12: note = alpha-keto-leucine
XISVKCEKISTX

SEQ ID NO: 351 - protein, 26 aa, synthetic construct
  SITE 1: note = 5-oxa-proline
  SITE 26: note = alpha-keto-leucine
XDMTDSDCRDNAPRTIFIISMYKDSX

SEQ ID NO: 352 - protein, 18 aa, synthetic construct
  SITE 1: note = 5-oxa-proline
  SITE 18: note = alpha-keto-leucine
XRGMAVTISVKCEKISTX

SEQ ID NO: 353 - protein, 19 aa, synthetic construct
  SITE 1: note = 5-oxa-proline
  SITE 19: note = alpha-keto-leucine
XDMTDSDCRDNAPRTIFIX

SEQ ID NO: 354 - protein, 25 aa, synthetic construct
  SITE 1: note = 5-oxa-proline
  SITE 25: note = alpha-keto-leucine
XMYKDSQPRGMAVTISVKCEKISTX

SEQ ID NO: 355 - protein, 36 aa, synthetic construct
  SITE 1: note = 5-oxa-proline
  SITE 36: note = alpha-keto-valine
XDMTDSDCRDNAPRTIFIISMYKDSQPRGMAVTISX

SEQ ID NO: 356 - protein, 8 aa, synthetic construct
  SITE 1: note = 5-oxa-proline
  SITE 8: note = alpha-keto-leucine
XCEKISTX

SEQ ID NO: 357 - protein, 46 aa, synthetic construct
  SITE 1: note = 5-oxa-proline
  SITE 46: note = alpha-keto-phenylalanine
XCENKIISFKEMNPPDNIKDTKSDIIFFQRSVPGHDNKMQFESSSX

SEQ ID NO: 358 - protein, 37 aa, synthetic construct
  SITE 1: note = 5-oxa-proline
XGYFLACEKERDLFKLILKKEDELGDRSIMFTVQNED

SEQ ID NO: 359 - protein, 10 aa, synthetic construct
  VARIANT 1..10: note = This sequence may encompass 1-10 residues
GGGGGGGGGG

SEQ ID NO: 360 - protein, 4 aa, synthetic construct
GGGG

SEQ ID NO: 361 - protein, 5 aa, synthetic construct
GGGGG

SEQ ID NO: 362 - protein, 15 aa, synthetic construct
  VARIANT 1..15: note = This sequence may encompass 1-15 residues
GGGGGGGGGGGGGGG

SEQ ID NO: 363 - protein, 4 aa, synthetic construct
  VARIANT 1..4: note = This sequence may encompass 1-4 residues
GGGG

SEQ ID NO: 364 - protein, 5 aa, synthetic construct
  VARIANT 1..5: note = This sequence may encompass 1-5 residues
GGGGG

SEQ ID NO: 365 - protein, 6 aa, synthetic construct
  VARIANT 1..6: note = This sequence may encompass 1-6 residues
GGGGGG

SEQ ID NO: 366 - protein, 7 aa, synthetic construct
  VARIANT 1..7: note = This sequence may encompass 1-7 residues
GGGGGGG

SEQ ID NO: 367 - protein, 8 aa, synthetic construct
  VARIANT 1..8: note = This sequence may encompass 1-8 residues
GGGGGGGG

SEQ ID NO: 368 - protein, 9 aa, synthetic construct
  VARIANT 1..9: note = This sequence may encompass 1-9 residues
GGGGGGGGG

SEQ ID NO: 369 - protein, 10 aa, synthetic construct
  VARIANT 1..10: note = This sequence may encompass 1-10 residues
GGGGGGGGGG

SEQ ID NO: 370 - protein, 12 aa, synthetic construct
  VARIANT 1..12: note = This sequence may encompass 1-12 residues
GGGGGGGGGGGG

SEQ ID NO: 371 - protein, 15 aa, synthetic construct
  VARIANT 1..15: note = This sequence may encompass 1-15 residues
GGGGGGGGGGGGGGG

SEQ ID NO: 372 - protein, 4 aa, synthetic construct
  VARIANT 1..4: note = This sequence may encompass 2-4 residues
GGGG

SEQ ID NO: 373 - protein, 5 aa, synthetic construct
  VARIANT 1..5: note = This sequence may encompass 2-5 residues
GGGGG

SEQ ID NO: 374 - protein, 6 aa, synthetic construct
  VARIANT 1..6: note = This sequence may encompass 2-6 residues
GGGGGG

SEQ ID NO: 375 - protein, 7 aa, synthetic construct
  VARIANT 1..7: note = This sequence may encompass 2-7 residues
GGGGGGG

SEQ ID NO: 376 - protein, 8 aa, synthetic construct
  VARIANT 1..8: note = This sequence may encompass 2-8 residues
GGGGGGGG

SEQ ID NO: 377 - protein, 9 aa, synthetic construct
  VARIANT 1..9: note = This sequence may encompass 2-9 residues
GGGGGGGGG

SEQ ID NO: 378 - protein, 10 aa, synthetic construct
  VARIANT 1..10: note = This sequence may encompass 2-10 residues
GGGGGGGGGG

SEQ ID NO: 379 - protein, 12 aa, synthetic construct
  VARIANT 1..12: note = This sequence may encompass 2-12 residues
GGGGGGGGGGGG

SEQ ID NO: 380 - protein, 15 aa, synthetic construct
  VARIANT 1..15: note = This sequence may encompass 2-15 residues
GGGGGGGGGGGGGGG

SEQ ID NO: 381 - protein, 4 aa, synthetic construct
  VARIANT 1..4: note = This sequence may encompass 3-4 residues
GGGG

SEQ ID NO: 382 - protein, 5 aa, synthetic construct
  VARIANT 1..5: note = This sequence may encompass 3-5 residues
GGGGG

SEQ ID NO: 383 - protein, 6 aa, synthetic construct
  VARIANT 1..6: note = This sequence may encompass 3-6 residues
GGGGGG

SEQ ID NO: 384 - protein, 7 aa, synthetic construct
  VARIANT 1..7: note = This sequence may encompass 3-7 residues
GGGGGGG

SEQ ID NO: 385 - protein, 8 aa, synthetic construct
  VARIANT 1..8: note = This sequence may encompass 3-8 residues
GGGGGGGG

SEQ ID NO: 386 - protein, 9 aa, synthetic construct
  VARIANT 1..9: note = This sequence may encompass 3-9 residues
GGGGGGGGG

SEQ ID NO: 387 - protein, 10 aa, synthetic construct
  VARIANT 1..10: note = This sequence may encompass 3-10 residues
GGGGGGGGGG

SEQ ID NO: 388 - protein, 12 aa, synthetic construct
  VARIANT 1..12: note = This sequence may encompass 3-12 residues
GGGGGGGGGGGG

SEQ ID NO: 389 - protein, 15 aa, synthetic construct
  VARIANT 1..15: note = This sequence may encompass 3-15 residues
GGGGGGGGGGGGGGG

SEQ ID NO: 390 - protein, 5 aa, synthetic construct
  VARIANT 1..5: note = This sequence may encompass 4-5 residues
GGGGG

SEQ ID NO: 391 - protein, 6 aa, synthetic construct
  VARIANT 1..6: note = This sequence may encompass 4-6 residues
GGGGGG

SEQ ID NO: 392 - protein, 7 aa, synthetic construct
  VARIANT 1..7: note = This sequence may encompass 4-7 residues
GGGGGGG

SEQ ID NO: 393 - protein, 8 aa, synthetic construct
  VARIANT 1..8: note = This sequence may encompass 4-8 residues
GGGGGGGG

SEQ ID NO: 394 - protein, 9 aa, synthetic construct
  VARIANT 1..9: note = This sequence may encompass 4-9 residues
GGGGGGGGG

SEQ ID NO: 395 - protein, 10 aa, synthetic construct
  VARIANT 1..10: note = This sequence may encompass 4-10 residues
GGGGGGGGGG

SEQ ID NO: 396 - protein, 12 aa, synthetic construct
  VARIANT 1..12: note = This sequence may encompass 4-12 residues
GGGGGGGGGGGG

SEQ ID NO: 397 - protein, 15 aa, synthetic construct
  VARIANT 1..15: note = This sequence may encompass 4-15 residues
GGGGGGGGGGGGGGG

SEQ ID NO: 398 - protein, 6 aa, synthetic construct
  VARIANT 1..6: note = This sequence may encompass 5-6 residues
GGGGGG

SEQ ID NO: 399 - protein, 7 aa, synthetic construct
  VARIANT 1..7: note = This sequence may encompass 5-7 residues
GGGGGGG

SEQ ID NO: 400 - protein, 8 aa, synthetic construct
  VARIANT 1..8: note = This sequence may encompass 5-8 residues
GGGGGGGG

SEQ ID NO: 401 - protein, 9 aa, synthetic construct
  VARIANT 1..9: note = This sequence may encompass 5-9 residues
GGGGGGGGG

SEQ ID NO: 402 - protein, 10 aa, synthetic construct
  VARIANT 1..10: note = This sequence may encompass 5-10 residues
GGGGGGGGGG

SEQ ID NO: 403 - protein, 12 aa, synthetic construct
  VARIANT 1..12: note = This sequence may encompass 5-12 residues
GGGGGGGGGGGG

SEQ ID NO: 404 - protein, 15 aa, synthetic construct
  VARIANT 1..15: note = This sequence may encompass 5-15 residues
GGGGGGGGGGGGGGG

SEQ ID NO: 405 - protein, 7 aa, synthetic construct
  VARIANT 1..7: note = This sequence may encompass 6-7 residues
GGGGGGG

SEQ ID NO: 406 - protein, 8 aa, synthetic construct
  VARIANT 1..8: note = This sequence may encompass 6-8 residues
GGGGGGGG

SEQ ID NO: 407 - protein, 9 aa, synthetic construct
  VARIANT 1..9: note = This sequence may encompass 6-9 residues
GGGGGGGGG

SEQ ID NO: 408 - protein, 10 aa, synthetic construct
  VARIANT 1..10: note = This sequence may encompass 6-10 residues
GGGGGGGGGG

SEQ ID NO: 409 - protein, 12 aa, synthetic construct
  VARIANT 1..12: note = This sequence may encompass 6-12 residues
GGGGGGGGGGGG

SEQ ID NO: 410 - protein, 15 aa, synthetic construct
  VARIANT 1..15: note = This sequence may encompass 6-15 residues
GGGGGGGGGGGGGGG

SEQ ID NO: 411 - protein, 8 aa, synthetic construct
  VARIANT 1..8: note = This sequence may encompass 7-8 residues
GGGGGGGG

SEQ ID NO: 412 - protein, 9 aa, synthetic construct
  VARIANT 1..9: note = This sequence may encompass 7-9 residues
GGGGGGGGG

SEQ ID NO: 413 - protein, 10 aa, synthetic construct
  VARIANT 1..10: note = This sequence may encompass 7-10 residues
GGGGGGGGGG

SEQ ID NO: 414 - protein, 12 aa, synthetic construct
  VARIANT 1..12: note = This sequence may encompass 7-12 residues
GGGGGGGGGGGG

SEQ ID NO: 415 - protein, 15 aa, synthetic construct
  VARIANT 1..15: note = This sequence may encompass 7-15 residues
GGGGGGGGGGGGGGG

SEQ ID NO: 416 - protein, 9 aa, synthetic construct
  VARIANT 1..9: note = This sequence may encompass 8-9 residues
GGGGGGGGG

SEQ ID NO: 417 - protein, 10 aa, synthetic construct
  VARIANT 1..10: note = This sequence may encompass 8-10 residues
GGGGGGGGGG

SEQ ID NO: 418 - protein, 12 aa, synthetic construct
  VARIANT 1..12: note = This sequence may encompass 8-12 residues
GGGGGGGGGGGG

SEQ ID NO: 419 - protein, 15 aa, synthetic construct
  VARIANT 1..15: note = This sequence may encompass 8-15 residues
GGGGGGGGGGGGGGG

SEQ ID NO: 420 - protein, 10 aa, synthetic construct
  VARIANT 1..10: note = This sequence may encompass 9-10 residues
GGGGGGGGGG

SEQ ID NO: 421 - protein, 12 aa, synthetic construct
  VARIANT 1..12: note = This sequence may encompass 9-12 residues
GGGGGGGGGGGG

SEQ ID NO: 422 - protein, 15 aa, synthetic construct
  VARIANT 1..15: note = This sequence may encompass 9-15 residues
GGGGGGGGGGGGGGG

SEQ ID NO: 423 - protein, 12 aa, synthetic construct
  VARIANT 1..12: note = This sequence may encompass 10-12 residues
GGGGGGGGGGGG

SEQ ID NO: 424 - protein, 15 aa, synthetic construct
  VARIANT 1..15: note = This sequence may encompass 10-15 residues
GGGGGGGGGGGGGGG

SEQ ID NO: 425 - protein, 15 aa, synthetic construct
  VARIANT 1..15: note = This sequence may encompass 12-15 residues
GGGGGGGGGGGGGGG

SEQ ID NO: 426 - protein, 15 aa, synthetic construct
  VARIANT 1..15: note = This sequence may encompass 1-10, 12, or 15 residues
GGGGGGGGGGGGGGG

SEQ ID NO: 427 - protein, 12 aa, synthetic construct
  VARIANT 1..12: note = This sequence may encompass 1-10 or 12 residues
GGGGGGGGGGGG

SEQ ID NO: 428 - protein, 15 aa, synthetic construct
  VARIANT 1..15: note = This sequence may encompass 2-10, 12, or 15 residues
GGGGGGGGGGGGGGG